SEQUENCE LISTING (94 sequences)

SEQ ID NO: 1 - DNA, 2169 bp, Homo sapiens
gaggttcagctggtggagtctggcggtggcctggtgcagccagggggctcactccgtttgtcctgtgcagcttctggcttcaacatcggttcttcttctatccactgggtgcgtcaggccccgggtaagggcctggaatgggttgcatctatttattctgcttttgcctctacttcttatgccgatagcgtcaagggccgtttcactataagcgcagacacatccaaaaacacagcctacctacaaatgaacagcttaagagctgaggacactgccgtctattattgtgctcgctaccatttcccgttcggttttgctttggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtgtttacctgttcacgttcggacagggtaccaaggtggagatcaaactcgaggacaaaactcacacaaaagtggagcccaaaacttctgataagacccatacttgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgcgaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccaatggtgtttgacctgcccccatcccgggaggagatgaccaagaaccaggtcagcctgtggtgcatggtcaagggcttctatcccagcgacatcgccgtggagtgggagagcaatgggcagccggagaacaactacaagaccacgcctcccgtgctggactccgacggctccttcttcctgtacagcaagctcaccgtggacaagagccgctggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttattcttctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgcgctcattacttcccgtgggctggtgctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactactggccgatcacgttcggacagggtaccaaggtggagatcaaa

SEQ ID NO: 2 - protein, 723 aa, Homo sapiens
EVQLVESGGGLVQPGGSLRLSCAASGFNIGSSSIHWVRQAPGKGLEWVASIYSAFASTSYADSVKGRFTISADTSKNTAYLQMNSLRAEDTAVYYCARYHFPFGFALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGVYLFTFGQGTKVEIKLEDKTHTKVEPKTSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKSGSETPGTSESATPESGGGEVQLVESGGGLVQPGGSLRLSCAASGFNISYSSIHWVRQAPGKGLEWVAYISSYYGYTYYADSVKGRFTISADTSKNTAYLQMNSLRAEDTAVYYCARAHYFPWAGAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYYWPITFGQGTKVEIK

SEQ ID NO: 3 - DNA, 2187 bp, Homo sapiens
gaggttcagctggtggagtctggcggtggcctggtgcagccagggggctcactccgtttgtcctgtgcagcttctggcttcaacatcggttcttcttctatccactgggtgcgtcaggccccgggtaagggcctggaatgggttgcatctatttattctgcttttgcctctacttcttatgccgatagcgtcaagggccgtttcactataagcgcagacacatccaaaaacacagcctacctacaaatgaacagcttaagagctgaggacactgccgtctattattgtgctcgctaccatttcccgttcggttttgctttggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtgtttacctgttcacgttcggacagggtaccaaggtggagatcaaactcgaggacaaaactcacacaaaagttgagcccaaatcttctgataagacccataattgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaatccgggagctgatgaccagcaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttcttattatatccactgggtgcgtcaggccccgggtaagggcctggaatgggttgcatctatttattcttcttatggctatacttcttatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactcttggggtccgttcacgttcggacagggtaccaaggtggagatcaaa

SEQ ID NO: 4 - protein, 729 aa, Homo sapiens
EVQLVESGGGLVQPGGSLRLSCAASGFNIGSSSIHWVRQAPGKGLEWVASIYSAFASTSYADSVKGRFTISADTSKNTAYLQMNSLRAEDTAVYYCARYHFPFGFALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGVYLFTFGQGTKVEIKLEDKTHTKVEPKSSDKTHNCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKSGSETPGTSESATPESGGGEVQLVESGGGLVQPGGSLRLSCAASGFNISSYYIHWVRQAPGKGLEWVASIYSSYGYTSYADSVKGRFTISADTSKNTAYLQMNSLRAEDTAVYYCARTVRGSKKPYFSGWAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYSWGPFTFGQGTKVEIK

SEQ ID NO: 5 - DNA, 2172 bp, Homo sapiens
gaggttcagctggtggagtctggcggtggcctggtgcagccagggggctcactccgtttgtcctgtgcagcttctggcttcaacatcggttcttcttctatccactgggtgcgtcaggccccgggtaagggcctggaatgggttgcatctatttattctgcttttgcctctacttcttatgccgatagcgtcaagggccgtttcactataagcgcagacacatccaaaaacacagcctacctacaaatgaacagcttaagagctgaggacactgccgtctattattgtgctcgctaccatttcccgttcggttttgctttggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtgtttacctgttcacgttcggacagggtaccaaggtggagatcaaactcgaggacaaaactcacacaaaagttgagcccaaatcttctgataagacccatacttgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttattcttctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgcgctcattacttcccgtgggctggtgctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactcttggggtccgttcacgttcggacagggtaccaaggtggagatcaaa

SEQ ID NO: 6 - protein, 724 aa, Homo sapiens
EVQLVESGGGLVQPGGSLRLSCAASGFNIGSSSIHWVRQAPGKGLEWVASIYSAFASTSYADSVKGRFTISADTSKNTAYLQMNSLRAEDTAVYYCARYHFPFGFALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGVYLFTFGQGTKVEIKLEDKTHTKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSGSETPGTSESATPESGGGEVQLVESGGGLVQPGGSLRLSCAASGFNISYSSIHWVRQAPGKGLEWVAYISSYYGYTYYADSVKGRFTISADTSKNTAYLQMNSLRAEDTAVYYCARAHYFPWAGAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYSWGPFTFGQGTKVEIK

SEQ ID NO: 7 - DNA, 2184 bp, Homo sapiens
gaggttcagctggtggagtctggcggtggcctggtgcagccagggggctcactccgtttgtcctgtgcagcttctggcttcaacatcggttcttcttctatccactgggtgcgtcaggccccgggtaagggcctggaatgggttgcatctatttattctgcttttgcctctacttcttatgccgatagcgtcaagggccgtttcactataagcgcagacacatccaaaaacacagcctacctacaaatgaacagcttaagagctgaggacactgccgtctattattgtgctcgctaccatttcccgttcggttttgctttggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtgtttacctgttcacgttcggacagggtaccaaggtggagatcaaactcgaggacaaaactcacacaaaagttgagcccaaatcttctgataagacccatacttgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttcttattatatccactgggtgcgtcaggccccgggtaagggcctggaatgggttgcatctatttattcttcttatggctatacttcttatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactactggccgatcacgttcggacagggtaccaaggtggagatcaaa

SEQ ID NO: 8 - protein, 728 aa, Homo sapiens
EVQLVESGGGLVQPGGSLRLSCAASGFNIGSSSIHWVRQAPGKGLEWVASIYSAFASTSYADSVKGRFTISADTSKNTAYLQMNSLRAEDTAVYYCARYHFPFGFALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGVYLFTFGQGTKVEIKLEDKTHTKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSGSETPGTSESATPESGGGEVQLVESGGGLVQPGGSLRLSCAASGFNISSYYIHWVRQAPGKGLEWVASIYSSYGYTSYADSVKGRFTISADTSKNTAYLQMNSLRAEDTAVYYCARTVRGSKKPYFSGWAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYYWPITFGQGTKVEIK

SEQ ID NO: 9 - DNA, 2175 bp, Homo sapiens
gaggttcagctggtggagtctggcggtggcctggtgcagccagggggctcactccgtttgtcctgtgcagcttctggcttcaacatctcttattattatatgcactgggtgcgtcaggccccgggtaagggcctggaatgggttgcatctatttattcttattatggctatacttattatgccgatagcgtcaagggccgtttcactataagcgcagacacatccaaaaacacagcctacctacaaatgaacagcttaagagctgaggacactgccgtctattattgtgctcgctcttctttctcttgggctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaacatccgtggtctggtggttacctgatcacgttcggacagggtaccaaggtggagatcaaactcgaggacaaaactcacacaaaagtggagcccaaaacttctgataagacccatacttgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgcgaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccaatggtgtttgacctgcccccatcccgggaggagatgaccaagaaccaggtcagcctgtggtgcatggtcaagggcttctatcccagcgacatcgccgtggagtgggagagcaatgggcagccggagaacaactacaagaccacgcctcccgtgctggactccgacggctccttcttcctgtacagcaagctcaccgtggacaagagccgctggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttattcttctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgcgctcattacttcccgtgggctggtgctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactactggccgatcacgttcggacagggtaccaaggtggagatcaaa

SEQ ID NO: 10 - protein, 725 aa, Homo sapiens
EVQLVESGGGLVQPGGSLRLSCAASGFNISYYYMHWVRQAPGKGLEWVASIYSYYGYTYYADSVKGRFTISADTSKNTAYLQMNSLRAEDTAVYYCARSSFSWAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQHPWSGGYLITFGQGTKVEIKLEDKTHTKVEPKTSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKSGSETPGTSESATPESGGGEVQLVESGGGLVQPGGSLRLSCAASGFNISYSSIHWVRQAPGKGLEWVAYISSYYGYTYYADSVKGRFTISADTSKNTAYLQMNSLRAEDTAVYYCARAHYFPWAGAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYYWPITFGQGTKVEIK

SEQ ID NO: 11 - DNA, 2193 bp, Homo sapiens
gaggttcagctggtggagtctggcggtggcctggtgcagccagggggctcactccgtttgtcctgtgcagcttctggcttcaacatctcttattattatatgcactgggtgcgtcaggccccgggtaagggcctggaatgggttgcatctatttattcttattatggctatacttattatgccgatagcgtcaagggccgtttcactataagcgcagacacatccaaaaacacagcctacctacaaatgaacagcttaagagctgaggacactgccgtctattattgtgctcgctcttctttctcttgggctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaacatccgtggtctggtggttacctgatcacgttcggacagggtaccaaggtggagatcaaactcgaggacaaaactcacacaaaagttgagcccaaatcttctgataagacccataattgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaatccgggagctgatgaccagcaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttcttattatatccactgggtgcgtcaggccccgggtaagggcctggaatgggttgcatctatttattcttcttatggctatacttcttatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactcttggggtccgttcacgttcggacagggtaccaaggtggagatcaaa

SEQ ID NO: 12 - protein, 731 aa, Homo sapiens
EVQLVESGGGLVQPGGSLRLSCAASGFNISYYYMHWVRQAPGKGLEWVASIYSYYGYTYYADSVKGRFTISADTSKNTAYLQMNSLRAEDTAVYYCARSSFSWAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQHPWSGGYLITFGQGTKVEIKLEDKTHTKVEPKSSDKTHNCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKSGSETPGTSESATPESGGGEVQLVESGGGLVQPGGSLRLSCAASGFNISSYYIHWVRQAPGKGLEWVASIYSSYGYTSYADSVKGRFTISADTSKNTAYLQMNSLRAEDTAVYYCARTVRGSKKPYFSGWAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYSWGPFTFGQGTKVEIK

SEQ ID NO: 13 - DNA, 2169 bp, Homo sapiens
gaggttcagctggtggagtctggcggtggcctggtgcagccagggggctcactccgtttgtcctgtgcagcttctggcttcaacctctcttcttattctatgcactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgcccggctccgggtcattggggttttgactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatggtactacgctccgatcacgttcggacagggtaccaaggtggagatcaaactcgaggacaaaactcacacaaaagtggagcccaaaacttctgataagacccatacttgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgcgaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccaatggtgtttgacctgcccccatcccgggaggagatgaccaagaaccaggtcagcctgtggtgcatggtcaagggcttctatcccagcgacatcgccgtggagtgggagagcaatgggcagccggagaacaactacaagaccacgcctcccgtgctggactccgacggctccttcttcctgtacagcaagctcaccgtggacaagagccgctggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttattcttctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgcgctcattacttcccgtgggctggtgctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactactggccgatcacgttcggacagggtaccaaggtggagatcaaa

SEQ ID NO: 14 - protein, 723 aa, Homo sapiens
EVQLVESGGGLVQPGGSLRLSCAASGFNLSSYSMHWVRQAPGKGLEWVAYISSYYGYTYYADSVKGRFTISADTSKNTAYLQMNSLRAEDTAVYYCARPAPGHWGFDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQWYYAPITFGQGTKVEIKLEDKTHTKVEPKTSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKSGSETPGTSESATPESGGGEVQLVESGGGLVQPGGSLRLSCAASGFNISYSSIHWVRQAPGKGLEWVAYISSYYGYTYYADSVKGRFTISADTSKNTAYLQMNSLRAEDTAVYYCARAHYFPWAGAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYYWPITFGQGTKVEIK

SEQ ID NO: 15 - DNA, 2187 bp, Homo sapiens
gaggttcagctggtggagtctggcggtggcctggtgcagccagggggctcactccgtttgtcctgtgcagcttctggcttcaacctctcttcttattctatgcactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgcccggctccgggtcattggggttttgactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatggtactacgctccgatcacgttcggacagggtaccaaggtggagatcaaactcgaggacaaaactcacacaaaagttgagcccaaatcttctgataagacccataattgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaatccgggagctgatgaccagcaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttcttattatatccactgggtgcgtcaggccccgggtaagggcctggaatgggttgcatctatttattcttcttatggctatacttcttatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactcttggggtccgttcacgttcggacagggtaccaaggtggagatcaaa

SEQ ID NO: 16 - protein, 729 aa, Homo sapiens
EVQLVESGGGLVQPGGSLRLSCAASGFNLSSYSMHWVRQAPGKGLEWVAYISSYYGYTYYADSVKGRFTISADTSKNTAYLQMNSLRAEDTAVYYCARPAPGHWGFDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQWYYAPITFGQGTKVEIKLEDKTHTKVEPKSSDKTHNCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKSGSETPGTSESATPESGGGEVQLVESGGGLVQPGGSLRLSCAASGFNISSYYIHWVRQAPGKGLEWVASIYSSYGYTSYADSVKGRFTISADTSKNTAYLQMNSLRAEDTAVYYCARTVRGSKKPYFSGWAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYSWGPFTFGQGTKVEIK

SEQ ID NO: 17 - DNA, 2175 bp, Homo sapiens
gaggttcagctggtggagtctggcggtggcctggtgcagccagggggctcactccgtttgtcctgtgcagcttctggcttcaacatctcttattattatatgcactgggtgcgtcaggccccgggtaagggcctggaatgggttgcatctatttcttcttattatggctctacttattatgccgatagcgtcaagggccgtttcactataagcgcagacacatccaaaaacacagcctacctacaaatgaacagcttaagagctgaggacactgccgtctattattgtgctcgctcttggtgggcttgggcttttgactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaacattactctgtttacgcttctctgatcacgttcggacagggtaccaaggtggagatcaaactcgaggacaaaactcacacaaaagtggagcccaaaacttctgataagacccatacttgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgcgaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccaatggtgtttgacctgcccccatcccgggaggagatgaccaagaaccaggtcagcctgtggtgcatggtcaagggcttctatcccagcgacatcgccgtggagtgggagagcaatgggcagccggagaacaactacaagaccacgcctcccgtgctggactccgacggctccttcttcctgtacagcaagctcaccgtggacaagagccgctggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttattcttctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgcgctcattacttcccgtgggctggtgctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactactggccgatcacgttcggacagggtaccaaggtggagatcaaa

SEQ ID NO: 18 - protein, 725 aa, Homo sapiens
EVQLVESGGGLVQPGGSLRLSCAASGFNISYYYMHWVRQAPGKGLEWVASISSYYGSTYYADSVKGRFTISADTSKNTAYLQMNSLRAEDTAVYYCARSWWAWAFDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQHYSVYASLITFGQGTKVEIKLEDKTHTKVEPKTSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKSGSETPGTSESATPESGGGEVQLVESGGGLVQPGGSLRLSCAASGFNISYSSIHWVRQAPGKGLEWVAYISSYYGYTYYADSVKGRFTISADTSKNTAYLQMNSLRAEDTAVYYCARAHYFPWAGAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYYWPITFGQGTKVEIK

SEQ ID NO: 19 - DNA, 2193 bp, Homo sapiens
gaggttcagctggtggagtctggcggtggcctggtgcagccagggggctcactccgtttgtcctgtgcagcttctggcttcaacatctcttattattatatgcactgggtgcgtcaggccccgggtaagggcctggaatgggttgcatctatttcttcttattatggctctacttattatgccgatagcgtcaagggccgtttcactataagcgcagacacatccaaaaacacagcctacctacaaatgaacagcttaagagctgaggacactgccgtctattattgtgctcgctcttggtgggcttgggcttttgactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaacattactctgtttacgcttctctgatcacgttcggacagggtaccaaggtggagatcaaactcgaggacaaaactcacacaaaagttgagcccaaatcttctgataagacccataattgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaatccgggagctgatgaccagcaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttcttattatatccactgggtgcgtcaggccccgggtaagggcctggaatgggttgcatctatttattcttcttatggctatacttcttatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactcttggggtccgttcacgttcggacagggtaccaaggtggagatcaaa

SEQ ID NO: 20 - protein, 731 aa, Homo sapiens
EVQLVESGGGLVQPGGSLRLSCAASGFNISYYYMHWVRQAPGKGLEWVASISSYYGSTYYADSVKGRFTISADTSKNTAYLQMNSLRAEDTAVYYCARSWWAWAFDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQHYSVYASLITFGQGTKVEIKLEDKTHTKVEPKSSDKTHNCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKSGSETPGTSESATPESGGGEVQLVESGGGLVQPGGSLRLSCAASGFNISSYYIHWVRQAPGKGLEWVASIYSSYGYTSYADSVKGRFTISADTSKNTAYLQMNSLRAEDTAVYYCARTVRGSKKPYFSGWAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYSWGPFTFGQGTKVEIK

SEQ ID NO: 21 - DNA, 2175 bp, Homo sapiens
gaggttcagctggtggagtctggcggtggcctggtgcagccagggggctcactccgtttgtcctgtgcagcttctggcttcaacatctcttattattatatccactgggtgcgtcaggccccgggtaagggcctggaatgggttgcatctatttattcttcttctagctatacttattatgccgatagcgtcaagggccgtttcactataagcgcagacacatccaaaaacacagcctacctacaaatgaacagcttaagagctgaggacactgccgtctattattgtgctcgctcttcttacgcttgggctattgactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatctggttggtggggtgtttctctgatcacgttcggacagggtaccaaggtggagatcaaactcgaggacaaaactcacacaaaagtggagcccaaaacttctgataagacccatacttgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgcgaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccaatggtgtttgacctgcccccatcccgggaggagatgaccaagaaccaggtcagcctgtggtgcatggtcaagggcttctatcccagcgacatcgccgtggagtgggagagcaatgggcagccggagaacaactacaagaccacgcctcccgtgctggactccgacggctccttcttcctgtacagcaagctcaccgtggacaagagccgctggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttattcttctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgcgctcattacttcccgtgggctggtgctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactactggccgatcacgttcggacagggtaccaaggtggagatcaaa

SEQ ID NO: 22 - protein, 725 aa, Homo sapiens
EVQLVESGGGLVQPGGSLRLSCAASGFNISYYYIHWVRQAPGKGLEWVASIYSSSSYTYYADSVKGRFTISADTSKNTAYLQMNSLRAEDTAVYYCARSSYAWAIDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQSGWWGVSLITFGQGTKVEIKLEDKTHTKVEPKTSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKSGSETPGTSESATPESGGGEVQLVESGGGLVQPGGSLRLSCAASGFNISYSSIHWVRQAPGKGLEWVAYISSYYGYTYYADSVKGRFTISADTSKNTAYLQMNSLRAEDTAVYYCARAHYFPWAGAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYYWPITFGQGTKVEIK

SEQ ID NO: 23 - DNA, 2193 bp, Homo sapiens
gaggttcagctggtggagtctggcggtggcctggtgcagccagggggctcactccgtttgtcctgtgcagcttctggcttcaacatctcttattattatatccactgggtgcgtcaggccccgggtaagggcctggaatgggttgcatctatttattcttcttctagctatacttattatgccgatagcgtcaagggccgtttcactataagcgcagacacatccaaaaacacagcctacctacaaatgaacagcttaagagctgaggacactgccgtctattattgtgctcgctcttcttacgcttgggctattgactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatctggttggtggggtgtttctctgatcacgttcggacagggtaccaaggtggagatcaaactcgaggacaaaactcacacaaaagttgagcccaaatcttctgataagacccataattgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaatccgggagctgatgaccagcaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttcttattatatccactgggtgcgtcaggccccgggtaagggcctggaatgggttgcatctatttattcttcttatggctatacttcttatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactcttggggtccgttcacgttcggacagggtaccaaggtggagatcaaa

SEQ ID NO: 24 - protein, 731 aa, Homo sapiens
EVQLVESGGGLVQPGGSLRLSCAASGFNISYYYIHWVRQAPGKGLEWVASIYSSSSYTYYADSVKGRFTISADTSKNTAYLQMNSLRAEDTAVYYCARSSYAWAIDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQSGWWGVSLITFGQGTKVEIKLEDKTHTKVEPKSSDKTHNCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKSGSETPGTSESATPESGGGEVQLVESGGGLVQPGGSLRLSCAASGFNISSYYIHWVRQAPGKGLEWVASIYSSYGYTSYADSVKGRFTISADTSKNTAYLQMNSLRAEDTAVYYCARTVRGSKKPYFSGWAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYSWGPFTFGQGTKVEIK

SEQ ID NO: 25 - DNA, 2169 bp, Homo sapiens
gaggttcagctggtggagtctggcggtggcctggtgcagccagggggctcactccgtttgtcctgtgcagcttctggcttcaacatctcttattattatatccactgggtgcgtcaggccccgggtaagggcctggaatgggttgcatctatttatccttcttctggctatacttattatgccgatagcgtcaagggccgtttcactataagcgcagacacatccaaaaacacagcctacctacaaatgaacagcttaagagctgaggacactgccgtctattattgtgctcgctcttctttctactgggctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcttacgctgcttacctgttcacgttcggacagggtaccaaggtggagatcaaactcgaggacaaaactcacacaaaagtggagcccaaaacttctgataagacccatacttgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgcgaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccaatggtgtttgacctgcccccatcccgggaggagatgaccaagaaccaggtcagcctgtggtgcatggtcaagggcttctatcccagcgacatcgccgtggagtgggagagcaatgggcagccggagaacaactacaagaccacgcctcccgtgctggactccgacggctccttcttcctgtacagcaagctcaccgtggacaagagccgctggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttattcttctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgcgctcattacttcccgtgggctggtgctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactactggccgatcacgttcggacagggtaccaaggtggagatcaaa

SEQ ID NO: 26 - protein, 723 aa, Homo sapiens
EVQLVESGGGLVQPGGSLRLSCAASGFNISYYYIHWVRQAPGKGLEWVASIYPSSGYTYYADSVKGRFTISADTSKNTAYLQMNSLRAEDTAVYYCARSSFYWAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQSYAAYLFTFGQGTKVEIKLEDKTHTKVEPKTSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKSGSETPGTSESATPESGGGEVQLVESGGGLVQPGGSLRLSCAASGFNISYSSIHWVRQAPGKGLEWVAYISSYYGYTYYADSVKGRFTISADTSKNTAYLQMNSLRAEDTAVYYCARAHYFPWAGAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYYWPITFGQGTKVEIK

SEQ ID NO: 27 - DNA, 2187 bp, Homo sapiens
gaggttcagctggtggagtctggcggtggcctggtgcagccagggggctcactccgtttgtcctgtgcagcttctggcttcaacatctcttattattatatccactgggtgcgtcaggccccgggtaagggcctggaatgggttgcatctatttatccttcttctggctatacttattatgccgatagcgtcaagggccgtttcactataagcgcagacacatccaaaaacacagcctacctacaaatgaacagcttaagagctgaggacactgccgtctattattgtgctcgctcttctttctactgggctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcttacgctgcttacctgttcacgttcggacagggtaccaaggtggagatcaaactcgaggacaaaactcacacaaaagttgagcccaaatcttctgataagacccataattgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaatccgggagctgatgaccagcaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttcttattatatccactgggtgcgtcaggccccgggtaagggcctggaatgggttgcatctatttattcttcttatggctatacttcttatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactcttggggtccgttcacgttcggacagggtaccaaggtggagatcaaa

SEQ ID NO: 28 - protein, 729 aa, Homo sapiens
EVQLVESGGGLVQPGGSLRLSCAASGFNISYYYIHWVRQAPGKGLEWVASIYPSSGYTYYADSVKGRFTISADTSKNTAYLQMNSLRAEDTAVYYCARSSFYWAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQSYAAYLFTFGQGTKVEIKLEDKTHTKVEPKSSDKTHNCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKSGSETPGTSESATPESGGGEVQLVESGGGLVQPGGSLRLSCAASGFNISSYYIHWVRQAPGKGLEWVASIYSSYGYTSYADSVKGRFTISADTSKNTAYLQMNSLRAEDTAVYYCARTVRGSKKPYFSGWAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYSWGPFTFGQGTKVEIK

SEQ ID NO: 29 - DNA, 2163 bp, Homo sapiens
gaggttcagctggtggagtctggcggtggcctggtgcagccagggggctcactccgtttgtcctgtgcagcttctggcttcaacatctcttattattctatgcactgggtgcgtcaggccccgggtaagggcctggaatgggttgcatctatttcttcttattatagctctacttcttatgccgatagcgtcaagggccgtttcactataagcgcagacacatccaaaaacacagcctacctacaaatgaacagcttaagagctgaggacactgccgtctattattgtgctcgcttctggtacccgggtatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaacattggtcttacccgatcacgttcggacagggtaccaaggtggagatcaaactcgaggacaaaactcacacaaaagtggagcccaaaacttctgataagacccatacttgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgcgaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccaatggtgtttgacctgcccccatcccgggaggagatgaccaagaaccaggtcagcctgtggtgcatggtcaagggcttctatcccagcgacatcgccgtggagtgggagagcaatgggcagccggagaacaactacaagaccacgcctcccgtgctggactccgacggctccttcttcctgtacagcaagctcaccgtggacaagagccgctggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttattcttctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgcgctcattacttcccgtgggctggtgctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactactggccgatcacgttcggacagggtaccaaggtggagatcaaa

SEQ ID NO: 30 - protein, 721 aa, Homo sapiens
EVQLVESGGGLVQPGGSLRLSCAASGFNISYYSMHWVRQAPGKGLEWVASISSYYSSTSYADSVKGRFTISADTSKNTAYLQMNSLRAEDTAVYYCARFWYPGMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQHWSYPITFGQGTKVEIKLEDKTHTKVEPKTSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKSGSETPGTSESATPESGGGEVQLVESGGGLVQPGGSLRLSCAASGFNISYSSIHWVRQAPGKGLEWVAYISSYYGYTYYADSVKGRFTISADTSKNTAYLQMNSLRAEDTAVYYCARAHYFPWAGAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYYWPITFGQGTKVEIK

SEQ ID NO: 31 - DNA, 2181 bp, Homo sapiens
gaggttcagctggtggagtctggcggtggcctggtgcagccagggggctcactccgtttgtcctgtgcagcttctggcttcaacatctcttattattctatgcactgggtgcgtcaggccccgggtaagggcctggaatgggttgcatctatttcttcttattatagctctacttcttatgccgatagcgtcaagggccgtttcactataagcgcagacacatccaaaaacacagcctacctacaaatgaacagcttaagagctgaggacactgccgtctattattgtgctcgcttctggtacccgggtatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaacattggtcttacccgatcacgttcggacagggtaccaaggtggagatcaaactcgaggacaaaactcacacaaaagttgagcccaaatcttctgataagacccataattgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaatccgggagctgatgaccagcaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttcttattatatccactgggtgcgtcaggccccgggtaagggcctggaatgggttgcatctatttattcttcttatggctatacttcttatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactcttggggtccgttcacgttcggacagggtaccaaggtggagatcaaa

SEQ ID NO: 32 - protein, 727 aa, Homo sapiens
EVQLVESGGGLVQPGGSLRLSCAASGFNISYYSMHWVRQAPGKGLEWVASISSYYSSTSYADSVKGRFTISADTSKNTAYLQMNSLRAEDTAVYYCARFWYPGMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQHWSYPITFGQGTKVEIKLEDKTHTKVEPKSSDKTHNCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKSGSETPGTSESATPESGGGEVQLVESGGGLVQPGGSLRLSCAASGFNISSYYIHWVRQAPGKGLEWVASIYSSYGYTSYADSVKGRFTISADTSKNTAYLQMNSLRAEDTAVYYCARTVRGSKKPYFSGWAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYSWGPFTFGQGTKVEIK

SEQ ID NO: 33 - DNA, 2166 bp, Homo sapiens
gaggttcagctggtggagtctggcggtggcctggtgcagccagggggctcactccgtttgtcctgtgcagcttctggcttcaacctctcttattattatatgcactgggtgcgtcaggccccgggtaagggcctggaatgggttgcatctatttattcttattctggctatacttattatgccgatagcgtcaagggccgtttcactataagcgcagacacatccaaaaacacagcctacctacaaatgaacagcttaagagctgaggacactgccgtctattattgtgctcgctcttctttcgcttgggcttttgactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtggttggggtccgttcacgttcggacagggtaccaaggtggagatcaaactcgaggacaaaactcacacaaaagtggagcccaaaacttctgataagacccatacttgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgcgaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccaatggtgtttgacctgcccccatcccgggaggagatgaccaagaaccaggtcagcctgtggtgcatggtcaagggcttctatcccagcgacatcgccgtggagtgggagagcaatgggcagccggagaacaactacaagaccacgcctcccgtgctggactccgacggctccttcttcctgtacagcaagctcaccgtggacaagagccgctggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttattcttctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgcgctcattacttcccgtgggctggtgctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactactggccgatcacgttcggacagggtaccaaggtggagatcaaa

SEQ ID NO: 34 - protein, 722 aa, Homo sapiens
EVQLVESGGGLVQPGGSLRLSCAASGFNLSYYYMHWVRQAPGKGLEWVASIYSYSGYTYYADSVKGRFTISADTSKNTAYLQMNSLRAEDTAVYYCARSSFAWAFDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGGWGPFTFGQGTKVEIKLEDKTHTKVEPKTSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKSGSETPGTSESATPESGGGEVQLVESGGGLVQPGGSLRLSCAASGFNISYSSIHWVRQAPGKGLEWVAYISSYYGYTYYADSVKGRFTISADTSKNTAYLQMNSLRAEDTAVYYCARAHYFPWAGAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYYWPITFGQGTKVEIK

SEQ ID NO: 35 - DNA, 2184 bp, Homo sapiens
gaggttcagctggtggagtctggcggtggcctggtgcagccagggggctcactccgtttgtcctgtgcagcttctggcttcaacctctcttattattatatgcactgggtgcgtcaggccccgggtaagggcctggaatgggttgcatctatttattcttattctggctatacttattatgccgatagcgtcaagggccgtttcactataagcgcagacacatccaaaaacacagcctacctacaaatgaacagcttaagagctgaggacactgccgtctattattgtgctcgctcttctttcgcttgggcttttgactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtggttggggtccgttcacgttcggacagggtaccaaggtggagatcaaactcgaggacaaaactcacacaaaagttgagcccaaatcttctgataagacccataattgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaatccgggagctgatgaccagcaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttcttattatatccactgggtgcgtcaggccccgggtaagggcctggaatgggttgcatctatttattcttcttatggctatacttcttatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactcttggggtccgttcacgttcggacagggtaccaaggtggagatcaaa

SEQ ID NO: 36 - protein, 728 aa, Homo sapiens
EVQLVESGGGLVQPGGSLRLSCAASGFNLSYYYMHWVRQAPGKGLEWVASIYSYSGYTYYADSVKGRFTISADTSKNTAYLQMNSLRAEDTAVYYCARSSFAWAFDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGGWGPFTFGQGTKVEIKLEDKTHTKVEPKSSDKTHNCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKSGSETPGTSESATPESGGGEVQLVESGGGLVQPGGSLRLSCAASGFNISSYYIHWVRQAPGKGLEWVASIYSSYGYTSYADSVKGRFTISADTSKNTAYLQMNSLRAEDTAVYYCARTVRGSKKPYFSGWAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYSWGPFTFGQGTKVEIK

SEQ ID NO: 37 - DNA, 2157 bp, Homo sapiens
gaggttcagctggtggagtctggcggtggcctggtgcagccagggggctcactccgtttgtcctgtgcagcttctggcttcaacatctcttattcttctatccactgggtgcgtcaggccccgggtaagggcctggaatgggttgcatctatttatccttcttatagctctacttattatgccgatagcgtcaagggccgtttcactataagcgcagacacatccaaaaacacagcctacctacaaatgaacagcttaagagctgaggacactgccgtctattattgtgctcgctactacgctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagctttctactacccgatcacgttcggacagggtaccaaggtggagatcaaactcgaggacaaaactcacacaaaagtggagcccaaaacttctgataagacccatacttgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgcgaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccaatggtgtttgacctgcccccatcccgggaggagatgaccaagaaccaggtcagcctgtggtgcatggtcaagggcttctatcccagcgacatcgccgtggagtgggagagcaatgggcagccggagaacaactacaagaccacgcctcccgtgctggactccgacggctccttcttcctgtacagcaagctcaccgtggacaagagccgctggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttattcttctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgcgctcattacttcccgtgggctggtgctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactactggccgatcacgttcggacagggtaccaaggtggagatcaaa

SEQ ID NO: 38 - protein, 719 aa, Homo sapiens
EVQLVESGGGLVQPGGSLRLSCAASGFNISYSSIHWVRQAPGKGLEWVASIYPSYSSTYYADSVKGRFTISADTSKNTAYLQMNSLRAEDTAVYYCARYYAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQAFYYPITFGQGTKVEIKLEDKTHTKVEPKTSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKSGSETPGTSESATPESGGGEVQLVESGGGLVQPGGSLRLSCAASGFNISYSSIHWVRQAPGKGLEWVAYISSYYGYTYYADSVKGRFTISADTSKNTAYLQMNSLRAEDTAVYYCARAHYFPWAGAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYYWPITFGQGTKVEIK

SEQ ID NO: 39 - DNA, 2175 bp, Homo sapiens
gaggttcagctggtggagtctggcggtggcctggtgcagccagggggctcactccgtttgtcctgtgcagcttctggcttcaacatctcttattcttctatccactgggtgcgtcaggccccgggtaagggcctggaatgggttgcatctatttatccttcttatagctctacttattatgccgatagcgtcaagggccgtttcactataagcgcagacacatccaaaaacacagcctacctacaaatgaacagcttaagagctgaggacactgccgtctattattgtgctcgctactacgctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagctttctactacccgatcacgttcggacagggtaccaaggtggagatcaaactcgaggacaaaactcacacaaaagttgagcccaaatcttctgataagacccataattgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaatccgggagctgatgaccagcaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttcttattatatccactgggtgcgtcaggccccgggtaagggcctggaatgggttgcatctatttattcttcttatggctatacttcttatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactcttggggtccgttcacgttcggacagggtaccaaggtggagatcaaa

SEQ ID NO: 40 - protein, 725 aa, Homo sapiens
EVQLVESGGGLVQPGGSLRLSCAASGFNISYSSIHWVRQAPGKGLEWVASIYPSYSSTYYADSVKGRFTISADTSKNTAYLQMNSLRAEDTAVYYCARYYAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQAFYYPITFGQGTKVEIKLEDKTHTKVEPKSSDKTHNCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKSGSETPGTSESATPESGGGEVQLVESGGGLVQPGGSLRLSCAASGFNISSYYIHWVRQAPGKGLEWVASIYSSYGYTSYADSVKGRFTISADTSKNTAYLQMNSLRAEDTAVYYCARTVRGSKKPYFSGWAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYSWGPFTFGQGTKVEIK

SEQ ID NO: 41 - DNA, 2196 bp, Homo sapiens
gaggttcagctggtggagtctggcggtggcctggtgcagccagggggctcactccgtttgtcctgtgcagcttctggcttcaacatcggttcttcttctatccactgggtgcgtcaggccccgggtaagggcctggaatgggttgcatctatttattctgcttttgcctctacttcttatgccgatagcgtcaagggccgtttcactataagcgcagacacatccaaaaacacagcctacctacaaatgaacagcttaagagctgaggacactgccgtctattattgtgctcgctaccatttcccgttcggttttgctttggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtgtttacctgttcacgttcggacagggtaccaaggtggagatcaaactcgaggacaaaactcacacaaaagtggagcccaaaacttctgataagacccatacttgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgcgaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccaatggtgtttgacctgcccccatcccgggaggagatgaccaagaaccaggtcagcctgtggtgcatggtcaagggcttctatcccagcgacatcgccgtggagtgggagagcaatgggcagccggagaacaactacaagaccacgcctcccgtgctggactccgacggctccttcttcctgtacagcaagctcaccgtggacaagagccgctggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacttttcttcttcttctatacactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcggtggttctggtgtttctcattacggttctgtttactactcttggtgggctttggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagcttcttacgctccgatcacgttcggacagggtaccaaggtggagatcaaa

SEQ ID NO: 42 - protein, 732 aa, Homo sapiens
EVQLVESGGGLVQPGGSLRLSCAASGFNIGSSSIHWVRQAPGKGLEWVASIYSAFASTSYADSVKGRFTISADTSKNTAYLQMNSLRAEDTAVYYCARYHFPFGFALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGVYLFTFGQGTKVEIKLEDKTHTKVEPKTSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKSGSETPGTSESATPESGGGEVQLVESGGGLVQPGGSLRLSCAASGFNFSSSSIHWVRQAPGKGLEWVASISSSYGYTYYADSVKGRFTISADTSKNTAYLQMNSLRAEDTAVYYCARGGSGVSHYGSVYYSWWALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQASYAPITFGQGTKVEIK

SEQ ID NO: 43 - DNA, 2175 bp, Homo sapiens
gaggttcagctggtggagtctggcggtggcctggtgcagccagggggctcactccgtttgtcctgtgcagcttctggcttcaacatcggttcttcttctatccactgggtgcgtcaggccccgggtaagggcctggaatgggttgcatctatttattctgcttttgcctctacttcttatgccgatagcgtcaagggccgtttcactataagcgcagacacatccaaaaacacagcctacctacaaatgaacagcttaagagctgaggacactgccgtctattattgtgctcgctaccatttcccgttcggttttgctttggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtgtttacctgttcacgttcggacagggtaccaaggtggagatcaaactcgaggacaaaactcacacaaaagttgagcccaaatcttctgataagacccataattgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaatccgggagctgatgaccagcaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacctctcttattattatatgcactgggtgcgtcaggccccgggtaagggcctggaatgggttgcatctatttattcttcttatggctatacttattatgccgatagcgtcaagggccgtttcactataagcgcagacacatccaaaaacacagcctacctacaaatgaacagcttaagagctgaggacactgccgtctattattgtgctcgctggtctcatgtttctggtcattactctggtatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcttcttattctctgatcacgttcggacagggtaccaaggtggagatcaaa

SEQ ID NO: 44 - protein, 725 aa, Homo sapiens
EVQLVESGGGLVQPGGSLRLSCAASGFNIGSSSIHWVRQAPGKGLEWVASIYSAFASTSYADSVKGRFTISADTSKNTAYLQMNSLRAEDTAVYYCARYHFPFGFALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQGVYLFTFGQGTKVEIKLEDKTHTKVEPKSSDKTHNCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKSGSETPGTSESATPESGGGEVQLVESGGGLVQPGGSLRLSCAASGFNLSYYYMHWVRQAPGKGLEWVASIYSSYGYTYYADSVKGRFTISADTSKNTAYLQMNSLRAEDTAVYYCARWSHVSGHYSGMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQSSYSLITFGQGTKVEIK

SEQ ID NO: 45 - DNA, 360 bp, Homo sapiens
gaggttcagctggtggagtctggcggtggcctggtgcagccagggggctcactccgtttgtcctgtgcagcttctggcttcaacatcggttcttcttctatccactgggtgcgtcaggccccgggtaagggcctggaatgggttgcatctatttattctgcttttgcctctacttcttatgccgatagcgtcaagggccgtttcactataagcgcagacacatccaaaaacacagcctacctacaaatgaacagcttaagagctgaggacactgccgtctattattgtgctcgctaccatttcccgttcggttttgctttggactactggggtcaaggaaccctggtcaccgtctcctcg

SEQ ID NO: 46 - DNA, 15 bp, Homo sapiens
ggtggaggtggcagt

SEQ ID NO: 47 - DNA, 318 bp, Homo sapiens
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtgtttacctgttcacgttcggacagggtaccaaggtggagatcaaa

SEQ ID NO: 48 - DNA, 780 bp, Homo sapiens
ctcgaggacaaaactcacacaaaagtggagcccaaaacttctgataagacccatacttgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgcgaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccaatggtgtttgacctgcccccatcccgggaggagatgaccaagaaccaggtcagcctgtggtgcatggtcaagggcttctatcccagcgacatcgccgtggagtgggagagcaatgggcagccggagaacaactacaagaccacgcctcccgtgctggactccgacggctccttcttcctgtacagcaagctcaccgtggacaagagccgctggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcgga

SEQ ID NO: 49 - DNA, 363 bp, Homo sapiens
gaggttcagctggtggagtctggcggtggcctggtgcagccagggggctcactccgtttgtcctgtgcagcttctggcttcaacatctcttattcttctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgcgctcattacttcccgtgggctggtgctatggactactggggtcaaggaaccctggtcaccgtctcctcg

SEQ ID NO: 50 - DNA, 321 bp, Homo sapiens
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactactggccgatcacgttcggacagggtaccaaggtggagatcaaatga

SEQ ID NO: 51 - DNA, 780 bp, Homo sapiens
ctcgaggacaaaactcacacaaaagttgagcccaaatcttctgataagacccatacttgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaaagcggcagcgagactcccgggacctcagagtccgccacacccgaaagtggtggcgga

SEQ ID NO: 52 - DNA, 324 bp, Homo sapiens
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactcttggggtccgttcacgttcggacagggtaccaaggtggagatcaaatga

SEQ ID NO: 53 - DNA, 378 bp, Homo sapiens
gaggttcagctggtggagtctggcggtggcctggtgcagccagggggctcactccgtttgtcctgtgcagcttctggcttcaacatctcttcttattatatccactgggtgcgtcaggccccgggtaagggcctggaatgggttgcatctatttattcttcttatggctatacttcttatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcg

SEQ ID NO: 54 - DNA, 354 bp, Homo sapiens
gaggttcagctggtggagtctggcggtggcctggtgcagccagggggctcactccgtttgtcctgtgcagcttctggcttcaacatctcttattattatatgcactgggtgcgtcaggccccgggtaagggcctggaatgggttgcatctatttattcttattatggctatacttattatgccgatagcgtcaagggccgtttcactataagcgcagacacatccaaaaacacagcctacctacaaatgaacagcttaagagctgaggacactgccgtctattattgtgctcgctcttctttctcttgggctatggactactggggtcaaggaaccctggtcaccgtctcctcg

SEQ ID NO: 55 - DNA, 330 bp, Homo sapiens
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaacatccgtggtctggtggttacctgatcacgttcggacagggtaccaaggtggagatcaaa

SEQ ID NO: 56 - DNA, 780 bp, Homo sapiens
ctcgaggacaaaactcacacaaaagttgagcccaaatcttctgataagacccataattgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaatccgggagctgatgaccagcaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcgga

SEQ ID NO: 57 - DNA, 357 bp, Homo sapiens
gaggttcagctggtggagtctggcggtggcctggtgcagccagggggctcactccgtttgtcctgtgcagcttctggcttcaacctctcttcttattctatgcactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgcccggctccgggtcattggggttttgactactggggtcaaggaaccctggtcaccgtctcctcg

SEQ ID NO: 58 - DNA, 321 bp, Homo sapiens
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatggtactacgctccgatcacgttcggacagggtaccaaggtggagatcaaa

SEQ ID NO: 59 - DNA, 354 bp, Homo sapiens
gaggttcagctggtggagtctggcggtggcctggtgcagccagggggctcactccgtttgtcctgtgcagcttctggcttcaacatctcttattattatatgcactgggtgcgtcaggccccgggtaagggcctggaatgggttgcatctatttcttcttattatggctctacttattatgccgatagcgtcaagggccgtttcactataagcgcagacacatccaaaaacacagcctacctacaaatgaacagcttaagagctgaggacactgccgtctattattgtgctcgctcttggtgggcttgggcttttgactactggggtcaaggaaccctggtcaccgtctcctcg

SEQ ID NO: 60 - DNA, 330 bp, Homo sapiens
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaacattactctgtttacgcttctctgatcacgttcggacagggtaccaaggtggagatcaaa

SEQ ID NO: 61 - DNA, 354 bp, Homo sapiens
gaggttcagctggtggagtctggcggtggcctggtgcagccagggggctcactccgtttgtcctgtgcagcttctggcttcaacatctcttattattatatccactgggtgcgtcaggccccgggtaagggcctggaatgggttgcatctatttattcttcttctagctatacttattatgccgatagcgtcaagggccgtttcactataagcgcagacacatccaaaaacacagcctacctacaaatgaacagcttaagagctgaggacactgccgtctattattgtgctcgctcttcttacgcttgggctattgactactggggtcaaggaaccctggtcaccgtctcctcg

SEQ ID NO: 62 - DNA, 330 bp, Homo sapiens
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatctggttggtggggtgtttctctgatcacgttcggacagggtaccaaggtggagatcaaa

SEQ ID NO: 63 - DNA, 354 bp, Homo sapiens
gaggttcagctggtggagtctggcggtggcctggtgcagccagggggctcactccgtttgtcctgtgcagcttctggcttcaacatctcttattattatatccactgggtgcgtcaggccccgggtaagggcctggaatgggttgcatctatttatccttcttctggctatacttattatgccgatagcgtcaagggccgtttcactataagcgcagacacatccaaaaacacagcctacctacaaatgaacagcttaagagctgaggacactgccgtctattattgtgctcgctcttctttctactgggctatggactactggggtcaaggaaccctggtcaccgtctcctcg

SEQ ID NO: 64 - DNA, 324 bp, Homo sapiens
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcttacgctgcttacctgttcacgttcggacagggtaccaaggtggagatcaaa

SEQ ID NO: 65 - DNA, 351 bp, Homo sapiens
gaggttcagctggtggagtctggcggtggcctggtgcagccagggggctcactccgtttgtcctgtgcagcttctggcttcaacatctcttattattctatgcactgggtgcgtcaggccccgggtaagggcctggaatgggttgcatctatttcttcttattatagctctacttcttatgccgatagcgtcaagggccgtttcactataagcgcagacacatccaaaaacacagcctacctacaaatgaacagcttaagagctgaggacactgccgtctattattgtgctcgcttctggtacccgggtatggactactggggtcaaggaaccctggtcaccgtctcctcg

SEQ ID NO: 66 - DNA, 321 bp, Homo sapiens
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaacattggtcttacccgatcacgttcggacagggtaccaaggtggagatcaaa

SEQ ID NO: 67 - DNA, 354 bp, Homo sapiens
gaggttcagctggtggagtctggcggtggcctggtgcagccagggggctcactccgtttgtcctgtgcagcttctggcttcaacctctcttattattatatgcactgggtgcgtcaggccccgggtaagggcctggaatgggttgcatctatttattcttattctggctatacttattatgccgatagcgtcaagggccgtttcactataagcgcagacacatccaaaaacacagcctacctacaaatgaacagcttaagagctgaggacactgccgtctattattgtgctcgctcttctttcgcttgggcttttgactactggggtcaaggaaccctggtcaccgtctcctcg

SEQ ID NO: 68 - DNA, 321 bp, Homo sapiens
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggtggttggggtccgttcacgttcggacagggtaccaaggtggagatcaaa

SEQ ID NO: 69 - DNA, 345 bp, Homo sapiens
gaggttcagctggtggagtctggcggtggcctggtgcagccagggggctcactccgtttgtcctgtgcagcttctggcttcaacatctcttattcttctatccactgggtgcgtcaggccccgggtaagggcctggaatgggttgcatctatttatccttcttatagctctacttattatgccgatagcgtcaagggccgtttcactataagcgcagacacatccaaaaacacagcctacctacaaatgaacagcttaagagctgaggacactgccgtctattattgtgctcgctactacgctatggactactggggtcaaggaaccctggtcaccgtctcctcg

SEQ ID NO: 70 - DNA, 321 bp, Homo sapiens
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagctttctactacccgatcacgttcggacagggtaccaaggtggagatcaaa

SEQ ID NO: 71 - DNA, 387 bp, Homo sapiens
gaggttcagctggtggagtctggcggtggcctggtgcagccagggggctcactccgtttgtcctgtgcagcttctggcttcaacttttcttcttcttctatacactgggtgcgtcaggccccgggtaagggcctggaatgggttgcatctatttcttcttcttatggctatacttattatgccgatagcgtcaagggccgtttcactataagcgcagacacatccaaaaacacagcctacctacaaatgaacagcttaagagctgaggacactgccgtctattattgtgctcgcggtggttctggtgtttctcattacggttctgtttactactcttggtgggctttggactactggggtcaaggaaccctggtcaccgtctcctcg

SEQ ID NO: 72 - DNA, 321 bp, Homo sapiens
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaagcttcttacgctccgatcacgttcggacagggtaccaaggtggagatcaaa

SEQ ID NO: 73 - DNA, 366 bp, Homo sapiens
gaggttcagctggtggagtctggcggtggcctggtgcagccagggggctcactccgtttgtcctgtgcagcttctggcttcaacctctcttattattatatgcactgggtgcgtcaggccccgggtaagggcctggaatgggttgcatctatttattcttcttatggctatacttattatgccgatagcgtcaagggccgtttcactataagcgcagacacatccaaaaacacagcctacctacaaatgaacagcttaagagctgaggacactgccgtctattattgtgctcgctggtctcatgtttctggtcattactctggtatggactactggggtcaaggaaccctggtcaccgtctcctcg

SEQ ID NO: 74 - DNA, 321 bp, Homo sapiens
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcttcttattctctgatcacgttcggacagggtaccaaggtggagatcaaa

SEQ ID NO: 75 - protein, 16 aa, Artificial Sequence
  Description of Artificial Sequence Synthetic peptide
GTTAASGSSGGSSSGA

SEQ ID NO: 76 - DNA, 2169 bp, Homo sapiens
gaggttcagctggtggagtctggcggtggcctggtgcagccagggggctcactccgtttgtcctgtgcagcttctggcttcaacatctattattcttctatccactgggtgcgtcaggccccgggtaagggcctggaatgggttgcatctatttatccttattatggctatacttattatgccgatagcgtcaagggccgtttcactataagcgcagacacatccaaaaacacagcctacctacaaatgaacagcttaagagctgaggacactgccgtctattattgtgctcgctactaccattacggtttggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcttactggcattcttacctgatcacgttcggacagggtaccaaggtggagatcaaactcgaggacaaaactcacacaaaagtggagcccaaaacttctgataagacccatacttgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgcgaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccaatggtgtttgacctgcccccatcccgggaggagatgaccaagaaccaggtcagcctgtggtgcatggtcaagggcttctatcccagcgacatcgccgtggagtgggagagcaatgggcagccggagaacaactacaagaccacgcctcccgtgctggactccgacggctccttcttcctgtacagcaagctcaccgtggacaagagccgctggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttattcttctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgcgctcattacttcccgtgggctggtgctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactactggccgatcacgttcggacagggtaccaaggtggagatcaaa

SEQ ID NO: 77 - protein, 723 aa, Homo sapiens
EVQLVESGGGLVQPGGSLRLSCAASGFNIYYSSIHWVRQAPGKGLEWVASIYPYYGYTYYADSVKGRFTISADTSKNTAYLQMNSLRAEDTAVYYCARYYHYGLDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQSYWHSYLITFGQGTKVEIKLEDKTHTKVEPKTSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKSGSETPGTSESATPESGGGEVQLVESGGGLVQPGGSLRLSCAASGFNISYSSIHWVRQAPGKGLEWVAYISSYYGYTYYADSVKGRFTISADTSKNTAYLQMNSLRAEDTAVYYCARAHYFPWAGAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYYWPITFGQGTKVEIK

SEQ ID NO: 78 - DNA, 2187 bp, Homo sapiens
gaggttcagctggtggagtctggcggtggcctggtgcagccagggggctcactccgtttgtcctgtgcagcttctggcttcaacatctattattcttctatccactgggtgcgtcaggccccgggtaagggcctggaatgggttgcatctatttatccttattatggctatacttattatgccgatagcgtcaagggccgtttcactataagcgcagacacatccaaaaacacagcctacctacaaatgaacagcttaagagctgaggacactgccgtctattattgtgctcgctactaccattacggtttggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcttactggcattcttacctgatcacgttcggacagggtaccaaggtggagatcaaactcgaggacaaaactcacacaaaagttgagcccaaatcttctgataagacccataattgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaatccgggagctgatgaccagcaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttcttattatatccactgggtgcgtcaggccccgggtaagggcctggaatgggttgcatctatttattcttcttatggctatacttcttatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactcttggggtccgttcacgttcggacagggtaccaaggtggagatcaaa

SEQ ID NO: 79 - protein, 729 aa, Homo sapiens
EVQLVESGGGLVQPGGSLRLSCAASGFNIYYSSIHWVRQAPGKGLEWVASIYPYYGYTYYADSVKGRFTISADTSKNTAYLQMNSLRAEDTAVYYCARYYHYGLDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQSYWHSYLITFGQGTKVEIKLEDKTHTKVEPKSSDKTHNCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKSGSETPGTSESATPESGGGEVQLVESGGGLVQPGGSLRLSCAASGFNISSYYIHWVRQAPGKGLEWVASIYSSYGYTSYADSVKGRFTISADTSKNTAYLQMNSLRAEDTAVYYCARTVRGSKKPYFSGWAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYSWGPFTFGQGTKVEIK

SEQ ID NO: 80 - DNA, 2166 bp, Homo sapiens
gaggttcagctggtggagtctggcggtggcctggtgcagccagggggctcactccgtttgtcctgtgcagcttctggcttcaacatctcttcttcttctatgcactgggtgcgtcaggccccgggtaagggcctggaatgggttgcatctatttattcttattatggctctacttattatgccgatagcgtcaagggccgtttcactataagcgcagacacatccaaaaacacagcctacctacaaatgaacagcttaagagctgaggacactgccgtctattattgtgctcgctggtacggtatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaccgggttcttggtacttcccgccgatcacgttcggacagggtaccaaggtggagatcaaactcgaggacaaaactcacacaaaagtggagcccaaaacttctgataagacccatacttgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgcgaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccaatggtgtttgacctgcccccatcccgggaggagatgaccaagaaccaggtcagcctgtggtgcatggtcaagggcttctatcccagcgacatcgccgtggagtgggagagcaatgggcagccggagaacaactacaagaccacgcctcccgtgctggactccgacggctccttcttcctgtacagcaagctcaccgtggacaagagccgctggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttattcttctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgcgctcattacttcccgtgggctggtgctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactactggccgatcacgttcggacagggtaccaaggtggagatcaaa

SEQ ID NO: 81 - protein, 722 aa, Homo sapiens
EVQLVESGGGLVQPGGSLRLSCAASGFNISSSSMHWVRQAPGKGLEWVASIYSYYGSTYYADSVKGRFTISADTSKNTAYLQMNSLRAEDTAVYYCARWYGMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQPGSWYFPPITFGQGTKVEIKLEDKTHTKVEPKTSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKSGSETPGTSESATPESGGGEVQLVESGGGLVQPGGSLRLSCAASGFNISYSSIHWVRQAPGKGLEWVAYISSYYGYTYYADSVKGRFTISADTSKNTAYLQMNSLRAEDTAVYYCARAHYFPWAGAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYYWPITFGQGTKVEIK

SEQ ID NO: 82 - DNA, 2184 bp, Homo sapiens
gaggttcagctggtggagtctggcggtggcctggtgcagccagggggctcactccgtttgtcctgtgcagcttctggcttcaacatctcttcttcttctatgcactgggtgcgtcaggccccgggtaagggcctggaatgggttgcatctatttattcttattatggctctacttattatgccgatagcgtcaagggccgtttcactataagcgcagacacatccaaaaacacagcctacctacaaatgaacagcttaagagctgaggacactgccgtctattattgtgctcgctggtacggtatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaccgggttcttggtacttcccgccgatcacgttcggacagggtaccaaggtggagatcaaactcgaggacaaaactcacacaaaagttgagcccaaatcttctgataagacccataattgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaatccgggagctgatgaccagcaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttcttattatatccactgggtgcgtcaggccccgggtaagggcctggaatgggttgcatctatttattcttcttatggctatacttcttatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactcttggggtccgttcacgttcggacagggtaccaaggtggagatcaaa

SEQ ID NO: 83 - protein, 728 aa, Homo sapiens
EVQLVESGGGLVQPGGSLRLSCAASGFNISSSSMHWVRQAPGKGLEWVASIYSYYGSTYYADSVKGRFTISADTSKNTAYLQMNSLRAEDTAVYYCARWYGMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQPGSWYFPPITFGQGTKVEIKLEDKTHTKVEPKSSDKTHNCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKSGSETPGTSESATPESGGGEVQLVESGGGLVQPGGSLRLSCAASGFNISSYYIHWVRQAPGKGLEWVASIYSSYGYTSYADSVKGRFTISADTSKNTAYLQMNSLRAEDTAVYYCARTVRGSKKPYFSGWAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYSWGPFTFGQGTKVEIK

SEQ ID NO: 84 - DNA, 351 bp, Homo sapiens
gaggttcagctggtggagtctggcggtggcctggtgcagccagggggctcactccgtttgtcctgtgcagcttctggcttcaacatctattattcttctatccactgggtgcgtcaggccccgggtaagggcctggaatgggttgcatctatttatccttattatggctatacttattatgccgatagcgtcaagggccgtttcactataagcgcagacacatccaaaaacacagcctacctacaaatgaacagcttaagagctgaggacactgccgtctattattgtgctcgctactaccattacggtttggactactggggtcaaggaaccctggtcaccgtctcctcg

SEQ ID NO: 85 - DNA, 327 bp, Homo sapiens
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcttactggcattcttacctgatcacgttcggacagggtaccaaggtggagatcaaa

SEQ ID NO: 86 - DNA, 345 bp, Homo sapiens
gaggttcagctggtggagtctggcggtggcctggtgcagccagggggctcactccgtttgtcctgtgcagcttctggcttcaacatctcttcttcttctatgcactgggtgcgtcaggccccgggtaagggcctggaatgggttgcatctatttattcttattatggctctacttattatgccgatagcgtcaagggccgtttcactataagcgcagacacatccaaaaacacagcctacctacaaatgaacagcttaagagctgaggacactgccgtctattattgtgctcgctggtacggtatggactactggggtcaaggaaccctggtcaccgtctcctcg

SEQ ID NO: 87 - DNA, 330 bp, Homo sapiens
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaccgggttcttggtacttcccgccgatcacgttcggacagggtaccaaggtggagatcaaa

SEQ ID NO: 88 - DNA, 360 bp, Homo sapiens
gaggttcagctggtggagtctggcggtggcctggtgcagccagggggctcactccgtttgtcctgtgcagcttctggcttcaactcctctttttattttatgcactgggtgcgtcaggccccgggtaagggcctggaatgggttgcaactgtttatccttatcttgactatacttattatgccgatagcgtcaagggccgtttcactataagcgcagacacatccaaaaacacagcctacctacaaatgaacagcttaagagctgaggacactgccgtctattattgtgctcgcgcgtttccgggttcttaccatcctatggactactggggtcaaggaaccctggtcaccgtctcctcg

SEQ ID NO: 89 - DNA, 2175 bp, Homo sapiens
gaggttcagctggtggagtctggcggtggcctggtgcagccagggggctcactccgtttgtcctgtgcagcttctggcttcaactcctctttttattttatgcactgggtgcgtcaggccccgggtaagggcctggaatgggttgcaactgtttatccttatcttgactatacttattatgccgatagcgtcaagggccgtttcactataagcgcagacacatccaaaaacacagcctacctacaaatgaacagcttaagagctgaggacactgccgtctattattgtgctcgcgcgtttccgggttcttaccatcctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcttcttattctctgatcacgttcggacagggtaccaaggtggagatcaaactcgaggacaaaactcacacaaaagtggagcccaaaacttctgataagacccatacttgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgcgaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccaatggtgtttgacctgcccccatcccgggaggagatgaccaagaaccaggtcagcctgtggtgcatggtcaagggcttctatcccagcgacatcgccgtggagtgggagagcaatgggcagccggagaacaactacaagaccacgcctcccgtgctggactccgacggctccttcttcctgtacagcaagctcaccgtggacaagagccgctggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttattcttctatccactgggtgcgtcaggccccgggtaagggcctggaatgggttgcatatatttcttcttattatggctatacttattatgccgatagcgtcaagggccgtttcactataagcgcagacacatccaaaaacacagcctacctacaaatgaacagcttaagagctgaggacactgccgtctattattgtgctcgcgctcattacttcccgtgggctggtgctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactactggccgatcacgttcggacagggtaccaaggtggagatcaaatga

SEQ ID NO: 90 - DNA, 360 bp, Homo sapiens; identical to SEQ ID NO: 88

SEQ ID NO: 91 - DNA, 321 bp, Homo sapiens; identical to SEQ ID NO: 74

SEQ ID NO: 92 - DNA, 2193 bp, Homo sapiens
gaggttcagctggtggagtctggcggtggcctggtgcagccagggggctcactccgtttgtcctgtgcagcttctggcttcaactcctctttttattttatgcactgggtgcgtcaggccccgggtaagggcctggaatgggttgcaactgtttatccttatcttgactatacttattatgccgatagcgtcaagggccgtttcactataagcgcagacacatccaaaaacacagcctacctacaaatgaacagcttaagagctgaggacactgccgtctattattgtgctcgcgcgtttccgggttcttaccatcctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcttcttattctctgatcacgttcggacagggtaccaaggtggagatcaaactcgaggacaaaactcacacaaaagttgagcccaaatcttctgataagacccataattgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccaatccgggagctgatgaccagcaaccaggtcagcctgagctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaaagcggcagcgagactcccgggacctcagagtccgccacacccgaaagtggtggcggagaggttcagctggtggagtctggcggtggcctggtgcagccagggggctcactccgtttgtcctgtgcagcttctggcttcaacatctcttcttattatatccactgggtgcgtcaggccccgggtaagggcctggaatgggttgcatctatttattcttcttatggctatacttcttatgccgatagcgtcaagggccgtttcactataagcgcagacacatccaaaaacacagcctacctacaaatgaacagcttaagagctgaggacactgccgtctattattgtgctcgcactgttcgtggatccaaaaaaccgtacttctctggttgggctatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatactcttggggtccgttcacgttcggacagggtaccaaggtggagatcaaatga

SEQ ID NO: 93 - protein, 730 aa, Homo sapiens
EVQLVESGGGLVQPGGSLRLSCAASGFNSSFYFMHWVRQAPGKGLEWVATVYPYLDYTYYADSVKGRFTISADTSKNTAYLQMNSLRAEDTAVYYCARAFPGSYHPMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQSSYSLITFGQGTKVEIKLEDKTHTKVEPKSSDKTHNCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKSGSETPGTSESATPESGGGEVQLVESGGGLVQPGGSLRLSCAASGFNISSYYIHWVRQAPGKGLEWVASIYSSYGYTSYADSVKGRFTISADTSKNTAYLQMNSLRAEDTAVYYCARTVRGSKKPYFSGWAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYSWGPFTFGQGTKVEIK

SEQ ID NO: 94 - protein, 724 aa, Homo sapiens
EVQLVESGGGLVQPGGSLRLSCAASGFNSSFYFMHWVRQAPGKGLEWVATVYPYLDYTYYADSVKGRFTISADTSKNTAYLQMNSLRAEDTAVYYCARAFPGSYHPMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQSSYSLITFGQGTKVEIKLEDKTHTKVEPKTSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKSGSETPGTSESATPESGGGEVQLVESGGGLVQPGGSLRLSCAASGFNISYSSIHWVRQAPGKGLEWVAYISSYYGYTYYADSVKGRFTISADTSKNTAYLQMNSLRAEDTAVYYCARAHYFPWAGAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYYWPITFGQGTKVEIK

(19) United States
(12) Patent Application Publication
(10) Pub. No.: US 2022/0315659 A1
(43) Pub. Date: Oct. 6, 2022

(54) Multivalent FZD and WNT Binding Molecules and Uses Thereof

(71) Applicant: AntlerA Therapeutics Inc,, Toronto, Ontario (CA)

(72) Inventors: Stephane ANGERS, Mississauga (CA); Sachdev SIDHU, Toronto (CA); Yuyong TAO, Hefei (CN)

(21) Appl. No.: 17/596,485

(22) PCT Filed: Jun. 10, 2020
(86) PCT No.: PCT/IB2020/055463

§ 371 Date: Dec. 10, 2021

Related U.S. Application Data
(60) Provisional application No. 62/860,161, filed on Jun. 11, 2019.

(51) Int. Cl. C07K 16/28

(52) U.S. Cl. CPC C07K 16/2863; C07K 2317/21; C07K 2317/31; C07K 2317/35; C07K 2317/526; C07K 2317/55; C07K 2317/622; C07K 2317/626; C07K 2317/64; C07K 2317/92

(57) ABSTRACT

Described herein are methods to affect binding by a multivalent binding molecule to a FZD receptor and a Wnt co-receptor on a cell wherein binding by the multivalent binding molecule to both FZD receptor and co-receptor on the cell activates a Wnt signaling pathway. Also described herein are multivalent binding molecules comprising a FZD receptor binding domain and a Wnt co-receptor biding domain on either end of an Fc domain that activate a Wnt signaling pathway and methods for their use.

This Application claims the benefit under 35 U.S.C. 119(e) of U.S. Provisional application No. 62/860,161 filed Jun. 11, 2019, the entirety of which is incorporated herein by reference.

### BACKGROUND

The instant application contains a Sequence Listing which has been submitted electronically in ASCII format and is hereby incorporated by reference in its entirety. Said ASCII copy, created on Jun. 10, 2020, is named 115773_PC424WO_SL.txt and is 279,203 bytes in size.

Wnt signaling pathways are critical for embryonic development and tissue homeostasis in adults. Wnt ligands are secreted growth factors that regulate various cellular processes such as proliferation, differentiation, survival and migration. Wnt ligands are universally important for the control of tissue stem cells self-renewal and regulation of many progenitor cell populations. The hydrophobicity and sensitive tertiary structure of Wnt proteins have rendered their biochemical purification challenging and their use in vitro and in vivo impracticable.

Nineteen Wnt ligands exist in humans that interact with a network of ten Frizzled cell surface receptors (FZD) and one of several co-receptors that guide the selective engagement of different intracellular signaling branches (Wodarz, A. and Nusse, R. Annu. Rev. Cell Dev. Biol. 14, 59-88 (1998); Angers, S and Moon, R. T., transduction. Nat. Rev. Mol. Cell Biol. 10, 468-477 (2009)). FZDs have conserved structural features including seven hydrophobic transmembrane domains and a cysteine-rich ligand-binding domain. FZDs are known to function in three distinct signaling pathways, known as the Wnt planar cell polarity (PCP) pathway, the canonical Wnt/0-catenin pathway, and the Wnt/calcium pathway. Activation of Wnt signaling pathways also require the presence of Wnt co-receptors to dictate the differential engagement of intracellular signaling cascades regulating the expression of genes effecting cellular machineries underlying the cellular processes listed above. For example, Wnt ligands bind to a Frizzled receptor and a member of the low-density lipoprotein receptor-related proteins 5 and 6 (LRP5/6) co-receptor family to activate the Wnt/8-catenin pathway, or with a receptor tyrosine kinase-like orphan receptors 1 and 2 (ROR1/2), related to receptor tyrosine kinase (RYK) or protein tyrosine kinase 7 (PTK7) co-receptor to initiate the Wnt/PCP pathway or alternate βcatenin-independent signaling pathways. The Wnt/β-catenin pathway, sometimes referred to as the canonical pathway, culminates in the post-translational accumulation of the transcriptional effector β-catenin that interacts with T-cell factor/lymphoid enhancer factor (LEF/TCF) family of transcription factors to regulate the expression of context-specific genes.

### SUMMARY OF THE INVENTION

Wnts require lipidation for function (Janda et al., Science. 337, 59-64 (2012); Kadowaki et al, Genes Dev. 10, 3116-3128 (1996)) and their hydrophobic nature complicates biochemical manipulation; consequently, only a few Wnts have been purified (Willert et al., Nature 423, 448-452 (2003). Furthermore, Wnts are inherently cross-reactive for multiple receptors, especially when overexpressed or applied at high dose (He et al. Science. 275, 1652-1654 (1997); Andres et al. Systematic mapping of Wnt-Frizzled interactions reveals functional selectivity by distinct Wnt-Frizzled pairs. Journal of Biological (2015) (available at http://www.jbc.org/content/early/2015/01/20/jbc.M114. 12648.short); Holmen et al., J. Biol. Chem. 277, 34727-34735 (2002)). As a result, it has been impossible to activate Frizzled receptor complexes selectively to determine the specific functions of each in different contexts or to evaluate their therapeutic potential for degenerative conditions. The multivalent binding molecules and methods described herein activate preselected Frizzled receptor-coreceptor complexes selectively. Administration of the multivalent binding molecules described herein are contemplated to treat degenerative conditions by activating the appropriate Frizzled co-receptor complexes.

Described herein are methods to affect binding by a peptide to a FZD receptor and a Wnt co-receptor on a cell wherein binding by the peptide to both FZD receptor and the co-receptor activates a Wnt signaling pathway.

Also described herein are multivalent binding molecules that activate a Wnt signaling pathway and methods for their use. The multivalent binding molecules bind to both an FZD receptor and a Wnt co-receptor thereby activating a Wnt signaling pathway. The multivalent binding molecules of this invention are also referred herein as “FZD agonists” or “FZDag”. In a particular embodiment wherein the molecules of this invention bind a FZD and a LRP5/6 the molecules maybe referred to as “Frizzled and LRP5/6 Agonists” or “FLAgs”. The multivalent binding molecules comprise an Fc domain, or fragment thereof comprising the CH3 domain, and a first binding domain that binds a FZD receptor and a second binding domain that binds a Wnt co-receptor wherein the FZD binding domain is linked to one terminus of the Fc domain and the co-receptor binding domain is linked to the other terminus of the Fe domain. Thus, the binding domain for the FZD receptor and the binding domain for the co-receptor are not directly linked rather they are separated by the Fc domain, or fragment thereof comprising the CH3 domain. This configuration of binding domains produces an unexpectedly high level of Wnt signaling pathway activation. The FZD binding domain may be monovalent, having a single binding site (paratope) for a FZD receptor, or may be multivalent having more than one binding site for a FZD receptor, e.g., the binding domain may be bivalent, trivalent or tetravalent. The Wnt co-receptor binding domain may be monovalent, having a single binding site (paratope) for a Wnt co-receptor, or may be multivalent having more than one binding site for a Wnt co-receptor, e.g., the binding domain may be bivalent, trivalent or tetravalent.

The methods described herein for producing the multivalent binding molecules enable selective and robust activation of any FZD receptor complex in vitro and in vivo. Leveraging a panel of hundreds of synthetic antibodies targeting FZDs and their co-receptors, we generated multivalent binding molecules for selective and rational activation of one, two or multiple FZD receptors. The multivalent binding molecules of this invention are highly stable, amenable to large-scale production and facile purification, have predictable pharmacokinetics, and are contemplated to exhibit low immunogenicity.

In an embodiment of this invention the binding domains of the multivalent binding molecules described herein bind to one or more FZD receptors and an LRP, e.g., LRP 5 and/or LRP6 and are alternatively referred to herein as FLAgs. FLAgs that target particular FZDs and their LRP co-receptors will improve directed differentiation and cell therapy, sustain tissue organoid growth, and mobilize endogenous stem cells in vivo and promote tissue repair after injury and restore function following tissue degeneration.

The Fc domain of the FZD agonists may be an Fc domain of an immunoglobulin. The immunoglobulin may be an IgG, e.g., an IgG₁. In an embodiment of this invention the multivalent binding molecule is a peptide dimer wherein the peptides are dimerized via the intrinsic ability of Fc domain to dimerize or via a knob-in-holes configuration within the Fc which allows for specific assembly of two different peptides to produce multivalent binding domains. Methods for dimerizing peptides via a knob-in-hole configuration are described in WO2018/026942, inventors Van Dyk et al. incorporated herein by reference,

One or both of the multivalent binding domains of the FZD agonists described herein may be bivalent and monospecific, having two binding sites for the same epitope of their respective receptor or co-receptor targets. One or both of binding domains may be bivalent and bispecific having two binding sites with each site binding a different epitope on their respective targets.

In an embodiment of this invention the FZD binding domain may comprise two single chain variable fragments (scFv) for binding to the same or different epitopes on the FZD receptor. In other embodiments of this invention the FZD binding domain comprises one or more heavy-chain variable domain (VH) fragments and/or one or more light-chain variable domain (VL) fragments that bind the FZD. In other embodiments of this invention the FZD binding domain consists of one or more single-domain antibody fragments that bind to FZD. In other embodiments of this invention the FZD binding domain comprises a FZD ligand or fragment thereof that binds the FZD receptor. In an embodiment of this invention the FZD binding domain comprises a synthetic peptide that binds the FZD, e.g., an affibody, an ankyrin repeat protein, a fibronectin repeat protein, a fynomer, or an anticalin. In an embodiment of this invention the FZD multivalent binding domain does not comprise scFv. The FZD ligand may be, e.g., a fragment of Wnt protein or of Norrin that binds the FZD receptor, or another natural or synthetic peptide that is affinity matured to interact with one or more FZD receptors. Norrin is a FZD4-specific ligand that, in complex with LRP5 and/or LRP6, is associated with activation of canonical Wnt signaling.

In an embodiment of this invention, the co-receptor binding domain may comprise two single chain variable fragments (scFv) for binding to the same or different epitopes on the co-receptor. In other embodiments of this invention the Wnt co-receptor binding domain comprises one or more heavy-chain variable domain (VH) fragments and/or one or more light-chain variable domain (VL) fragments that bind the Wnt co-receptor. In other embodiments of this invention the co-receptor binding domain consists of one or more single-domain antibody fragments that bind to the co-receptor. In an embodiment of this invention the Wnt co-receptor binding domain comprises a peptide that binds the Wnt co-receptor wherein the peptide is a fragment of a naturally occurring ligand that binds the Wnt co-receptor or is a synthetic peptide that binds the Wnt co-receptor, e.g., an affibody, an ankyrin repeat protein, a fibronectin repeat protein, a fynomer, or an anticalin. In another embodiment of this invention the co-receptor binding domain comprises a co-receptor ligand or fragment thereof that binds the co-receptor (for example the ligand Dkk1 for the co-receptor LRP5/6) or another natural or synthetic peptide affinity matured to interact with one or more co-receptors.

In an embodiment of this invention the co-receptor multivalent binding domains do not comprise scFv.

In an embodiment of this invention each binding domain of the molecules described herein may be formed by two peptides each peptide comprising a heavy-chain variable domain (VH) linked to a light-chain variable domain (VL) wherein the VH and the VL from one peptide pair with the VL and VH of the other peptide forming a diabody. In this configuration, the binding domain has two binding sites that bind to its target, i.e., the FZD binding domain has two binding sites for the FZD receptor and the co-receptor binding domain has two binding sites for the co-receptor. Using a knobs-in-holes Fc configuration, the peptides comprising the VH and VL can be engineered such that they are non-identical but still pair to form a bispecific binding domain capable of binding to two different sites on the FZD receptor or co-receptor (see FIG. 3A).

In an embodiment of this invention one or both of the multivalent binding domains comprise two peptides forming a diabody on each terminus of the Fc domain. Each diabody has two binding sites for an epitope on their respective FZD receptor or co-receptor targets. The diabody may be monospecific wherein the binding sites bind the same epitope on the FZD receptor or co-receptor, or the diabody may be bispecific binding to two different epitopes on the FZD receptor or co-receptor.

The peptides forming the scFv or diabodies may be derived from an antibody that binds to a FZD receptor or from an antibody that binds to a Wnt co-receptor. For the FZD binding domain, the antibody may be an antibody that binds to one or more FZD receptors and antagonizes Wnt signaling or inhibits Wnt binding to given FZD receptor(s), or the antibody may be an antibody that binds to one or more FZD receptors without inhibiting Wnt binding to the FZD receptor. For the co-receptor binding domain, the antibody may be an antibody that binds to the co-receptor and antagonizes Wnt signaling or inhibits Wnt binding to the co-receptor or the antibody may be an antibody that binds to a co-receptor without inhibiting Wnt binding to the co-receptor.

The FZD binding domain may bind to one or more members of the FZD receptor family, e.g., Frizzled Class Receptor 1 (FZD1), Frizzled Class Receptor 2(FZD2), Frizzled Class Receptor 3 (FZD3), Frizzled Class Receptor 4 (FZD4), Frizzled Class Receptor 5 (FZD5), Frizzled Class Receptor 6 (FZD6), Frizzled Class Receptor & (FZD7), Frizzled Class Receptor 1 Frizzled Class Receptor 8 (FZD8), Frizzled Class Receptor 9 (FZD9), or Frizzled Class Receptor 10 (FZD10). The co-receptor binding domain may bind to any Wnt co-receptor, e.g., LRP5/6, PTK7, ROR1/2, RYK, GPR124, TSPAN12, or CD133. In an embodiment of this invention the co-receptor binding domain binds to LRP5 and/or LRP6. In an embodiment of this invention the co-receptor binding domain binds to a single epitope on a co-receptor, e.g., an epitope of the LRP protein that binds Wnt1 or Wnt3a. In an embodiment of this invention the co-receptor binding domain binds to two epitopes on a co-receptor, e.g., an epitope on an LRP that binds to Wnt1 and an epitope that binds to Wnt3a.

An embodiment of this invention includes methods for producing induced pluripotent stem (iPS) cells comprising culturing a somatic cell under conditions suitable for reprogramming the somatic cells in the presence of an effective amount of a multivalent binding molecule described herein. The multivalent binding molecule may be included in an amount to accelerate the generation of iPS cells as compared to the generation of iPS cells in the same culture conditions without the multivalent binding molecule.

Also an embodiment of this invention are methods for directing differentiation of iPS or other pluripotent stem cells (PSCs) towards various lineages by culturing these cells in the presence of an effective amount of a multivalent binding molecule described herein.

An embodiment of this invention includes methods for generating tissue organoids comprising culturing a tissue sample under conditions suitable for the generation of organoids in the presence of an effective amount of a multivalent binding molecule described herein as part of the culture cocktail. In an embodiment or this invention, the frequency of generating tissue organoids cultured in a medium comprising the multivalent binding molecule is enhanced as compared to organoids cultured in the same medium without the multivalent binding molecule. In an embodiment or this invention, the tissue organoids are generated more rapidly when cultured in a medium comprising the multivalent binding molecules as compared to tissue samples cultured in the same medium without the multivalent binding molecules.

An embodiment of this invention includes methods for enhancing the maintenance of tissue organoids comprising culturing an organoid in the presence of an effective amount of a multivalent binding molecule described herein as part of the culture cocktail. As described herein, the survival of tissue organoids cultured in a medium comprising the multivalent binding molecules is prolonged as compared to organoids cultured in the same medium without the multivalent binding molecule.

An aspect of this invention is a method for making the multivalent binding molecules described herein. In an embodiment of this invention the multivalent binding molecule is generated by,
a) selecting an Fe domain having a C-terminus and an N-terminus,
b) identifying an antibody that binds to one or more FZD receptors and
c) identifying an antibody that binds to one or more Wnt co-receptors,
d) generating a nucleic acid molecule comprising a nucleotide sequence that encodes (i) the Fc domain of step a, (ii) a nucleotide sequence that encodes a VL and/or a VH of the antibody of step b, or a VL and/or a VH derived from the antibody of step b that binds the one or more FZD, and (iii) a nucleotide sequence that encodes a VL and a VH of the antibody of step c, or a VL and a VH derived from the antibody of step c that binds to the one or more Wnt receptors of step c,
e) expressing the nucleic acid molecule of (d) to produce a polypeptide wherein the polypeptide dimerizes to form a multivalent binding molecule comprising an Fc domain, a FZD binding domain and a Wnt co-receptor binding domain wherein, the FZD binding domain comprises of the VL and VH of the antibody of step b or derived from the antibody of step b and is linked to one terminus of the Fc domain, and the Wnt co-receptor binding domain comprises the VL and VH of the antibody of step c or derived from the antibody of step c and is linked to the other terminus of the Fc domain thereby forming the multi-specific binding molecule.
   The antibody in step (b) may be an antibody or antibody fragment that binds to one or more FZD receptors and antagonizes Wnt signaling or inhibits Wnt binding to the receptor. The antibody in step (b) may be an antibody or antibody fragment that binds to one or more FZD receptors without antagonizing Wnt signaling or inhibiting Wnt binding to the receptor. The antibody in step (c) may be an antibody or antibody fragment that binds to one or more of the Wnt co-receptors and antagonizes Wnt signaling or inhibits Wnt binding to the co-receptor, or binds to the co-receptor without antagonizing Wnt signaling or inhibiting Wnt binding to the co-receptor. The binding domains may be linked to the Fc domain via a linker. The modular aspects of this invention allows for mixing and matching binding domains of antibodies for any given FZD receptor and co-receptor on the termini of the Fc domain to generate a multivalent binding molecule that can engage multiple Frizzled receptor—co-receptor complexes or to selectively engage a single Frizzled receptor-co-receptor complex to activate Wnt signaling.

The multivalent binding molecule comprises a peptide dimer configured to have an Fc domain and a binding domain that binds one or more FZD receptors and a second binding domain that binds one or more Wnt co-receptors wherein the FZD binding domain is linked to one terminus of the Fc and the co-receptor binding domain is linked to the other terminus of the Fc. Each binding domain may be monovalent or multivalent, e.g. bivalent, trivalent or tetravalent.

Also an embodiment of this invention are methods using the multivalent binding molecules, e.g., for producing induced pluripotent stem (iPS) cells, for directed differentiation of pluripotent stem cells, and for generating and/or maintaining tissue organoids, or to enhance tissue regeneration in a subject in need thereof.

Additional embodiments of this invention are methods for activating Wnt signaling pathways for the mobilization of endogenous stem/progenitor cell pools for regenerative medicine and for disorders or diseases associated with insufficient Wnt signaling.

### BRIEF DESCRIPTION OF THE FIGURES

FIG. 1A depicts the binding specificity of five antibodies selected for their binding to the extracellular domain (ECD) of human LRP6. LRP6-binding antibodies were selected from a synthetic antibody library by selecting for antibodies that bound the recombinant extracellular domain (ECD) of human LRP6. The antibodies were assayed by ELISA for binding to human LRP6, mouse LRP6, and mouse LRP5. Binding to an Fc peptide and bovine serum albumin (BSA) were included as negative controls.

FIG. 1B depicts the results of a luciferase reporter assay monitoring Wnt signaling activation demonstrating that IgG 2539 and IgG 2542 (100 μM) bind different sites on LRP6 ECD by their opposite effects on Wnt1 (Transient transfection) and Wnt3a (0.5 μg/ml purified protein) stimulation. Anti-MBP antibody acts as control.

FIG. 2A depicts a representative bispecific IgGs (Bi-IgG) and bispecific diabody (bi-diabody) comprising of a FZD binding domain (5019) and an LRP6-W1 (2942, L6¹) or -W3 (2539, L6³) binding domains on the same end of the Fc domain.

FIG. 2B demonstrates that the bispecific IgGs (5019-2539 Bi-IgG and 5019-2542 Bi-IgG) do not activate Wnt signaling but rather act as antagonists of Wnt signaling, as determined in a TOPFlash luciferase reporter assay in HEK293 cells.

FIG. 2C-2G depict the binding of bispecific diabodies wherein the Fc domains are in a knob/hole configuration (K/H). Two resultant diabodies 5019-2539-K/H (FZD/LRP6-W3) and 5019-2542-K/H (FZD/LRP6-W1) retain the FZD binding profile of the original IgG as well as the LRP6 binding activity though very weak. FIG. 2C depicts the purified FZD-LRP6 diabodies: 5019-2539-K/H and 5019-2542-K/H. FIG. 2D depicts the FZD receptor binding profile of the 5019-diabody to FZD4, FZD5, and FZD7. The 5019 FZD IgG was previously characterized to bind to FZD1, 2, 4, 5, 7, 8. FIG. 2E depicts the FZD receptor binding profile of the bi-specific FZD/LRP6 diabody 5019-2539-K/H. FIG. 2F depicts the FZD receptor binding profile of the bi-specific FZD/LRP6 diabody 5019-2542-K/H. FIG. 2G demonstrates that the homo (2539-Fc and 2542-Fc) and hetero-diabodies (5019-2539-Fc and 5019-2542-Fc) having the binding domains on one terminus of the Fc domain interact with the LRP6 extra-cellular domain. FIG. 2H demonstrates co-binding of the diabodies 5019-2539-K/H and 5019-2542-K/H to FZD CRD and LRP6 ECD in solution as determined in Bio-Layer Interferometry (BLI) assays.

FIG. 2I demonstrates neither 5019-2539-K/H or 5019-2542-K/H, wherein the FZD and LRP6 receptors diabodies forming the binding domains are present on the same side of the Fc, are FZD agonists that activate a Wnt mediated pathways. The results demonstrate the 5019-2539-K/H diabody (selective for the Wnt3 site on LRP6) completely blocks the Wnt3-mediated pathway activation at 10 nM and 50 nM whereas the 5019-2542-K/H is less effective as revealed using the TOPFlash luciferase reporter assay in HEK293 cells.

FIG. 2J depicts a comparison of the luciferase activity of a tetravalent binding molecule having binding domains comprising diabodies or scFvs. The molecules having binding domains comprising anti-FZD scFvs and anti-LRP diabodies (F^{P*+P*}-L6¹⁺³) exhibited similar activity to the molecules having binding domains comprising anti-FZD diabody and anti-LRP diabodies (F^{P+P}-L6¹⁺³). In contrast, as compared to the F^{P+P}-L6¹⁺³ molecules activity was significantly reduced for the molecules that contained anti-FZD diabodies but anti-LRP6 scFvs (F^{P+P}-L6^{1*+3*}) or scFvs at both ends (F^{P*+P*}-L6^{1*+3*})

FIG. 2K and FIG. 2L demonstrate the differences in activity between a tetravalent binding molecule having binding domains comprising diabodies or scFvs were not due to differences in affinity, as BLI measurements showed comparable, high-affinity binding to LRP6 and FZD isoforms regardless of whether paratopes were presented in the diabody or scFv format.

FIG. 3A a schematic representation of a tetravalent binding molecule wherein two FZD binding domains comprised of homo (recognizing the same epitope) or hetero (recognizing separate epitopes) diabodies are linked to one end of an Fe domain and two LRP6 binding domains comprised of homo or hetero diabodies are linked to the other end of the Fc domain.

FIG. 3B depicts binding by the multivalent binding molecule 5019-Fc-2539 (F^{P+P}-L6³⁺³). and 5019-Fc-2542 (F^{P+P}-L6¹⁺¹). to FZD4, FZD5 and FZD7 ECDs. Binding to FZD receptors is detected using BLI assays.

FIG. 3C depicts activation of the Wnt-βcatenin signaling pathway by tetravalent binding molecules 5019-Fc-2539 (F^{P+P}-L6³⁺³), 5019-Fc-2542 (F^{P+P}-L6¹⁺¹), 5019-K/H-2539-2542 (F^{P+P}-L6¹⁺³), and purified Wnt3A (0.5 μg/ml). The concentration of the molecules is indicated. The tetravalent binding molecules are agonists that robustly activate the Wnt-βcatenin pathway in HEK293T cells as measured using the pBAR luciferase reporter assay. The 5019-Fc-2539 homodiabody binds to multiple FZD receptors (5019: FZD1, 2, 4, 5, 7, 8) and to the Wnt3a site on LRP6 (2539) and activates the reporter to levels comparable to purified Wnt ligands. The 5019-K/H-2539:2542 heterodiabody, which binds to both Wnt binding sites on LRP6 is more effective.

FIG. 3D depicts Wnt-βcatenin pathway activation by multivalent binding molecules having a FZD homodiabody (5019) linked through the Fc to either monospecific LRP6 homodiabody (5019-Fc-2539, 5019-Fc-2542) or bispecfic LRP6 heterodiabody (5019-K/H-2539-2542, also known as 5019Ag or F^{P+P}-L6¹⁺³)

FIG. 3E depicts the activation of Wnt-βcatenin signaling by molecules comprising a monovalent binding domain for either the FZD receptor or the LRP6 co-receptor. Wnt-βcatenin pathway activation was detected using pBAR luciferase reporter assays performed in HEK293T cells. 5019-MBP-K/H-2539-2542 contains one monovalent binding domain for FZD and still activates the Wnt pathway, but showing an 8-fold decrease in efficacy with respect to 5019Ag (which contains two FZD binding domains to the same epitope). 5019-K/H-2539-MBP, which retains only one LRP6-W3 binding domain in the C-terminus, exhibits much less efficacy. Importantly, minimal agonistic activity was detected for the two mono-FZD:mono-LRP6 diabodies 5019-MBP-K/H-2539-MBP and 5019-MBP-K/H-MBP-2542 as well as the one LRP6-W1 site diabody 5019-K/H-MBP-2542.

FIG. 3F depicts Wnt-βcatenin pathway activation by a tetravalent binding molecule in which an anti-LRP5 paratope targeting the WNT3A binding site was substituted for the anti-LRP6 paratope targeting the WNT1 binding site to generate a molecule (F^{P+P}-L5/6³) that could recruit both co-receptors and observed activity similar to that of F^{P+P}-L6¹⁺³ (EC₅₀=4 nM).

FIG. 4A depicts Wnt-βcatenin pathway activation in reporter cells without an endogenous FZD4 receptor (-FZD4) or modified to express the FZD4 receptor (+FZD4) by a multivalent binding molecules having FZD binding domains (homodiabodies in this case) specific for FZD4 on one side of the Fc domain and a co-receptor binding domain for LRP6 (2539 and 2542) on the other side of the Fc domain (FZD4Ag: 5038Ag/5038-K/H-2539-2542, 5044Ag/5044-K/H-2539-2542, 5048Ag/5048-K/H-2539-2542, 5063Ag/5063-K/H-2539-2542, 5080Ag/50180-K/H-2539-2542, 5081Ag/5081-K/H-2539-2542). Controls are the multivalent binding molecule 5019Ag (5019-K/H-2539-2542), and Norrin, an endogenous agonist of FZD4. The results demonstrate that replacing the 5019 FZD binding domain (recognizing FZD1, 2, 4, 5, 7, 8) within the 5019Ag/5019-K/H-2539:2542 (a pan-FZD agonist) with selective binding domains for FZD4, enabled the development of selective FZD4 agonists. HEK293T cells were transfected with pBARL (Wnt-βcatenin luciferase reporter) and Rluc (normalization control), plasmids coding for the listed FZD agonists and with or without FZD4 and LRP6 cDNA. Norrin was used as a positive control for activation of FZD4. HEK293T cells express low to not detectable levels of FZD4 therefore FZD4 agonists were only able to activate the reporter gene in the presence of transfected FZD4 cDNA. In contrasts, the pan-FZDag 5019-K/H-2539:2542 robustly activates Wnt-βcatenin signaling in the absence or presence of FZD4 through activation of other endogenously expressed Frizzled in these cells.

FIG. 4B depicts Wnt-βcatenin pathway activation by multivalent binding molecules having binding domains (homodiabodies) specific for FZD2 (2876, 2890), FZD2/7 (2886) FZD6 (2747), or FZD9/10 (2969, 2974) on one side of the Fc and the LRP6 heterodiabody formed by 2539 and 2542 antibody fragments on the other side of the Fc. Wnt-βcatenin pathway activation was evaluated using the pBARL assay in HEK293T cells.

FIG. 4C depicts Wnt pathway activation by multivalent binding molecules having FZD binding domains that are pan-specific for FZD and derived from IgG that block Wnt binding to FZD and Wnt-βcatenin signaling. The LRP6 binding domains in these molecules are on the c-terminus of the Fc and consist of a diabody formed by antibody 2539 and 2542, which have paratopes recognizing the Wnt3 and Wnt1 binding sites on LRP6 respectively.

FIG. 4D depicts Wnt pathway activation by multivalent binding molecules having FZD binding domains that are pan-specific for FZD and derived from IgG that do not block Wnt binding to FZD and do not antagonize Wnt3-induced pathway activation. The LRP6 binding domains in these molecules are on the c-terminus of the Fc and consists of a diabody formed by antibody 2539 and 2542, which have paratopes recognizing the Wnt3 and Wnt1 binding sites on LRP6 respectively.

FIG. 5 depicts a comparison of the FZD/LRP6 binding behavior of three tetravalent binding molecules of this invention. 5019-Fc-2539, 5019-Fc-2542, 5019-Fc-2539-2542 bind tightly to FZD but exhibit weaker LRP6 interaction (left graph) or FZD/LRP6 co-binding (middle graph). The FZD binding profile of 5019-K/H-2539-2542 (right graph) shows it recognizes FZD4, FZD5 and FZD7.

FIG. 6A is an illustration of the top two propellers (E1-E2) of LRP5/6 known to mediate binding with Wnt1, and binding of the bottom 2 propellers (E3-E4) of LRP5/6 that are proximal to the plasma membrane and known to mediate interaction with Wnt3. FIG. 6A also illustrates Wnt1 interacting with LRP5/6 and the FZD receptor and Wnt3 interacting with LRP5/6 and the FZD receptor.

FIG. 6B is an illustration of a possible interaction of the FZD receptor and LRP5/6 receptor by the multivalent binding molecules 5019-Fc-2539, 5019-Fc-2542 and 5019-K/H-2539-2542.

FIG. 6C demonstrates the multivalent binding molecules are agonists that robustly activate the Wnt-βcatenin pathway in HEK293T cells as measured using the pBAR luciferase reporter assay. 5019-Fc-2539 homodiabody binds to multiple FZD receptors (5019 binds FZD1, 2, 4, 5, 7, 8) and to the Wnt3a site on LRP6 (2539) and activates the reporter to levels comparable to purified Wnt ligands. The 5019-K/H-2539:2542 heterodiabody, which binds to both Wnt3a and Wnt1 binding sites on LRP6, is more effective.

FIG. 6D demonstrates 5019-K/H-2459:2460, a tetravalent binding molecule having an Fc domain in a knob-in-hole configuration and having a FZD binding domain (homodiabody) that is pan FZD-specific (5019) and a co-receptor binding domain that is bispecific (heterodiabody) for two sites on LRP5 (2459 binds Wnt1 binding site and 2460 binds Wnt3 binding site), also activates the Wnt-βcatenin pathway in HEK293T cells.

FIG. 7A demonstrates that by replacing the FZD binding domain within the 5019-K/H-2539:2542 (a pan-FZD agonist recognizing FZD1, 2, 4, 5, 7, 8) with a FZD binding domain specific for FZD5 (#2928), a selective FZD5 agonist was generated. HPAF-II cells have been shown to depend on FZD5 signaling for their proliferation. Blocking Wnt-FZD5 signaling using the Wnt secretion inhibitor LGK974 (targeting the acyl-transferase Porcupine) leads to cell cycle arrest and inhibition of proliferation. Proliferation can be rescued with addition of exogenous Wnt3a conditioned media or with the addition of the FZD5 selective agonists (2928-K/H-2539:2542) or pan-FZD agonist (5019-K/H-2539:2542) described herein. The FZD4 selective agonist 5038-K/H-2539:2542 only has modest rescue ability.

FIG. 7B demonstrates stimulation of C3H10T1/2 cells with a FZD2-specific FLag led to robust induction of the osteogenic marker alkaline phosphatase (ALPL) to levels similar to those achieved with a Pan-FZD FLAg, whereas a FZD5-specific FLAg exhibited minimal activity.

FIGS. 8A and 8B demonstrates that the pan-FZDag (F^{P+P}-L6¹⁺³) of this invention fully substitute for exogenous Wnt3A conditioned media to rescue the growth inhibition of intestinal organoids when Wnt secretion is blocked with LGK974, a small molecule inhibitor of Porcupine (lower left photograph). Intestinal organoids isolated from mice grow in the presence of recombinant R-Spondin and require the presence of Wnt ligands secreted by the paneth cells. FIG. 8A depicts inhibition of Wnt production using LGK974 leads to organoid death (upper right photograph). Exogenous application of Wnt3A conditioned media (lower right photograph) or FZDag (lower left photograph) rescues organoid growth in the presence of LGK974. The upper left photograph depicts organoids treated with DMSO without LGK974 as control. FIG. 8B demonstrates that inhibition of Wnt production by LGK974, leading to organoid death, can be rescued by application of Wnt3A conditioned media or FZDag (F^{P+P}-L6¹⁺³), as quantified using CellTiter Glow® Assay, Promega.

FIGS. 9A and 9B depict an example of the plasmids encoding the peptides that dimerize in a knob-into-hole conformation to form the pan-FZDag 5019-KH-2539-2542(F^{P+P}-L6¹⁺³). FIG. 9A depicts a plasmid encoding the peptide comprising an Fc region comprising a “knob” mutation, the VH and VL of panFZD antibody #5019, and the VL of LRP antibody #2542 and the VH of LRP antibody #2539. FIG. 9B depicts a plasmid encoding the peptide comprising a nucleic acid encoding Fc region comprising a “hole” mutation, the VH and VL of pan-FZD antibody #5019, and the VH of LRP antibody #2542 and the VL of LRP antibody #2539. The peptides encoded by these plasmids form a heterodimer having tetravalent binding domains comprising a homo-diabody produced by pairing of the VH and VL of the pan specific FZD antibody #5019 and a bispecific heterodiabody produced by the pairing of VL of LRP6 antibody #2539 and VH of LRP antibody #2542 from one peptide with the VH of LRP antibody #2539 and the VL of LRP antibody #2542 of the other peptide.

FIG. 9C is a schematic representation of the heterodimer knob-into-hole configuration 5019-K/H-2539:2542 (F^{P+P}-L6¹⁺³) Using a knob-in-hole configuration within the Fc it is possible to increase the modularity of the molecule up to 4 different binding sites. For this molecule (5019-K/H-2539:2542) a pan-FZD homodiabody is engineered on one side of the Fc domain and an heterodiabody containing Wnt3 (2539) and Wnt1 (2542) LRP6 binding sites on the other side of the Fc domain

FIGS. 10A and 10B is an annotation of the domains of the nucleic acid sequence of the 5019-knob-2539:2542 multivalent binding molecule (SEQ ID NO: 21 plus an additional 3′ TGA, and its complementary sequence).

FIG. 11 A-F depict the design and validation of tetravalent binding molecules that bind FZD and LRP6 Wnt1 and Wnt3 binding sites (FLAgs) as activators of the Wnt-βcatenin pathway. FIG. 11A depicts anti-FZD Fab inhibitory (top) and specific activity (bottom). FIG. 11B depicts inhibition of Wnt1 or Wnt3A signaling by the indicated LRP6 Abs in the diabody-Fc format. FIG. 11C depicts molecular architecture of tetravalent FLAgs.

FIG. 11D shows dose response curves for the activation of a LEF/TCF reporter gene (y-axis) in HEK293T cells by serial dilutions of pan-specific FLAg proteins (F^{P+P}-L6¹⁺¹, F^{P+P}-L6³⁺³ and F^{P+P}-L6¹⁺³) (x-axis). FIG. 11E depicts the levels of βcatenin protein in RKO cells after 30 min treatment with indicated concentrations of pan-FLAg (F^{P+P}-L6¹⁺³). FIG. 11F depicts the time course of βcatenin and phosphorylated Dishevelled-2 (p-Dvl2) protein levels in RKO cells treated with 10 nM pan-FLAg (F^{P+P}-L6¹⁺³)

FIG. 12A-FIG. 12D depict the characterization and dissection of the FLAG F^{P+P}-L6¹⁺³ binding and activity. FIGS. 12A and 12B depicts binding kinetics of F^{P+P}-L6¹⁺³ to nine of 10 human FZD CRDs to human LRP6 ECD. FIG. 12C demonstrates F^{P+P}-L6¹⁺³ behaved similarly to a conventional IgG and interacted with FcRn in a dose and pH dependent manner. FIG. 12D demonstrates F^{P+P}-L6¹⁺³ also behaved similarly to the IgG for interaction with other Fc effectors including complement (C1q), the natural killer cell marker CD16a, the B cell marker CD32a, and the monocyte and macrophage marker CD64.

FIGS. 13A and 13B demonstrate that treatment of with 30 nM F^{P+P}-L6¹⁺³ for three days caused robust induction of the mesoderm marker BRACHYURY and decreased expression of the pluripotency marker OCT4 to levels comparable to treatment with the GSK3 inhibitor CHIR99021 at 6 μM.

FIG. 14 displays representative fluorescence images of small intestinal sections from LGR5-GFP mice treated with vehicle, C59 or pan-FLAg(F^{P+P}-L6¹⁺³)+C59. LGR5-GFP is expressed in the stem cells at the bottom of crypts. Cell nuclei were counterstained with DAPI.

### DETAILED DESCRIPTION OF THE INVENTION

Described herein are multivalent binding molecules comprising an Fc domain, a FZD binding domain and a binding domain for a Wnt co-receptor wherein the binding domains are attached to opposite ends of the Fc domain. The multivalent binding molecules of this invention are agonists of a Wnt signaling pathway and are alternately referred to herein as FZD agonists or FZDag. Wnt ligands function by promoting the clustering of FZD receptors with co-receptors. Without wishing to be bound by theory it is contemplated that the multispecific molecules described herein simultaneously bind to a FZD receptor and a Wnt co-receptor and thereby activate Wnt signaling pathways.

The modularity and effectiveness of the multivalent binding molecules for activating Wnt signaling pathways described herein contrasts with the Wnt surrogates described in the prior art which consists of monovalent FZD and LRP5/6 binding ligands, wherein the binding ligands are not attached to opposite ends of an Fc domain. In an embodiment of this invention the FZD binding domain comprise a binding moiety that is derived from antibodies or polypeptides that bind specifically to one or more FZD receptors and the co-receptor binding domain comprises a binding moiety that binds to a co-receptor, e.g., an LRP5/6, ROR1/2, RYK or PTK7. In an embodiment of the invention the antibodies or polypeptides that specifically bind to one or more FZD receptors bind to a cysteine rich domain (CRD) of one or more of the FZD receptor.

The amino acid sequences of FZD receptors and nucleotide sequences encoding FZD receptors, and antibodies and libraries of antibodies that bind FZD or the Wnt co-receptors LRP5/6, ROR1/2, RYK or PTK7 are readily available or can be generated using methods well known in the art (see e.g., U.S. publication no. 2015/0232554, inventors Gurney et al. and US publication no. 2016/0194394, inventors Sidhu et al. and US 20190040144, inventors Pan et al; U.S. publication no. 2017/0166636, inventors Wu et al.; U.S. publication no. 2016/0208018, inventors Chen et al.; U.S. publication no. 2016/0053022, inventors Macheda et al.; U.S. publication no. 2015/031293, inventors Damelin et al.).

Methods for generating peptides or polypeptides that bind to a selected target are well known in the art, see for example Sidhu et al. Methods in Enzymology (2000) 328: 333-336. For example, a library of affibodies that bind a FZD or Wnt co-receptor may be obtained according to protocols known in the art (see, e.g., U.S. Pat. No. 5,831,012 and Lofblom et al., FEBS Letters 584 (2010) 2670-2680); a library of ankyrin repeat proteins used for the selection of a peptide that binds a FZD or Wnt co-receptor may be obtained according to protocols known in the art (see e.g., WO 02/020565, inventors Stumpp et al.) and a library of fibronectin repeat proteins used for the selection of a peptide that binds a FZD or a Wnt co-receptor may also be obtained according to protocols known in the art (see e.g., U.S. Pat. No. 9,200,273, inventors Diem and Jacobs. The peptides that bind to a FZD or a Wnt co-receptors may also be fynomers, small binding proteins derived from the human Fyn SH3 domain or artificial receptor proteins, “anticalins”, based on human apoliprotein D, and may be generated using methods known in the art, see e.g., Silacci et al., J. Biol. Chem (2014) 289(20):14392-8 and Vogt and Skerra, ChemBioChem (2004) 5, 191-199).

Antibodies suitable as the source for antigen binding peptides as described herein may be isolated by screening combinatorial libraries for polypeptides with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004). In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360. Antibodies or antibody fragments isolated from human antibody libraries are considered human antibodies or human antibody fragments herein.

Thus one of skill in the art would readily prepare an Fc domain and mix and match multivalent FZD binding domains and Wnt co-receptor binding domains having a desired specificity on the N and C terminals of the Fc domain to prepare the multivalent binding molecules to bind the desired FZD receptors and co-receptors and thereby activate specific Wnt pathways. These specific agonists would serve as powerful tools in enhancing cell proliferation, differentiation, organoid survival and maintenance, and tissue regeneration in vivo. These specific agonists also serve as powerful tools for profiling the FZD specificity involved in these processes. For example, as shown herein, the FZD5Ag but not FZD4Ag rescues the growth defect of LGK974-treated RNF43 mutant PDAC cell lines, highlighting the importance of FZD5 over FZD4 receptor in this process.

An embodiment of this invention is a method to effect binding by a peptide to a FZD receptor and a Wnt co-receptor on a cell wherein binding by said peptide to both FZD receptor and co-receptor activates a Wnt signaling pathway in cell. The method comprises selecting an Fc domain, or fragment thereof comprising a CH3 domain, having a C-terminus and an N-terminus, linking a first multivalent binding domain that binds the FZD receptor on one terminus of the Fc domain, and linking a second multivalent binding domain that binds to the Wnt co-receptor on the other terminus of the Fc domain thereby forming a multivalent binding molecule and then contacting the multivalent binding molecule with a cell expressing said FZD receptor and co-receptor under conditions to activate the Wnt signaling pathway.

In an embodiment of the invention the multivalent binding domains may comprise single chain variable fragments (ScFv) that bind to one or more FZD receptor, a ligand of the FZD receptor or co-receptor, or a fragment thereof that binds to the FZD receptor or the co-receptor. In another embodiment the binding domains do not comprise single chain variable fragments (ScFv) that bind to one or more FZD receptor, a ligand of the FZD receptor or co-receptor, or a fragment thereof that binds to the FZD receptor or the co-receptor.

In an embodiment of the invention at least one of the FZD or co-receptor multivalent binding domain comprises a diabody having two peptides each peptide having a heavy-chain variable domain (VH) linked to a light-chain variable domain (VL), wherein the VH and the VL from one peptide pairs with the VL and VH of the other peptide such that the binding domain has two epitope-binding sites. The VH and VL domains may be the VH and VL of an antibody that binds to a Wnt binding site on the FZD receptor or co-receptor. A VH or VL derived from an antibody, the source antibody, may be 50%, 55%, 60%, 75%. 80%, 85%, 90%, 95%, 96%, 97%, 98% or 99% identical to the VH and VL of the source antibody and still retain binding to the FZD receptor or co-receptor site bound by the antibody.

In an embodiment of this invention the multivalent binding molecules of this invention comprise the multivalent binding molecules of Table 1 (Table 1 comprises Tables 1A and 1B: Table 1A indicates nucleotide sequences and amino acid sequences of exemplified multivalent binding molecules of this invention; Table 1B indicates the nucleotide sequences encoding the various domains of the exemplified multivalent binding molecules). In an embodiment of this invention the multivalent binding molecules of this invention consist essentially of the multivalent binding molecules of Table 1. In an embodiment of this invention the multivalent binding molecules of this invention consist of the multivalent binding molecules of Table 1. In an embodiment of this invention the multivalent binding molecule comprises a first polypeptide comprising SEQ ID NO: 77 and a second peptide comprising SEQ ID NO: 79. In an embodiment of this invention the multivalent binding molecule comprises a first polypeptide comprising SEQ ID NO: 81, or and a second peptide comprising 83. In an embodiment of this invention the multivalent binding molecule consists essentially of a first peptide comprising SEQ ID NO: 77 and a second peptide comprising SEQ ID NO: 79 and binds to FZD2 and LRP 5/6. In an embodiment of this invention the multivalent binding molecule consists essentially of a first peptide comprising SEQ ID NO: 81 and a second peptide comprising SEQ ID NO: 83 and binds to FZD7 and LRP 5/6. In an embodiment of this invention the multivalent binding molecule consists of a first polypeptide consisting of SEQ ID NO: 77 and a second polypeptide consisting of SEQ ID NO: 79. In an embodiment of this invention the multivalent binding molecule consists of a first polypeptide consisting of SEQ ID NO: 81 and a second polypeptide consisting of SEQ ID NO:83.

In an embodiment of this invention the multivalent binding domains comprise one or more of the VL and VH domains of the molecules of Table 1. In an embodiment of this invention the multivalent binding domains of the multivalent molecules consist essentially of one or more the VL and VH domains of the molecules of Table 1. In an embodiment of this invention the multivalent binding domains of the multivalent molecules consist of one or more of the VL and VH domains of the molecules of Table 1. In an embodiment of this invention the binding domains of the multivalent molecules described herein comprise VH and VL domains that are at least 75%, 80%, 85%, 90%, 95%, 98% or 99% identical to VH and VL of the molecules set forth in Table 1 and retain binding to the antigen bound by the molecules set forth in Table 1. In an embodiment of this invention the multivalent binding domains comprise one or more of the VL and VH domains of SEQ ID NOS: 77 and 79 that bind FZD2. In an embodiment of this invention the multivalent binding domains comprise one or more of the VL and VH domains of SEQ ID NOS: 81 and 83 bind FZD7.

In an embodiment of this invention the multivalent binding domains of the multivalent molecules consist essentially of one or more of the VL and VH domains of SEQ ID NOS:77 and 79 that bind FZD2. In an embodiment of this invention the multivalent binding domains of the multivalent molecules consist essentially of one or more of the VL and VH domains of SEQ ID NOS:81 and 83 that binds FZD7.

In an embodiment of this invention the multivalent binding domains of the multivalent molecules consist of one or more of the VL and VH domains of SEQ ID NO: 77 and 79 that binds FZD2. In an embodiment of this invention the multivalent binding domains of the multivalent molecules consist of one or more of the VL and VH domains of SEQ ID NO: 81 and 83 that binds FZD7.

In an embodiment of this invention the binding domains of the multivalent molecules described herein comprise VH and VL domains that are at least 75%, 80%, 85%, 90%, 95%, 98% or 99% identical to VH and VL domains of SEQ ID NOS: 77 and 79 and retain binding of FZD2. In an embodiment of this invention the binding domains of the multivalent molecules described herein comprise VH and VL domains that are at least 75%, 80%, 85%, 90%, 95%, 98% or 99% identical to VH and VL domains of SEQ ID NOS: 81 and 83, and retain binding to FZD7.

In an embodiment of this invention the binding domains of the multivalent molecules described herein comprise one or more complementarity determining regions (CDRs) of the molecules set forth in Table 1. In an embodiment of this invention the binding domains of the multivalent molecules described herein comprise CDRs that are at least 75%, 80%, 85%, 90%, 95%, 98% or 99% identical to CDRs of the molecules set forth in Table 1 and retain binding to the antigen bound by the molecules set forth in Table 1. In an embodiment of this invention the binding domains of the multivalent molecules described herein comprise one or more complementarity determining regions (CDRs) of SEQ ID NO: 77, 79, 81, or 83. In an embodiment of this invention the binding domains of the multivalent molecules described herein comprise CDRs that are at least 75%, 80%, 85%, 90%, 95%, 98% or 99% identical to CDRs of SEQ ID NO: 77 or 79 and retain binding to FZD2 or comprise CDRs that are at least 75%, 80%, 85%, 90%, 95%, 98% or 99% identical to CDRs of SEQ ID NO: 81, or 83 and retain binding to FZD7.

The FZD receptor bound by the multivalent binding molecules of this invention may be FZD1, FZD2, FZD3, FZD4, FZD5, FZD6, FZD7, FZD8, FZD9, or FZD10. The FZD receptor may be FZD1, FZD2, FZD4, FZD5, FZD7 or FZD8. The multivalent binding molecules may bind to only one FZD receptor or may be pan-specific binding to more than one FZD receptor. The FZD multivalent binding domain may bind to e.g., FZD1, FZD2, FZD4, FZD5, FZD7 and FZD8. The FZD multivalent binding domain may specifically bind to one FZD receptor, e.g., FZD2, FZD4, FZD5, or FZD6.

In an embodiment of this invention the FZD binding domain is monospecific and binds to a single epitope on a FZD receptor. In an embodiment of this invention the FZD binding domain is bispecific and binds to two epitopes on an FZD receptor.

The co-receptor binding domain may bind to any Wnt co-receptor, e.g., LRP5/6, or ROR1/2. The multivalent co-receptor binding domain may bind to, e.g., LRP5/6, PTK7, ROR1/2, RYK, GPR12, TSPAN12 or CD133. In an embodiment of this invention the co-receptor multivalent binding domain binds to LRP5 or LRP6.

In an embodiment of this invention the co-receptor multivalent binding domain binds to a single epitope on a co-receptor, e.g., an epitope of LRP5/6 that binds Wnt1 or Wnt3. In an embodiment of this invention the co-receptor multivalent binding domain binds to two epitopes within a co-receptor, e.g., an epitope on LRP5/6 that binds to Wnt1 and an epitope that binds to Wnt3. The Wnt co-receptor bound by the multivalent binding molecules of this invention may be LRP5 or LRP6, PTK7, ROR1, ROR2, RYK, GPR124, TSPAN12 or CD133.

In an embodiment of this invention the multivalent binding molecule comprises a Fc domain, wherein the Fc domain is the Fc domain of an immunoglobulin or a fragment thereof comprising the CH3 domain. In an embodiment of the invention the immunoglobulin is an IgG. In an embodiment of this invention the IgG is an IgG₁.

An embodiment of this invention is a method for activating a Wnt signaling pathway in a cell, comprising contacting a cell having a FZD receptor and a Wnt co-receptor with a multivalent binding molecule of this invention in an amount effective to activate Wnt signaling

In an embodiment of this invention at least one of the multivalent binding domains comprises an scFv that binds the FZD receptor or co-receptor, or comprises a ligand of the FZD receptor or co-receptor or a fragment of said ligand. In an embodiment of this invention at least one of the multivalent binding domains does not comprise an scFv that binds the FZD receptor or co-receptor and does not comprise a ligand of the FZD receptor or co-receptor or a fragment of said ligand.

In an embodiment of this invention the FZD multivalent binding domains comprise a FZD diabody and the co-receptor multivalent binding domain comprises a co-receptor diabody wherein the diabodies comprises two peptides each comprising a heavy-chain variable domain (VH) linked to a light-chain variable domain (VL) wherein the binding domain is formed by pairing of the VH and the VL from one peptide to the VL and VH of the other peptide thereby forming the binding domains.

The VH and VL of the FZD binding domain may be derived from an antibody that binds the FZD receptor and antagonizes Wnt signaling or inhibits binding of a Wnt ligand to the FZD receptor. The VH and VL of the FZD binding domain may be derived from an antibody that binds the FZD receptor without antagonizing or inhibiting binding of a Wnt ligand to the FZD receptor.

The VH and VL of the co-receptor binding domain may be derived from an antibody that binds the co-receptor and antagonizes Wnt signaling or inhibits binding of a Wnt ligand to the co-receptor. The VH and VL of the co-receptor binding domain may be derived from an antibody that binds the co-receptor without antagonizing Wnt signaling or inhibiting binding of a Wnt ligand to the co-receptor.

In the multivalent binding molecules of this invention one or both of the binding domains may be bivalent and one or both of the bivalent binding domains may be bispecific for the FZD receptor or for the co-receptor. In an embodiment of this invention both binding domains are bivalent and bispecific, each binding domain binding to two different epitopes on their respective target FZD receptor or co-receptor. For example, the binding molecule may comprise a FZD binding domain that is bivalent and bispecific (binding to two different epitopes) for FZD receptors, or the binding molecule may comprise a co-receptor binding domain that is bivalent and bispecific for a co-receptor.

In an embodiment of this invention the FZD binding domain is attached to the N-terminus of the Fc domain of the multivalent binding molecule and the co-receptor binding domain is attached to the C-terminus of the Fc domain. In an embodiment of this invention the FZD binding domain is attached to the C-terminus of the Fc domain of the multivalent binding molecule and the co-receptor binding domain is attached to the N-terminus of the Fc domain.

Also an embodiment of this invention are the nucleic acid molecules encoding the multivalent biding molecules described herein, e.g. the multivalent binding molecules of Table 1, e.g. SEQ ID NO: 76 and SEQ ID NO: 78, or SEQ ID NO: 80 and SEQ ID NO: 82, their VH and VL domains (e.g., SEQ ID NO: 84, 85, 86 and 87), and diabodies comprising the VL and VH domains, including expression cassettes and vectors comprising the nucleic acid molecules that encode the multivalent binding molecules, their VH, and Fc domains, and diabodies comprising such VL and VH. The nucleic acid molecules can be inserted into a vector and expressed in an appropriate host cell and then the multivalent binding molecules isolated from the cells using methods well known in the art. As used in this invention, the term “vector” refers to a nucleic acid delivery vehicle or plasmid that can be engineered to contain a nucleic acid molecule, e.g., a nucleic acid sequence encoding the multivalent binding molecules described herein. The vector that can express protein when inserted with a polynucleotide is called an expression vector. Vectors can be inserted into the host cell by transformation, transduction, or transfection, so that the carried genetic substances can be expressed in the host cell. Vectors are well known to the technical personnel in the field, including but not limited to: plasmid; phagemid; cosmid; artificial chromosome such as yeast artificial chromosome (YAC), bacterial artificial chromosome (BAC), or P1 derived artificial chromosome (PAC); phage such as λphage or M13 phage and animal viruses etc. Animal viruses may include but not limited to, reverse transcriptase virus (including lentivirus), adenovirus, adeno-associated virus, herpes virus (e. g. herpes simplex virus), chicken pox virus, baculovirus, papilloma virus, and papova virus (such as SV40). A vector can contain multiple components that control expression of the multivalent binding molecules described herein, including but not limited to, promoters, e.g., viral or eukaryotic promoters, e.g., a CMV promoter, signal peptides, e.g., TRYP2 signal peptide, transcription initiation factor, enhancer, selection element, and reporter gene. In addition, the vector may also contain replication initiation site(s).

As used in this invention, the term “host cell” refers to cells that can import vectors, including but not limited to, prokaryotic cells such as Escherichia coli and Bacillus subtilis, fungal cells such as yeast and Aspergillus, insect cells such as S2 drosophila cells and Sf9, or animal cells, including human cells, e.g., fibroblast cells, CHO cells, COS cells, NSO cells, HeLa cells, BHK cells, or HEK293 cells.

An embodiment of this invention is a pharmaceutical composition comprising a FZD agonist described herein and a pharmaceutically acceptable excipient. The pharmaceutical composition may further comprise an additional agent that activates a Wnt pathway, e.g., a Norrin or R-Spondin. The pharmaceutical composition may consist or consist essentially of the multivalent binding molecules described herein and a pharmaceutically acceptable carrier or excipient. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of the FZD agonists being administered.

Wnt-signaling is a ubiquitous pathway that modulates cellular and tissue differentiation. For example, in regards to eye development a particular Wnt-pathway, the Norrin-FZD4 pathway, has been identified as playing a role in retinal angiogenesis. Signaling through Norrin-FZD4 pathway is necessary for development and maintenance of retinal vasculature. Mutations affecting genes of this pathway may result in several pediatric vitreoretinopathies, such as Norrie Disease, Familial Exudative Vitreoretinopathy (FEVR), and Pseudoglioma and Osteoporosis Syndrome. Additionally, Retinopathy of Prematurity (ROP) has been associated with mutations in this pathway, and Wnt-pathway mutations have been reported in Coats Disease and Persistent Fetal Vasculature (PFV). The Norrin-FZD pathway is also associated with CNS blood vessel development. Genetic ablation of the Norrin, FZD4, Lrp5 and the co-receptor Tetraspanin-12 (Tspan-12) result in defective angiogenesis and barrier disruption in both retinal and cerebellar vessels (Cho et al. (2017) Neuron 95, 1056-1073; Zhou et al., (2014) J Clin Invest 124:3825-3846). It is specifically contemplated herein that the FZD4 agonists of this invention, particularly the FZD4 FLAgs comprising a FZD4 binding domain on one end of the Fc receptor and a binding domain for LRP5 and/or LRP6 on the other side of the Fc domain will strengthen barrier function and facilitate angiogenesis, e.g., treatment with the FZD4 FLAgs will facilitate the development and maintenance of retinal vasculature and/or the blood retinal barrier (BRB) and the blood brain barrier (BBB). Thus an aspect of this invention is a method for promoting and/or maintaining retinal vasculature by treating eye tissue, e.g., retinal tissue, with an effective amount of a FZD4 FLAgs through local or systemic administration. Also an aspect of this invention is a method for promoting and/or maintaining BBB vasculature by treating the BBB with an effective amount of a FZD4 FLAgs following systemic administration. A further aspect of this invention is a method for treating a subject having a disorder characterized by reduced retinal or brain angiogenesis by administering to such subject an effective amount of a FZD4 FLAgs, wherein the effective amount is an amount sufficient to increase retinal or brain angiogenesis in such subject. The subject may be a fetus.

Pathologically low levels of Wnt signaling have been associated with osteoporosis, polycystic kidney disease and neurodegenerative diseases. Controlled activation of Wnt pathway has been shown to promote regenerative processes such as tissue repair and wound-healing. Zhao J, Kim K A, and Abo A, Trends Biotechnol. 27(3):131-6 (March 2009). See also, Logan C Y and Nusse R, Annu. Rev. Cell. Dev. Biol. 20:781-810 (2004); Nusse R., Cell Res. 15(1):28-32 (January 2005); Clevers H, Cell 127(3):469-80 (3 Nov. 2006). Proof-of-concept experiments have been done to show the role of Wnt signaling in osteoporosis or mucositis. Furthermore, it has been suggested that increasing of Wnt signaling might be beneficial for the treatment of diabetes and other metabolic diseases. Decreased Wnt signaling has been associated with metabolic disease. Loss-of-function LRP6^{R611C} mutation results in early coronary artery disease, metabolic syndrome and osteoporosis in human. Main A et al, Science 315:1278 (2007). “LRP5 loss-of-function mutation is associated with osteoporosis, impaired glucose metabolism and hypercholesterolaemia in human.” Saarinnen et al., Clin Endocrinol 72:481 (2010). Severe hypercholesterolemia, impaired fat tolerance, and advanced atherosclerosis in mice lacking both LRP5 and apoE. Magoori K. et al., JBC 1 1331 (2003). LRP5 is essential for normal cholesterol metabolism and glucose-induced insulin secretion in mice. Fujino et al., PNAS 100:229 (2003). TCF7L2 variant confers risk of type 2 diabetes. Grant et al., Nat Genet 38:320 (2006); Florez et al., N Engl J Med 355:241 (2006). An increase of Wnt signaling can be beneficial for treating metabolic diseases. Accordingly, the administration of the multivalent binding molecules of this invention to a subject with metabolic disease is useful for treating the subject's metabolic disease.

Inflammatory bowel disease (IBP) is a group of inflammatory conditions of the colon and small intestine. The major types of IBD are Crohn's disease and ulcerative colitis. RSP01 protein has been shown to ameliorate inflammatory bowel disease in an animal model. Zhao J et al., Gastroenterology 132:1331 (2007). Accordingly, the administration of the multivalent binding molecule of this invention, e.g., a multivalent binding molecule that binds to FZD7, e.g., 12735-K/H-2539-2542, to a subject with IBD is useful for treating the subject's IBD.

Thus, an embodiment of this invention is a method for treating a subject having a condition associated with reduced Wnt signaling comprising administering to a subject in need thereof an effective amount of the FZD agonists of this invention. The condition may be e.g., osteoporosis, polycystic kidney disease, neurodegenerative diseases, mucositis, short bowel syndrome, bacterial translocation in the gastrointestinal mucosa, enterotoxigenic or enteropathic infectious diarrhea, celiac disease, non-tropical sprue, lactose intolerance and other conditions where dietary exposures cause blunting of the mucosal villi and malabsorption, atrophic gastritis and diabetes, bone fracture, tissue regeneration, e.g. tissue repair and wound healing, as well as metabolic diseases such as diabetes, and melanoma, Examples of damaged tissue that can be treated using methods of the invention include, but are not limited to, intestinal tissue, cardiac tissue, liver tissue, kidney tissue, skeletal muscle, brain tissue, bone tissue, connective tissue, and skin tissue. The multivalent binding molecules of this invention can be administered to a subject with a disease or condition characterized by a low Wnt signaling. The multivalent binding molecules of the invention are administered to the subject in an amount effective to increase Wnt signaling and to ameliorate the disease or condition in the subject.

Mucositis is a clinical complication of cancer therapy. Mucositis is caused by the cytotoxic effects of irradiation or chemotherapy on fast proliferating cells. Mucositis consists of epithelial damage mainly affecting the intestinal and oral mucosa. Clinical signs are severe pain of the oral cavity, nausea, diarrhea, malnutrition, and, in severe cases, sepsis and death. The symptoms can often lead to dose limitation of cancer therapy. There are no currently available treatments for oral or gastrointestinal-mucositis associated with chemotherapy or radiation therapy for solid tumors.

Oral mucositis is a common and often debilitating complication of cancer treatment. 50% of patients undergoing radiotherapy for head and neck cancer and 10-15% of patients treated with 5-FU get grade 3-4 oral mucositis. RSP01 has been shown to ameliorate oral mucositis in an animal model. Zhao J et al., PNAS 106:2331 (2010).

Short bowel syndrome (SBS) results from functional or anatomic loss of extensive segments of small intestine, so that digestive and absorptive capacities are severely compromised. Each year, many people undergo resection of long segments of small intestine for various disorders, including trauma, inflammatory bowel disease, malignancy, mesenteric ischemia and others. Various nonoperative procedures such as radiation can cause functional short-bowel syndrome. Current therapies for short-bowel syndrome include dietary approaches, total parenteral nutrition (TPN), intestinal transplantation, and nontransplantation abdominal operations. Although these treatments have contributed to the improved outcome of SBS patients, they only partially correct the underlying problem of reduced bowel function. No current therapy can accelerate the recovery of remaining small intestine in SBS patients. See, Seetharam and Rodrigues, The Saudi Journal of Gastroenterology 17, 229-235 (2011).

The adult mammalian gut constitutes one of the most rapidly self-renewing tissues, in which the intestinal mucosa comprises a continuous structure folded into the proliferative crypts and the differentiated villi. In response to mucosal disruption, the host initiates a healing response resulting in restoration of mucosal integrity and regeneration of the mucosal architecture. This process is heavily dependent on the proliferation of intestinal stem cells. Neal et al., Journal of Surgical Research 167, 1-8 (2010); van der Flier and Clevers, Annual Review of Physiology 71, 241-261 (2009).

Therefore, the factors that regulate the activity of intestinal stem cells play a dominant role in the ability of the host to respond to injury within the intestinal tract. Because Wnt proteins are the most important growth factors that support the proliferation of intestinal stem cells, enhancing Wnt signaling will increase the proliferation of intestinal epithelium. This will lead to increased number of small bowel villi and increased mucosal absorptive surface area.

Thus, in one embodiment, the multivalent binding molecules of this invention are administered to a person with short bowel syndrome. In an embodiment of this invention the multivalent binding molecule of this invention binds FZD7, e.t, 12735-K/H-2539-2542 described herein. The multivalent binding molecules is administered in an amount sufficient to increase gastrointestinal mucosal absorptive surface area. The administration of the multivalent binding molecules of this invention has a successful outcome when the person with incident short bowel syndrome adapts to enteral feeding, or when the person with prevalent SBS absorbs nutrients from enteral feeds, or when the person decreases the amount of total parenteral nutrition required daily for the person to maintain weight.

Prevention of bacterial translocation. In one embodiment, the antibody of the invention is administered to a person at risk of septicemia caused by enteric bacteria. The multivalent binding molecules is administered in an amount sufficient to increase gastrointestinal mucosal integrity, thus preventing enteric bacteria from passing into the bloodstream of the person. Decreased gastrointestinal mucosal integrity (as compared with the gastrointestinal mucosal integrity that is normal for the human population) is a major source of bloodstream infections and sepsis in critically ill patients. The administration of the multivalent binding molecules has a successful outcome when fewer cases of bacteremia and sepsis are observed in intensive care unit (ICU) patients than in patients to whom the multivalent binding molecules of this invention is not administered.

Accelerated recovery during or after enterotoxigenic or enteropathic infectious diarrhea. Infectious diarrhea is a major pediatric problem. In one embodiment, the multivalent binding molecules of the invention is administered in an amount sufficient to shorten the time to the end of diarrhea or the time to normal bowel movements. The multivalent binding molecules of this invention can be administered in addition to the standard of care, which includes oral or parenteral rehydration and sometimes, antibiotics. The administration of the multivalent binding molecules has a successful outcome when decrease hospitalizations, shorten hospitalizations, or a decrease the incidence of complications of dehydration and electrolyte abnormalities are observed in pediatric patients as compared with pediatric patients to whom the multivalent binding molecules of the invention is not administered.

Celiac disease, non-tropical sprue, lactose intolerance and other conditions where dietary exposures cause blunting of the mucosal villi and malabsorption. In one embodiment, the multivalent binding molecules of this invention is administered in an amount sufficient to increase mucosal absorptive surface area. The multivalent binding molecules of this invention can be administered in addition to the standard of care, which is primarily avoiding the offending foods and sometimes, dietary supplements. The administration of the multivalent binding molecules of the invention has a successful outcome when the person with celiac disease, non-tropical sprue, lactose intolerance or other condition adapts to enteral feeding, or when the person with any of the conditions absorbs nutrients from enteral feeds, or when the person decreases the amount of total parenteral nutrition required daily for the person to maintain weight.

Atrophic gastritis, specifically the form termed environmental metaplastic atrophic gastritis. Atrophic gastritis is a common condition in the elderly, currently treated with vitamin B12 injections. The patients have an increased risk of carcinoid tumors and adenocarcinoma. The administration of the multivalent binding molecules has a successful outcome when decreased the tumor incidence, in the case of carcinoid by decreasing gastrin production from the metaplastic G cells, is observed by a medical expert. The multivalent binding molecules should not be administered to the subject if a medical expert determined that if the tumors are activated by increases in the Wnt pathway.

The FZD agonists of the present invention may be administered, e.g., by injection (e.g. subcutaneous, intravenous, intraperitoneal, etc.), topically, or orally. Depending on the route of administration, the active compound may be coated in a material to protect the compound from the action of acids and other natural conditions which may inactivate the compound. The multivalent binding molecules described herein may be dissolved or suspended in a pharmaceutically acceptable, preferably aqueous carrier. In addition, the composition can contain excipients, such as buffers, binding agents, blasting agents, diluents, flavors, lubricants, etc. An extensive listing of excipients that can be used in such a composition, can be, for example, taken from A. Kibbe, Handbook of Pharmaceutical Excipients (Kibbe, 2000). The multivalent binding molecules can also be administered together with immune stimulating substances, such as cytokines.

An embodiment of this invention includes a method for producing induced pluripotent stem (iPS) cells comprising culturing a somatic cell under conditions suitable for reprogramming the somatic cell wherein said culturing conditions further comprise a multivalent binding molecule described herein. Method for generating pluripotent stem cells are well known in the art, see e.g., Takahashi and Yamanaka, (2006), Induction of Pluripotent Stem Cells from Mouse Embryonic and Adult Fibroblast Cultures by Defined Factors, Cell 126, 663-676; Takahashi et al. (2007) Induction of Pluripotent Stem Cells from Adult Human Fibroblasts by Defined Factors Cell 131, 861-872; Yu et al. (2007). Induced pluripotent stem cell lines derived from human somatic cells. Science 318, 1917-1920; U.S. Pat. No. 8,546,140, and; U.S. Pat. No. 8,268,620. In an embodiment of this invention the multivalent binding molecules of this invention are included in the culture media in an amount sufficient to accelerate the generation of iPS cells.

An embodiment of this invention includes a method for directed differentiation of multipotent or pluripotent stem cells (PSC) or induced pluripotent stem (iPS) cells comprising culturing the cells under conditions suitable for directed differentiation wherein said culturing conditions further comprise an effective amount of a multivalent binding molecule described herein. Studies in mouse and human PSCs have identified specific approaches to the addition of growth factors, including Wnt, which can induce PSC differentiation into different lineages. Methods for directed differentiation of PSCs comprising the activation of Wnt signaling are known in the art see e.g. Lam et al. (2014) Semin Nephol 34(4); 445-461; Yucer et al. (Sep. 6, 2017) Scientific Reports 7, Article number 10741. It is contemplated that the multivalent binding molecules described herein can be used to effect activation of Wnt signaling pathways to direct differentiation of the PSCs.

An embodiment of this invention is a method for enhancing tissue regeneration in a subject in need thereof by activating Wnt signaling in such subject by administering to the subject in need thereof an effective amount of a multivalent binding peptide described herein.

An embodiment of this invention includes a method for enhancing bone healing and/or regeneration in a subject in need thereof, e.g., a subject with osteoporosis or fracture, by administering an effective amount of a multivalent binding molecule described herein. In a particular embodiment the multivalent biding molecule of this invention comprises a binding domain that binds to FZD2 and a binding domain that binds to LRP5 or/and LRP6. The binding domains may be monovalent or multivalent, e.g., bivalent, trivalent or tetravalent, and monospecific or multispecific, e.g., bispecific. In an embodiment of this invention the multivalent binding molecules for enhancing bone healing and/or regeneration in a subject in need thereof comprise, e.g., 2890-knob-2539-2542 (SEQ ID NO: 77) and 2890-hole-2539-2542 (SEQ ID NO: 79) (together forming 2890-K/H-2539:2542 or 2890Ag).

A subject may be any animal (e.g., a mammal), including, but not limited to, humans, non-human primates, horses, cows, dogs, cats, rodents, and the like. Typically, the subject is human.

Effective dosages and schedules for administering the multivalent binding molecules described herein may be determined empirically, and making such determinations is within the skill in the art. Those skilled in the art will understand that the dosage of such FZD agonists that must be administered will vary depending on, for example, the subject that will receive the antibody, the route of administration, the particular type of FZD agonists used and other drugs being administered. Guidance in selecting appropriate doses for FZD agonists is found in the literature on therapeutic uses of antibodies, e.g., Handbook of Monoclonal Antibodies, Ferrone, eds., Noges Publications, Park Ridge, N.J., (1985) ch. 22 and pp. 303-357; Smith, Antibodies in Human Diagnosis and Therapy, Haber, eds., Raven Press, New York (1977) pp. 365-389. The dosage ranges for the administration of the compositions are those large enough to produce the desired effect. The dosage should not be so large as to cause adverse side effects, such as unwanted cross-reactions, anaphylactic reactions, and the like. Generally, the dosage will vary with the age, condition, sex and extent of the inflammation in the patient and can be determined by one of skill in the art. The dosage can be adjusted by the individual physician in the event of any contraindications. Dosage can vary, and can be administered in one or more dose administrations daily, for one or several days. While individual needs vary, determination of optimal ranges of effective amounts of the vector is within the skill of the art.

In recent years, methods have been developed for culturing mini-organs called “organoids” that recapitulate the gross anatomy and cell type composition of different tissues. Remarkably, full organoids can be generated from a single tissue stem cell as first demonstrated with intestinal LGR5+ stem cells isolated from a mouse. It is known that components within the media that activate the Wnt-βcatenin pathway are required for organoid derivatization, growth, survival and maintenance. Therefore, R-spondin and Wnt ligands, purified or provided as conditioned media are universally required to grow organoids from different tissues. However, purified Wnt proteins have generally low specific activity and are not able to sustain growth of organoids. As such those of skill in the art rely on the addition of Wnt3A conditioned media, or to the addition of small molecules, e.g., GSK3 inhibitors, to generate organoids. But the production of Wnt3A conditioned media is labor intensive, the characteristics of the conditioned media are inconsistent, and small molecule GSK3 inhibitors may robustly activate the pathway to levels that are toxic. The multivalent binding molecules described herein solve these problems as they are easy to produce and purify, have consistent reproducible characteristics, and activate Wnt specifically by selectively engaging the desired FZD receptor(s) and co-receptor(s) combination.

An embodiment of this invention includes a method for generating tissue organoids comprising culturing tissue in an effective amount of a multivalent binding molecule described herein. An organoid is a 3D multicellular in vitro tissue construct that mimics its corresponding in vivo organ, such that it can be used to study aspects of that organ in the tissue culture dish. Methods for generating organoids are well known in the art and epithelial organoids derived from adult stem cells in the various organs of the gastrointestinal tract, for example, almost all need agonists of Wnt signaling (among other signaling factors, including embedding in Matrigel) to both maintain the cells and to generate an in vivo-like complement of cell types. Wnt signaling also enhances inner ear organoid development in 3D culture, and has been used in the generation of kidney organoids, see e.g., Natalie de Souza (2018) Nature Methods 15(1): 23; DeJonge et al. (2016) PLosOne 11(9), e0162508; Akkerman and Defize, (2017) Bioessays 39, 4, 1600244. The multivalent binding molecules of this invention can be included in the culture media of organoids in an amount sufficient to enhance their growth, survival and maintenance in culture. As such, an embodiment of this invention includes a method for enhancing the culture of tissue organoids comprising a culture medium comprising an effective amount of a multivalent binding molecule described herein.

Also an aspect of this invention is a method for making the multivalent binding molecules described herein. In an embodiment of this invention the multivalent binding molecule is generated by,
a) selecting an Fc domain having a C-terminus and an N-terminus
b) identifying a peptide that binds to one or more FZD receptors, or identifying an antibody that binds to one or more FZD receptors, and
c) identifying a peptide that binds to one or more Wnt co-receptors or identifying an antibody that binds to one or more Wnt co-receptors,
d) generating a nucleic acid molecule comprising a nucleotide sequence that encodes (i) the Fc domain of step a, (ii) a nucleotide sequence that encodes the peptide of step b, or a nucleotide sequence that encodes a VL and/or a VH of the antibody of step b, or a nucleotide sequence that encodes a VL and/or a VH derived from the antibody of step b, that binds the one or more FZD receptors, and (iii) a nucleotide sequence that encodes the peptide of step c, or a nucleotide sequence that encodes the VL and/or the VH of the antibody of step c, or a nucleotide sequence that encodes the VL and/or the VH derived from the antibody of step c, that binds to the one or more Wnt co-receptors,
e) expressing the nucleic acid molecule of (d) to produce a polypeptide wherein the polypeptide dimerizes to form a tetravalent binding molecule comprising (i) an Fc domain, (ii) a FZD binding domain and (iii) a Wnt co-receptor binding domain wherein, such that the FZD binding domain comprises of the peptide of step b, or the VL and/or VH of step b, and is linked to one terminus of the Fc domain, and the Wnt co-receptor binding domain comprises the peptide of step c or the VL and/or VH of step c and is linked to the other terminus of the Fc domain thereby forming the multi-specific binding molecule.

The peptide that binds to one or more of the FZD receptor may be a synthetic polypeptide, e.g., a synthetic peptide, an affibody, an ankyrin repeat protein, a fibronectin repeat protein, a fynomer, or an anticalin or a peptide of a naturally occurring protein that binds the FZD receptor. The naturally occurring protein may be, e.g., a Wnt, e.g., Wnt-1, Wnt-2, Wnt-2b, Wnt-3a, Wnt-4, Wnt-5a, Wnt-5b, Wnt-6, Wnt-7a, Wnt-7a/b, Wnt-7b, Wnt-8a, Wnt-8b, Wnt-9a, Wnt-9b, Wnt-10a, Wnt-10b, Wnt-11, Wnt-16b. The peptide of step b may be multivalent, binding to more than one site on the FZD, e.g., bivalent, trivalent of tetravalent, and may be monospecific, binding to a single epitope, or multispecific, binding to more than one epitope on the FZD.

The peptide that binds to one or more of the Wnt co receptor may be a synthetic peptide, e.g., an affibody, an ankyrin repeat protein, a fibronectin repeat protein, a fynomer, or an anticalin, or a peptide of a naturally occurring protein that binds the Wnt co-receptor. The naturally occurring protein may be for example, a Wnt, e.g., Wnt-1, Wnt-2, Wnt-2b, Wnt-3a, Wnt-4, Wnt-5a, Wnt-5b, Wnt-6, Wnt-7a, Wnt-7a/b, Wnt-7b, Wnt-8a, Wnt-8b, Wnt-9a, Wnt-9b, Wnt-10a, Wnt-10b, Wnt-11 or Wnt-16b, or Dickkopf-1.

The peptide of step c may be multivalent binding to more than one epitope on the Wnt co-receptor, e.g., bivalent, trivalent of tetravalent, and may be monospecific binding to a single epitope or multispecific binding to more than one epitope on the Wnt co-receptor.

The naturally occurring protein that binds the FZD receptor and the naturally occurring protein that binds the Wnt co-receptor may be the same protein.

In an embodiment the peptide or antibody of step b may bind FZD2 and the peptide of step c may be a peptide of Wnt5a and the antibody of step c may be an antibody that binds to a site on the co-receptor that binds to Wnt5a.

In an embodiment the peptide or antibody of step b may bind FZD4 and the peptide of step c may be a peptide of one of more of Norrin, Wnt1, Wnt8, or Wnt5a and the antibody of step c may be antibody that binds to a site on the co-receptor that binds to Norrin, Wnt1, Wnt8, or Wnt5a.

In an embodiment the peptide or antibody of step b may bind FZD5 and the peptide of step c may be a peptide of one or more of Wnt7a, Wnt5a, Wnt10b, or Wnt2 and the antibody of step c may be an antibody that binds to a site on the co-receptor which site binds to one or more of Wnt7a, Wnt5a, Wnt10b, or Wnt2.

In an embodiment the peptide or antibody of step c binds LRP6 and/or LRP5, e.g., the peptide may be a peptide of Norrin, Wnt1 and/or Wnt3a, and the antibody of step c may be an antibody that binds to a site on LRP6/LRP5 which site binds to Norrin, Wnt1 and/or Wnt3a.

In an embodiment the peptide or antibody of step c may bind LRP6, e.g., the peptide may be a peptide of Wnt1 or Wnt3a, or both, and the antibody may be an antibody that binds a site on LRP6 that binds Wnt1 or Wnt3a.

In an embodiment the peptide or antibody of step c binds ROR1 and/or ROR2.

In an embodiment the peptide or antibody of step c may bind RYK.

In an embodiment the peptide or antibody of step c may bind PTK7.

In an embodiment, the peptide or antibody in step (b) may be a peptide or antibody that binds to one or more FZD receptors and antagonizes Wnt signaling or inhibits Wnt binding to the receptor. In an embodiment, the peptide or antibody in step (b) may be a peptide or antibody that binds to one or more FZD receptors without antagonizing Wnt signaling or inhibiting Wnt binding to the receptor. In an embodiment, the peptide or antibody in step (c) may be a peptide or antibody that binds to one or more of the Wnt co-receptors and antagonizes Wnt signaling or inhibits Wnt binding to the co-receptor. In an embodiment, the peptide or antibody of step (c) may be a peptide or antibody that binds to the Wnt co-receptor without antagonizing Wnt signaling or inhibiting Wnt binding to the co-receptor. The binding domains may be linked to the Fc domain via a linker. The modular aspects of this invention allows for mixing and matching of peptide or antibody VH and VL that bind to any given FZD receptor and Wnt co-receptor on the opposite termini of the Fe domain to generate a multivalent binding molecule that can engage multiple Frizzled receptor—co-receptor complexes or to selectively engage a single Frizzled receptor-co-receptor complex to activate Wnt signaling.

An embodiment of this invention is a method of making a multivalent binding molecule that activates a Wnt signaling pathway comprising
a) selecting an Fc domain having a C-terminus and an N-terminus, e.g. an Fc domain of an immunoglobulin comprising a CH3 domain, e.g., an IgG, e.g., an IgG1,
b) identifying an antibody having a binding specificity for one or more FZD receptor and
c) identifying an antibody having a binding specificity for a Wnt co-receptor;
d) generating a nucleic acid molecule comprising
   (i) a nucleotide sequence that encodes the selected Fc domain,
   (ii) a nucleotide sequence that encodes a VL and/or a VH derived from the antibody of step b, and
   (iii) a nucleotide sequence that encodes a VL and/or a VH derived from the antibody of step c,
d) expressing the nucleic acid molecule of (d) to produce a polypeptide which dimerizes via the Fc domain to form a multivalent binding molecule comprising (i) the Fc domain, (ii) a FZD binding domain and (iii) a Wnt co-receptor binding domain, such that the FZD binding domain is linked to one terminus of the Fc domain and the Wnt co-receptor binding domain is linked to the other terminus of the Fc domain thereby forming a multivalent binding molecule. In a preferred embodiment the multivalent binding molecule is a dimer of two polypeptides encoded by the nucleic acid molecule wherein the Fc domain is in a knob in hole configuration. One or both of the binding domains may be multivalent binding domains. The antibody of step b may be an antibody fragment that binds the FZD receptor. The VH and/or VL in step d)(ii) may be identical to the VH and/or VL of the antibody of step b). The antibody of step c may be an antibody fragment that binds the Wnt co-receptor. The VH and/or VL in step d)(iii) may be identical to the VH and/or VL of the antibody of step c).

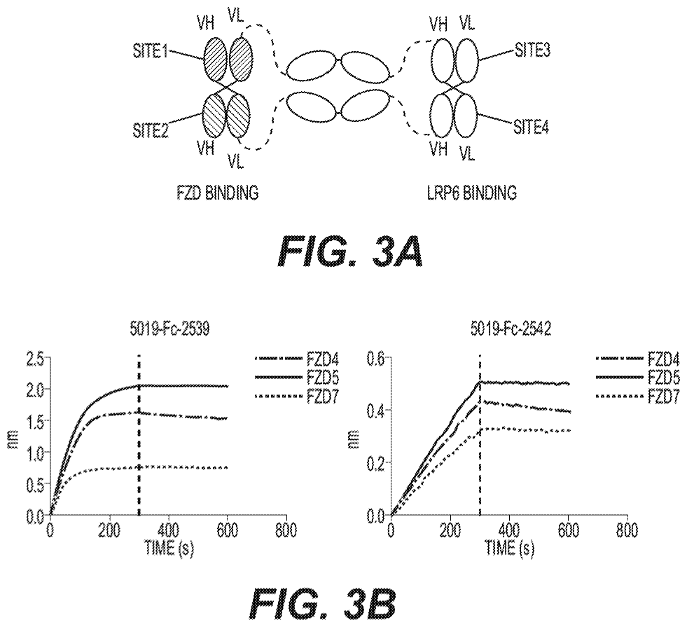

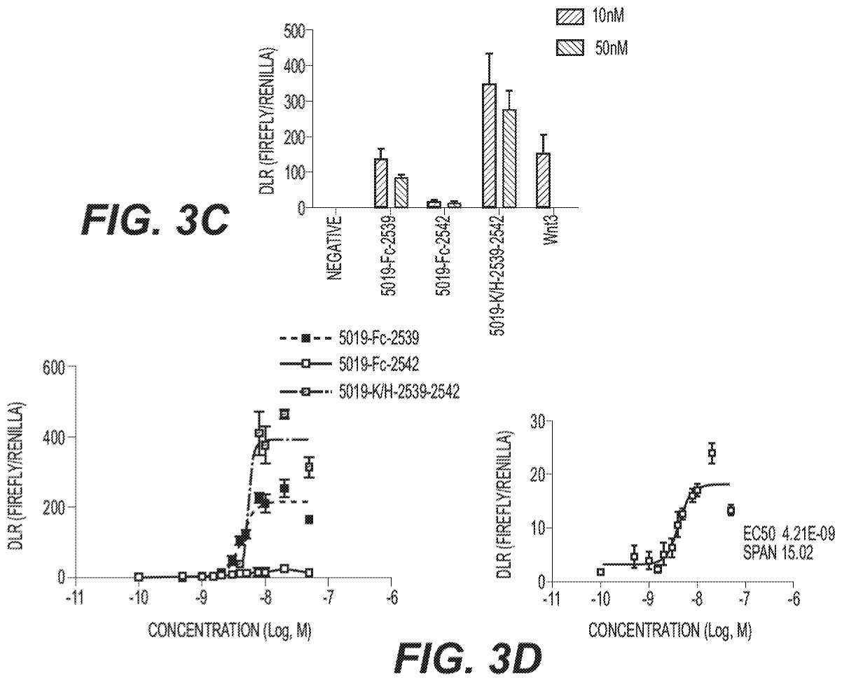

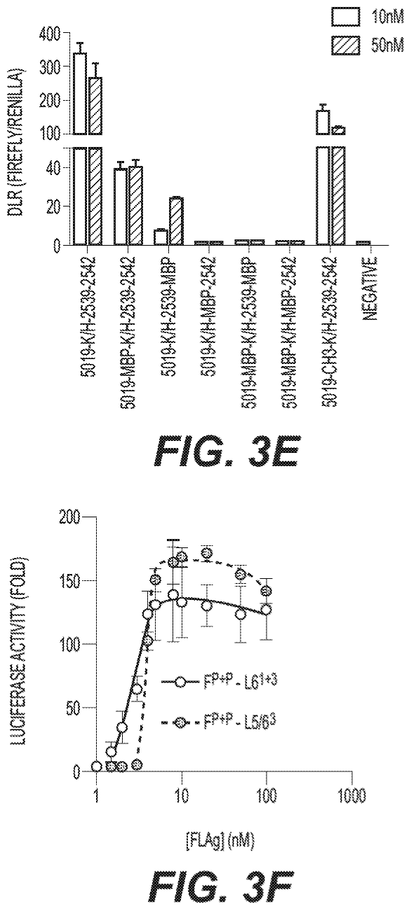

The multivalent molecules of this invention may be generated by dimerizing two polypeptides in a “knob-in-hole” configuration. The knob-in-hole configuration increases the modularity of this invention by facilitating the association of peptides that comprise binding moieties that bind different epitopes on a FZD receptor or co-receptor or to different members of the same FZD receptor or co-receptor family, see e.g., FIG. 3A. Methods for engineering Fc molecules via the knobs into holes design are well known in the art, see e.g., WO2018/026942, inventors Van Dyk et al., Carter P. (2001) J. Immunol. Methods 248, 7-15; Ridgway et al. (1996) Protein Eng. 9, 617-621; Merchant A. M., et al.. (1998) Nat. Biotechnol. 16, 677-681 and; et al., (1997) J. Mol. Biol. 270, 26-35.

Another embodiment of this invention is a method for facilitating the interaction of a FZD receptor and a co-receptor on a cell thereby activating a Wnt signaling pathway in the cell comprising, a) selecting an Fc domain, or fragment thereof comprising a CH3 domain, having a C-terminus and an N-terminus b) linking a first multivalent binding domain, which binds the FZD receptor, on one terminus of the Fc domain and linking a second binding domain, which binds to the Wnt co-receptor, on the other terminus of the Fc domain thereby forming a binding molecule; c) contacting said multivalent binding molecule with the cell expressing said FZD receptor and Wnt co-receptor under conditions wherein the FZD receptor and co-receptor both bind to the multivalent binding molecule thereby activating the Wnt signaling pathway. One or both of the binding domains may be monovalent or multivalent, e.g., bivalent, trivalent, or tetravalent. The FZD binding domain may comprise a peptide of a naturally occurring protein that binds FZD, a synthetic peptide, e.g., a affibody, an ankyrin repeat protein, a fibronectin repeat protein, a fynomer, or an anticalin, that binds FZD, VH and/or VL fragments that bind FZD, a scFV that binds FZD, or a diabody that binds FZD. The Wnt co-receptor binding domain may comprise a peptide of a naturally occurring protein that binds the Wnt co-receptor, a synthetic peptide, e.g., an affibody, an ankyrin repeat protein, a fibronectin repeat protein, a fynomer or an anticalin, that binds to the Wnt co-receptor, VH and/or VL fragments that bind the Wnt co-receptor, a scFV that binds the Wnt co-receptor, or a diabody that binds the Wnt co-receptor.

An embodiment of this invention is a molecule comprising an Fc domain and two binding domains, the first domain binds to a FZD receptor and the second domain binds to a Wnt co-receptor, and these two moieties are linked together by a Fc domain, or fragment thereof comprising the CH3 domain, wherein one domain is linked to the N-terminus of the Fc receptor, and the other domain is linked to the C-terminus of the Fc receptor. The binding domains may be linked to the Fc receptor either directly or via a peptide linker, e.g. a polypeptide linker, or a non-peptidic linker. Suitable linkers are well known in the art, e.g., an XTEN linker (see WO2013120683, inventors Schellenberger et al.)

An embodiment of this invention is a method for activating a Wnt signaling pathway comprising contacting a cell expressing a FZD receptor and its co-receptor with an effective amount of the multivalent molecules of this invention. Without wishing to be bound by theory, it is contemplated that the multivalent molecules described herein bind both the FZD receptor and its co-receptor thereby forming a complex that mimics the binding of a Wnt molecule to the FZD receptor and co-receptor(s), which in turn activates Wnt signaling pathways.

The multivalent binding molecules of this invention may be made recombinantly, e.g., by Gibson assembly (see Gibson et al. (2009). Nature Methods. 6 (5): 343-345 and Gibson D G. (2011). Methods in Enzymology. 498: 349-361), or the molecules may be made synthetically e.g., using a commercial synthetic apparatuses, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like. If desired, various groups may be introduced into the peptide during synthesis or during expression, which allow for linking to other molecules or to a surface.

In some embodiments, the binding domains are attached to the Fc domain via a peptide linker, e.g., an XTEN linker. In some embodiments, the peptide linker comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or at least 100 amino acids. In some embodiments, the peptide linker is between 5 to 75, 5 to 50, 5 to 25, 5 to 20, 5 to 15, or 5 to 10 amino acids in length. The Fc domain with or without the linker are of a length and flexibility that allows for the multivalent binding molecule to bind both the FZD receptor and its co-receptor thereby activating a Wnt signaling pathway. In an embodiment of this invention the Fc domain, or fragment thereof comprising the CH3 domain, with or without the linker is greater than 100 amino acids, greater than 125 amino acids greater than 150 amino acids, greater than 175 amino acids or greater than 200 amino acids.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

An “affinity matured” antibody or “maturation of an antibody” refers to an antibody with one or more alterations in one or more hypervariable regions (HVRs), compared to a parent or source antibody which does not possess such alterations, such alterations resulting in an improvement in the affinity of the antibody for antigen or to other desired properties of the molecule.

By “comprising” it is meant that the recited elements are required in the composition/method/kit, but other elements may be included to form the composition/method/kit etc. within the scope of the claim. For example, a composition comprising multivalent binding molecules is a composition that may comprise other elements in addition to multivalent binding molecules, e.g. functional moieties such as polypeptides, small molecules, or nucleic acids bound, e.g. covalently bound, to the multivalent binding molecules; agents that promote the stability of the multivalent binding molecule composition, agents that promote the solubility of the multivalent binding molecule composition, adjuvants, etc. as will be readily understood in the art, with the exception of elements that are encompassed by any negative provisos.

By “consisting essentially of”, it is meant a limitation of the scope of composition or method described to the specified materials or steps that do not materially affect the basic and novel characteristic(s) of the subject invention. For example, a multivalent binding molecule “consisting essentially of” a disclosed sequence has the amino acid sequence of the disclosed sequence plus or minus about 5 amino acid residues at the boundaries of the sequence based upon the sequence from which it was derived, e.g. about 5 residues, 4 residues, 3 residues, 2 residues or about 1 residue less than the recited bounding amino acid residue, or about 1 residue, 2 residues, 3 residues, 4 residues, or 5 residues more than the recited bounding amino acid residue.

By “consisting of”, it is meant the exclusion from the composition, method, or kit of any element, step, or ingredient not specified in the claim. For example, a multivalent binding molecule “consisting of” a disclosed sequence consists only of the disclosed amino acid sequence.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

Three highly divergent stretches within each of the heavy chain variable domain, VH, and light chain variable domain, VL, referred to as complementarity determining regions (CDRs), are interposed between more conserved flanking stretches known as “framework regions”, or “FRs”. Thus, the term “FR” refers to amino acid sequences which are naturally found between, and adjacent to, CDRs in immunoglobulins. A VH domain typically has four FRs, referred to herein as VH framework region 1 (FR1), VH framework region 2 (FR2), VH framework region 3 (FR3), and VH framework region 4 (FR4). Similarly, a VL domain typically has four FRs, referred to herein as VL framework region 1 (FR1), VL framework region 2 (FR2), VL framework region 3 (FR3), and VL framework region 4 (FR4). In an antibody molecule, the three CDRs of a VL domain (CDR-L1, CDR-L2 and CDR-L3) and the three CDRs of a VH domain (CDR-H1, CDR-H2 and CDR-H3) are disposed relative to each other in three dimensional space to form an antigen-binding site within the antibody variable region. The surface of the antigen-binding site is complementary to a three-dimensional surface of a bound antigen. The amino acid sequences of VL and VH domains may be numbered, and CDRs and FRs therein identified/defined, according to the Kabat numbering system (Kabat et al., 1991, Sequences of Proteins of Immunological Interest, 5th Ed. Public Health Service, National Institutes of Health, Bethesda, Md.) or the INTERNATIONAL IMMUNOGENETICS INFORMATION SYSTEM (IMGT numbering system; Lefranc et al., 2003, Development and Comparative Immunology 27:55-77). One of ordinary skill in the art would possess the knowledge for numbering amino acid residues of a VL domain and of a VH domain, and identifying CDRs and FRs therein, according to a routinely employed numbering system such as the IMGT numbering system, the Kabat numbering system, and the like.

The term “antigen-binding portion” or “antigen-binding fragment” of an antibody (or simply “antibody portion” or “antibody fragment”), as used herein, refers to one or more fragments, portions or domains of an antibody that retain the ability to specifically bind to an antigen. It has been shown that fragments of a full-length antibody can perform the antigen-binding function of an antibody. Examples of binding fragments encompassed within the term “antigen-binding portion” of an antibody include (i) an Fab fragment, a monovalent fragment consisting of the VL, VH, CL1 and CH1 domains; (ii) an F(ab′)₂ fragment, a bivalent fragment comprising two F(ab)′ fragments linked by a disulfide bridge at the hinge region; (iii) an Fd fragment consisting of the VH and CH1 domains; (iv) an Fv fragment consisting of the VL and VH domains of a single arm of an antibody; (v) a dAb fragment (Ward et al. (1989) Nature 241:544-546), which consists of a VH domain; and (vi) an isolated complementary determining region (CDR). Furthermore, although the two domains of the Fv fragment, VL and VH, are coded for by separate genes, they can be joined, using recombinant methods, by a synthetic linker that enables them to be made as a single contiguous chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies, are also encompassed (see e.g., Holliger et al. (1993) PNAS. USA 90:6444-6448).

“Affibodies” are small, single domain proteins engineered to bind to a large number of target proteins or peptides with high affinity, imitating monoclonal antibodies. They are composed of a three-helix bundle based on the scaffold of one of the IgG-binding domains of staphylococcal protein A. This scaffold domain consists of 58 amino acids, 13 of which are randomized to generate affibody libraries with a large number of ligand variants. See, e.g., U.S. Pat. No. 5,831,012 and Lofblom et al. FEBS Letters 584 (2010) 2670-2680. Affibody molecules mimic antibodies have a molecular weight of about 6 kDa.

“Diabodies” as used herein are dimeric antibody fragments. In each polypeptide of the diabody, a heavy-chain variable domain (VH) is linked to a light-chain variable domain (VL) but unlike single-chain Fv fragments, the linker between the VL and VH is too short for intramolecular pairing and as such each antigen-binding site is formed by pairing of the VH and VL of one polypeptide with the VH and VL of the other polypeptide, see e.g. FIG. 3A. Diabodies thus have two antigen-binding sites, and can be monospecific or bispecific. (see e.g., Holliger, P., et al. (1993) Proc. Natl. Acad. Sci. USA 90:6444-6448; Poljak, R. J., et al. (1994) Structure 2:1121-1123; Kontermann and Dubel eds., Antibody Engineering (2001) Springer-Verlag. New York. 790 pp. (ISBN 3-540-41354-5).

As used herein an “effective amount” of an agent, e.g., the multivalent binding molecules or a pharmaceutical composition comprising the molecules, refers to an amount effective, at dosages and for periods of time necessary, to achieve the desired result. In some embodiments, a therapeutically effective amount is one that reduces the incidence and/or severity of, stabilizes one or more characteristics of, and/or delays onset of, one or more symptoms of the disease, disorder, and/or condition.

As used herein, the term “epitope” includes any protein determinant capable of specific binding to an immunoglobulin or fragment thereof, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is <10 μM; e.g., <100 nM, preferably <10 nM and more preferably <1 nM.

The constant region of immunoglobulin molecules is also called the fragment crystallizable region, the “Fc region” or “Fc domain.” The Fc domain is composed of two identical protein fragments, derived from the second and third constant domains of the antibody's two heavy chains and the Fc domains of IgGs bear a highly conserved N-glycosylation site. Glycosylation of the Fc fragment is essential for Fc receptor-mediated activity. In an embodiment of the invention the Fc domain of the multivalent molecule is engineered such that it does not target the cell binding the multivalent molecule for ADCC or CDC-dependent death. In an embodiment of the invention the Fc domain of the multivalent binding molecule is a peptide dimer in a knob-in-hole configuration. The peptide dimer may be a heterodimer.

The terms “individual,” “subject,” “host,” and “patient,” are used interchangeably herein and refer to any mammalian subject for whom diagnosis, treatment, or therapy is desired, particularly humans.

“LRP”, “LRP proteins” and “LRP receptors” is used herein to refer to members of the low density lipoprotein receptor-related protein family. These receptors are single-pass transmembrane proteins that bind and internalize ligands in the process of receptor-mediated endocytosis. LRP proteins LRP5 (GenBank Accession No. NM 002335.2) and LRP6 (GenBank Accession No. NM 002336.2) are included in a Wnt receptor complex required for activation on the Wnt-βcatenin signaling pathway.

The term “polypeptide fragment” as used herein refers to a polypeptide that has an amino terminal and/or carboxy-terminal deletion, but where the remaining amino acid sequence is identical to the corresponding positions in the naturally-occurring sequence deduced, for example, from a full length cDNA sequence.

As used herein the term “paratope” includes the antigen binding site in the variable region of an antibody that binds to an epitope.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

The ability of the multivalent binding molecules of this invention to activate Wnt signaling can be confirmed by a number of assays. The multivalent binding molecules of this invention typically initiate a reaction or activity that is similar to or the same as that initiated by the FZD receptor's natural ligand. The multivalent binding molecules of this invention activates the Wnt signaling pathways, e.g., the canonical Wnt-βcatenin signaling pathway. As used herein, the term “activates” refers to a measurable increase in the intracellular level of a Wnt signaling pathway, e.g., the Wnt-βcatenin signaling pathway, compared with the level in the absence of a FZD agonist of the invention.

Various methods are known in the art for measuring the level of Wnt-βcatenin activation. These include but are not limited to assays that measure: Wnt-βcatenin target gene expression; LEF/TCF reporter gene expression (such as TopFLASH, superTopFLASH, pBAR); βcatenin stabilization; LRP5/6 phosphorylation; Axin translocation from cytoplasm to cell membrane and binding to LRP5/6. The canonical Wnt-βcatenin signaling pathway ultimately leads to changes in gene expression through the transcription factors TCF1, TCF7L1, TCF7L2 and LEF. The transcriptional response to Wnt activation has been characterized in a number of cells and tissues. As such, global transcriptional profiling by methods well known in the art can be used to assess Wnt-βcatenin signaling activation.

Changes in Wnt-responsive gene expression are generally mediated by TCF and LEF transcription factors. A TCF reporter assay assesses changes in the transcription of TCF/LEF controlled genes to determine the level of Wnt-βcatenin signaling. A TCF reporter assay was first described by Korinek, V. et al., 1997. Also known as TOP/FOP this method involves the use of three copies of the optimal TCF motif CCTTTGATC, or three copies of the mutant motif CCTTTGGCC, upstream of a minimal c-Fos promoter driving luciferase expression (pTOPFLASH and pFOPFLASH, respectively) to determine the transactivational activity of endogenous βcatenin/TCF. A higher ratio of these two reporter activities (TOP/FOP) indicates higher βcatenin/TCF activity. A newer and more sensitive version of this reporter is called pBAR and contains 12 repeats of the TCF motifs (Biechele and Moon, Methods Mol Biol. 2008; 468:99-110, PMID: 19099249).

General methods in molecular and cellular biochemistry can be found in such standard textbooks as Molecular Cloning: A Laboratory Manual, 3rd Ed. (Sambrook et al., CSH Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998).

“Single-chain Fv” or “scFv” antibody fragments comprise the VH and VL domains of antibody, wherein these domains are present in a single polypeptide chain. Generally, the Fv polypeptide further comprises a polypeptide linker between the VH and VL domains which enables the scFv to form the desired structure for antigen binding. For a review of scFv and other antibody fragments, see James D. Marks, Antibody Engineering, Chapter 2, Oxford University Press (1995) (Carl K. Borrebaeck, Ed.).

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

### Example I

### 1. Development of Multivalent FZD Agonists.

To make a multivalent binding molecule having a first binding domain comprising a FZD diabody and a second binding domain comprising a co-receptor diabody, we identified FZD specific antibodies from a synthetic Fab phage library (Library F; see US publication no. 2016/0194394, inventors Sidhu et al.) by selecting for those that bound to the cysteine rich domain (CRD) of FZD receptors using conventional phage-display technologies. Affinity or specificity maturation was carried out as needed. For example, a pan-FZD binding antibody, #5019 (which recognizes FZD1, 2, 4, 5, 7 and 8), was maturated from a FZD7-derived antibody using the FZD4 CRD as antigen. Our previous work also identified several antibodies that are completely specific for FZD4 (5038, 5044, 5048, 5062, 5063, 5080, 5081) or for FZD5 (2928) (see, e.g., US20160194394, inventors Sidhu et al. and WO2017127933A1, inventors Pan et al.).

These FZD antibodies were used to prepare a FZD specific diabody. A diabody is an antibody form similar to single chain variable fragment (scFv), but it is a dimer of two peptides each encoding a VL and VH, however, unlike a scFv the linker between the VH and VL within the polypeptides is too short to allow for intramolecular complementation between the VH and VL domains. Therefore, the VH-VL fragment of one polypeptide dimerizes with the VH-VL fragment of another polypeptide in such a way to functionally reconstitute two antigen binding paratopes. Diabodies were generated having paratopes that were identical or non-identical, by forming dimers of the polypeptides having the same VL and VH thereby forming homodiabodies, or forming dimers from two polypeptides having different VL and VH domain thereby forming heterodiabodies.

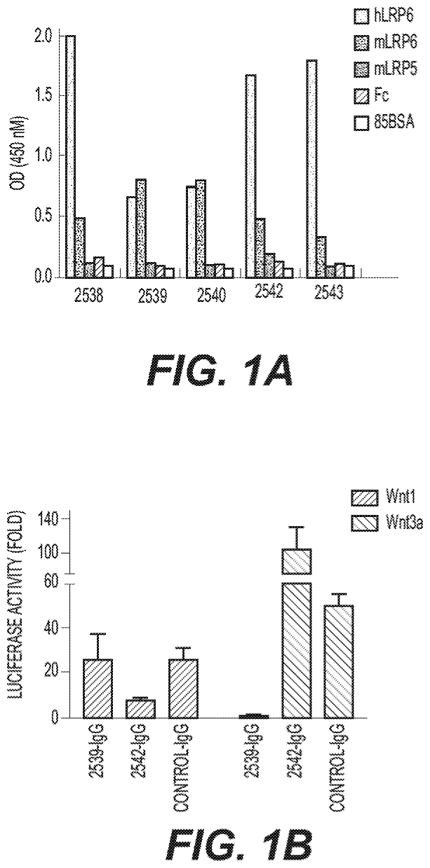

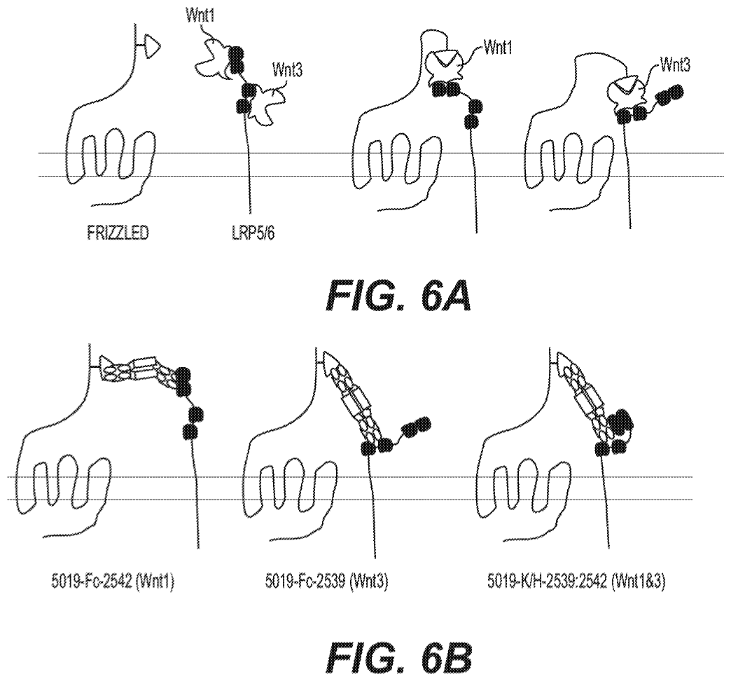

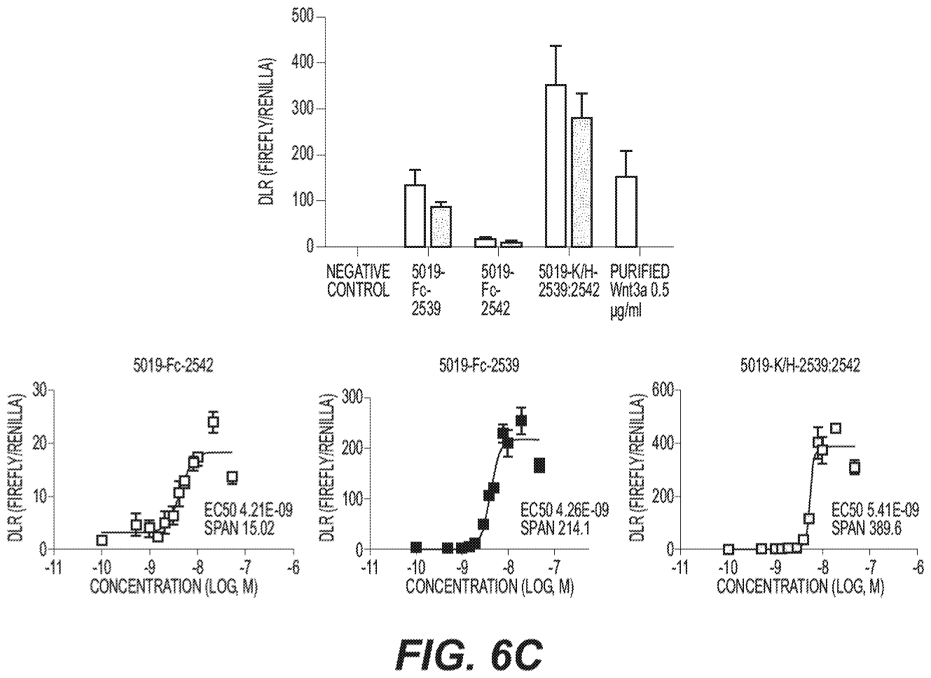

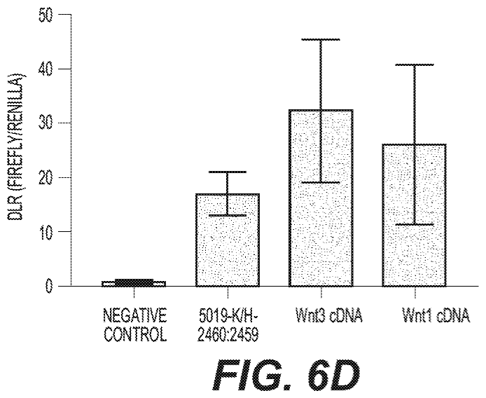

LRP6 antibodies were also selected from a synthetic antibody library by selecting those that bound the recombinant extracellular domain (ECD) of human LRP6. Five Fab with unique CDR regions were identified. After converting to IgG forms, they all display human LRP6 binding as well as mouse LRP6 binding. No LRP5 binding was detected via ELISA, demonstrating these antibodies are LRP6 specific (FIG. 1A). LRP6 ECD contains four 0-propeller motifs that alternate with four epidermal growth factor (EGF) like repeats. The first two β-propeller motifs are thought to be involved in Wnt1 binding and the last two are thought to be involved in Wnt3 binding, thus creating two potential epitopes for antibody binding. See FIG. 6A. Epitope binding results suggest that these five antibodies bind two separate sites on LRP6 and could be divided into two groups with antibodies 2538, 2542, and 2543 binding the Wnt1 binding site and 2539 and 2540 binding the Wnt3 binding site on LRP6. In general, an antibody binding to the LRP6-Wnt1 site would be expected to block Wnt1-induced Wnt pathway activation.

To prepare the Fc N-terminal binding domain containing a homodiabody specific for a FZD, the VH and VL fragments, VH-1, VH-2, VL-1 and VL-2, of the selected FZD antibodies were amplified by PCR from the corresponding phagemid templates and isolated. Gibson assembly was then utilized to introduce the isolated fragments (VH-1 and VL-2) into an EcoRI/XhoI precut vector containing an Fc-knob region (pSCST backbone) (see Gibson et al. (2009). Nature Methods. 6 (5): 343-345 and Gibson D G. (2011). Methods in Enzymology. 498: 349-361). Gibson assembly was also utilized to introduce the fragments (VH-2 and VL-1) into the EcoRI/XhoI precut vector containing an Fc-hole region. Correct assembly was validated using DNA sequencing. The two plasmids (one pair, Fc-knob and Fc-hole) were then used to introduce the second binding domain at the C-terminus of the Fc domain.

The Fc-knob and Fc-hole configuration was needed to generate multivalent binding domains wherein one of the binding domains was a heterodiabody. However, the Fc-knob and Fc-hole configuration was not needed to prepare binding molecules that comprise homodiabodies on both the N and C termini of the Fc domain and thus for such binding molecules the VHs and VLs were linked to a wild-type Fc region and only one plasmid was used to generate the VH-VL containing polypeptides to form the homodimer. Optionally, a linker, e.g. a peptide linker, or a non-peptidic linker, can be present between the binding domains and the Fc domain.

To generate the C-terminal binding domain, an LRP5/6 antibody was identified and an LRP5/6 diabody was generated following the same protocol as described above for generating the FZD diabody. The C-terminal binding domain was generated by PCR amplifying the VH-3, VH-4, VL-3 and VL-4 fragments from the corresponding phagemid template for the LRP antibody and then isolating the amplified fragments. As described above, Gibson assembly was then utilized to introduce the VH-3 and VL-4 fragments into the PpuMI/BamHI site of the Fc-knob plasmid described above. Gibson assembly was used to insert the other VH-4 and VL-3 fragments into the PpuMI/BamHI cut of the Fc-hole plasmid

Two plasmids (one pair, Fc-knob and Fc-hole) with differing VL and VH sequences were used to generate a FZD or co-receptor binding domain that was bispecific, i.e., capable of binding to two different sites. Because a knob-into-hole configuration was not needed to generate a dimer having monospecific binding domains, only a single plasmid containing the wild-type Fc sequence was used if each of the binding domains were to be monospecific.

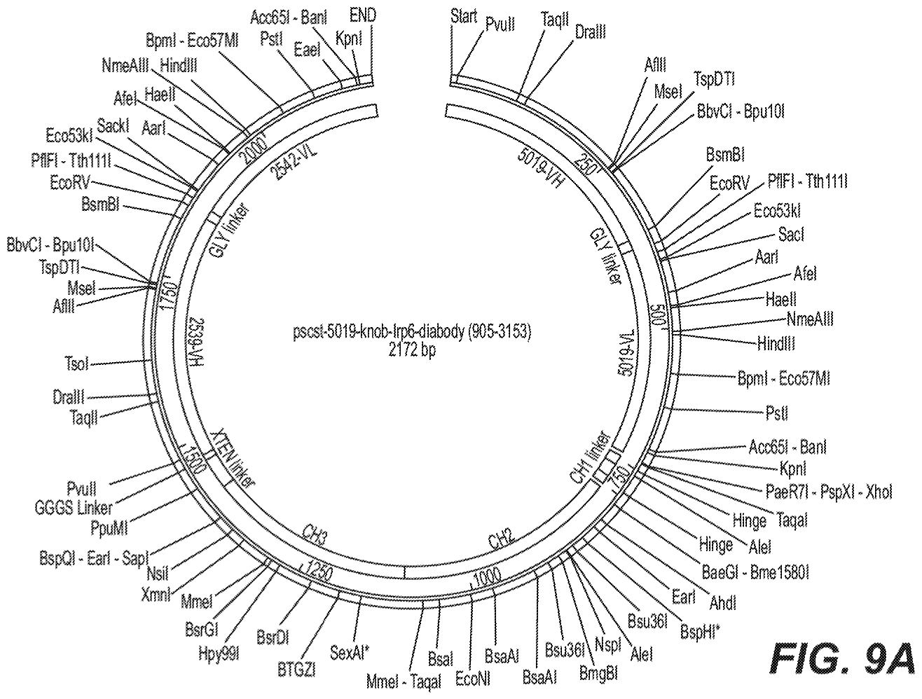

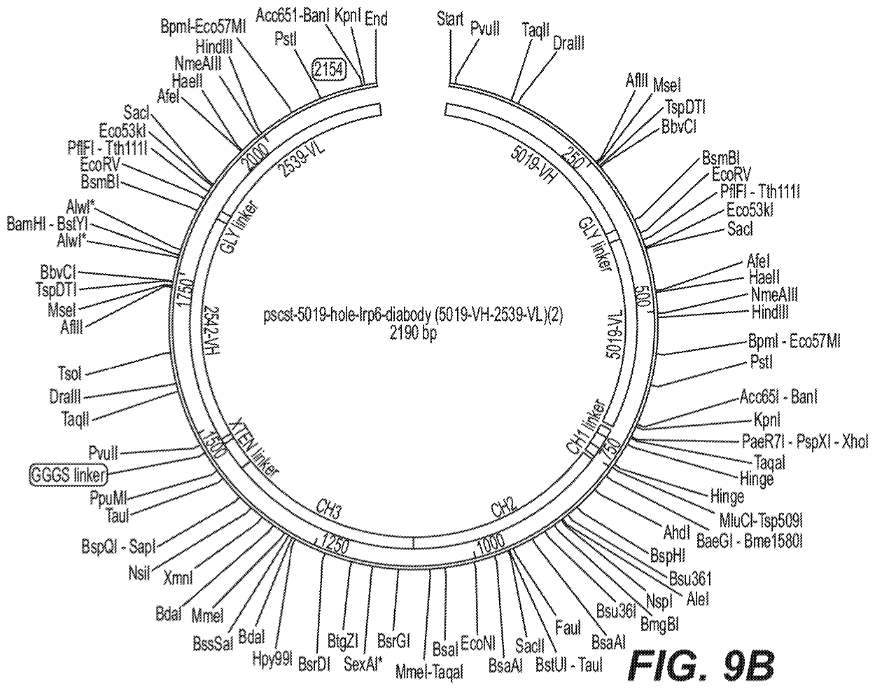

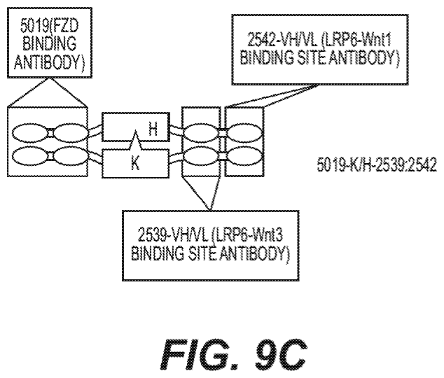

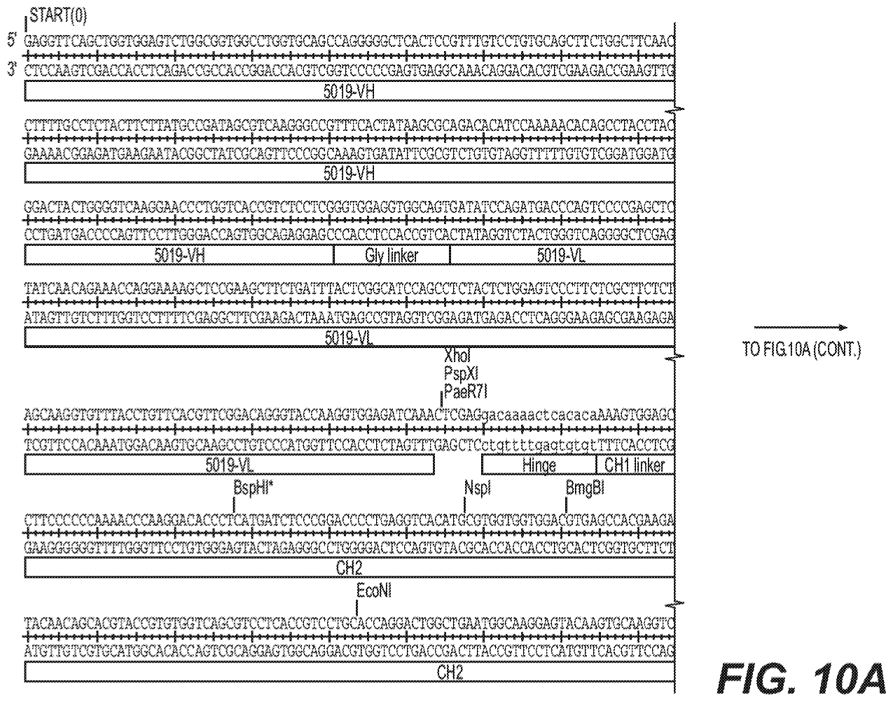

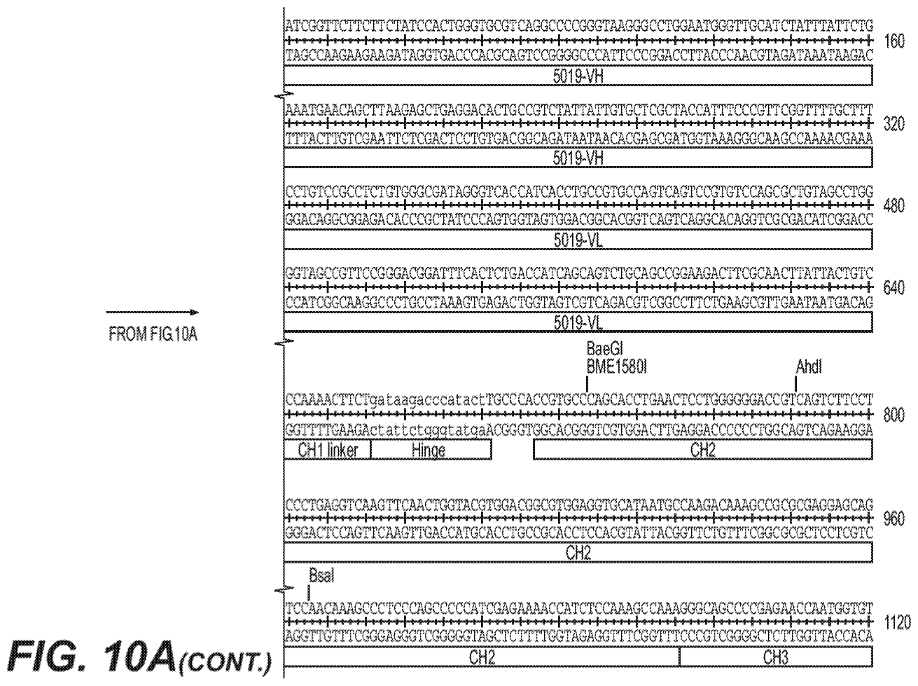

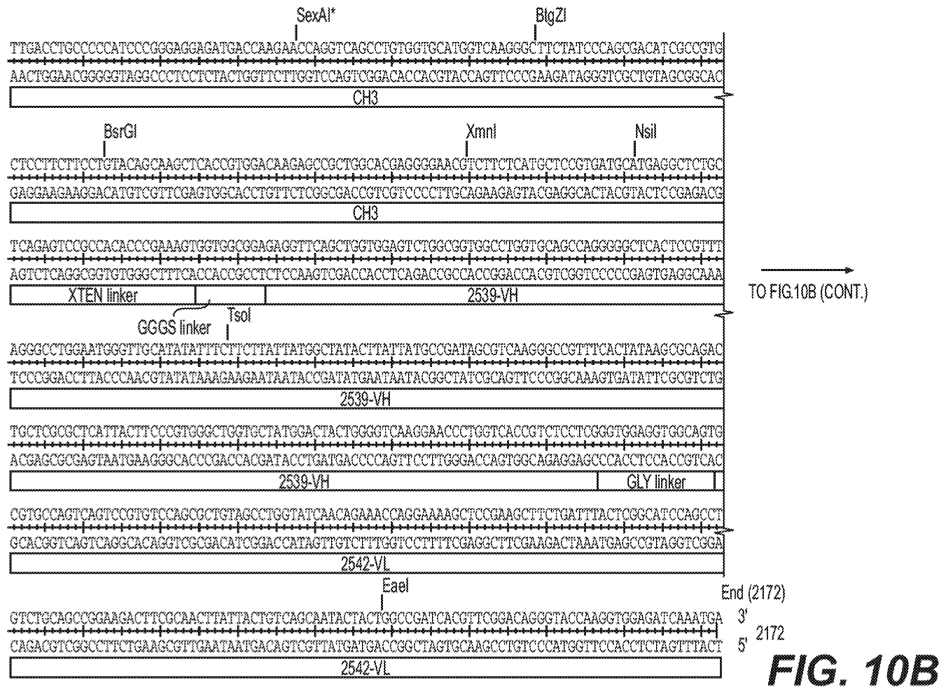

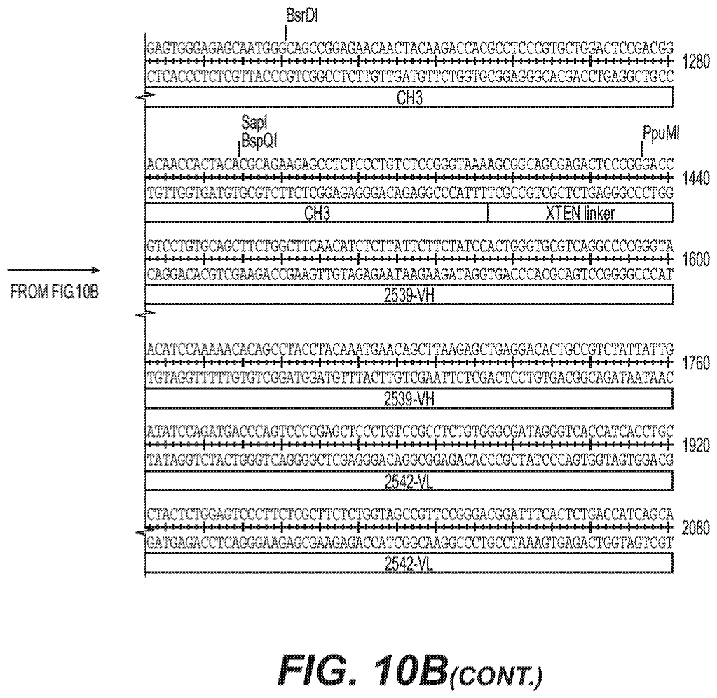

FIG. 9A depicts a plasmid encoding the peptide comprising an Fc region comprising a “knob” mutation, the VH and VL of panFZD antibody #5019, and the VL of LRP antibody #2542 and the VH of LRP antibody #2539. FIG. 9B depicts a plasmid encoding the peptide comprising a nucleic acid encoding Fc region comprising a “hole” mutation, the VH and VL of panFZD antibody #5019, and the VH of LRP antibody #2542 and the VL of LRP antibody #2539. The peptides encoded by these plasmids form a heterodimer having a multivalent binding site comprising a homodiabody derived from the pan specific FZD antibody #5019 and a multivalent binding site comprising a bispecific heterodiabody produced by the pairing of VL of LRP antibody #2539 and VH of LRP antibody #2542 from one peptide with the VH of LRP antibody #2539 and the VL of LRP antibody #2542 of the other peptide.

The resulting plasmids were then sequenced and the sequenced-verified plasmids were prepared using a PureLink HiPure Plasmid Filter Maxiprep Kit (Invitrogen) according to manufacturer's instructions. The plasmids were then transfected into Expi293F cells (Thermo Fisher Scientific) and FectoPRO Reagent (Polyplus) was used for antibody expression according to the manufactory's instructions. Typically, a scale of 200 ml cell was used for a small batch antibody production.

Typically, 80 h after transfection, the Expi 293F cell culture medium was harvested by centrifugation to pellet the cells and cellular debris. The supernatant was transferred to a clean bottle and buffered with 10×PBS buffer. After 1 h incubation with appropriate amount of Protein A beads (GE Healthcare), the beads were washed and the binding molecules were eluted according to the manufacturer instruction. Finally, the buffer was exchanged into PBS.

### 2. Heterodimeric Multivalent Binding Molecules

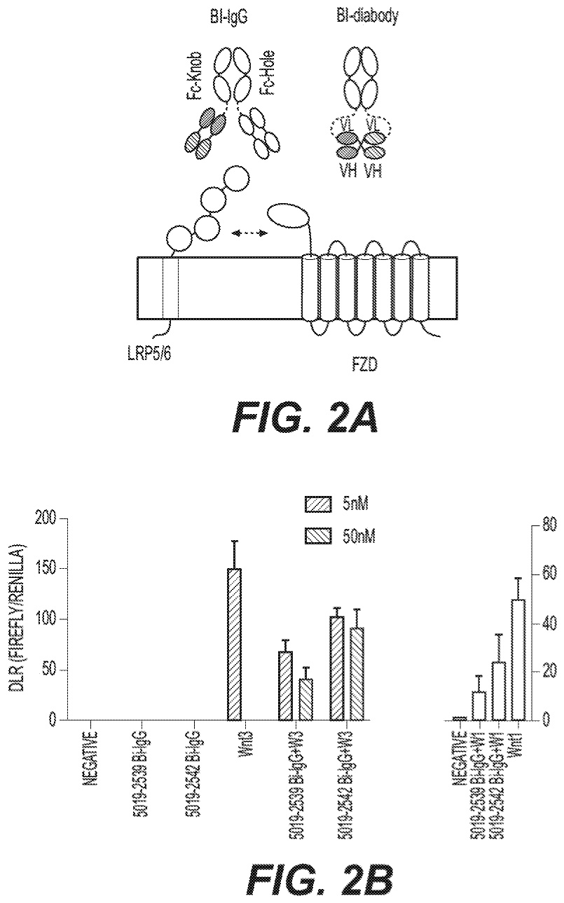

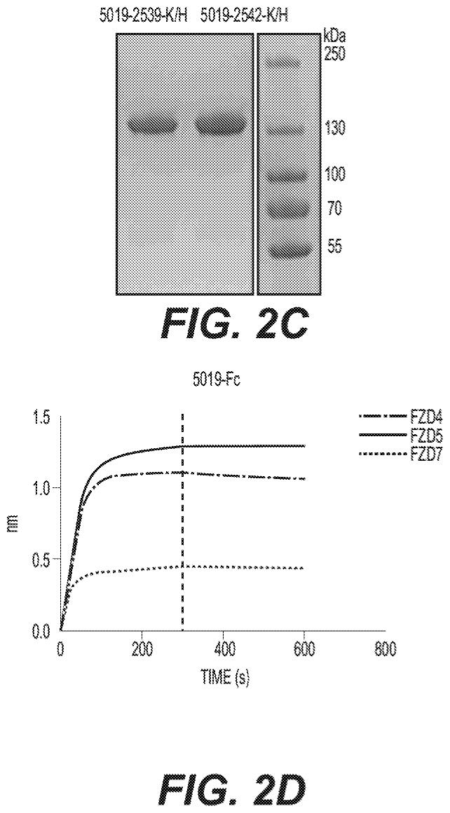

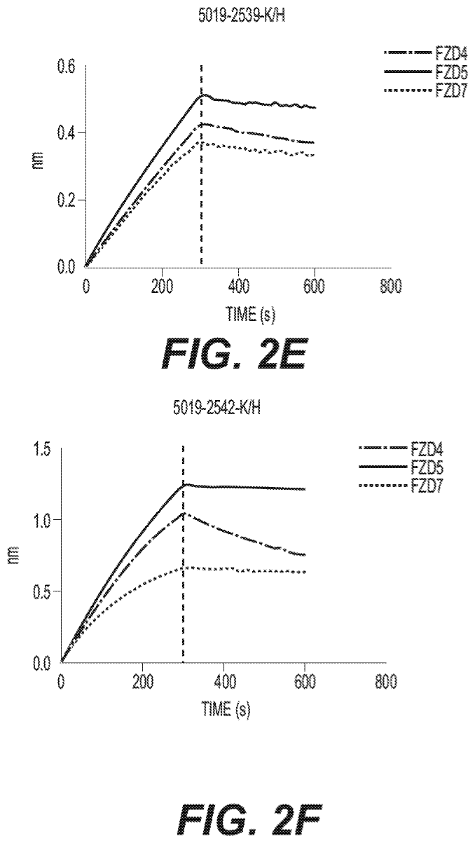

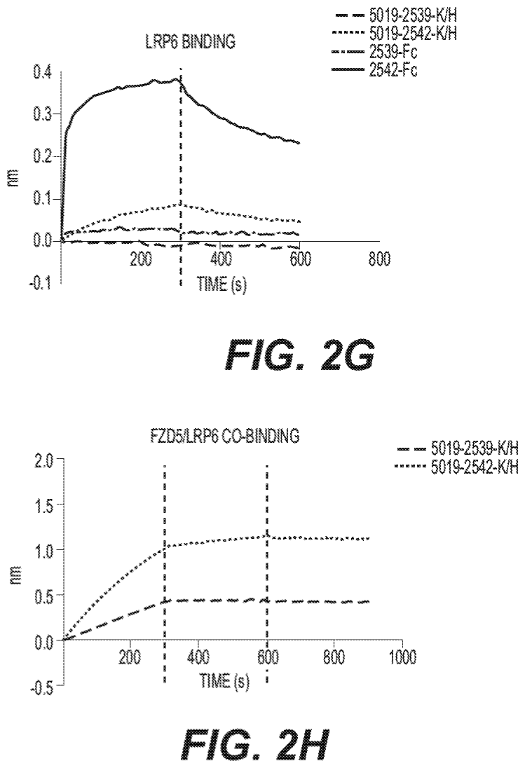

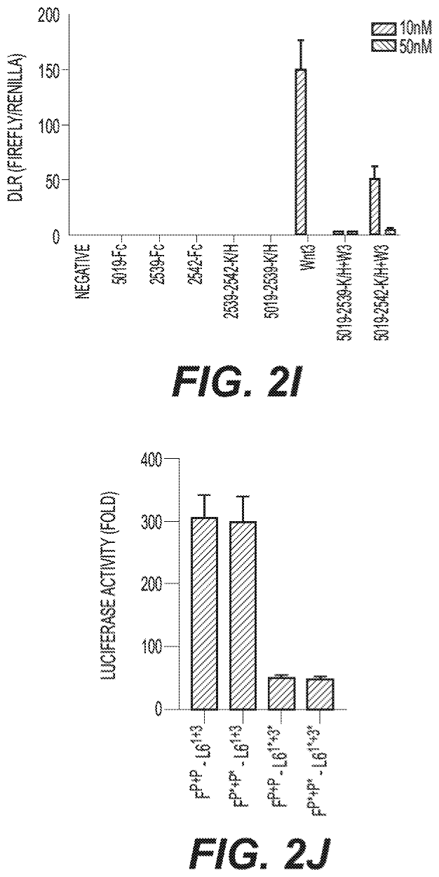

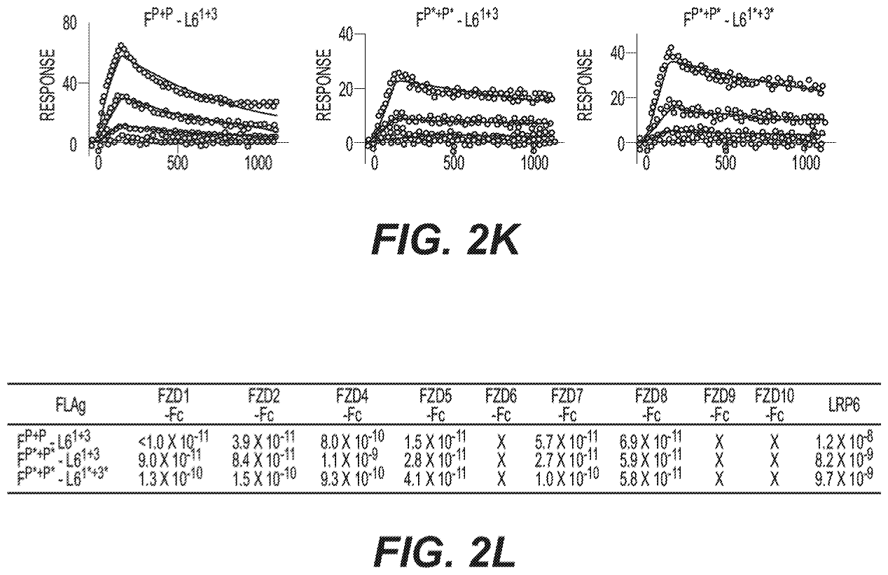

Using the methods described above, we also generated tetravalent heterodimeric molecules which contain intact bispecific diabodies fused to each of the N-terminus and C-terminus of Fc domain (Knob/Hole), (FIGS. 2A and 3A). In particular, we generated a tetravalent binding molecule having a FZD binding homodiabody derived from antibody 5019 on the N-terminus of the Fc domain and a homodiabody derived from LRP6-W1 antibody 2542 (5019-Fc-2542) or LRP6-W3 antibody 2539 (5019-Fc-2539) on the C-terminus of the Fc domain. Surprisingly, both tetravalent molecules activated the Wnt pathway, but 5019-Fc-2542 had much less efficacy (FIG. 3C). Without wishing to be bound by theory this difference may reflect differences in capacity of LRP6-W1 and LRP6-W3 binding to activate Wnt signaling. Wnt binding of the LRP6-W3 site has been observed to be more effective in activating Wnt signaling than Wnt binding to the LRP6-W1 site.

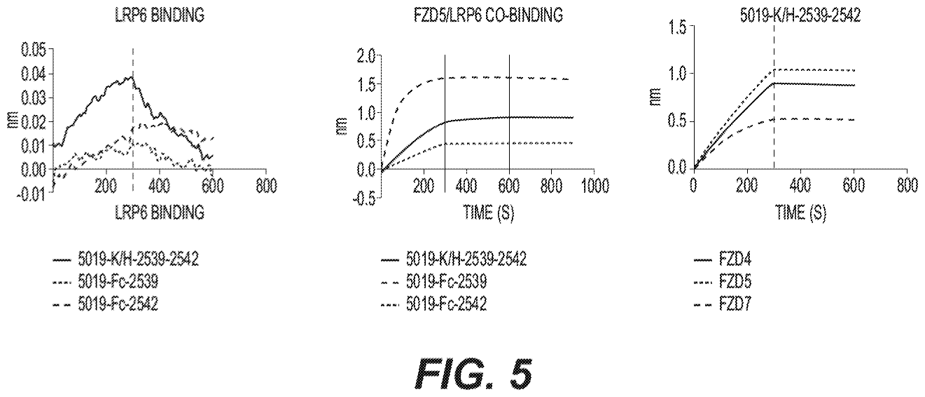

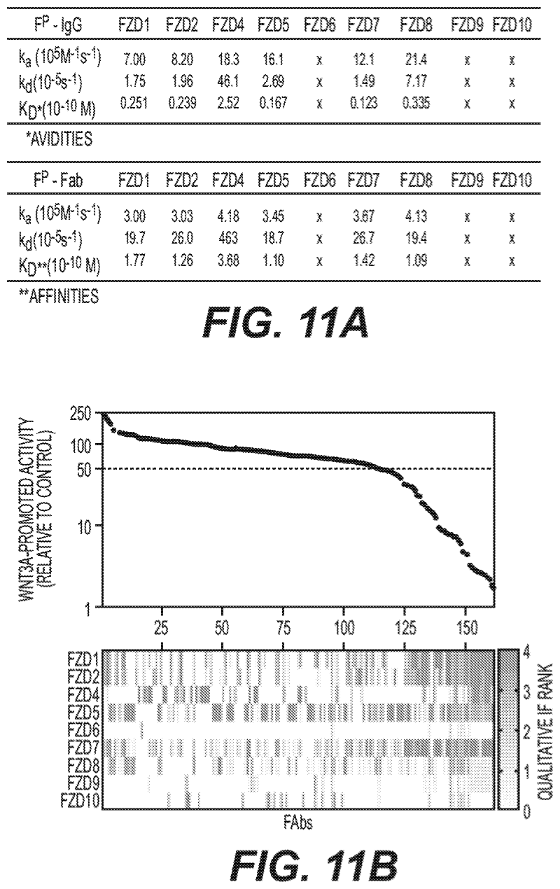

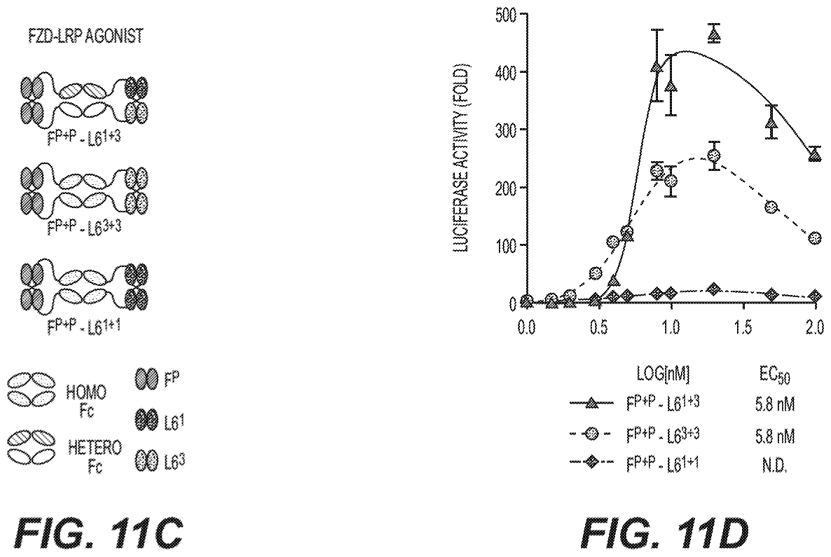

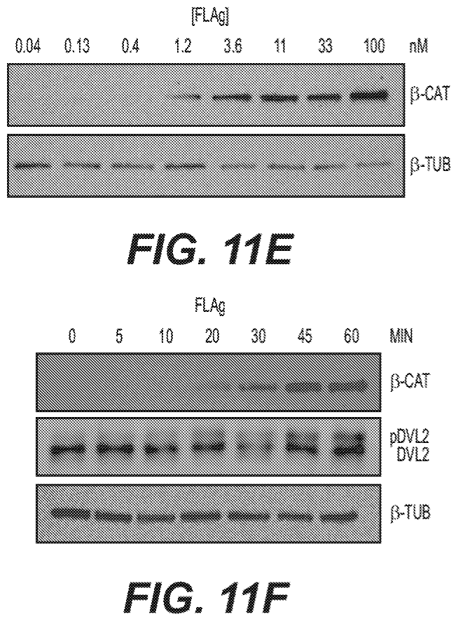

We also generated a tetravalent trispecific binding molecule having a FZD binding homodiabody derived from antibody 5019 on the N-terminus of the Fe domain and a LRP heterodiabody derived from LRP6-W1 antibody 2542 and LRP6-W3 antibody 2539 on the C-terminus of the Fe domain (5019-K/H-2539-2542, named as 5019Ag) (FIG. 5). 5019Ag is unexpectedly effective in activating Wnt signaling as compared to the molecules having a monospecific LRP6 homodiabody (FIG. 3C). Nanomolar amounts of all three forms activate Wnt signaling determined by pBAR luciferase reporter assays (FIG. 3D), indicating they are effective Wnt mimics. Without wishing to be bound by theory it is contemplated that engagement of a strong Wnt3A site and a weak Wnt1 site together is more effective than engagement of two strong Wnt3A sites. The two best multivalent binding molecules having an FZD binding domain and a LRP binding domain, “FLAgs”, had single-digit nanomolar potency (EC₅₀˜5 nM), which was virtually identical to the potency of purified Wnt3A, and displayed a bell-shaped dose response profile (FIG. 11D). We interpret this as indicating that maximal stimulation requires multivalent binding of the FLAg and that decreased efficacy at higher concentrations is likely attributable to monovalent binding to either FZD or LRP6. We treated RKO cells, which express low levels of βcatenin (Major et al. Science. 316, 1043-1046 (2007)), with F^{P+P}-L6¹⁺³ which caused dose- and time-dependent increases in βcatenin protein levels and phosphorylation of DVL2, a hallmark of Wnt-FZD pathway activation (FIG. 11E and FIG. 11F). Thus, tetravalent FLAgs are modular, engineerable, human Ab modalities that function as synthetic agonists of FZD and LRP6.

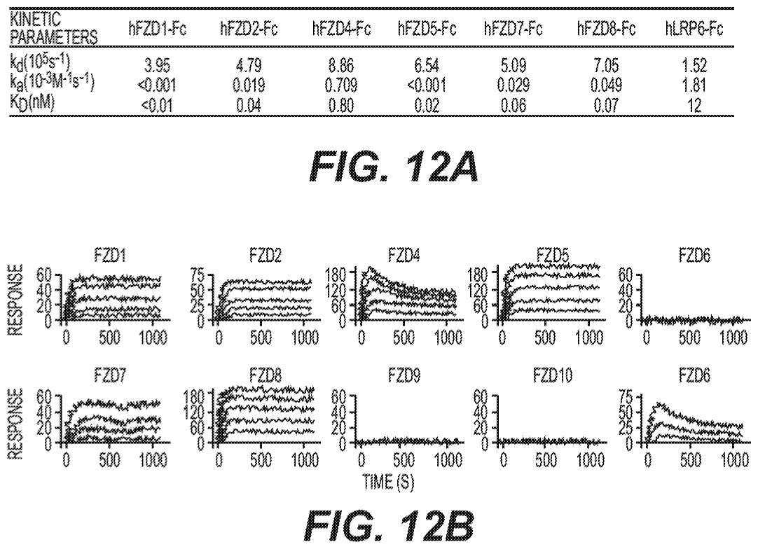

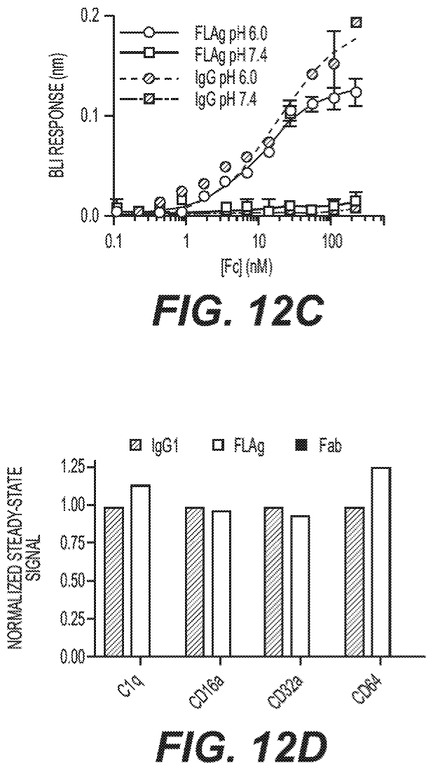

To confirm the engineered affinity and specificity of the optimal FLAg F^{P+P}-L6¹⁺³, we used Bio-Layer Interferometry (BLI) to measure its binding kinetics to nine of the 10 human FZD CRDs and to human LRP6 ECD (FIG. 12A and FIG. 12B). The FLAg bound with affinities in the picomolar range (KD=10-800 μM) to the six FZDs recognized by the FZD diabodies derived from the parent pan-FZD paratope (Pavlovic et al. 2018) but did not bind detectably to the other three FZDs. Moreover, affinity for LRP6 was in the nanomolar range (KD=12 nM) (FIG. 12B). We then used BLI to assess FLAg binding to various Fc receptors.

The FLAg behaved similarly to a conventional IgG and interacted with FcRn in a dose and pH dependent manner (FIG. 12C). Natural IgGs bind to FcRn at pH 6 but not at pH 7.4, and this enables recycling during pinocytosis and consequent long half-life in vivo. The FLAg also behaved similarly to the IgG for interaction with other Fc effectors including complement (C1q), the natural killer cell marker CD16a, the B cell marker CD32a, and the monocyte and macrophage marker CD64 (FIG. 12D. We conclude that the FLAg contains a functional Fc moiety that should confer effector functions and long half-life in vivo.

The modular design of the tetravalent F^{P+P}-L6¹⁺³ FLAg allowed us to dissect the contributions of each of the four paratopes to the intrinsic agonist activity by replacing each with a null paratope binding to the irrelevant antigen maltose-binding protein (MBP). We generated “mono-binding” molecules comprising an Fc domain and an FZD binding domain attached to one Fc domain terminus and LRP binding domain attached the other Fc domain terminus, but rather than having two binding sites for FZD or LRP within the diabodies, the binding domains have only a single or mono binding site, and one control maltose-binding-protein binding site, “MBP”. One MBP binding site was introduced into at least one binding domain of the molecules to generate five mono binding molecules. The 5019-MBP-K/H-2539-2542, which contains one FZD and one MBP binding site in the N-terminus, still activates the Wnt pathway, but has an 8-fold decrease in efficacy as compared to 5019Ag (FIG. 3E). Similarly, the 5019-K/H-2539-MBP, which retains only one LRP6-W3 site in the C-terminus, exhibits much less Wnt activation as compared to 5019Ag (FIG. 3E). Minimal agonistic activity was detected for the two MBP-FZD/MBP-LRP6 molecules 5019-MBP-K/H-2539-MBP and 5019-MBP-K/H-MBP-2542 and the molecules having one LRP6-W1 diabody, 5019-K/H-MBP-2542 (FIG. 3E). The results of these βcatenin signaling assays showed that maximal stimulation was reduced significantly by disabling one anti-FZD paratope or the anti-LRP6 paratope for the WNT1 binding site and was completely ablated by disabling the anti-LRP6 paratope for the WNT3A binding site or by simultaneously disabling one anti-FZD paratope and either of the anti-LRP6 paratopes. We also substituted an anti-LRP5 paratope targeting the WNT3A binding site for the anti-LRP6 paratope targeting the WNT1 binding site to generate a molecule (F^{P+P}-L5/6³) that could recruit both co-receptors and observed activity similar to that of F^{P+P}-L6¹⁺³(FIG. 3F, EC₅₀=4 nM). Taken together, these data showed that optimal agonist activity is achieved with a molecule capable of recruiting two FZDs through a common epitope and LRP6 through two distinct epitopes, but activity can be modulated to intermediate levels by disabling one of the anti-FZD or anti-LRP6 paratopes. Moreover, molecules that could recruit FZD and two different co-receptors were generated by combining two anti-FZD paratopes with one paratope each for LRP5 and LRP6.

We also explored the requirements for geometric and spatial constraints imposed by the intermolecular diabody format by substituting diabody pairs with pairs of less constrained intramolecular single-chain variable fragments (scFvs) (FIG. 2J). Compared with F^{P+P}-L6¹⁺³, a FLAg that contained anti-FZD scFvs (F^{P*+P*}-L6¹⁺³) exhibited similar activity, whereas activity was significantly reduced for FLAgs that contained anti-LRP6 scFvs (F^{P+P}-L6^{1*+3*}) or scFvs at both ends (F^{P*+P*}-L₆^{1*+3*}). These differences in activity were not due to differences in affinity, as BLI measurements showed comparable, high-affinity binding to LRP6 and FZD isoforms regardless of whether paratopes were presented in the diabody or scFv format (FIG. 2K and FIG. 2L). Taken together, these results showed that particular stoichiometries and geometries are required for the assembly of optimal FZD/LRP6 signaling complexes, and constraints are especially precise for LRP6, which requires engagement of two distinct epitopes in a specific geometry dictated by the diabody format. Notably, the looser constraints for FZD engagement enabled significant activation with a single anti-FZD paratope (FIG. 2D), which opens the door for further enhancing specificity or altering signaling by recruiting a different cell surface protein through an additional paratope in conjunction with an anti-FZD paratope at the N-termini of the heterodimeric Fc.

### 3. Other Bispecific Antibody Forms

Bispecific molecules comprising a FZD binding domain of antibody #5019 and LRP6-W1 binding domain of antibody #2942 (5019/2942) or LRP6-W3 binding domain of antibody #2539 (5019/2539) on the same terminus of an Fc domain were constructed and the corresponding proteins were purified (FIG. 2A) and assayed for activation of Wnt signaling using pBAR luciferase reporter assays. These molecules failed to activate Wnt signaling. Notably, both bispecific molecules antagonized the activity of the Wnt ligand (FIG. 2B). Without wishing to be bound by theory, the distance and flexibility between the two paratopes of these bispecific molecules might not recruit the FZD and LRP6 receptor in a suitable geometry for activation.

Bispecific molecules comprising a FZD diabody and an LRP diabody attached to the same terminus of an Fc domain were also generated using a knob in hole configuration. These diabodies designated 5019-2539-K/H (FZD/LRP-W3) and 5019-2542-K/H (FZD/LRP-W1) were assayed for FZD and LRP binding and Wnt pathway activation. Both diabodies retained the FZD binding profile of the original antibody as well as the LRP6 binding activity (FIGS. 2D-2G). Both molecules bound individually to the FZD receptor and the LRP co-receptor. 5019-2542-K/H displayed co-binding to both FZD and LRP in solution as determined with BLI assays (FIG. 2H) but no significant co-binding was observed with 5019-2539-K/H. Neither 5019-2539-K/H nor 5019-2542-K/H activated Wnt signaling as determined in pBAR luciferase reporter assays, similar to the results obtained with the homo-diabodies that bound to only a FZD receptor (5019-Fc) or co-receptor (2539-Fc) (FIG. 2I). Moreover, both 5019-2539-K/H (FZD/LRP-W3) and 5019-2542-K/H (FZD/LRP-W1) effectively inhibited Wnt3a mediated pathway activation (FIG. 2I).

### 4. Wnt Pathway Signaling Assay

Wnt pathway activation was assayed in HEK293 cells using the pBAR luciferase reporter system that faithfully monitor the transcriptional activation of βcatenin (Biechele and Moon, Methods Mol Biol. 2008; 468:99-110, PMID: 19099249). Briefly, HEK293T cells stably expressing pBARLS and pSL9 Ef1α-Renilla Luciferase constructs were seeded in 96-well plates at 1.5E4 cells/well. 24 hours following seeding, cells were treated with the indicated FZD agonists in triplicate at indicated concentrations or PBS vehicle control. 16.5 hours after treatment cells were lysed and luminescence was measured using Dual-Luciferase Reporter Assay System (Promega #E1960), according to manufacturer's protocol. Firefly luminescence was normalized to Renilla luminescence for each well, to control for cell number.

We assayed the agonist activity of multivalent molecules containing an N-terminal FZD diabody derived from an antibody fragment (Antibody #5019) that recognized several of the FZD receptors (FZD 1, 2, 4, 5, 7, and 8) joined via the Fc domain to an LRP binding domain on the C-terminus of the Fc domain. The C-terminal LRP binding domains comprised a diabody derived from one of two LRP6 antibodies, #2539 and #2542, which bind to the Wnt3 site and Wnt1 site respectively (FIG. 6B). Nanomolar amounts of these multivalent binding molecules, denoted 5019-Fc-2539 and 5019-Fc-2542 activated the Wnt-βcatenin pathway (FIG. 6C), however, treatment of cells with the molecule harboring the LRP6 antibody targeting the Wnt3 site, 5019-Fc-2539, led to an approximately 10 folds higher activation when compared to 5019-Fc-2542 (200 folds vs 20 folds over background respectively) (FIG. 6C).

Importantly, using a knob-hole system engineered within the Fc moiety we generated multivalent binding molecules (FIG. 1C) that contained a homodiabody for the pan-FZD binding domain on one end (#5019) and an heterodiabody forming LRP6 binding domain with binding sites for Wnt1 (#2542) and Wnt3 (#2539) 5019-K/H-2539:2542 on the other end (FIG. 6B). This configuration enabled the incorporation of 4 different binding sites within the molecule with different selectivity and affinity profiles, i.e., tetravalent and trispecific. When tested in the 8-catenin luciferase reporter assay in HEK293 cells, this molecule has a 2 folds higher activation than 5019-Fc-2539 or approximately 400 folds over background (FIG. 6C).

We also substituted the binding sites for LRP6 for equivalent LRP5 binding sites (diabodies derived from 2459 and 2460 antibodies which both bind LRP5) within the knob-in-hole system along with the same pan-FZD diabody that binds FZD1, 2, 4, 5, 7, 8 (5019). This molecule 5019-K/H-2459:2460 was also able to activate the Wnt-βcatenin pathway in HEK293T cells (FIG. 6D), albeit with lower efficacy than the agonist harboring the LRP6 diabodies.

5. Characterization of Selective FZD Agonists (Agonist Modularity with Binding Domains Derived from Selective FZD and Co-Receptor Antibody Fragments)

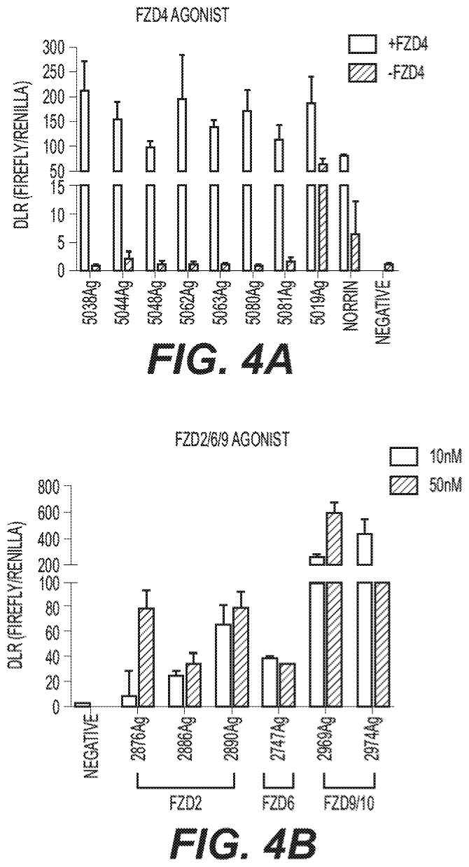

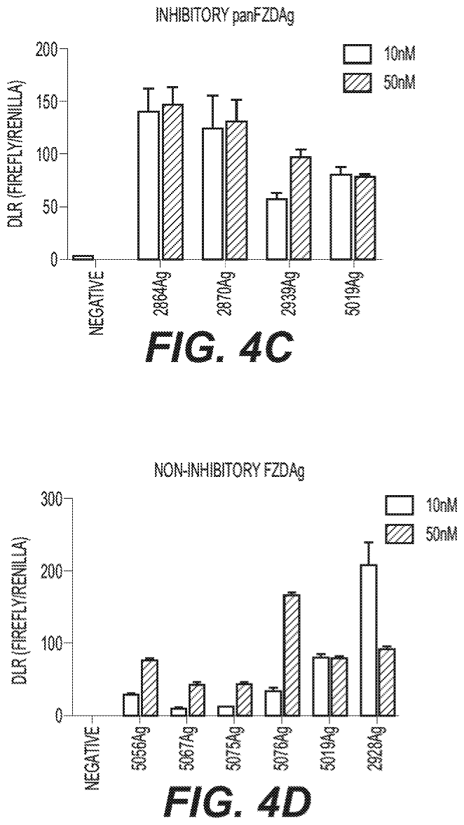

To assess activities of our monospecific FZD agonists, we used cell-based assays that depend on particular FZD isforms. We prepared multivalent binding molecules that only bound to one of the ten FZD receptors. Our previous work identified several antibodies that are completely specific for FZD4 (5038, 5044, 5048, 5062, 5063, 5080, 5081) (see, e.g., US20160194394, inventors Sidhu et al. and WO2017127933A1, inventors Pan et al.). Multivalent binding molecules comprised a FZD binding domain that was FZD4 specific and an LRP6 binding domain comprising a bispecific heterodiabody derived from antibodies 2539 and 2542 were generated using the Fc knob-in-hole system. These molecules could activate FZD4 signaling through the β-catenin pathway but only when co-transfected into HEK293 cells along with FZD4 cDNA. These FZD4 binding molecules could not activate FZD4 signaling or the β-catenin pathway in non-modified HEK293T cells, which express low levels of FZD4. Thus, this experiment demonstrates the specificity of the molecules for FZD4. 5019-K/H-2539-2542 (the pan-FZD agonist described above) can activate signaling in HEK293T cells even in the absence of FZD4 (FIG. 4A). This result is not surprising as Wnt-mediated activation of β-catenin signaling HEK293T cells occurs through FZD1, 2 and 7 (Voloshanenko et al. FASEB 2017 FASEB J. 2017 November; 31(11):4832-4844; PMID: 28733458) and the 5919 FZD antibody binds to all three receptors.

In addition, we generated a FZD5 specific multivalent binding molecule using the binding domain of the FZD5 specific antibody 2928, which we previously characterized to bind only to FZD5 (Steinhart et al. Nat Med. 2017 January; 23(1):60-68, PMID: 27869803; WO2017127933A1, inventors Pan et al.). We previously demonstrated that several RNF43 mutant pancreatic ductal adenocarcinoma (PDAC) cell lines are dependent only on FZD5 signaling for their proliferation (Steinhart et al. 2017, PMID: 27869803). Indeed, genome-wide CRISPR essentiality/fitness screens in three RNF43 mutant PDAC lines showed that FZD5 was one of the most essential genes for their growth whereas PDAC cell lines with WT RNF43 did not exhibit this requirement for FZD5. When RNF43 mutant cells are treated with a Porcupine inhibitor (PORCNi; such as LGK-974) that inhibits the palmitoylation and activity of Wnt ligands, RNF43 mutant cells stop proliferating.

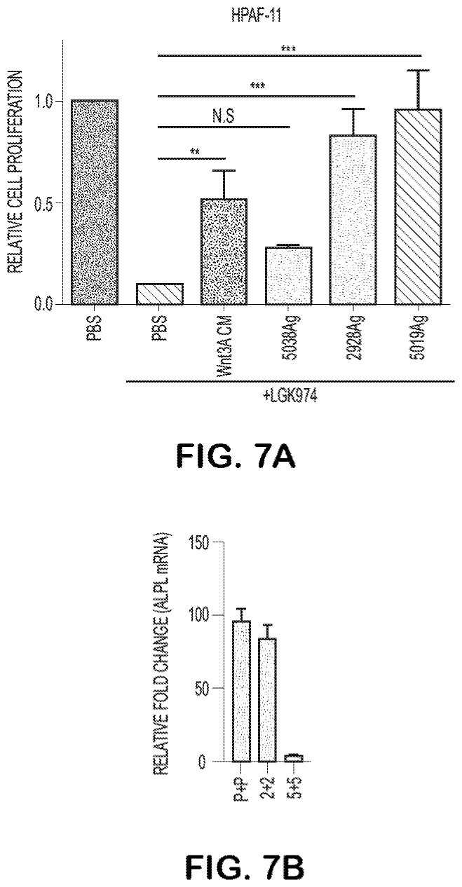

Co-treatment of RNF43 mutant cells with the pan-FZDag 5019-K/H-2539-2542 or with the selective FZD5 agonist 2928-K/H-2539-2542 led to robust rescue of cell proliferation blocked by LGK974. These results demonstrate that these two molecules were capable of activating FZD5 and induced Wnt signaling in these cells, thereby mimicking the action of endogenous Wnt ligands (FIG. 7B). In contrast, addition of the FZD4 specific agonist 5038-K/H-2539-2542 or a FZD2 specific agonist were unable to rescue the inhibition of proliferation mediated by LGK974.

RNAseq analysis has shown that FZD2 is the predominant isoform in the mesenchymal stem cell line CH3H10T1/2 (Mouse ENCODE), suggesting that FZD2 may be responsible for the established role of Wnt proteins during osteogenic differentiation of mesenchymal cells (Day et al. Dev. Cell. 8, 739-750 (2005)). Stimulation of C3H10T1/2 cells with a FZD2-specific FLAg led to robust induction of the osteogenic marker alkaline phosphatase (ALPL) to levels similar to those achieved with a Pan-FZD FLAg, whereas a FZD5-specific FLAg exhibited minimal activity (FIG. 7B).

6. Co-Targeting with the Tetravalent Binding Molecules

In addition to mixing and matching FZD multivalent binding domains and co-receptor binding domains with an Fc domain to achieve desired combinations, the existence of tetravalent paratopes in the current system provides an opportunity for targeting two FZD receptors and two co-receptors simultaneously with one molecule, ensuring the co-localization when applying in vivo. Considering the agonistic activity of 5019-MBP-K/H-2539:2542 shown above, the generation of a multivalent binding molecule having binding domains for selective FZD receptors by combining the binding regions within an heterodiabody at the N-terminus of the Fc domain. For example, the binding domains derived from antibody 5038 (binds FZD4) and 2928 (binds FZD5) would yield a FZD4 and FZD5 co-targeting molecule. The binding molecules can also be generated to have a co-receptor binding domain for specific or multiple co-receptors. For example, an LRP6/LRP5 co-targeting binding domain could be produced by combining the binding domains derived from the 2459 (binds Wnt1 binding site of LRP6) and 2539 (binds Wnt3a binding site of LRP6) antibodies on the C-terminal of the Fc domain. Likewise, the co-receptor binding domain may comprise a binding site for an LRP6 in combination with another co-receptor, e.g., ROR1/2, to initiate activation of both canonical and non-canonical Wnt signaling pathways in a single cell.

Also contemplated herein is a multivalent binding molecule having a tissue specific binding domain derived from a tissue specific antibody which would recruit the multivalent binding molecule to a desired tissue where it would then activate Wnt signaling by binding a FZD receptor and co-receptor. This is contemplated to be particularly useful when using the multivalent binding molecules in regenerative therapeutics when desired effects may need to be restricted to a specific tissue. To summarize, the tetravalent mode allows more designing flexibility to meet versatile functional requirements.

### 7. Multivalent Binding Molecules Having a FZD Binding Domain and Co-Receptor Binding Domain can Replace Wnt Ligands to Sustain Intestinal Organoid Cultures.

The effect of the FZD agonists described herein on organoid survival and maintenance was assayed as follows. An 8-week old female C57BL/6 mouse was sacrificed, and small intestine crypts were harvested for organoid isolation (O'Rourke et al. 2016. Isolation, Culture, and Maintenance of Mouse Intestinal Stem Cells. Bio Protoc. 20:4). Organoid cultures were passed by mechanical dissociation (O'Rourke 2016) and embedded in 25 μl of Growth Factor Reduced Matrigel (Corning, 356231) in a 48 well plate. Organoids were plated in triplicates for each experimental condition. Complete organoid media (O'Rourke 2016) with experimental conditions (1 μM LGK-974+/−40% Wnt3a conditioned media or +/−50 nM panFzd-5056 (a FZDag targeting FZD1, 2, 4, 6, 7, 8 but binding to an epitope that does not compete with Wnt ligands)) was added to each well on day of passaging and changed every 2-3 days. After one week, 150 μl of Cell Titer Glo 3D (Promega) was added to 150 μl of media in each well. Organoids were lysed on a rocking platform for 30 min at RT. The luminescence reading was measured in duplicates for 20 μl of lysate from each well. The average luminescence reading for each condition was normalized to the DMSO condition to calculate viability.

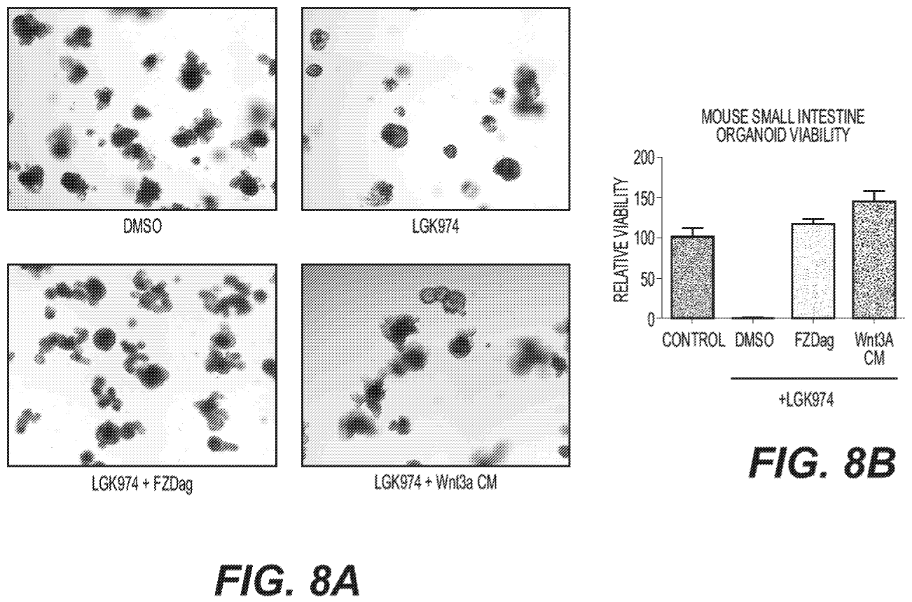

Being pervasive stem cell niche factors, Wnts and R-spondins are required for the derivatization and maintenance of three-dimensional culture organoids from many tissues. In vitro, Wnt proteins secreted by paneth cells are sufficient to support the growth of mouse small intestine organoids in the presence of R-spondins. However, if Wnt release and activity is blocked with the PORCNi LGK974, the organoids can't proliferate and eventually die. Herein we demonstrate that a pan-FZD multivalent binding molecule of this invention, FZDag (F^{P+P}-L6¹⁺³) can rescue and sustain the growth of organoids in the presence of LGK974, suggesting that the molecule functionally mimics Wnt ligands (FIG. 8) and can substitute for Wnt proteins to support growth of tissue organoids. Because Wnt ligands are integral components of the media required to grow many human tissue organoids, the antibody-derived FZD agonists of this invention are expected to promote the derivatization, survival and maintenance of organoids of different tissues when included in the culture media and thereby alleviate limitations associated with the use of conditioned media or purified Wnt proteins.

### 8. Multivalent Binding Molecules Promote Bone Regeneration

A rat closed femoral fracture model is used to evaluate the regenerative properties of multivalent binding molecules of this invention having a first multivalent binding domain that binds FZD2, and a co-receptor binding domain that binds to LRP5 or LRP 6. The first multivalent binding domain may specifically bind FZD2, e.g., the binding domains of 2890-hole-2539-2542 and 2890-knob-2539-2542 (e.g. encoded by SEQ ID NO: 84 and 85) or may bind FZD2 and other FZD receptors.

Rats are administered vehicle or the multivalent binding molecule following unilateral closed femoral mid-diaphyseal fractures (see Bonnarens, and Einhorn, J. Orthop. Res. 2, 97-101 (1984)). Briefly, an 18-gauge syringe needle is inserted into the medullary canal through the condyles. A transverse fracture of the femur is then created via blunt impact loading at the anterior (lateral) aspect of the thigh. One day after the fracture, rats are injected subcutaneously with either saline vehicle or multivalent binding molecules twice per week for 7 weeks. At termination, the intramedullary pins are removed and the fractured femurs will be analyzed by microCT.

The multivalent binding molecules having a multivalent domain that binds FZD2, and a second multivalent binding domain that binds to LRP5 or LRP6 significantly increases regeneration of bone in this model in comparison to bone regeneration by the vehicle alone.

### Example II—Synthetic Antibodies Targeting FZD and LRP6

We previously applied phage display to derive hundreds of synthetic Abs using nine recombinant FZD CRDs as antigens (FZD3 CRD could not be purified) (Steinhart et al. Nat. Med. 23, 60 (2016); Pavlovic et al. MAbs (2018), doi: 10.1080/19420862.2018. 1515565). Systematic characterization revealed a continuum of specificity profiles with some Abs displaying broad specificities, exemplified by a pan-FZD Ab (FP) that recognized FZD1/2/4/5/7/8 (FIG. 11A), others displaying more restricted specificities, and some being monospecific (FIG. 11B). Functional characterization revealed that some antibodies competed with Wnt and inhibited βcatenin signaling, whereas others were non-competitive and did not interfere with Wnt signaling (FIG. 11B). In total, we fully characterized 161 anti-FZD antibodies, including 47 inhibitors of Wnt signaling. Unexpectedly as discussed herein, regardless of whether or not they competed with Wnt and inhibited Wnt signaling, all the multivalent binding molecules that we generated by using these anti-FZD antibodies as the source of the FZD binding domains in conjunction with an LRP binding domain, e.g., a binding domain that bound to Wnt1 and/or Wnt3a binding sites on LRP 5/6, were agonists of the Wnt pathways.

### Example III—Phenotypic Effects of FLAgs in Cells, Organoids and Animals

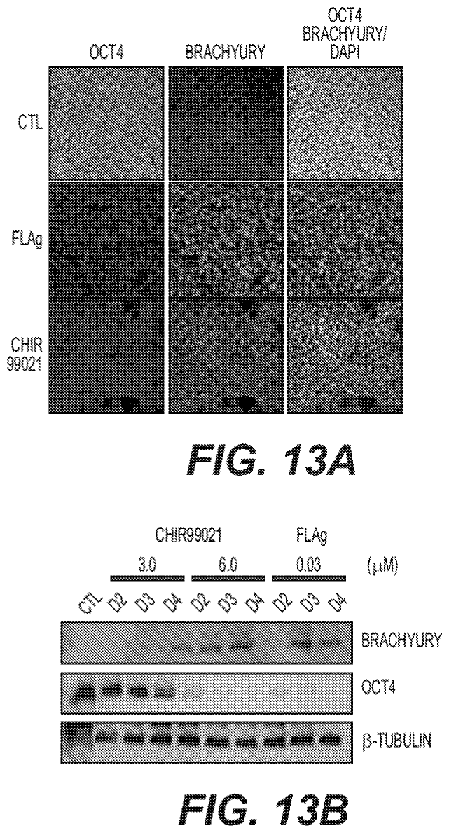

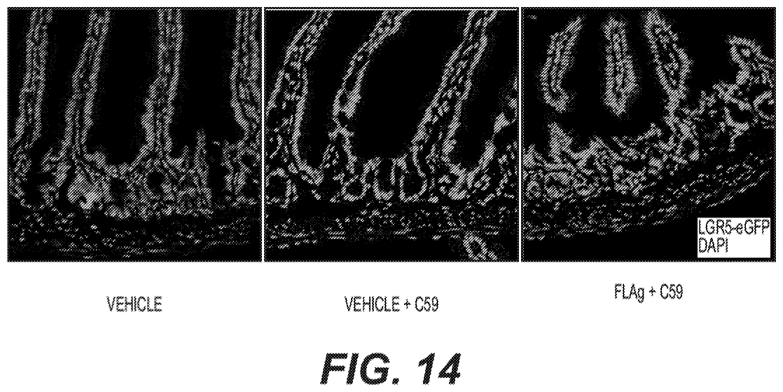

Having established that FLAgs selectively engage FZD and LRP to activate Wnt-associated signaling pathways, we explored the phenotypic effects of these signals in progenitor stem cells (PSCs), organoids and animals. Modulation of Wnt-βcatenin signaling activity is integral to most PSCs differentiation protocols (Huggins et al. Methods Mol. Biol. 1481, 161-181(2016)). Treatment of human PSCs with WNT3A conditioned media, or small molecule inhibitors of GSK3, activates βcatenin signaling, leads to primitive streak induction, and promotes mesodermal fate specification (Davidson et al. PNAS U.S.A. 109, 4485-4490 (2012)). We evaluated FLAg activity in this context and found that treatment of human PSCs with 30 nM F^{P+P}-L6¹⁺³ for three days caused robust induction of the mesoderm marker BRACHYURY and decreased expression of the pluripotency marker OCT4 to levels comparable to treatment with the GSK3 inhibitor CHIR99021 at 6 μM (FIG. 13A and FIG. 13B).

F^{P+P}-L6¹⁺³ recognizes mouse FZDs and LRP6, and it contains an Fc that interacts with the FcRn. It is contemplated that the Fc endows the molecule with a long, Ab-like, half-life in vivo. Thus, we tested whether F^{P+P}-L6¹⁺³ could interact with endogenous receptors in mice and accumulate to levels that would be sufficient to activate βcatenin signaling and mobilize endogenous stem cell activity. Within the intestinal stem cell niche, Wnt proteins secreted by mesenchymal cells induce expression of βcatenin target genes in stem cells at the bottom of the crypt to direct their self-renewal, and the target gene LGR5 is frequently used as a marker of stem cells in various tissues. Treatment of LGR5-GFP mice with LGK974 ablated Wnt production and caused rapid extinction of LGR5 expression and the linked GFP signal in crypt stem cells. Strikingly, GFP expression was rescued upon co-treatment with F^{P+P}-L6¹⁺³ by intraperitoneal injection (FIG. 14 right panel. We conclude that F^{P+P}-L6¹⁺³ has a sufficient half-life and bioavailability to enable βcatenin activation at levels that promote self-renewal of intestinal stem cells in the absence of endogenous Wnt.

### Example IV—Materials and Methods

1. Ab Selections and Screens

The phage-displayed synthetic library F was used to select for Fabs that bound to Wnt receptors, as described (Persson et al. J. Mol. Biol. 425, 803-811 (2013)). Briefly, Fc-tagged ECD protein (R&D Systems) was immobilized on Maxisorp immunoplates (ThermoFisher, catalog number 12-565-135) and used for positive binding selections with library phage pools that were first exposed to similarly immobilized Fc protein to deplete non-specific binders. After 4 rounds of binding selections, clonal phage were prepared and evaluated by phage ELISA (Birtalan et al. J. Mol. Biol. 377, 1518-1528 (2008)). Clones that displayed at least 10-fold greater signal for binding to antigen compared with Fc were considered to be specific binders that were subjected to further characterization.

2. Recombinant Proteins and Reagents

Fc-tagged fusions of FZD1 (5988-FZ-050), FZD2 (1307-FZ-050), FZD4 (5847-FZ-050), FZD5 (1617-FZ-050), FZD7 (6178-FZ-050), FZD8 (6129-FZ-050), FZD9 (9175-FZ-050), FZD10 (3459-FZ-050) were purchased from R&D Systems. The Fc-tagged ECD of FZD6 (residues 19-132, UniprotO60353-1) was expressed and purified from Expi293 cells using the pFUSE-hIgG1-Fc2 vector (Invivogen) and the single protomer species was separated from aggregated protein by size exclusion chromatography on a Superdex 200 (10/300) column (GE Healthcare). Fc-tagged ECD fusion proteins of human (1505-LR-025) and mouse (2960-LR-025) LRP6 and mouse LRP5 (7344-LR-025/CF) were purchased from R&D Systems. WNT1 (SRP4754-10 ug), WNT2b (3900-WN-010/CF), WNT5a (645-WN-010/CF) and WNT3A (5036-WN-010/CF) were purchased from R&D Systems, and WNT3A conditioned media was prepared as described (PMID:12717451). Other proteins and chemicals were purchased from the following suppliers: FcRN (R&D, 8693-FC), C1q (Sigma, C1740), CD16a (R&D, 4325-FC), CD32a (R&D, 1330-CD/CF), CD64 (R&D, 1257-FC), LGK974 (Cayman Chemicals), the Porcupine Inhibitor C59 (Dalriada Therapeutics), and CHIR99021 (Sigma Aldrich).

3. Tetravalent Binding Molecules for FZD and LRP, “FLAgs”, and Antibody Cloning

DNA fragments encoding antibody (Ab) variable domains were either amplified by the PCR from phagemid DNA template or were constructed by chemical synthesis (Twist Biosciences). The DNA fragments were cloned into mammalian expression vectors (pSCSTa) designed for production of kappa light chains and human IgG1 heavy chains. Bispecific diabodies and IgGs contained an optimized version of a “knobs-in-holes” heterodimeric Fc (Ridgway et al. Protein Eng. 9, 617-621 (1996)). FLAgs and diabody-Fc fusions were arranged a VH-VL orientation with the variable domains separated by a short GGGGS (e.g. amino acids 121-125 of SEQ ID NO: 2) linker, which favors intermolecular association between VH and VL domains and thus favors diabody formation. To produce diabody-Fc fusion constructs, diabody chains were fused to human IgG1 Fc. FLAg proteins were constructed as VH-x-VL-y-[human IgG1 Fc]-z-VH-x-VL where linkers are x=GGGGS (e.g. amino acids 121-125 of SEQ ID NO: 2), y=LEDKTHTKVEPKSS (amino acids 232 to 245 of SEQ ID NO: 4), and z=SGSETPGTSESATPESGGG (amino acids 473 to 501 of SEQ ID NO: 4). In this format, the human IgG1 Fc or knob-in-hole IgG1 Fc fragments spanned from position 234-478 (Kabat numbering). For scFv-Fc fusions, the variable domains were arranged in a VL-VH orientation and were connected by a long GTTAASGSSGGSSSGA (SEQ ID NO: 75) linker, which favors intramolecular association between VH and VL domains and thus favors scFv formation. For all constructs, the entire coding region was cloned into a mammalian expression vector in frame with the secretion signal peptide.

4. Protein Expression and Purification

Antigen, Ab, and FLAg proteins were produced in Expi293F (ThermoFisher) cells by transient transfection. Briefly, cells were grown to a density of approximately 2.5×10⁶ cells/ml in Expi293 Expression Media (Gibco) in baffled cell culture flasks and transfected with the appropriate vectors using FectoPRO transfection reagent (Polyplus-transfection) using standard manufacture protocols (ThermoFisher). Expression was allowed to proceed for 5 days at 37° C. and 8% CO₂ with shaking at 125 rpm. After expression, cells were removed by centrifugation and protein was purified from the conditioned media using rProtein A Sepharose (GE Healthcare). Purified protein was buffer exchanged into either PBS or a formulated stabilization buffer (36.8 mM citric acid, 63.2 mM Na₂HPO₄, 10% trehalose, 0.2 M L-arginine, 0.01% Tween-80, pH 6.0) for storage. Proteins concentrations were determined by absorbance at 280 nm and purity was confirmed by SDS-PAGE analysis.

5. In Vitro Binding Assays

BLI assays were performed using an Octet HTX instrument (ForteBio). For measuring binding to antigen, Fc-tagged fusions of FZD receptors (FZD-Fc proteins) were captured on AHQ BLI sensors (18-5001, ForteBio) to achieve a BLI response of 0.6-1 nm and remaining Fc-binding sites were saturated with human Fe (009-000-008, Jackson ImmunoResearch). FZD-coated or control (Fc-coated) sensors were transferred into 100 nM Ab or FLAg in assay buffer (PBS, 1% BSA, 0.05% Tween20) and association was monitored for 300 seconds. Sensors were then transferred into assay buffer and dissociation was monitored for an additional 300 seconds. Shake speed was 1000 rpm and temperature was 25° C. End-point response values were taken after 295 seconds of association time. End-point data were analyzed by subtracting the Fc signal from the FZD-Fc signal and then normalizing the data to the highest binding signal.

For measuring binding to Fc receptors, Abs or FLAgs were immobilized on AR2G sensors (18-5092, ForteBio) by amine coupling to achieve a BLI response of 0.6-3 nm and remaining sites were quenched with ethanolamine. Coated sensors were equilibrated in assay buffer (PBS, 1% BSA, 0.05% Tween20) and transferred into Fc receptor solutions. Association was monitored for 600 seconds, the sensors were transferred to assay buffer, and dissociation was monitored for 600 seconds. CD64 and all other Fc receptors were assayed at 50 nM or 300 nM, respectively, at pH 7.4, unless as indicated. Shake speed was 1000 rpm and temperature was 25° C. End-point response values were taken at the end of the association phase and were normalized to isotype controls. Steady-state FcRN binding assays were performed in a similar manner, except that FcRN was immobilized and serial dilutions (0.1-225 nM) of Ab or FLAg were assessed in solution. The association and disassociation times were 600 or 1200 seconds, respectively.

Surface plasmon resonance (SPR) assays were performed using a ProteOn XPR36 system (Bio-Rad). FZD-Fc or LRP-Fc proteins were immobilized to GLC sensor surface (176-5011) using standard amine coupling chemistry. Abs or FLAgs in assay buffer (PBS, 0.05% Tween20, 0.5% BSA) were injected at 40 l/min and association was monitored for 150 seconds. Assay buffer was then injected at 100 l/min and dissociation was monitored for 900 seconds. Assays were performed at 25° C. Analysis was performed using a 1:1 Langmuir model and globally fit to determine kon and koff values using ProteOn Manager software. KD was calculated as the ratio of koff/kon.

6. Epitope Binning

BLI epitope binning experiments were performed using an Octet HTX instrument (ForteBio). Fc fusions with FZD (FZD-Fc) or with LRP6 (LRP6-Fc) protein were immobilized on AHQ (18-5001, ForteBio) or AR2G (18-5092, ForteBio) BLI sensors, respectively. Coated sensors were transferred into 100 nM Ab in assay buffer (PBS, 1% BSA, 0.05% Tween20) for 240 seconds to achieve saturation of binding sites. Sensors were then transferred into 100 nM competing Ab in assay buffer for 180 seconds. Response at 20 seconds after exposure to competing Ab was measured and normalized to binding signal on unblocked antigen-coated sensors. Shake speed was 1000 rpm and temperature was 25° C.

7. Cell Lines

HPAF-II and HEK293T cell lines were maintained in DMEM containing 4.5 g/L D-glucose, Sodium pyruvate, L-glutamine (ThermoFisher #12430-054) and supplemented with 10% FBS (ThermoFisher) and Penicillin/Streptomycin (ThermoFisher #15140-163). CHO cells were maintained in DMEM/F12 (ThermoFisher #11320-033) supplemented with 10% FBS and penicillin/streptomycin. Cells were maintained at 37° C. and 5% CO₂.

8. Flow Cytometry

Indirect immunofluorescence staining of cells was performed with 10 nM anti-FZD Fab for the CHO cell lines as previously described (Steinhart et al. 2017 Nat Med. January; 23(1):60-68, PMID: 27869803). Alexa Fluor 488 AffiniPure F(ab′)₂ was used as the secondary antibody (Jackson ImmunoResearch, 109-545-097). Anti-c-Myc IgG1 9E10 (primary antibody, ThermoFisher, MA1-980) and Alexa Fluor 488 IgG (secondary antibody, Life technologies, A11001) were used as controls for expression. All reagents were used as per manufacturer's instructions.

9. Luciferase Reporter Assay

HEK293T cells were transduced with lentivirus coding for the pBARls reporter (Biechele and Moon in Wnt Signaling: Pathway Methods and Mammalian Models, E. Vincan, Ed. (Humana Press, Totowa, N.J., 2008), pp. 99-110) and with Renilla Luciferase as a control to generate a Wnt-βcatenin signaling reporter cell line. 1-2×10³ cells in 120 μl were seeded in each well of 96-well plates for 24 hours prior to transfection or stimulation. The following day, FLAg or Ab protein was added, and following 15-20 hours of stimulation, cells were lysed and luminescence was measured in accordance with the dual luciferase protocol (Promega) using an Envision plate reader (PerkinElmer). For the FZD4-specific agonist assay, FZD4 cDNA was transfected for 6 hours prior to adding FLAg protein. For the Wnt inhibition assays, Wnt1 was introduced by cDNA transfection or WNT3A protein was applied for 6 hours prior to the addition of Ab protein. All assays were repeated at least three times.

10. Western Blot Assay

H1 ESCs were solubilized with lysis buffer (1% Nonidet P-40, 0.1% sodium dodecyl sulfate (SDS), 0.1% deoxycholic acid, 50 mM Tris (pH 7.4), 0.1 mM EGTA, 0.1 mM EDTA, 20 mM sodium fluoride (NaF), 1:500 protease inhibitors (Sigma) and 1 mM sodium orthovanadate (Na₃VO₄)). Lysate was incubated for 30 min at 4° C., centrifuged at 14,000×g for 10 min, boiled in SDS sample buffer, separated by SDS-polyacrylamide gel electrophoresis, transferred onto a nitrocellulose membrane and Western blotted using indicated Abs. Ab detection was performed by a chemiluminescence-based detection system (ECL; ThermoFisher).

11. Crystal Violet Proliferation Assay

HPAF-II cells were seeded at 500 cells per well, and after 24 hours, 100 nM LGK974 was added with or without 100 nM FLAg. Medium was changed and drug treatment was renewed every other day. Cells were fixed with ice-cold methanol after 7 days treatment. Cells were stained with 0.5% crystal violet solution in 25% methanol, destained in 10% acetic acid and quantified by measuring absorbance at 590 nm.

12. Immunofluorescence

H1 hES treated with FLAg and CHIR99021 for 3 days were washed with cold PBS, and fixed for 20 min with 4% PFA. Fixed cells were rinsed with PBS, permeabilized with 0.3% triton for 10 min, and blocked with 1% BSA for 1 hour. Cells were incubated for 2 hours with primary Abs for BRACHYURY (R&D systems AF2085; goat; dilution 1:100) or OCT3/4 (Santa Cruz sc5279; mouse; dilution 1:100) in 1% BSA and 1 hour with Alexa Fluor 488-labeled donkey anti-goat or Alexa Fluor 568-labeled donkey anti-mouse Ab (FIG. 13A). Coverslips were mounted using Fluoromount (Sigma-Aldrich) and analyzed on a Zeiss LSM700 confocal microscope using a 60× oil objective (FIG. 13B). Images were assembled using ImageJ and Photoshop CS6 (Adobe Systems, Mountain View, Calif.).

13. Intestinal Crypt Self-Renewal Assay

8-10 week-old Lgr5-EGFP-IRES-creERT2 (B6.129P2-Lgr5tm1(cre/ERT2)Cle/J) mice were purchased from The Jackson Laboratory (Bar Harbor, Me.). All experiments were performed according to protocols approved by the Animal Care and Use Committee at the University of Toronto, and complied with the regulations of the Canadian Council on Animal Care and with the ARRIVE guidelines (Animal Research: Reporting in Vivo Experiments). F^{P+P}-L6¹⁺³ or a negative control Ab was reconstituted in 37 mM Citric Acid, 63 mM Na2HPO4, 10% trehalose, 0.2M L-Arginine, 0.01% polysorbate 80, pH 6.0. The Porcupine Inhibitor C59 was reconstituted with 0.5% methylcellulose mixed with 0.1% Tween 80 in ddH₂O. The mice (male and female) were divided into three groups (5-7 per group): vehicle, control (C59 and control Ab) or FLAg (C59 and F^{P+P}-L6¹⁺³). On day 1, mice were treated by intraperitoneal injection with vehicle, or 10 mg/kg control Ab or F^{P+P}-L6¹⁺³. The treatments were blinded to the investigators until the end of the experiment and were repeated every two days for a total three treatments. Starting on day 2, vehicle or 50 mg/kg C59 was administered by gavage to the vehicle group or the two experimental groups, respectively, twice a day with 8 hours interval for 4 days. On day 6, the mice were sacrificed. The whole intestinal tissue was harvested, cleaned with cold PBS, dehydrated with PBS, 30% sucrose, fixed with 4% paraformaldehyde and embedded in optimal cutting temperature compound (OCT). 8 m OCT frozen sections were used for immunohistology. The intestinal EGFP crypts were analyzed using confocal microscopy (Zeiss LSM700). Representative fluorescence images of small intestinal sections from LGR5-GFP mice treated with vehicle, C59 or pan-FLAg(F^{P+P}-L6¹⁺³)+C59 are depicted in FIG. 14. LGR5-GFP is expressed in the stem cells at the bottom of crypts. Cell nuclei were counterstained with DAPI.

Those skilled in the art will recognize, or be able to ascertain, using no more than routine experimentation, numerous equivalents to the specific procedures described herein. Such equivalents are considered to be within the scope of the inventions. Various substitutions, alterations and modifications may be made to the invention without departing from the spirit and scope of the invention. Other aspects, advantages, and modifications are within the scope of the invention. The contents of all references, issued patents, and published patent applications cited through this application are hereby incorporated by reference. The appropriate component, process and methods of those patents, applications and other documents may be selected for the invention and embodiments thereof.

TABLE 1A

| | | SEQ | | SEQ |
|---|---|---|---|---|
| ID | DNA | ID | Protein | ID |
| 5019- | GAGGTTCAGCTGGTGGAGTCTGGCGGTGGCCTGGTGCAGCCAGGGGGCTCACTCCGTTTGTCCTGTGCAGCTTCTGGCTTCAACA | 1 | EVQLVESGGGLVQPGGSLRLSCAASGFNIG | 2 |
| knob- | TCGGTTCTTCTTCTATCCACTGGGTGCGTCAGGCCCCGGGTAAGGGCCTGGAATGGGTTGCATCTATTTATTCTGCTTTTGCCTCTA | | SSSIHWVRQAPGKGLEWVASIYSAFASTSY | |
| 2539- | CTTCTTATGCCGATAGCGTCAAGGGCCGTTTCACTATAAGCGCAGACACATCCAAAAACACAGCCTACCTACAAATGAACAGCTTA | | ADSVKGRFTISADTSKNTAYLQMNSLRAED | |
| 2542 | AGAGCTGAGGACACTGCCGTCTATTATTGTGCTCGCTACCATTTCCCGTTCGGTTTTGCTTTGGACTACTGGGGTCAAGGAACCCT | | TAVYYCARYHFPFGFALDYWGQGTLVTVS | |
| | GGTCACCGTCTCCTCGGGTGGAGGTGGCAGTGATATCCAGATGACCCAGTCCCCGAGCTCCCTGTCCGCCTCTGTGGGCGATAGG | | SGGGGSDICWITQSPSSLSASVGDRVTITCR | |
| | GTCACCATCACCTGCCGTGCCAGTCAGTCCGTGTCCAGCGCTGTAGCCTGGTATCAACAGAAACCAGGAAAAGCTCCGAAGCTTTT | | ASQSVSSAVAWYQQKPGKAPKLLIYSASSL | |
| | GATTTACTCGGCATCCAGCCTCTACTCTGGAGTCCCTTCTCGCTTCTCTGGTAGCCGTTCCGGGACGGATTTCACTCTGACCATCAG | | YSGVPSRFSGSRSGTDFTLTISSLQPEDFAT | |
| | CAGTCTGCAGCCGGAAGACTTCGCAACTTATTACTGTCAGCAAGGTGTTTACCTGTTCACGTTCGGACAGGGTACCAAGGTGGAG | | YYCQQGVYLFTFGQGTKVEIKLEDKTHTKV | |
| | ATCAAACTCGAGgacaaaactcacacaAAAGTGGAGCCCAAAACTTCTgataagacccatacttgcccaCCGTGCCCAGCACCTGAACTCCTG | | EPKTSDKTHTCPPCPAPELLGGPSVFLFPPK | |
| | GGGGGACCGTCAGTCTTCCTCTTCCCCCCAAAACCCAAGGACACCCTCATGATCTCCCGGACCCCTGAGGTCACATGCGTGGTGGT | | PKDTLMISRTPEVTCVVVDVSHEDPEVKFN | |
| | GGACGTGAGCCACGAAGACCCTGAGGTCAAGTTCAACTGGTACGTGGACGGCGTGGAGGTGCATAATGCCAAGACAAAGCCGCG | | WYVDGVEVHNAKTKPREEQYNSTYRVVS | |
| | CGAGGAGCAGTACAACAGCACGTACCGTGTGGTCAGCGTCCTCACCGTCCTGCACCAGGACTGGCTGAATGGCAAGGAGTACAA | | VLTVLHQDWLNGKEYKCKVSNKALPAPIE | |
| | GTGCAAGGTCTCCAACAAAGCCCTCCCAGCCCCCATCGAGAAAACCATCTCCAAAGCCAAAGGGCAGCCCCGAGAACCAATGGTG | | KTISKAKGQPREPMVFDLPPSREEMTKNQ | |
| | TTTGACCTGCCCCCATCCCGGGAGGAGATGACCAAGAACCAGGTCAGCCTGTGGTGCATGGTCAAGGGCTTCTATCCCAGCGACA | | VSLWCMVKGFYPSDIAVEWESNGQPENN | |
| | TCGCCGTGGAGTGGGAGAGCAATGGGCAGCCGGAGAACAACTACAAGACCACGCCTCCCGTGCTGGACTCCGACGGCTCCTTCTT | | YKTTPPVLDSDGSFFLYSKLTVDKSRWQQG | |
| | CCTGTACAGCAAGCTCACCGTGGACAAGAGCCGCTGGCAGCAGGGGAACGTCTTCTCATGCTCCGTGATGCATGAGGCTCTGCAC | | NVFSCSVMHEALHNHYTQKSLSLSPGKSG | |
| | AACCACTACACGCAGAAGAGCCTCTCCCTGTCTCCGGGTAAAAGCGGCAGCGAGACTCCCGGGACCTCAGAGTCCGCCACACCCG | | SETPGTSESATPESGGGEVQLVESGGGLVQ | |
| | AAAGTGGTGGCGGAGAGGTTCAGCTGGTGGAGTCTGGCGGTGGCCTGGTGCAGCCAGGGGGCTCACTCCGTTTGTCCTGTGCAG | | PGGSLRLSCAASGFNISYSSIHWVRQAPGK | |
| | CTTCTGGCTTCAACATCTCTTATTCTTCTATCCACTGGGTGCGTCAGGCCCCGGGTAAGGGCCTGGAATGGGTTGCATATATTTCTT | | GLEWVAYISSYYGYMADSVKGRFTISADT | |
| | CTTATTATGGCTATACTTATTATGCCGATAGCGTCAAGGGCCGTTTCACTATAAGCGCAGACACATCCAAAAACACAGCCTACCTAC | | SKNTAYLQMNSLRAEDTAVYYCARAHYFP | |
| | AAATGAACAGCTTAAGAGCTGAGGACACTGCCGTCTATTATTGTGCTCGCGCTCATTACTTCCCGTGGGCTGGTGCTATGGACTAC | | WAGAMDYWGQGTLVIVSSGGGGSDIQ | |
| | TGGGGTCAAGGAACCCTGGTCACCGTCTCCTCGGGTGGAGGTGGCAGTGATATCCAGATGACCCAGTCCCCGAGCTCCCTGTCCG | | MTQSPSSLSASVGDRVTITCRASQSVSSAV | |
| | CCTCTGTGGGCGATAGGGTCACCATCACCTGCCGTGCCAGTCAGTCCGTGTCCAGCGCTGTAGCCTGGTATCAACAGAAACCAGG | | AWYQQKPGKAPKLUYSASSLYSGVPSRFS | |
| | AAAAGCTCCGAAGCTTCTGATTTACTCGGCATCCAGCCTCTACTCTGGAGTCCCTTCTCGCTTCTCTGGTAGCCGTTCCGGGACGGA | | GSRSGTDFTLTISSLQPEDFATYYCQQYYW | |
| | TTTCACTCTGACCATCAGCAGTCTGCAGCCGGAAGACTTCGCAACTTATTACTGTCAGCAATACTACTGGCCGATCACGTTCGGACA | | PITFGQGTKVEIK | |
| | GGGTACCAAGGTGGAGATCAAA | | | |
| 5019- | GAGGTTCAGCTGGTGGAGTCTGGCGGTGGCCTGGTGCAGCCAGGGGGCTCACTCCGTTTGTCCTGTGCAGCTTCTGGCTTCAACA | 3 | EVQLVESGGGLVQPGGSLRLSCAASGFNIG | 4 |
| hole- | TCGGTTCTTCTTCTATCCACTGGGTGCGTCAGGCCCCGGGTAAGGGCCTGGAATGGGTTGCATCTATTTATTCTGCTTTTGCCTCTA | | SSSIHWVRCIAPGKGLEWVASIYSAFASTSY | |
| 2539- | CTTCTTATGCCGATAGCGTCAAGGGCCGTTTCACTATAAGCGCAGACACATCCAAAAACACAGCCTACCTACAAATGAACAGCTTA | | ADSVKGRFTISADTSKNTAYLQMNSLRAED | |
| 2542 | AGAGCTGAGGACACTGCCGTCTATTATTGTGCTCGCTACCATTTCCCGTTCGGTTTTGCTTTGGACTACTGGGGTCAAGGAACCCT | | TAVYYCARYHFPFGFALDYWGQGTLVTVS | |
| | GGTCACCGTCTCCTCGGGTGGAGGTGGCAGTGATATCCAGATGACCCAGTCCCCGAGCTCCCTGTCCGCCTCTGTGGGCGATAGG | | SGGGGSDIQMTQSPSSLSASVGDRVTITCR | |
| | GTCACCATCACCTGCCGTGCCAGTCAGTCCGTGTCCAGCGCTGTAGCCTGGTATCAACAGAAACCAGGAAAAGCTCCGAAGCTTCT | | ASQSVSSAVAWYQQKPGKAPKLLIYSASSL | |
| | GATTTACTCGGCATCCAGCCTCTACTCTGGAGTCCCTTCTCGCTTCTCTGGTAGCCGTTCCGGGACGGATTTCACTCTGACCATCAG | | YSGVPSRFSGSRSGTDFTLTISSLQPEDFAT | |
| | CAGTCTGCAGCCGGAAGACTTCGCAACTTATTACTGTCAGCAAGGTGTTTACCTGTTCACGTTCGGACAGGGTACCAAGGTGGAG | | YYCQQGVYLFTFGQGTKVEIKLEDKTHTKV | |
| | ATCAAACTCGAGgacaaaactcacacaAAAGTTGAGCCCAAATCTTCTgataagacccataatTGCCCACCGTGCCCAGCACCTGAACTCCT | | EPKSSDKTHNCPPCPAPELLGGPSVFLFPPK | |
| | GGGGGGACCGTCAGTCTTCCTCTTCCCCCCAAAACCCAAGGACACCCTCATGATCTCCCGGACCCCTGAGGTCACATGCGTGGTGG | | PKDTLMISRTPEVTCVVVDVSHEDPEVKFN | |
| | TGGACGTGAGCCACGAAGACCCTGAGGTCAAGTTCAACTGGTACGTGGACGGCGTGGAGGTGCATAATGCCAAGACAAAGCCGC | | WYVDGVEVHNAKTKPREEQYNSTYRVVS | |
| | GGGAGGAGCAGTACAACAGCACGTACCGTGTGGTCAGCGTCCTCACCGTCCTGCACCAGGACTGGCTGAATGGCAAGGAGTACA | | VLTVLHQDWLNGKEYKCKVSNKALPAPIE | |
| | AGTGCAAGGTCTCCAACAAAGCCCTCCCAGCCCCCATCGAGAAAACCATCTCCAAAGCCAAAGGGCAGCCCCGAGAACCACAGGT | | KTISKAKGQPREPQVYTLPPIRELMTSNQV | |
| | GTACACCCTGCCCCCAATCCGGGAGCTGATGACCAGCAACCAGGTCAGCCTGAGCTGCGCCGTCAAAGGCTTCTATCCCAGCGAC | | SLSCAVKGFYPSDIAVEWESNGQPENNYK | |
| | ATCGCCGTGGAGTGGGAGAGCAATGGGCAGCCGGAGAACAACTACAAGACCACGCCTCCCGTGCTGGACTCCGACGGCTCCrrCT | | TTPPVLDSDGSFFLVSKLTVDKSRWQQGN | |
| | TCCTCGTGAGCAAGCTCACCGTGGACAAGAGCAGGTGGCAGCAGGGGAACGTCTTCTCATGCTCCGTGATGCATGAGGCTCTGCA | | VFSCSVMHEALHNHYTQKSLSLSPGKSGSE | |
| | CAACCACTACACGCAGAAGAGCCTCTCCCTGTCTCCGGGTAAAAGCGGCAGCGAGACTCCCGGGACCTCAGAGTCCGCCACACCC | | TPGTSESATPESGGGEVQLVESGGGLVQP | |
| | GAAAGTGGTGGCGGAGAGGTTCAGCTGGTGGAGTCTGGCGGTGGCCTGGTGCAGCCAGGGGGCTCACTCCGTTTGTCCTGTGCA | | GGSLRLSCAASGFNISSYYIHWVRQAPGKG | |
| | GCTTCTGGCTTCAACATCTCTTCTTATTATATCCACTGGGTGCGTCAGGCCCCGGGTAAGGGCCTGGAATGGGTTGCATCTATTTAT | | LEWVASIYSSYGYTSYADSVKGRFTISADTS | |
| | TCTTCTTATGGCTATACTTCTTATGCCGATAGCGTCAAGGGCCGTTTCACTATAAGCGCAGACACATCCAAAAACACAGCCTACCTA | | KNTAYLQMNSLRAEDTAVYYCARTVRGSK | |
| | CAAATGAACAGCTTAAGAGCTGAGGACACTGCCGTCTATTATTGTGCTCGCACTGTTCGTGGATCCAAAAAACCGTACTTCTCTGG | | KPYFSGWAMDYWGQGTLVTVSSGGGGS | |
| | TTGGGCTATGGACTACTGGGGTCAAGGAACCCTGGTCACCGTCTCCTCGGGTGGAGGTGGCAGTGATATCCAGATGACCCAGTCC | | DIQMTQSPSSLSASVGDRVTITCRASQSVS | |
| | CCGAGCTCCCTGTCCGCCTCTGTGGGCGATAGGGTCACCATCACCTGCCGTGCCAGTCAGTCCGTGTCCAGCGCTGTAGCCTGGTA | | SAVAWYQQKPGKAPKLLIYSASSLYSGVPS | |
| | TCAACAGAAACCAGGAAAAGCTCCGAAGCTTCTGATTTACTCGGCATCCAGCCTCTACTCTGGAGTCCCTCTCGCTTCTCTGGTAG | | RFSGSRSGTDFTLTISSLQPEDFATYYCQQY | |
| | CCGTTCCGGGACGGATTTCACTCTGACCATCAGCAGTCTGCAGCCGGAAGACTTCGCAACTTATTACTGTCAGCAATACTCTTGGG | | SWGPFTFGQGTKVEIK* | |
| | GTCCGTTCACGTTCGGACAGGGTACCAAGGTGGAGATCAAA | | | |
| 5019- | GAGGTTCAGCTGGTGGAGTCTGGCGGTGGCCTGGTGCAGCCAGGGGGCTCACTCCGTTTGTCCTGTGCAGCTTCTGGCTTCAACA | 5 | EVQLVESGGGLVQPGGSLRLSCAASGFNIG | 6 |
| Fc- | TCGGTTCTTCTTCTATCCACTGGGTGCGTCAGGCCCCGGGTAAGGGCCTGGAATGGGTTGCATCTATTTATTCTGCTTTTGCCTCTA | | SSSIHWVRQAPGKGLEWVASIYSAFASTSY | |
| 2539 | CTTCTTATGCCGATAGCGTCAAGGGCCGTTTCACTATAAGCGCAGACACATCCAAAAACACAGCCTACCTACAAATGAACAGCTTA | | ADSVKGRFTISADTSKNTAYLQMNSLRAED | |
| | AGAGCTGAGGACACTGCCGTCTATTATTGTGCTCGCTACCATTTCCCGTTCGGTTTTGCTTTGGACTACTGGGGTCAAGGAACCCT | | TAVYYCARYHFPFGFALDYWGQGTLVTVS | |
| | GGTCACCGTCTCCTCGGGTGGAGGTGGCAGTGATATCCAGATGACCCAGTCCCCGAGCTCCCTGTCCGCCTCTGTGGGCGATAGG | | SGGGGSDIQMTQSPSSLSASVGDRVTITCR | |
| | GTCACCATCACCTGCCGTGCCAGTCAGTCCGTGTCCAGCGCTGTAGCCTGGTATCAACAGAAACCAGGAAAAGCTCCGAAGCTTCT | | ASQSVSSAVAWYQQKPGKAPKLLIYSASSL | |
| | GATTTACTCGGCATCCAGCCTCTACTCTGGAGTCCCTTCTCGCTTCTCTGGTAGCCGTTCCGGGACGGATTTCACTCTGACCATCAG | | YSGVPSRFSGSRSGTDFTLTISSLQPEDFAT | |
| | CAGTCTGCAGCCGGAAGACTTCGCAACTTATTACTGTCAGCAAGGTGTTTACCTGTTCACGTTCGGACAGGGTACCAAGGTGGAG | | YYCQQGVYLFTFGQGTKVEIKLEDKTHTKV | |
| | ATCAAACTCGAGgacaaaactcacacaAAAGTTGAGCCCAAATCTTCTgataagacccatacttgcccaccgtgcccagcacctg | | EPKSSDKTHTCPPCPAPELLGGPSVFLFPPK | |
| | aactcctggggggaccgtcagtcttcctcttccccccaaaacccaaggacaccctcatgatctcccggacccctgaggtcacatg | | PKDTLMISRTPEVTCVVVDVSHEDPEVKFN | |
| | cgtggtggtggacgtgagccacgaagaccctgaggtcaagttcaactggtacgtggacggcgtggaggtgcataatgccaagaca | | WYVDGVEVHNAKTKPREEQYNSTYRVVS | |
| | aagccgcgggaggagcagtacaacagcacgtaccgtgtggtcagcgtcctcaccgtcctgcaccaggactggctgaatggcaagg | | VLTVLHQDWLNGKEYKCKVSNKALPAPIE | |
| | agtacaagtgcaaggtctccaacaaagccctcccagcccccatcgagaaaaccatctccaaagccaaagggcagccccgagaacc | | KTISKAKGQPREPQVYTLPPSREEMTKNQ | |
| | acaggtgtacaccctgcccccatcccgggaggagatgaccaagaaccaggtcagcctgacctgcctggtcaaaggcttctatccc | | VSLTCLVKGFYPSDIAVEWESNGQPENNY | |
| | agcgacatcgccgtggagtgggagagcaatgggcagccggagaacaactacaagaccacgcctcccgtgctggactccgacggct | | KTTPPVLDSDGSFFLYSKLTVDKSRWQQG | |
| | ccttcttcctctacagcaagctcaccgtggacaagagcaggtggcagcaggggaacgtcttctcatgctccgtgatgcacgaggc | | NVFSCSVMHEALHNHYTQKSLSLSPGKSG | |
| | tctgcacaaccactacacgcagaagagcctctccctgtctccgggtaaaAGCGGCAGCGAGACTCCCGGGAC | | SETPGTSESATPESGGGEVQLVESGGGLVQ | |
| | CTCAGAGTCCGCCACACCCGAAAGTGGTGGCGGAGAGGTTCAGCTGGTGGAGTCTGGCGGTGGCCTGGTGCAGCCAGGGGGCTC | | PGGSLRLSCAASGFNISYSSIHWVRQAPGK | |
| | ACTCCGTTTGTCCTGTGCAGCTTCTGGCTTCAACATCTCTTATTCTTCTATCCACTGGGTGCGTCAGGCCCCGGGTAAGGGCCTGGA | | GLEWVAYISSYYGYTYYADSVKGRFTISADT | |
| | ATGGGTTGCATATATTTCTTCTTATTATGGCTATACTTATTATGCCGATAGCGTCAAGGGCCGTTTCACTATAAGCGCAGACACATC | | SKNTAYLQMNSLRAEDTAVYYCARAHYFP | |
| | CAAAAACACAGCCTACCTACAAATGAACAGCTTAAGAGCTGAGGACACTGCCGTCTATTATTGTGCTCGCGCTCATTACTTCCCGT | | WAGAMDYWGQGTLVTVSSGGGGSDIQ | |
| | GGGCTGGTGCTATGGACTACTGGGGTCAAGGAACCCTGGTCACCGTCTCCTCGGGTGGAGGTGGCAGTGATATCCAGATGACCC | | MTQSPSSLSASVGDRVTITCRASQSVSSAV | |
| | AGTCCCCGAGCTCCCTGTCCGCCTCTGTGGGCGATAGGGTCACCATCACCTGCCGTGCCAGTCAGTCCGTGTCCAGCGCTGTAGCC | | AWYQQKPGKAPKLLIYSASSLYSGVPSRFS | |
| | TGGTATCAACAGAAACCAGGAAAAGCTCCGAAGCTTCTGATTTACTCGGCATCCAGCCTCTACTCTGGAGTCCCTTCTCGCTTCTCT | | GSRSGTDFTLTISSLCIPEDFATYYCQQYSW | |
| | GGTAGCCGTTCCGGGACGGATTTCACTCTGACCATCAGCAGTCTGCAGCCGGAAGACTTCGCAACTTATTACTGTCAGCAATACTC | | GPFTFGQGTKVEIK | |
| | TTGGGGTCCGTTCACGTTCGGACAGGGTACCAAGGTGGAGATCAAA | | | |
| 5019- | GAGGTTCAGCTGGTGGAGTCTGGCGGTGGCCTGGTGCAGCCAGGGGGCTCACTCCGTTTGTCCTGTGCAGCTTCTGGCTTCAACA | 7 | EVQLVESGGGIVQPGGSLRLSCAASGFNIG | 8 |
| Fc- | TCGGTTCTTCTTCTATCCACTGGGTGCGTCAGGCCCCGGGTAAGGGCCTGGAATGGGTTGCATCTATTTATTCTGCTTTTGCCTCTA | | SSSIHWVRQAPGKGLEWVASIYSAFASTSY | |
| 2542 | CTTCTTATGCCGATAGCGTCAAGGGCCGTTTCACTATAAGCGCAGACACATCCAAAAACACAGCCTACCTACAAATGAACAGCTTA | | ADSVKGRFTISADTSKNTAYLQMNSLRAED | |
| | AGAGCTGAGGACACTGCCGTCTATTATTGTGCTCGCTACCATTTCCCGTTCGGTTTTGCTTTGGACTACTGGGGTCAAGGAACCCT | | TAVYYCARYHFPFGFALDYWGQGTLVIVS | |
| | GGTCACCGTCTCCTCGGGTGGAGGTGGCAGTGATATCCAGATGACCCAGTCCCCGAGCTCCCTGTCCGCCTCTGTGGGCGATAGG | | SGGGGSDIQMTQSPSSLSASVGDRVTITCR | |
| | GTCACCATCACCTGCCGTGCCAGTCAGTCCGTGTCCAGCGCTGTAGCCTGGTATCAACAGAAACCAGGAAAAGCTCCGAAGCTTCT | | ASQSVSSAVAWYQQKPGKAPKLLIYSASSL | |
| | GATTTACTCGGCATCCAGCCTCTACTCTGGAGTCCCTTCTCGCTTCTCTGGTAGCCGTTCCGGGACGGATTTCACTCTGACCATCAG | | YSGVPSRFSGSRSGTDFTLTISSLQPEDFAT | |
| | CAGTCTGCAGCCGGAAGACTTCGCAACTTATTACTGTCAGCAAGGTGTTTACCTGTTCACGTTCGGACAGGGTACCAAGGTGGAG | | YYCQQGVYLFTFGQGTKVEIKLEDKTHTKV | |
| | ATCAAACTCGAGgacaaaactcacacaAAAGTTGAGCCCAAATCTTCTgataagacccatacttgcccaccgtgcccagcacctg | | EPKSSDKTHTCPPCPAPELLGGPSVFLFPPK | |
| | aactcctggggggaccgtcagtcttcctcttccccccaaaacccaaggacaccctcatgatctcccggacccctgaggtcacatg | | PKDTLMISRTPEVTCVVVDVSHEDPEVKFN | |
| | cgtggtggtggacgtgagccacgaagaccctgaggtcaagttcaactggtacgtggacggcgtggaggtgcataatgccaagaca | | WYVDGVEVHNAKTKPREEQYNSTYRVVS | |
| | aagccgcgggaggagcagtacaacagcacgtaccgtgtggtcagcgtcctcaccgtcctgcaccaggactggctgaatggcaagg | | VLIVLHQDWLNGKEYKCKVSNKALPAPIE | |
| | agtacaagtgcaaggtctccaacaaagccctcccagcccccatcgagaaaaccatctccaaagccaaagggcagccccgagaacc | | KTISKAKGQPREPQVYTLPPSREEMTKNQ | |
| | acaggtgtacaccctgcccccatcccgggaggagatgaccaagaaccaggtcagcctgacctgcctggtcaaaggcttctatccc | | VSLTCLVKGFYPSDIAVEWESNGQPENNY | |
| | agcgacatcgccgtggagtgggagagcaatgggcagccggagaacaactacaagaccacgcctcccgtgctggactccgacggct | | KTTPPVLDSDGSFFLYSKLTVDKSRWQQG | |
| | ccttcttcctctacagcaagctcaccgtggacaagagcaggtggcagcaggggaacgtcttctcatgctccgtgatgcacgaggc | | NVFSCSVMHEALHNHYTQKSLSLSPGKSG | |
| | tctgcacaaccactacacgcagaagagcctctccctgtctccgggtaaaAGCGGCAGCGAGACTCCCGGGAC | | SETPGTSESATPESGGGEVQLVESGGGLVQ | |
| | CTCAGAGTCCGCCACACCCGAAAGTGGTGGCGGAGAGGTTCAGCTGGTGGAGTCTGGCGGTGGCCTGGTGCAGCCAGGGGGCTC | | PGGSLRLSCAASGFNISSYYIHWVRQAPGK | |
| | ACTCCGTTTGTCCTGTGCAGCTTCTGGCTTCAACATCTCTTCTTATTATATCCACTGGGTGCGTCAGGCCCCGGGTAAGGGCCTGGA | | GLEWVASIYSSYGYTSYADSVKGRFTISADT | |
| | ATGGGTTGCATCTATTTATTCTTCTTATGGCTATACTTCTTATGCCGATAGCGTCAAGGGCCGTTTCACTATAAGCGCAGACACATC | | SKNTAYLQMNSLRAEDTAVYYCARTVRGS | |
| | CAAAAACACAGCCTACCTACAAATGAACAGCTTAAGAGCTGAGGACACTGCCGTCTATTATTGTGCTCGCACTGTTCGTGGATCCA | | KKPYFSGWAMDYWGQGTLVIVSSGGGG | |
| | AAAAACCGTACTTCTCTGGTTGGGCTATGGACTACTGGGGTCAAGGAACCCTGGTCACCGTCTCCTCGGGTGGAGGTGGCAGTGA | | SDIQMTQSPSSLSASVGDRVTITCRASQSV | |
| | TATCCAGATGACCCAGTCCCCGAGCTCCCTGTCCGCCTCTGTGGGCGATAGGGTCACCATCACCTGCCGTGCCAGTCAGTCCGTGT | | SSAVAWYQQKPGKAPKWYSASSLYSGVP | |
| | CCAGCGCTGTAGCCTGGTATCAACAGAAACCAGGAAAAGCTCCGAAGCTTCTGATTTACTCGGCATCCAGCCTCTACTCTGGAGTC | | SRFSGSRSGTDFTLTISSLQPEDFATYYCQQ | |
| | CCTTCTCGCTTCTCTGGTAGCCGTTCCGGGACGGATTTCACTCTGACCATCAGCAGTCTGCAGCCGGAAGACTTCGCAACTTATTAC | | YYWPITFGQGTKVEIK | |
| | TGTCAGCAATACTACTGGCCGATCACGTTCGGACAGGGTACCAAGGTGGAGATCAAA | | | |
| 5038- | GAGGTTCAGCTGGTGGAGTCTGGCGGTGGCCTGGTGCAGCCAGGGGGCTCACTCCGTTTGTCCTGTOCAGCTTCTGGCTTCAACA | 9 | EVQLVESGGGLVQPGGSLRLSCAASGFNIS | 10 |
| knob- | TCTCTTATTATTATATGCACTGGGTGCGTCAGGCCCCGGGTAAGGGCCTGGAATGGGTTGCATCTATTTATTCTTATTATGGCTATA | | YYYMHWVRQAPGKGLEWVASIYSYYGYT | |
| 2539- | CTTATTATGCCGATAGCGTCAAGGGCCGTTTCACTATAAGCGCAGACACATCCAAAAACACAGCCTACCTACAAATGAACAGCTTA | | YYADSVKGRFTISADTSKNTAYLQMNSLRA | |
| 2542 | AGAGCTGAGGACACTGCCGTCTATTATTGTGCTCGCTCTTCTTTCTCTTGGGCTATGGACTACTGGGGTCAAGGAACCCTGGTCAC | | EDTAVYYCARSSFSWAMDYWGQGTLVTV | |
| | CGTCTCCTCGGGTGGAGGTGGCAGTGATATCCAGATGACCCAGTCCCCGAGCTCCCTGTCCGCCTCTGTGGGCGATAGGGTCACC | | SSGGGGSDIQMTQSPSSLSASVGDRVTITC | |
| | ATCACCTGCCGTGCCAGTCAGTCCGTGTCCAGCGCTGTAGCCTGGTATCAACAGAAACCAGGAAAAGCTCCGAAGCTTCTGATTTA | | RASQSVSSAVAWYQQKPGKAPKLLIYSASS | |
| | CTCGGCATCCAGCCTCTACTCTGGAGTCCCTTCTCGCTTCTCTGGTAGCCGTTCCGGGACGGATTTCACTCTGACCATCAGCAGTCT | | LYSGVPSRFSGSRSGTDFTLTISSLQPEDFAT | |
| | GCAGCCGGAAGACTTCGCAACTTATTACTGTCAGCAACATCCGTGGTCTGGTGGTTACCTGATCACGTTCGGACAGGGTACCAAG | | YYCQQHPWSGGYLITFGQGTKVEIKLEDKT | |
| | GTGGAGATCAAACTCGAGgacaaaactcacacaAAAGTGGAGCCCAAAACTTCTgataagacccatactTGCCCACCGTGCCCAGCACCTG | | HTKVEPKTSDKTHTCPPCPAPELLGGPSVF | |
| | AACTCCTGGGGGGACCGTCAGTCTFCCTCTTCCCCCCAAAACCCAAGGACACCCTCATGATCTCCCGGACCCCTGAGGTCACATGC | | LFPPKPKDTLMISRTPEVTCVVVDVSHEDP | |
| | GTGGTGGTGGACGTGAGCCACGAAGACCCTGAGGTCAAGTTCAACTGGTACGTGGACGGCGTGGAGGTGCATAATGCCAAGACA | | EVKFNWYVDGVEVHNAKTKPREEQYNST | |
| | AAGCCGCGCGAGGAGCAGTACAACAGCACGTACCGTGTGGTCAGCGTCCTCACCGTCCTGCACCAGGACTGGCTGAATGGCAAG | | YRVVSVLTVLHQDWLNGKEYKCKVSNKAL | |
| | GAGTACAAGTGCAAGGTCTCCAACAAAGCCCTCCCAGCCCCCATCGAGAAAACCATCTCCAAAGCCAAAGGGCAGCCCCGAGAAC | | PAPIEKTISKAKGQPREPMVFDLPPSREEM | |
| | CAATGGTGTTTGACCTGCCCCCATCCCGGGAGGAGATGACCAAGAACCAGGTCAGCCTGTGGTGCATGGTCAAGGGCTTCTATCC | | TKNQVSLWCMVKGFYPSDIAVEWESNGQ | |
| | CAGCGACATCGCCGTGGAGTGGGAGAGCAATGGGCAGCCGGAGAACAACTACAAGACCACGCCTCCCGTGCTGGACTCCGACGG | | PENNYKTIPPVLDSDGSFFLYSKLTVDKSR | |
| | CTCCTTCTTCCTGTACAGCAAGCTCACCGTGGACAAGAGCCGCTGGCAGCAGGGGAACGTCTTCTCATGCTCCGTGATGCATGAGG | | WQQGNVFSCSVMHEALHNHYTQKSLSLS | |
| | CTCTGCACAACCACTACACGCAGAAGAGCCTCTCCCTGTCTCCGGGTAAAAGCGGCAGCGAGACTCCCGGGACCTCAGAGTCCGC | | PGKSGSETPGTSESATPESGGGEVQLVESG | |
| | CACACCCGAAAGTGGTGGCGGAGAGGTTCAGCTGGTGGAGTCTGGCGGTGGCCTGGTGCAGCCAGGGGGCTCACTCCGTTTGTC | | GGLVQPGGSLRLSCAASGFNISYSSIHWVR | |
| | CTGTGCAGCTTCTGGCTTCAACATCTCTTATTCTTCTATCCACTGGGTGCGTCAGGCCCCGGGTAAGGGCCTGGAATGGGTTGCAT | | QAPGKGLEWVAYISSYYGYTYYADSVKGRF | |
| | ATATTTCTTCTTATTATGGCTATACTTATTATGCCGATAGCGTCAAGGGCCGTTTCACTATAAGCGCAGACACATCCAAAAACACAG | | TISADTSKNTAYLQMNSLRAEDTAVYYCAR | |
| | CCTACCTACAAATGAACAGCTTAAGAGCTGAGGACACTGCCGTCTATTATTGTGCTCGCGCTCATTACTTCCCGTGGGCTGGTGCT | | AHYFPWAGAMDYWGQGTLVTVSSGGGG | |
| | ATGGACTACTGGGGTCAAGGAACCCTGGTCACCGTCTCCTCGGGTGGAGGTGGCAGTGATATCCAGATGACCCAGTCCCCGAGCT | | SDIQMTQSPSSLSASVGDRVTITCRASQSV | |
| | CCCTGTCCGCCTCTGTGGGCGATAGGGTCACCATCACCTGCCGTGCCAGTCAGTCCGTGTCCAGCGCTGTAGCCTGGTATCAACAG | | SSAVAWYQQKPGKAPKLLIYSASSLYSGVP | |
| | AAACCAGGAAAAGCTCCGAAGCTTCTGATTTACTCGGCATCCAGCCTCTACTCTGGAGTCCCTTCTCGCTTCTCTGGTAGCCGTTCC | | SRFSGSRSGTDFTLTISSLQPEDFATYYCQQ | |
| | GGGACGGATTTCACTCTGACCATCAGCAGTCTGCAGCCGGAAGACTTCGCAACTTATTACTGTCAGCAATACTACTGGCCGATCAC | | YYWPITFGQGTKVEIK | |
| | GTTCGGACAGGGTACCAAGGTGGAGATCAAA | | | |
| 5038- | GAGGTTCAGCTGGTGGAGTCTGGCGGTGGCCTGGTGCAGCCAGGGGGCTCACTCCGTTTGTCCTGTGCAGCTTCTGGCTTCAACA | 11 | EVQLVESGGGLVQPGGSLRLSCAASGFNIS | 12 |
| hole- | TCTCTTATTATTATATGCACTGGGTGCGTCAGGCCCCGGGTAAGGGCCTGGAATGGGTTGCATCTATTTATTCTTATTATGGCTATA | | YYYMHWVRQAPGKGLEWVASIYSYYGYT | |
| 2539- | CTTATTATGCCGATAGCGTCAAGGGCCGTTTCACTATAAGCGCAGACACATCCAAAAACACAGCCTACCTACAAATGAACAGCTTA | | YYADSVKGRFTISADTSKNTAYLQMNSLRA | |
| 2542 | AGAGCTGAGGACACTGCCGTCTATTATTGTGCTCGCTCTTCTTTCTCTTGGGCTATGGACTACTGGGGTCAAGGAACCCTGGTCAC | | EDTAVYYCARSSFSWAMDYWGQGTLVTV | |
| | CGTCTCCTCGGGTGGAGGTGGCAGTGATATCCAGATGACCCAGTCCCCGAGCTCCCTGTCCGCCTCTGTGGGCGATAGGGTCACC | | SSGGGGSDIQMTQSPSSLSASVGDRVTITC | |
| | ATCACCTGCCGTGCCAGTCAGTCCGTGTCCAGCGCTGTAGCCTGGTATCAACAGAAACCAGGAAAAGCTCCGAAGCTTCTGATTTA | | RASQSVSSAVAWYQQKPGKAPKWYSASS | |
| | CTCGGCATCCAGCCTCTACTCTGGAGTCCCTTCTCGCTTCTCTGGTAGCCGTTCCGGGACGGATTTCACTCTGACCATCAGCAGTCT | | LYSGVPSRFSGSRSGTDFTLTISSLCIPEDFAT | |
| | GCAGCCGGAAGACTTCGCAACTTATTACTGTCAGCAACATCCGTGGTCTGGTGGTTACCTGATCACGTTCGGACAGGGTACCAAG | | YYCQQHPWSGGYLITFGQGTKVEIKLEDKT | |
| | GTGGAGATCAAACTCGAGgacaaaactcacacaAAAGTTGAGCCCAAATCTTCTgataagacccataatTGCCCACCGTGCCCAGCACCTGA | | HTKVEPKSSDKTHNCPPCPAPELLGGPSVF | |
| | ACTCCTGGGGGGACCGTCAGTCTTCCTCTTCCCCCCAAAACCCAAGGACACCCTCATGATCTCCCGGACCCCTGAGGTCACATGCG | | LFPPKPKDTLMISRTPEVTCVVVDVSHEDP | |
| | TGGTGGTGGACGTGAGCCACGAAGACCCTGAGGTCAAGTTCAACTGGTACGTGGACGGCGTGGAGGTGCATAATGCCAAGACAA | | EVKFNWYVDGVEVHNAKTKPREEQYNST | |
| | AGCCGCGGGAGGAGCAGTACAACAGCACGTACCGTGTGGTCAGCGTCCTCACCGTCCTGCACCAGGACTGGCTGAATGGCAAGG | | YRVVSVLTVLHQDWLNGKEYKCKVSNKAL | |
| | AGTACAAGTGCAAGGTCTCCAACAAAGCCCTCCCAGCCCCCATCGAGAAAACCATCTCCAAAGCCAAAGGGCAGCCCCGAGAACC | | PAPIEKTISKAKGQPREPQVYTLPPIRELMT | |
| | ACAGGTGTACACCCTGCCCCCAATCCGGGAGCTGATGACCAGCAACCAGGTCAGCCTGAGCTGCGCCGTCAAAGGCTTCTATCCC | | SNQVSLSCAVKGFYPSDIAVEWESNGQPE | |
| | AGCGACATCGCCGTGGAGTGGGAGAGCAATGGGCAGCCGGAGAACAACTACAAGACCACGCCTCCCGTGCTGGACTCCGACGGC | | NNYKTIPPVLDSDGSFFLVSKLTVDKSRW | |
| | TCCTTCTTCCTCGTGAGCAAGCTCACCGTGGACAAGAGCAGGTGGCAGCAGGGGAACGTCTTCTCATGCTCCGTGATGCATGAGG | | QQGNVFSCSVMHEALHNHYTQKSLSLSPG | |
| | CTCTGCACAACCACTACACGCAGAAGAGCCTCTCCCTGTCTCCGGGTAAAAGCGGCAGCGAGACTCCCGGGACCTCAGAGTCCGC | | KSGSETPGTSESATPESGGGEVQLVESGGG | |
| | CACACCCGAAAGTGGTGGCGGAGAGGTTCAGCTGGTGGAGTCTGGCGGTGGCCTGGTGCAGCCAGGGGGCTCACTCCGTTTGTC | | LVQPGGSLRLSCAASGFNISSYYIHWVRQA | |
| | CTGTGCAGCTTCTGGCTTCAACATCTCTTCTTATTATATCCACTGGGTGCGTCAGGCCCCGGGTAAGGGCCTGGAATGGGTTGCAT | | PGKGLEWVASIYSSYGYTSYADSVKGRFTIS | |
| | CTATTTATTCTTCTTATGGCTATACTTCTTATGCCGATAGCGTCAAGGGCCGTTTCACTATAAGCGCAGACACATCCAAAAACACAG | | ADTSKNTAYLQMNSLRAEDTAVYYCARTV | |
| | CCTACCTACAAATGAACAGCTTAAGAGCTGAGGACACTGCCGTCTATTATTGTGCTCGCACTGTTCGTGGATCCAAAAAACCGTAC | | RGSKKPYFSGWAMDYWGQGTLVTVSSG | |
| | TTCTCTGGTTGGGCTATGGACTACTGGGGTCAAGGAACCCTGGTCACCGTCTCCTCGGGTGGAGGTGGCAGTGATATCCAGATGA | | GGGSDIQMTQSPSSLSASVGDRVTITCRAS | |
| | CCCAGTCCCCGAGCTCCCTGTCCGCCTCTGTGGGCGATAGGGTCACCATCACCTGCCGTGCCAGTCAGTCCGTGTCCAGCGCTGTA | | QSVSSAVAWYQQKPGKAPKLLIYSASSLYS | |
| | GCCTGGTATCAACAGAAACCAGGAAAAGCTCCGAAGCTTCTGATTTACTCGGCATCCAGCCTCTACTCTGGAGTCCCTTCTCGCTTC | | GVPSRFSGSRSGTDFTLTISSLOPEDFATYY | |
| | TCTGGTAGCCGTTCCGGGACGGATTTCACTCTGACCATCAGCAGTCTGCAGCCGGAAGACTTCGCAACTTATTACTGTCAGCAATA | | CQQYSWGPFTFGQGTKVEIK | |
| | CTCTTGGGGTCCGTTCACGTTCGGACAGGGTACCAAGGTGGAGATCAAA | | | |
| 5044- | GAGGTTCAGCTGGTGGAGTCTGGCGGTGGCCTGGTGCAGCCAGGGGGCTCACTCCGTTTGTCCTGTGCAGCTTCTGGCTTCAACC | 13 | EVQLVESGGGLVQPGGSLRLSCAASGFNLS | 14 |
| knob- | TCTCTTCTTATTCTATGCACTGGGTGCGTCAGGCCCCGGGTAAGGGCCTGGAATGGGTTGCATATATTTCTTCTTATTATGGCTATA | | SYSMHWVROAPGKGLEWVAYISSYYGYT | |
| 2539- | CTTATTATGCCGATAGCGTCAAGGGCCGTTTCACTATAAGCGCAGACACATCCAAAAACACAGCCTACCTACAAATGAACAGCTTA | | YYADSVKGRFTISADTSKNTAYLQMNSLRA | |
| 2542 | AGAGCTGAGGACACTGCCGTCTATTATTGTGCTCGCCCGGCTCCGGGTCATTGGGGTTTTGACTACTGGGGTCAAGGAACCCTGG | | EDTAVYYCARPAPGHWGFDYWGQGTLV | |
| | TCACCGTCTCCTCGGGTGGAGGTGGCAGTGATATCCAGATGACCCAGTCCCCGAGCTCCCTGTCCGCCTCTGTGGGCGATAGGGT | | TVSSGGGGSDIQMTQSPSSLSASVGDRVTI | |
| | CACCATCACCTGCCGTGCCAGTCAGTCCGTGTCCAGCGCTGTAGCCTGGTATCAACAGAAACCAGGAAAAGCTCCGAAGCTTCTGA | | TCRASQSVSSAVAWYQQKPGKAPKWYSA | |
| | TTTACTCGGCATCCAGCCTCTACTCTGGAGTCCCTTCTCGCTTCTCTGGTAGCCGTTCCGGGACGGATTTCACTCTGACCATCAGCA | | SSLYSGVPSRFSGSRSGTDFILTISSLCIPEDF | |
| | GTCTGCAGCCGGAAGACTTCGCAACTTATTACTGTCAGCAATGGTACTACGCTCCGATCACGTTCGGACAGGGTACCAAGGTGGA | | ATYYCQQWYYAPITFGQGTKVEIKLEDKTH | |
| | GATCAAACTCGAGgacaaaactcacacaAAAGTGGAGCCCAAAACTTCTgataagacccatactTGCCCACCGTGCCCAGCACCTGAACTCC | | TKVEPKTSDKTHTCPPCPAPELLGGPSVFLF | |
| | TGGGGGGACCGTCAGTCTTCCTCTTCCCCCCAAAACCCAAGGACACCCTCATGATCTCCCGGACCCCTGAGGTCACATGCGTGGTG | | PPKPKDTLMISRTPEVTCVVVDVSHEDPEV | |
| | GTGGACGTGAGCCACGAAGACCCTGAGGTCAAGTTCAACTGGTACGTGGACGGCGTGGAGGTGCATAATGCCAAGACAAAGCCG | | KFNWYVDGVEVHNAKTKPREEQYNSTYR | |
| | CGCGAGGAGCAGTACAACAGCACGTACCGTGTGGTCAGCGTCCTCACCGTCCTGCACCAGGACTGGCTGAATGGCAAGGAGTAC | | VVSVLTVLHQDWLNGKEYKCKVSNKALPA | |
| | AAGTGCAAGGTCTCCAACAAAGCCCTCCCAGCCCCCATCGAGAAAACCATCTCCAAAGCCAAAGGGCAGCCCCGAGAACCAATGG | | PIEKTISKAKGQPREPMVFDLPPSREEMTK | |
| | TGTTTGACCTGCCCCCATCCCGGGAGGAGATGACCAAGAACCAGGTCAGCCTGTGGTGCATGGTCAAGGGCTTCTATCCCAGCGA | | NQVSLWCMVKGFYPSDIAVEWESNGQPE | |
| | CATCGCCGTGGAGTGGGAGAGCAATGGGCAGCCGGAGAACAACTACAAGACCACGCCTCCCGTGCTGGACTCCGACGGCTCCTTC | | NNYKTTPPVLDSCIGSFFLYSKLIVDKSRWQ | |
| | TTCCTGTACAGCAAGCTCACCGTGGACAAGAGCCGCTGGCAGCAGGGGAACGTCTTCTCATGCTCCGTGATGCATGAGGCTCTGC | | QGNVFSCSVMHEALHNHYTQKSLSLSPGK | |
| | ACAACCACTACACGCAGAAGAGCCTCTCCCTGTCTCCGGGTAAAAGCGGCAGCGAGACTCCCGGGACCTCAGAGTCCGCCACACC | | SGSETPGTSESATPESGGGEVQLVESGGGL | |
| | CGAAAGTGGTGGCGGAGAGGTTCAGCTGGTGGAGTCTGGCGGTGGCCTGGTGCAGCCAGGGGGCTCACTCCGTTTGTCCTGTGC | | VQPGGSLRLSCAASGFNISYSSIHWVRQAP | |
| | AGCTTCTGGCTTCAACATCTCTTATTCTTCTATCCACTGGGTGCGTCAGGCCCCGGGTAAGGGCCTGGAATGGGTTGCATATATTTC | | GKGLEWVAYISSYYGYTYYADSVKGRFTISA | |
| | TTCTTATTATGGCTATACTTATTATGCCGATAGCGTCAAGGGCCGTTTCACTATAAGCGCAGACACATCCAAAAACACAGCCTACCT | | DTSKNTAYLQMNSLRAEDTAVYYCARAHY | |
| | ACAAATGAACAGCTTAAGAGCTGAGGACACTGCCGTCTATTATTGTGCTCGCGCTCATTACTTCCCGTGGGCTGGTGCTATGGACT | | FPWAGAMDYWGQGTLVTVSSGGGMI | |
| | ACTGGGGTCAAGGAACCCTGGTCACCGTCTCCTCGGGTGGAGGTGGCAGTGATATCCAGATGACCCAGTCCCCGAGCTCCCTGTC | | QMTQSPSSLSASVGDRVTITCRASSQSVSSA | |
| | CGCCTCTGTGGGCGATAGGGTCACCATCACCTGCCGTGCCAGTCAGTCCGTGTCCAGCGCTGTAGCCTGGTATCAACAGAAACCA | | VAWYQQKPGKAPKWYSASSLYSGVPSRF | |
| | GGAAAAGCTCCGAAGCTTCTGATTTACTCGGCATCCAGCCTCTACTCTGGAGTCCCTTCTCGCTTCTCTGGTAGCCGTTCCGGGACG | | SGSRSGTDFTLTISSLQPEDFATYYCQQYY | |
| | GATTTCACTCTGACCATCAGCAGTCTGCAGCCGGAAGACTTCGCAACTTATTACTGTCAGCAATACTACTGGCCGATCACGTTCGG | | WPITFGQGTKVEIK | |
| | ACAGGGTACCAAGGTGGAGATCAAA | | | |
| 5044- | GAGGTTCAGCTGGTGGAGTCTGGCGGTGGCCTGGTGCAGCCAGGGGGCTCACTCCGTTTGTCCTGTGCAGCTTCTGGCTTCAACC | 15 | EVQLVESGGGLVQPGGSLRLSCAASGENLS | 16 |
| hole- | TCTCTTCTTATTCTATGCACTGGGTGCGTCAGGCCCCGGGTAAGGGCCTGGAATGGGTTGCATATATTTCTTCTTATTATGGCTATA | | SYSMHWVROAPGKGLEWVAYISSYYGYT | |
| 2539- | CTTATTATGCCGATAGCGTCAAGGGCCGTTTCACTATAAGCGCAGACACATCCAAAAACACAGCCTACCTACAAATGAACAGCTTA | | YYADSVKGRFTISADTSKNTAYLQMNSLRA | |
| 2542 | AGAGCTGAGGACACTGCCGTCTATTATTGTGCTCGCCCGGCTCCGGGTCATTGGGGTTTTGACTACTGGGGTCAAGGAACCCTGG | | EDTAVYYCARPAPGHWGFDYWGQGTLV | |
| | TCACCGTCTCCTCGGGTGGAGGTGGCAGTGATATCCAGATGACCCAGTCCCCGAGCTCCCTGTCCGCCTCTGTGGGCGATAGGGT | | TVSSGGGGSDIQMTQSPSSLSASVGDRVTI | |
| | CACCATCACCTGCCGTGCCAGTCAGTCCGTGTCCAGCGCTGTAGCCTGGTATCAACAGAAACCAGGAAAAGCTCCGAAGCTTCTGA | | TCRASQSVSSAVAWYQQKPGKAPKWYSA | |
| | TTTACTCGGCATCCAGCCTCTACTCTGGAGTCCCTTCTCGCTTCTCTGGTAGCCGTTCCGGGACGGATTTCACTCTGACCATCAGCA | | SSLYSGVPSRFSGSRSGTDFTLTISSLQPEDF | |
| | GTCTGCAGCCGGAAGACTTCGCAACTTATTACTGTCAGCAATGGTACTACGCTCCGATCACGTTCGGACAGGGTACCAAGGTGGA | | ATYYCQQWYYAPITFGQGTKVEIKLEDKTH | |
| | GATCAAACTCGAGgacaaaactcacacaAAAGTTGAGCCCAAATCTTCTgataagacccataatTGCCCACCGTGCCCAGCACCTGAACTCC | | TKVERKSSDKTFINCPPCPAPELLGGPSVFLF | |
| | TGGGGGGACCGTCAGTCTTCCTCTTCCCCCCAAAACCCAAGGACACCCTCATGATCTCCCGGACCCCTGAGGTCACATGCGTGGTG | | PPKPKDILMISRTPEVTCVVVDVSHEDPEV | |
| | GTGGACGTGAGCCACGAAGACCCTGAGGTCAAGTTCAACTGGTACGTGGACGGCGTGGAGGTGCATAATGCCAAGACAAAGCCG | | KFNWYVDGVEVHNAKTKPREEQYNSTYR | |
| | CGGGAGGAGCAGTACAACAGCACGTACCGTGTGGTCAGCGTCCTCACCGTCCTGCACCAGGACTGGCTGAATGGCAAGGAGTAC | | VVSVLTVLHQDWLNGKEYKCKVSNKALPA | |
| | AAGTGCAAGGTCTCCAACAAAGCCCTCCCAGCCCCCATCGAGAAAACCATCTCCAAAGCCAAAGGGCAGCCCCGAGAACCACAGG | | PIEKTISKAKGQPREPQVYTLPPIRELMTSN | |
| | TGTACACCCTGCCCCCAATCCGGGAGCTGATGACCAGCAACCAGGTCAGCCTGAGCTGCGCCGTCAAAGGCTTCTATCCCAGCGAC | | QVSLSCAVKGFYPSDIAVEWESNGQPENN | |
| | ATCGCCGTGGAGTGGGAGAGCAATGGGCAGCCGGAGAACAACTACAAGACCACGCCTCCCGTGCTGGACTCCGACGGCTCCTTCT | | YKTTPPVLDSDGSFFLVSKLTVDKSRWQQ | |
| | TCCTCGTGAGCAAGCTCACCGTGGACAAGAGCAGGTGGCAGCAGGGGAACGTCTTCTCATGCTCCGTGATGCATGAGGCTCTGCA | | GNVFSCSVMHEALHNHYTQKSLSLSPGKS | |
| | CAACCACTACACGCAGAAGAGCCTCTCCCTGTCTCCGGGTAAAAGCGGCAGCGAGACTCCCGGGACCTCAGAGTCCGCCACACCC | | GSETPGTSESATPESGGGEVQLVESGGGLV | |
| | GAAAGTGGTGGCGGAGAGGTTCAGCTGGTGGAGTCTGGCGGTGGCCTGGTGCAGCCAGGGGGCTCACTCCGTTTGTCCTGTGCA | | QPGGSLRLSCAASGFNISSYYIHWVRQAPG | |
| | GCTTCTGGCTTCAACATCTCTTCTTATTATATCCACTGGGTGCGTCAGGCCCCGGGTAAGGGCCTGGAATGGGTTGCATCTATTTAT | | KGLEWVASIYSSYGYTSYADSVKGRFTISAD | |
| | TCTTCTTATGGCTATACTTCTTATGCCGATAGCGTCAAGGGCCGTTTCACTATAAGCGCAGACACATCCAAAAACACAGCCTACCTA | | TSKNTAYLQMNSIRAEDTAVYYCARTVRG | |
| | CAAATGAACAGOTAAGAGCTGAGGACACTGCCGTCTATTATTGTGCTCGCACTGTTCGTGGATCCAAAAAACCGTACTTCTCTGG | | SKKPYFSGWAMDYWGQGTLVTVSSGGG | |
| | TTGGGCTATGGACTACTGGGGTCAAGGAACCCTGGTCACCGTCTCCTCGGGTGGAGGTGGCAGTGATATCCAGATGACCCAGTCC | | GSDIQMTQSPSSLSASVGDRVTITCRASQS | |
| | CCGAGCTCCCTGTCCGCCTCTGTGGGCGATAGGGTCACCATCACCTGCCGTGCCAGTCAGTCCGTGTCCAGCGCTGTAGCCTGGTA | | VSSAVAWYQQKPGKAPKLLIYSASSLYSGV | |
| | TCAACAGAAACCAGGAAAAGCTCCGAAGCTTCTGATTTACTCGGCATCCAGCCTCTACTCTGGAGTCCCTTCTCGCTTCTCTGGTAG | | PSRFSGSRSGTDFTLTISSLQPEDFATYYCQ | |
| | CCGTTCCGGGACGGATTTCACTCTGACCATCAGCAGTCTGCAGCCGGAAGACTTCGCAACTTATTACTGTCAGCAATACTCTTGGG | | QYSWGPFTFGQGTKVEIK | |
| | GTCCGTTCACGTTCGGACAGGGTACCAAGGTGGAGATCAAA | | | |
| 5048- | GAGGTTCAGCTGGTGGAGTCTGGCGGTGGCCTGGTGCAGCCAGGGGGCTCACTCCGTTTGTCCTGTGCAGCTTCTGGCTTCAACA | 17 | EVQLVESGGGLVQPGGSLRLSCAASGFNIS | 18 |
| knob- | TCTCTTATTATTATATGCACTGGGTGCGTCAGGCCCCGGGTAAGGGCCTGGAATGGGTTGCATCTATTTCTTCTTATTATGGCTCTA | | YYYMHWVRQAPGKGLEWVASISSYYGST | |
| 2539- | CTTATTATGCCGATAGCGTCAAGGGCCGTTTCACTATAAGCGCAGACACATCCAAAAACACAGCCTACCTACAAATGAACAGCTTA | | YYADSVKGRFTISADTSKNTAYLQMNSLRA | |
| 2542 | AGAGCTGAGGACACTGCCGTCTATTATTGTGCTCGCTCTTGGTGGGCTTGGGCTTTTGACTACTGGGGTCAAGGAACCCTGGTCAC | | EDTAVYYCARSWWAWAFDYWGQGTLVT | |
| | CGTCTCCTCGGGTGGAGGTGGCAGTGATATCCAGATGACCCAGTCCCCGAGCTCCCTGTCCGCCTCTGTGGGCGATAGGGTCACC | | VSSGGGGSDIQMTQSPSSLSASVGDRVTIT | |
| | ATCACCTGCCGTGCCAGTCAGTCCGTGTCCAGCGCTGTAGCCTGGTATCAACAGAAACCAGGAAAAGCTCCGAAGCTTCTGATTTA | | CRASQSVSSAVAWYQQKPGKAPKLLIYSAS | |
| | CTCGGCATCCAGCCTCTACTCTGGAGTCCCTTCTCGCTTCTCTGGTAGCCGTTCCGGGACGGATTTCACTCTGACCATCAGCAGTCT | | SLYSGVPSRFSGSRSGTDFTLTISSLQPEDFA | |
| | GCAGCCGGAAGACTTCGCAACTTATTACTGTCAGCAACATTACTCTGTTTACGCTTCTCTGATCACGTTCGGACAGGGTACCAAGG | | TYYCQQHYSVYASLITFGQGTKVEIKLEDKT | |
| | TGGAGATCAAACTCGAGgacaaaactcacacaAAAGTGGAGCCCAAAACTTCTgataagacaatactTGCCCACCGTGCCCAGCACCTGAA | | HTKVEPKTSDKTHTCPPCPAPELLGGPSVF | |
| | CTCCTGGGGGGACCGTCAGTCTTCCTCTTCCCCCCAAAACCCAAGGACACCCTCATGATCTCCCGGACCCCTGAGGTCACATGCGT | | LFPPKPKDTLMISRTPEVTCVVVDVSHEDP | |
| | GGTGGTGGACGTGAGCCACGAAGACCCTGAGGTCAAGTTCAACTGGTACGTGGACGGCGTGGAGGTGCATAATGCCAAGACAAA | | EVKFNWYVDGVEVHNAKTKPREEQYNST | |
| | GCCGCGCGAGGAGCAGTACAACAGCACGTACCGTGTGGTCAGCGTCCTCACCGTCCTGCACCAGGACTGGCTGAATGGCAAGGA | | YRWSVLTVLHQDWLNGKEYKCKVSNKAL | |
| | GTACAAGTGCAAGGTCTCCAACAAAGCCCTCCCAGCCCCCATCGAGAAAACCATCTCCAAAGCCAAAGGGCAGCCCCGAGAACCA | | PAPIEKTISKAKGQPREPMVFDLPPSREEM | |
| | ATGGTGTTTGACCTGCCCCCATCCCGGGAGGAGATGACCAAGAACCAGGTCAGCCTGTGGTGCATGGTCAAGGGCTTCTATCCCA | | TKNQVSLWCMVKGFYPSDIAVEWESNGQ | |
| | GCGACATCGCCGTGGAGTGGGAGAGCAATGGGCAGCCGGAGAACAACTACAAGACCACGCCTCCCGTGCTGGACTCCGACGGCT | | PENNYKTTPPVLDSDGSFFLYSKLTVDKSR | |
| | CCTTCTTCCTGTACAGCAAGCTCACCGTGGACAAGAGCCGCTGGCAGCAGGGGAACGTCTTCTCATGCTCCGTGATGCATGAGGCT | | WQQGNVFSCSVMHEALHNHYTQKSLSLS | |
| | CTGCACAACCACTACACGCAGAAGAGCCTCTCCCTGTCTCCGGGTAAAAGCGGCAGCGAGACTCCCGGGACCTCAGAGTCCGCCA | | PGKSGSETPGTSESATPESGGGEVQLVESG | |
| | CACCCGAAAGTGGTGGCGGAGAGGTTCAGCTGGTGGAGTCTGGCGGTGGCCTGGTGCAGCCAGGGGGCTCACTCCGTTTGTCCT | | GGLVQPGGSLRLSCAASGFNISYSSIHWVR | |
| | GTGCAGCTTCTGGCTTCAACATCTCTTATTCTTCTATCCACTGGGTGCGTCAGGCCCCGGGTAAGGGCCTGGAATGGGTTGCATAT | | QAPGKGLEWVAYISSYYGYMADSVKGRF | |
| | ATTTCTTCTTATTATGGCTATACTTATTATGCCGATAGCGTCAAGGGCCGTTTCACTATAAGCGCAGACACATCCAAAAACACAGCC | | TISADTSKNTAYLQMNSLRAEDTAVYYCAR | |
| | TACCTACAAATGAACAGCTTAAGAGCTGAGGACACTGCCGTCTATTATTGTGCTCGCGCTCATTACTTCCCGTGGGCTGGTGCTAT | | AHYFPWAGAMDYWGQGTLVTVSSGGGG | |
| | GGACTACTGGGGTCAAGGAACCCTGGTCACCGTCTCCTCGGGTGGAGGTGGCAGTGATATCCAGATGACCCAGTCCCCGAGCTCC | | SDIQMTQSPSSLSASVGDRVTITCRASQSV | |
| | CTGTCCGCCTCTGTGGGCGATAGGGTCACCATCACCTGCCGTGCCAGTCAGTCCGTGTCCAGCGCTGTAGCCTGGTATCAACAGAA | | SSAVAWYQQKPGKAPKLLIYSASSLYSGVP | |
| | ACCAGGAAAAGCTCCGAAGCTTCTGATTTACTCGGCATCCAGCCTCTACTCTGGAGTCCCTTCTCGCTTCTCTGGTAGCCGTTCCGG | | SRFSGSRSGTDFTLTISSLCIPEDFATYYCQQ | |
| | GACGGATTTCACTCTGACCATCAGCAGTCTGCAGCCGGAAGACTTCGCAACTTATTACTGTCAGCAATACTACTGGCCGATCACGT | | YYWPITFGQGTKVEIK | |
| | TCGGACAGGGTACCAAGGTGGAGATCAAA | | | |
| 5048- | GAGGTTCAGCTGGTGGAGTCTGGCGGTGGCCTGGTGCAGCCAGGGGGCTCACTCCGTTTGTCCTGTGCAGCTTCTGGCTTCAACA | 19 | EVQLVESGGGLVQPGGSLRLSCAASGFNIS | 20 |
| hole- | TCTCTTATTATTATATGCACTGGGTGCGTCAGGCCCCGGGTAAGGGCCTGGAATGGETTGCATCTATTTCTTCTTATTATGGCTCTA | | YYYMHWVRQAPGKGLEWVASISSYYGST | |
| 2539- | CTTATTATGCCGATAGCGTCAAGGGCCGTTTCACTATAAGCGCAGACACATCCAAAAACACAGCCTACCTACAAATGAACAGCTTA | | YYADSVKGRFTISADTSKNTAYLQMNSLRA | |
| 2542 | AGAGCTGAGGACACTGCCGTCTATTATTGTGCTCGCTCTTGGTGGGCTTGGGCTTTTGACTACTGGGGTCAAGGAACCCTGGTCAC | | EDTAVYYCARSWWAWAFDYWGQGTLVT | |
| | CGTCTCCTCGGGTGGAGGTGGCAGTGATATCCAGATGACCCAGTCCCCGAGCTCCCTGTCCGCCTCTGTGGGCGATAGGGTCACC | | VSSGGGGSDIQMTQSPSSLSASVGDRVTIT | |
| | ATCACCTGCCGTGCCAGTCAGTCCGTGTCCAGCGCTGTAGCCTGGTATCAACAGAAACCAGGAAAAGCTCCGAAGCTTCTGATTTA | | CRASQSVSSAVAWYQQKPGKAPKLLIYSAS | |
| | CTCGGCATCCAGCCTCTACTCTGGAGTCCCTTCTCGCTTCTCTGGTAGCCGTTCCGGGACGGATTTCACTCTGACCATCAGCAGTCT | | SLYSGVPSRFSGSRSGTDFTLTISSLQPEDFA | |
| | GCAGCCGGAAGACTTCGCAACTTATTACTGTCAGCAACATTACTCTGTTTACGCTTCTCTGATCACGTTCGGACAGGGTACCAAGG | | TYYCQQHYSVYASLITFGQGTKVEIKLEDKT | |
| | TGGAGATCAAACTCGAGgacaaaactcacacaAAAGTTGAGCCCAAATCTTCTgataagacccataatTGCCCACCGTGCCCAGCACCTGAA | | HTKVEPKSSDKTHNCPPCPAPELLGGPSVF | |
| | CTCCTGGGGGGACCGTCAGTCTTTCTCTTCCCCCCAAAACCCAAGGACACCCTCATGATCTCCCGGACCCCTGAGGTCACATGCGT | | LFPPKPKDILMISRTPEVTCVVVDVSHEDP | |
| | GGTGGTGGACGTGAGCCACGAAGACCCTGAGGTCAAGTTCAACTGGTACGTGGACGGCGTGGAGGTGCATAATGCCAAGACAAA | | EVKFNWYVDGVEVHNAKTKPREEQYNST | |
| | GCCGCGGGAGGAGCAGTACAACAGCACGTACCGTGTGGTCAGCGTCCTCACCGTCCTGCACCAGGACTGGCTGAATGGCAAGGA | | YRWSVLTVLHQDWLNGKEYKCKVSNKAL | |
| | GTACAAGTGCAAGGTCTCCAACAAAGCCCTCCCAGCCCCCATCGAGAAAACCATCTCCAAAGCCAAAGGGCAGCCCCGAGAACCA | | PAPIEKTISKAKGQPREPQVYTLPPIRELMT | |
| | CAGGTGTACACCCTGCCCCCAATCCGGGAGCTGATGACCAGCAACCAGGTCAGCCTGAGCTGCGCCGTCAAAGGCTTCTATCCCA | | SNQVSLSCAVKGFYPSDIAVEWESNGQPE | |
| | GCGACATCGCCGTGGAGTGGGAGAGCAATGGGCAGCCGGAGAACAACTACAAGACCACGCCTCCCGTGCTGGACTCCGACGGCT | | NNYKTTPPVLDSDGSFFLVSKLTVDKSRW | |
| | CCTTCTTCCTCGTGAGCAAGCTCACCGTGGACAAGAGCAGGTGGCAGCAGGGGAACGTCTTCTCATGCTCCGTGATGCATGAGGC | | QQGNVFSCSVMHEALHNHYTQKSLSLSPG | |
| | TCTGCACAACCACTACACGCAGAAGAGCCTCTCCCTGTCTCCGGGTAAAAGCGGCAGCGAGACTCCCGGGACCTCAGAGTCCGCC | | KSGSETPGTSESATPESGGGEVQLVESGGG | |
| | ACACCCGAAAGTGGTGGCGGAGAGGTTCAGCTGGTGGAGTCTGGCGGTGGCCTGGTGCAGCCAGGGGGCTCACTCCGTTTGTCC | | LVQPGGSLRLSCAASGFNISSYYIHWVRQA | |
| | TGTGCAGCTTCTGGCTTCAACATCTCTTCTTATTATATCCACTGGGTGCGTCAGGCCCCGGGTAAGGGCCTGGAATGGGTTGCATC | | PGKGLEWVASIYSSYGYTSYADSVKGRFTIS | |
| | TATTTATTTTTCTTATGGCTATACTTCTTATGCCGATAGCGTCAAGGGCCGTTTCACTATAAGCGCAGACACATCCAAAAACACAGC | | ADTSKNTAYLQMNSLRAEDTAVYYCARTV | |
| | CTACCTACAAATGAACAGCTTAAGAGCTGAGGACACTGCCGTCTATTATTGTGCTCGCACTGTTCGTGGATCCAAAAAACCGTACT | | RGSKKPYFSGWAMDYWGQGTLVTVSSG | |
| | TCTCTGGTTGGGCTATGGACTACTGGGGTCAAGGAACCCTGGTCACCGTCTCCTCGGGTGGAGGTGGCAGTGATATCCAGATGAC | | GGGSDIQMTQSPSSISASVGDRVTITCRAS | |
| | CCAGTCCCCGAGCTCCCTGTCCGCCTCTGTGGGCGATAGGGTCACCATCACCTGCCGTGCCAGTCAGTCCGTGTCCAGCGCTGTAG | | QSVSSAVAWYQQKPGKAPKWYSASSLYS | |
| | CCTGGTATCAACAGAAACCAGGAAAAGCTCCGAAGCTTCTGATTTACTCGGCATCCAGCCTCTACTCTGGAGTCCCTTCTCGCTTCT | | GVPSRFSGSRSGTDFTLTISSLQPEDFATYY | |
| | CTGGTAGCCGTTCCGGGACGGATTTCACTCTGACCATCAGCAGTCTGCAGCCGGAAGACTTCGCAACTTATTACTGTCAGCAATAC | | CQQYSWGPFTFGQGTKVEIK | |
| | TCTTGGGGTCCGTTCACGTTCGGACAGGGTACCAAGGTGGAGATCAAA | | | |
| 5062- | GAGGTTCAGCTGGTGGAGTCTGGCGGTGGCCTGGTGCAGCCAGGGGGCTCACTCCGTTTGTCCTGTGCAGCTTCTGGCTTCAACat | 21 | EVQLVESGGGLVQPGGSLRLSCAASGFNIS | 22 |
| knob- | ctcttattattatatcCACTGGGTGCGTCAGGCCCCGGGTAAGGGCCTGGAATGGGTTGCATCTATTTATTCTTCTTCTAGCTATACTTAT | | YYYIHWVRQAPGKGLEWVASIYSSSSYTYY | |
| 2539- | TATGCCGATAGCGTCAAGGGCCGTTTCACTATAAGCGCAGACACATCCAAAAACACAGCCTACCTACAAATGAACAGCTTAAGAGC | | ADSVKGRFTISADTSKNTAYLQMNSLRAED | |
| 2542 | TGAGGACACTGCCGTCTATTATTGTGCTCGCTCTTCTTACGCTTGGGCTATTGACTACTGGGGTCAAGGAACCCTGGTCACCGTCTC | | TAVYYCARSSYAWAIDYWGQGTLVTVSSG | |
| | CTCGGGTGGAGGTGGCAGTGATATCCAGATGACCCAGTCCCCGAGCTCCCTGTCCGCCTCTGTGGGCGATAGGGTCACCATCACC | | GGGSDIQMTQSPSSLSASVGDRVTITCRAS | |
| | TGCCGTGCCAGTCAGTCCGTGTCCAGCGCTGTAGCCTGGTATCAACAGAAACCAGGAAAAGCTCCGAAGCTTCTGATTTACTCGGC | | QSVSSAVAWYQQKPGKAPKLLIYSASSLYS | |
| | ATCCAGCCTCTACTCTGGAGTCCCTTCTCGCTTCTCTGGTAGCCGTTCCGGGACGGATTTCACTCTGACCATCAGCAGTCTGCAGCC | | GVPSRFSGSRSGTDFTLTISSLQPEDFATYY | |
| | GGAAGACTTCGCAACTTATTACTGTCAGCAATCTGGTTGGTGGGGTGTTTCTCTGATCACGTTCGGACAGGGTACCAAGGTGGAG | | CQQSGWWGVSLITFGQGTKVEIKLEDKTH | |
| | ATCAAACTCGAGgacaaaactcacacaAAAGTGGAGCCCAAAACTTCTgataagacccatactTGCCCACCGTGCCCAGCACCTGAACTCCT | | TKVEPKTSDKTHTCPPCPAPELLGGPSVFLF | |
| | GGGGGGACCGTCAGTCTTCCTCTTCCCCCCAAAACCCAAGGACACCCTCATGATCTCCCGGACCCCTGAGGTCACATGCGTGGTGG | | PPKPKDTLMISRTPEVICVVVDVSHEDPEV | |
| | TGGACGTGAGCCACGAAGACCCTGAGGTCAAGTTCAACTGGTACGTGGACGGCGTGGAGGTGCATAATGCCAAGACAAAGCCGC | | KFNWYVDGVEVHNAKTKPREEQYNSTYR | |
| | GCGAGGAGCAGTACAACAGCACGTACCGTGTGGTCAGCGTCCTCACCGTCCTGCACCAGGACTGGCTGAATGGCAAGGAGTACA | | VVSVLTVLHQDWLNGKEYKCKVSNKALPA | |
| | AGTGCAAGGTCTCCAACAAAGCCCTCCCAGCCCCCATCGAGAAAACCATCTCCAAAGCCAAAGGGCAGCCCCGAGAACCAATGGT | | PIEKTISKAKGQPREPMVFDLPPSREEMTK | |
| | GTTTGACCTGCCCCCATCCCGGGAGGAGATGACCAAGAACCAGGTCAGCCTGTGGTGCATGGTCAAGGGCTTCTATCCCAGCGAC | | NQVSLWCMVKGFYPSDIAVEWESNGQPE | |
| | ATCGCCGTGGAGTGGGAGAGCAATGGGCAGCCGGAGAACAACTACAAGACCACGCCTCCCGTGCTGGACTCCGACGGCTCCTTCT | | NNYKTTPPVLDSDGSFFLYSKLTVDKSRWQ | |
| | TCCTGTACAGCAAGCTCACCGTGGACAAGAGCCGCTGGCAGCAGGGGAACGTCTTCTCATGCTCCGTGATGCATGAGGCTCTGCA | | QGNVFSCSVMHEALHNHYTQKSLSLSPGK | |
| | CAACCACTACACGCAGAAGAGCCTCTCCCTGTCTCCGGGTAAAAGCGGCAGCGAGACTCCCGGGACCTCAGAGTCCGCCACACCC | | SGSETPGTSESATPESGGGEVQLVESGGGL | |
| | GAAAGTGGTGGCGGAGAGGTTCAGCTGGTGGAGTCTGGCGGTGGCCTGGTGCAGCCAGGGGGCTCACTCCGTTTGTCCTGTGCA | | VQPGGSLRLSCAASGFNISYSSIHWVRQAP | |
| | GCTTCTGGCTTCAACATCTCTTATTCTTCTATCCACTGGGTGCGTCAGGCCCCGGGTAAGGGCCTGGAATGGGTTGCATATATTTCT | | GKGLEWVAYISSYYGYTYYADSVKGRFTISA | |
| | TCTTATTATGGCTATACTTATTATGCCGATAGCGTCAAGGGCCGTTTCACTATAAGCGCAGACACATCCAAAAACACAGCCTACCTA | | DTSKNTAYLQMNSLRAEDTAVYYCARAHY | |
| | CAAATGAACAGCTTAAGAGCTGAGGACACTGCCGTCTATTATTGTGCTCGCGCTCATTACTTCCCGTGGGCTGGTGCTATGGACTA | | FPWAGAMDYWGQGTLVTVSSGGGGSDI | |
| | CTGGGGTCAAGGAACCCTGGTCACCGTCTCCTCGGGTGGAGGTGGCAGTGATATCCAGATGACCCAGTCCCCGAGCTCCCTGTCC | | QMTQSPSSLSASVGDRVTITCRASQSVSSA | |
| | GCCTCTGTGGGCGATAGGGTCACCATCACCTGCCGTGCCAGTCAGTCCGTGTCCAGCGCTGTAGCCTGGTATCAACAGAAACCAG | | VAWYQQKPGKAPKLLIYSASSLYSGVPSRF | |
| | GAAAAGCTCCGAAGCTTCTGATTTACTCGGCATCCAGCCTCTACTCTGGAGTCCCTTCTCGCTTCTCTGGTAGCCGTTCCGGGACGG | | SGSRSGTDFTLTISSLQPEDFATYYCQQYY | |
| | ATTTCACTCTGACCATCAGCAGTCTGCAGCCGGAAGACTTCGCAACTTATTACTGTCAGCAATACTACTGGCCGATCACGTTCGGAC | | WPITFGQGTKVEIK | |
| | AGGGTACCAAGGTGGAGATCAAA | | | |
| 5062- | GAGGTTCAGCTGGTGGAGTCTGGCGGTGGCCTGGTGCAGCCAGGGGGCTCACTCCGTTTGTCCTGTGCAGCTTCTGGCTTCAACat | 23 | EVQLVESGGGLVQPGGSLRLSCAASGFNIS | 24 |
| hole- | ctcttattattatatcCACTGGGTGCGTCAGGCCCCGGGTAAGGGCCTGGAATGGGTTGCATCTATTTATTCTTCTTCTAGCTATACTTAT | | YYYIHWVROAPGKGLEWVASIYSSSSYTYY | |
| 2539- | TATGCCGATAGCGTCAAGGGCCGTTTCACTATAAGCGCAGACACATCCAAAAACACAGCCTACCTACAAATGAACAGCTTAAGAGC | | ADSVKGRFTISADTSKNTAYLQMNSLRAED | |
| 2542 | TGAGGACACTGCCGTCTATTATTGTGCTCGCTCTTCTTACGCTTGGGCTATTGACTACTGGGGTCAAGGAACCCTGGTCACCGTCTC | | TAVYYCARSSYAWAIDYWGQGTLVTVSSG | |
| | CTCGGGTGGAGGTGGCAGTGATATCCAGATGACCCAGTCCCCGAGCTCCCTGTCCGCCTCTGTGGGCGATAGGGTCACCATCACC | | GGGSDIQMTQSPSSLSASVGDRVTITCRAS | |
| | TGCCGTGCCAGTCAGTCCGTGTCCAGCGCTGTAGCCTGGTATCAACAGAAACCAGGAAAAGCTCCGAAGCTTCTGATTTACTCGGC | | QSVSSAVAWYQQKPGKAPKLLIYSASSLYS | |
| | ATCCAGCCTCTACTCTGGAGTCCCTTCTCGCTTCTCTGGTAGCCGTTCCGGGACGGATTTCACTCTGACCATCAGCAGTCTGCAGCC | | GVPSRFSGSRSGTDFTLTISSLQPEDFATYY | |
| | GGAAGACTTCGCAACTTATTACTGTCAGCAATCTGGTTGETGGGGTGTTTCTCTGATCACGTTCGGACAGGGTACCAAGGTGGAG | | CQQSGWWGVSLITFGQGTKVEIKLEDKTH | |
| | ATCAAACTCGAGgacaaaacttacacaAAAGTTGAGCCCAAATCTTCTgataagacccataatTGCCCACCGTGCCCAGCACCTGAACTCCT | | TKVEPKSSDKTHNCPPCPAPELLGGPSVFLF | |
| | GGGGGGACCGTCAGTCTTCCTCTTCCCCCCAAAACCCAAGGACACCCTCATGATCTCCCGGACCCCTGAGGTCACATGCGTGGTGG | | PPKPKDTLMISRTPEVICVVVDVSHEDPEV | |
| | TGGACGTGAGCCACGAAGACCCTGAGGTCAAGTTCAACTGGTACGTGGACGGCGTGGAGGTGCATAATGCCAAGACAAAGCCGC | | KFNWYVDGVEVHNAKTKPREEQYNSTYR | |
| | GGGAGGAGCAGTACAACAGCACGTACCGTGTGGTCAGCGTCCTCACCGTCCTGCACCAGGACTGGCTGAATGGCAAGGAGTACA | | VVSVLTVLHQDWLNGKEYKCKVSNKALPA | |
| | AGTGCAAGGTCTCCAACAAAGCCCTCCCAGCCCCCATCGAGAAAACCATCTCCAAAGCCAAAGGGCAGCCCCGAGAACCACAGGT | | PIEKTISKAKGQPREPQVYTLPPIRELMTSN | |
| | GTACACCCTGCCCCCAATCCGGGAGCTGATGACCAGCAACCAGGTCAGCCTGAGCTGCGCCGTCAAAGGCTTCTATCCCAGCGAC | | QVSLSCAVKGFYPSDIAVEWESNGQPENN | |
| | ATCGCCGTGGAGTGGGAGAGCAATGGGCAGCCGGAGAACAACTACAAGACCACGCCTCCCGTGCTGGACTCCGACGGCTCCTTCT | | YKTIPPVLDSDGSFFLVSKLTVDKSRWQQ | |
| | TCCTCGTGAGCAAGCTCACCGTGGACAAGAGCAGGTGGCAGCAGGGGAACGTCTTCTCATGCTCCGTGATGCATGAGGCTCTGCA | | GNVFSCSVMHEALHNHYTQKSLSLSPGKS | |
| | CAACCACTACACGCAGAAGAGCCTCTCCCTGTCTCCGGGTAAAAGCGGCAGCGAGACTCCCGGGACCTCAGAGTCCGCCACACCC | | GSETPGTSESATPESGGGEVQLVESGGGLV | |
| | GAAAGTGGTGGCGGAGAGGTTCAGCTGGTGGAGTCTGGCGGTGGCCTGGTGCAGCCAGGGGGCTCACTCCGTTTGTCCTGTGCA | | QPGGSLRLSCAASGFNISSYYIHWVRQAPG | |
| | GCTTCTGGCTTCAACATCTCTTCTTATTATATCCACTGGGTGCGTCAGGCCCCGGGTAAGGGCCTGGAATGGGTTGCATCTATTTAT | | KGLEWVASIYSSYGYTSYADSVKGRFTISAD | |
| | TCTTCTTATGGCTATACTTCTTATGCCGATAGCGTCAAGGGCCGTTTCACTATAAGCGCAGACACATCCAAAAACACAGCCTACCTA | | TSKNTAYLQMNSLRAEDTAVYYCARTVRG | |
| | CAAATGAACAGCTTAAGAGCTGAGGACACTGCCGTCTATTATTGTGCTCGCACTGTTCGTGGATCCAAAAAACCGTACTTCTCTGG | | SKKPYFSGWAMDYWGQGTLVTVSSGGG | |
| | TTGGGCTATGGACTACTGGGGTCAAGGAACCCTGGTCACCGTCTCCTCGGGTGGAGGTGGCAGTGATATCCAGATGACCCAGTCC | | GSDIQMTQSPSSLSASVGDRVTITCRASQS | |
| | CCGAGCTCCCTGTCCGCCTCTGTGGGCGATAGGGTCACCATCACCTGCCGTGCCAGTCAGTCCGTGTCCAGCGCTGTAGCCTGGTA | | VSSAVAWYQQKPGKAPKLLIYSASSLYSGV | |
| | TCAACAGAAACCAGGAAAAGCTCCGAAGCTTCTGATTTACTCGGCATCCAGCCTCTACTCTGGAGTCCCTTCTCGCTTCTCTGGTAG | | PSRFSGSRSGTDFTLTISSLQPEDFATYYCQ | |
| | CCGTTCCGGGACGGATTTCACTCTGACCATCAGCAGTCTGCAGCCGGAAGACTTCGCAACTTATTACTGTCAGCAATACTCTTGGG | | QYSWGPFTFGQGTKVEIK | |
| | GTCCGTTCACGTTCGGACAGGGTACCAAGGTGGAGATCAAA | | | |
| 5063- | GAGGTTCAGCTGGTGGAGTCTGGCGGTGGCCTGGTGCAGCCAGGGGGCTCACTCCGTTTGTCCTGTGCAGCTTCTGGCTTCAACA | 25 | EVQLVESGGGLVQPGGSLRLSCAASGFNIS | 26 |
| knob- | TCTCTTATTATTATATCCACTGGGTGCGTCAGGCCCCGGGTAAGGGCCTGGAATGGGTTGCATCTATTTATCCTTCTTCTGGCTATA | | YYYIHWVRQAPGKGLEWVASIYPSSGYTYY | |
| 2539- | CTTATTATGCCGATAGCGTCAAGGGCCGTTTCACTATAAGCGCAGACACATCCAAAAACACAGCCTACCTACAAATGAACAGCTTA | | ADSVKGRFTISADISKNTAYLQMNSLRAED | |
| 2542 | AGAGCTGAGGACACTGCCGTCTATTATTGTGCTCGCTCTTCTTTCTACTGGGCTATGGACTACTGGGGTCAAGGAACCCTGGTCAC | | TAVYYCARSSFYWAMDYWGQGTLVTVSS | |
| | CGTCTCCTCGGGTGGAGGTGGCAGTGATATCCAGATGACCCAGTCCCCGAGCTCCCTGTCCGCCTCTGTGGGCGATAGGGTCACC | | GGGGSDIQMTQSPSSLSASVGDRVIITCRA | |
| | ATCACCTGCCGTGCCAGTCAGTCCGTGTCCAGCGCTGTAGCCTGGTATCAACAGAAACCAGGAAAAGCTCCGAAGCTTCTGATTTA | | SQSVSSAVAWYQQKPGKAPKLLIYSASSLY | |
| | CTCGGCATCCAGCCTCTACTCTGGAGTCCCTTCTCGCTTCTCTGGTAGCCGTTCCGGGACGGATTTCACTCTGACCATCAGCAGTCT | | SGVPSRFSGSRSGTDFILTISSLQPEDFATY | |
| | GCAGCCGGAAGACTTCGCAACTTATTACTGTCAGCAATCTTACGCTGCTTACCTGTTCACGTTCGGACAGGGTACCAAGGTGGAGA | | YCQQSYAAYLFTFGQGTKVEIKLEDKTHTK | |
| | TCAAACTCGAGgacaaaactcacacaAAAGTGGAGCCCAAAACTTCTgataagacccatactTGCCCACCGTGCCCAGCACCTGAACTCCTG | | VEPKTSDKTHTCPPCPAPELLGGPSVFLFPP | |
| | GGGGGACCGTCAGTCTTCCTCTTCCCCCCAAAACCCAAGGACACCCTCATGATCTCCCGGACCCCTGAGGTCACATGCGTGGTGGT | | KPKDTLMISRTPEVTCVVVDVSHEDPEVKF | |
| | GGACGTGAGCCACGAAGACCCTGAGGTCAAGTTCAACTGGTACGTGGACGGCGTGGAGGTGCATAATGCCAAGACAAAGCCGCG | | NWYVDGVEVHNAKTKPREEQYNSTYRVV | |
| | CGAGGAGCAGTACAACAGCACGTACCGTGTGGTCAGCGTCCTCACCGTCCTGCACCAGGACTGGCTGAATGGCAAGGAGTACAA | | SVLTVLHQDWLNGKEYKCKVSNKALPAPIE | |
| | GTGCAAGGTCTCCAACAAAGCCCTCCCAGCCCCCATCGAGAAAACCATCTCCAAAGCCAAAGGGCAGCCCCGAGAACCAATGGTG | | KTISKAKGQPREPMVFDLPPSREEMTKNQ | |
| | TTTGACCTGCCCCCATCCCGGGAGGAGATGACCAAGAACCAGGTCAGCCTGTGGTGCATGGTCAAGGGCTTCTATCCCAGCGACA | | VSLWCMVKGFYPSDIAVEWESNGQPENN | |
| | TCGCCGTGGAGTGGGAGAGCAATGGGCAGCCGGAGAACAACTACAAGACCACGCCTCCCGTGCTGGACTCCGACGGCTCCTTCTT | | YKTTPPVLDSDGSFFLYSKLTVDKSRWQQG | |
| | CCTGTACAGCAAGCTCACCGTGGACAAGAGCCGCTGGCAGCAGGGGAACGTCTTCTCATGCTCCGTGATGCATGAGGCTCTGCAC | | NVFSCSVMHEALHNHYTQKSLSLSPGKSG | |
| | AACCACTACACGCAGAAGAGCCTCTCCCTGTCTCCGGGTAAAAGCGGCAGCGAGACTCCCGGGACCTCAGAGTCCGCCACACCCG | | SETPGISESATPESGGGEVQLVESGGLVQ | |
| | AAAGTGGTGGCGGAGAGGTTCAGCTGGTGGAGTCTGGCGGTGGCCTGGTGCAGCCAGGGGGCTCACTCCGTTTGTCCTGTGCAG | | PGGSLRLSCAASGFNISYSSIHWVRQAPGK | |
| | CTTCTGGCTTCAACATCTCTTATTCYTCTATCCACTGGGTGCGTCAGGCCCCGGGTAAGGGCCTGGAATGGGTTGCATATATTTCTT | | GLEWVAYISSYYGYTYYADSVKGRFTISADT | |
| | CTTATTATGGCTATACTTATTATGCCGATAGCGTCAAGGGCCGTTTCACTATAAGCGCAGACACATCCAAAAACACAGCCTACCTAC | | SKNTAYLQMNSLRAEDTAVYYCARAHYFP | |
| | AAATGAACAGCTTAAGAGCTGAGGACACTGCCGTCTATTATTGTGCTCGCGCTCATTACTTCCCGTGGGCTGGTGCTATGGACTAC | | WAGAMDYWGQGILVTVSSGGGGSDIQ | |
| | TGGGGTCAAGGAACCCTGGTCACCGTCTCCTCGGGTGGAGGTGGCAGTGATATCCAGATGACCCAGTCCCCGAGCTCCCTGTCCG | | MTQSPSSLSASVGDRVTITCRASQSVSSAV | |
| | CCTCTGTGGGCGATAGGGTCACCATCACCTGCCGTGCCAGTCAGTCCGTGTCCAGCGCTGTAGCCTGGTATCAACAGAAACCAGG | | VAWYQQKPGKAPKLLIYSASSLYSGVPSRFS | |
| | AAAAGCTCCGAAGCTTCTGATTTACTCGGCATCCAGCCTCTACTCTGGAGTCCCTTCTCGCTTCTCTGGTAGCCGTTCCGGGACGGA | | GSRSGTDFTLTISSLQPEDFATYYCQQYYW | |
| | TTTCACTCTGACCATCAGCAGTCTGCAGCCGGAAGACTTCGCAACTTATTACTGTCAGCAATACTACTGGCCGATCACGTTCGGACA | | PITFGQGTKVEIK | |
| | GGGTACCAAGGTGGAGATCAAA | | | |
| 5063- | GAGGTTCAGCTGGTGGAGTCTGGCGGTGGCCTGGTGCAGCCAGGGGGCTCACTCCGTTTGTCCTGTGCAGCTTCTGGCTTCAACA | 27 | EVQLVESGGGLVQPGGSLRLSCAASGFNIS | 28 |
| hole- | TCTCTTATTATTATATCCACTGGGTGCGTCAGGCCCCGGGTAAGGGCCTGGAATGGGTTGCATCTATTTATCCTTCTTCTGGCTATA | | YYYIHWVRQAPGKGLEWVASIYPSSGYTYY | |
| 2539- | CTTATTATGCCGATAGCGTCAAGGGCCGTTTCACTATAAGCGCAGACACATCCAAAAACACAGCCTACCTACAAATGAACAGCTTA | | ADSVKGRFTISADTSKNTAYLQMNSLRAED | |
| 2542 | AGAGCTGAGGACACTGCCGTCTATTATTGTGCTCGCTCTTCTTTCTACTGGGCTATGGACTACTGGGGTCAAGGAACCCTGGTCAC | | TAVYYCARSSFYWAMDYWGQGTLVTVSS | |
| | CGTCTCCTCGGGTGGAGGTGGCAGTGATATCCAGATGACCCAGTCCCCGAGCTCCCTGTCCGCCTCTGTGGGCGATAGGGTCACC | | GGGGSDIQMTQSPSSLSASVGDRVTITCRA | |
| | ATCACCTGCCGTGCCAGTCAGTCCGTGTCCAGCGCTGTAGCCTGGTATCAACAGAAACCAGGAAAAGCTCCGAAGCTTCTGATTTA | | SQSVSSAVAWYQQKPGKAPKLLIYSASSLY | |
| | CTCGGCATCCAGCCTCTACTCTGGAGTCCCTTCTCGCTTCTCTGGTAGCCGTTCCGGGACGGATTTCACTCTGACCATCAGCAGTCT | | SGVPSRFSGSRSGTDFTLTISSLCIPEDFATY | |
| | GCAGCCGGAAGACTTCGCAACTTATTACTGTCAGCAATCTTACGCTGCTTACCTGTTCACGTTCGGACAGGGTACCAAGGTGGAGA | | YCQQSYAAYLFTFGQGTKVEIKLEDKTHTK | |
| | TCAAACTCGAGgacaaaactcacacaAAAGTTGAGCCCAAATCTTCTgataagacccataatTGCCCACCGTGCCCAGCACCTGAACTCCTG | | VEPKSSDKTHNCPPCPAPELLGGPSVFLFP | |
| | GGGGGACCGTCAGTCTTCCTCTTCCCCCCAAAACCCAAGGACACCCTCATGATCTCCCGGACCCCTGAGGTCACATGCGTGGTGGT | | PKPKDTLMISRTPEVTCVVVDVSHEDPEVK | |
| | GGACGTGAGCCACGAAGACCCTGAGGTCAAGTTCAACTGGTACGTGGACGGCGTGGAGGTGCATAATGCCAAGACAAAGCCGCG | | FNWYVDGVEVHNAKTKPREEQYNSTYRV | |
| | GGAGGAGCAGTACAACAGCACGTACCGTGTGGTCAGCGTCCTCACCGTCCTGCACCAGGACTGGCTGAATGGCAAGGAGTACAA | | VSVLTVLHQDWLNGKEYKCKVSNKALPAP | |
| | GTGCAAGGTCTCCAACAAAGCCCTCCCAGCCCCCATCGAGAAAACCATCTCCAAAGCCAAAGGGCAGCCCCGAGAACCACAGGTG | | IEKTISKAKGQPREPQVYTLPPIRELMTSNQ | |
| | TACACCCTGCCCCCAATCCGGGAGCTGATGACCAGCAACCAGGTCAGCCTGAGCTGCGCCGTCAAAGGCTTCTATCCCAGCGACAT | | VSLSCAVKGFYPSDIAVEWESNGQPENNY | |
| | CGCCGTGGAGTGGGAGAGCAATGGGCAGCCGGAGAACAACTACAAGACCACGCCTCCCGTGCTGGACTCCGACGGCTCCTTCTTC | | KTTPPVLDSDGSFFLVSKLTVDKSRWQQG | |
| | CTCGTGAGCAAGCTCACCGTGGACAAGAGCAGGTGGCAGCAGGGGAACGTCTTCTCATGCTCCGTGATGCATGAGGCTCTGCACA | | NVFSCSVMHEALHNHYTQKSLSLSPGKSG | |
| | ACCACTACACGCAGAAGAGCCTCTCCCTGTCTCCGGGTAAAAGCGGCAGCGAGACTCCCGGGACCTCAGAGTCCGCCACACCCGA | | SETPGTSESATPESGGGEVQLVESGGGLVQ | |
| | AAGTGGTGGCGGAGAGGTTCAGLTGGTGGAGTCTGGCGGTGGCCTGGTGCAGCCAGGGGGCTCACTCCGTTTGTCCTGTGCAGC | | PGGSLRLSCAASGFNISSYYIHWVRQAPGK | |
| | TTCTGGCTTCAACATCTCTTCTTATTATATCCACTGGGTGCGTCAGGCCCCGGGTAAGGGCCTGGAATGGGTTGCATCTATTTATTC | | GLEWVASIYSSYGYTSYADSVKGRFTISADT | |
| | TTCTTATGGCTATACTTCTTATGCCGATAGCGTCAAGGGCCGTTTCACTATAAGCGCAGACACATCCAAAAACACAGCCTACCTACA | | SKNTAYLQMNSLRAEDTAVYYCARTVRGS | |
| | AATGAACAGCTTAAGAGCTGAGGACACTGCCGTCTATTATTGTGCTCGCACTGTTCGTGGATCCAAAAAACCGTACTTCTCTGGTT | | KKPYFSGWAMDYWGQGTLVTVSSGGGG | |
| | GGGCTATGGACTACTGGGGTCAAGGAACCCTGGTCACCGTCTCCTCGGGTGGAGGTGGCAGTGATATCCAGATGACCCAGTCCCC | | SDIQMTQSPSSISASVGDRVTITCRASQSV | |
| | GAGCTCCCTGTCCGCCTCTGTGGGCGATAGGGTCACCATCACCTGCCGTGCCAGTCAGTCCGTGTCCAGCGCTGTAGCCTGGTATC | | SSAVAWYQQKPGKAPKWYSASSLYSGVP | |
| | AACAGAAACCAGGAAAAGCTCCGAAGCTTCTGATTTACTCGGCATCCAGCCTCTACTCTGGAGTCCCTTCTCGCTTCTCTGGTAGCC | | SRFSGSRSGTDFTLTISSLQPEDFATYYCQQ | |
| | GTTCCGGGACGGATTTCACTCTGACCATCAGCAGTCTGCAGCCGGAAGACTTCGCAACTTATTACTGTCAGCAATACTCTTGGGGT | | YSWGPFTFGQGTKVEIK | |
| | CCGTTCACGTTCGGACAGGGTACCAAGGTGGAGATCAAA | | | |
| 5080- | GAGGTTCAGCTGGTGGAGTCTGGCGGTGGCCTGGTGCAGCCAGGGGGCTCACTCCGTTTGTCCTGTGCAGCTTCTGGCTTCAACA | 29 | EVQLVESGGGLVQPGGSLRLSCAASGFNIS | 30 |
| knob- | TCTCTTATTATTCTATGCACTGGGTGCGTCAGGCCCCGGGTAAGGGCCTGGAATGGGTTGCATCTATTTCTTCTTATTATAGCTCTA | | YYSMHWVRQAPGKGLEWVASISSYYSSTS | |
| 2539- | CTTCTTATGCCGATAGCGTCAAGGGCCGTTTCACTATAAGCGCAGACACATCCAAAAACACAGCCTACCTACAAATGAACAGCTTA | | YADSVKGRFTISADTSKNTAYLQMNSLRAE | |
| 2542 | AGAGCTGAGGACACTGCCGTCTATTATTGTGCTCGCTTCTGGTACCCGGGTATGGACTACTGGGGTCAAGGAACCCTGGTCACCG | | DTAVYYCARFWYPGMDYWGQGTLVTVSS | |
| | TCTCCTCGGGTGGAGGTGGCAGTGATATCCAGATGACCCAGTCCCCGAGCTCCCTGTCCGCCTCTGTGGGCGATAGGGTCACCAT | | GGGGSDIQMTQSPSSLSASVGDRVTITCRA | |
| | CACCTGCCGTGCCAGTCAGTCCGTGTCCAGCGCTGTAGCCTGGTATCAACAGAAACCAGGAAAAGCTCCGAAGCTTCTGATTTACT | | SQSVSSAVAWYQQKPGKAPKLLIYSASSLY | |
| | CGGCATCCAGCCTCTACTCTGGAGTCCCTTCTCGCTTCTCTGGTAGCCGTTCCGGGACGGATTTCACTCTGACCATCAGCAGTCTGC | | SGVPSRFSGSRSGTDFTLTISSLQPEDFATY | |
| | AGCCGGAAGACTTCGCAACTTATTACTGTCAGCAACATTGGTCTTACCCGATCACGTTCGGACAGGGTACCAAGGTGGAGATCAA | | YCQQHWSYPITFGQGTKVEIKLEDKTHTKV | |
| | ACTCGAGgacaaaactcacacaAAAGTGGAGCCCAAAACTTCTgataagacccatactTGCCCACCGTGCCCAGCACCTGAACrCCTGGGG | | EPKISDKTHTCPPCPAPELLGGPSVFLFPPK | |
| | GGACCGTCAGTCTTCCTCTTCCCCCCAAAACCCAAGGACACCCTCATGATCTCCCGGACCCCTGAGGTCACATGCGTGGTGGTGGA | | PKDTLMISRTPEVTCVVVDVSHEDPEVKFN | |
| | CGTGAGCCACGAAGACCCTGAGGTCAAGTTCAACTGGTACGTGGACGGCGTGGAGGTGCATAATGCCAAGACAAAGCCGCGCGA | | WYVDGVEVHNAKTKPREEQYNSTYRVVS | |
| | GGAGCAGTACAACAGCACGTACCGTGTGGTCAGCGTCCTCACCGTCCTGCACCAGGACTGGCTGAATGGCAAGGAGTACAAGTGC | | VLTVLHQDWLNGKEYKCKVSNKALPAPIE | |
| | AAGGTCTCCAACAAAGCCCTCCCAGCCCCCATCGAGAAAACCATCTCCAAAGCCAAAGGGCAGCCCCGAGAACCAATGGTGTTTG | | KTISKAKGQPREPMVFDLPPSREEMTKNQ | |
| | ACCTGCCCCCATCCCGGGAGGAGATGACCAAGAACCAGGTCAGCCTGTGGTGCATGGTCAAGGGCTTCTATCCCAGCGACATCGC | | VSLWCMVKGFYPSDIAVEWESNGQPENN | |
| | CGTGGAGTGGGAGAGCAATGGGCAGCCGGAGAACAACTACAAGACCACGCCTCCCGTGCTGGACTCCGACGGCTCCTTCTTCCTG | | YKTTPPVLDSDGSFFLYSKLIVDKSRWQQG | |
| | TACAGCAAGCTCACCGTGGACAAGAGCCGCTGGCAGCAGGGGAACGTCTTCTCATGCTCCGTGATGCATGAGGCTCTGCACAACC | | NVFSCSVMHEALHNHYTQKSLSLSPGKSG | |
| | ACTACACGCAGAAGAGCCTCTCCCTGTCTCCGGGTAAAAGCGGCAGCGAGACTCCCGGGACCTCAGAGTCCGCCACACCCGAAAG | | SETPGTSESATPESGGGEVQLVESGGGLVQ | |
| | TGGTGGCGGAGAGGTTCAGCTGGTGGAGTCTGGCGGTGGCCTGGTGCAGCCAGGGGGCTCACTCCGTTTGTCCTGTGCAGCTTCT | | PGGSLRLSCAASGFNISYSSIHWVRQAPGK | |
| | GGCTTCAACATCTCTTATTCTTCTATCCACTGGGTGCGTCAGGCCCCGGGTAAGGGCCTGGAATGGGTTGCATATATTTCTTCTTAT | | GLEWVAYISSYYGYTYYADSVKGRFTISADT | |
| | TATGGCTATACTTATTATGCCGATAGCGTCAAGGGCCGTTTCACTATAAGCGCAGACACATCCAAAAACACAGCCTACCTACAAAT | | SKNTAYLQMNSLRAEDTAVYYCARAHYFP | |
| | GAACAGCTTAAGAGCTGAGGACACTGCCGTCTATTATTGTGCTCGCGCTCATTACTTCCCGTGGGCTGGTGCTATGGACTACTGGG | | WAGAMDYWGQGTLVTVSSGGGGSDIQ | |
| | GTCAAGGAACCCTGGTCACCGTCTCCTCGGGTGGAGGTGGCAGTGATATCCAGATGACCCAGTCCCCGAGCTCCCTGTCCGCCTCT | | MTQSPSSLSASVGDRVTITCRASQSVSSAV | |
| | GTGGGCGATAGGGTCACCATCACCTGCCGTGCCAGTCAGTCCGTGTCCAGCGCTGTAGCCTGGTATCAACAGAAACCAGGAAAAG | | AWYQQKPGKAPKLLIYSASSLYSGVPSRFS | |
| | CTCCGAAGCTTCTGATTTACTCGGCATCCAGCCTCTACTCTGGAGTCCCTTCTCGCTTCTCTGGTAGCCGTTCCGGGACGGATTTCA | | GSRSGTDFTLTISSLQPEDFATYYCQQYYW | |
| | CTCTGACCATCAGCAGTCTGCAGCCGGAAGACTTCGCAACTTATTACTGTCAGCAATACTACTGGCCGATCACGTTCGGACAGGGT | | PITFGQGTKVEIK | |
| | ACCAAGGTGGAGATCAAA | | | |
| 5080- | GAGGTTCAGCTGGTGGAGTCTGGCGGTGGCCTGGTGCAGCCAGGGGGCTCACTCCGTTTGTCCTGTGCAGCTTCTGGCTTCAACA | 31 | EVQLVESGGGLVC1PGGSLRLSCAASGFNIS | 32 |
| hole- | TCTCTTATTATTCTATGCACTGGGTGCGTCAGGCCCCGGGTAAGGGCCTGGAATGGGTTGCATCTATTTCTTCTTATTATAGCTCTA | | YYSMHWVRQAPGKGLEWVASISSYYSSTS | |
| 2539- | CTTCTTATGCCGATAGCGTCAAGGGCCGTTTCACTATAAGCGCAGACACATCCAAAAACACAGCCTACCTACAAATGAACAGCTTA | | YADSVKGRFTISADTSKNTAYLOMNSLRAE | |
| 2542 | AGAGCTGAGGACACTGCCGTCTATTATTGTGCTCGCTTCTGGTACCCGGGTATGGACTACTGGGGTCAAGGAACCCTGGTCACCG | | DTAVYYCARFWYPGMDYWGQGTLVTVSS | |
| | TCTCCTCGGGTGGAGGTGGCAGTGATATCCAGATGACCCAGTCCCCGAGCTCCCTGTCCGCCTCTGTGGGCGATAGGGTCACCAT | | GGGGSDIQMTQSPSSLSASVGDRVTITCRA | |
| | CACCTGCCGTGCCAGTCAGTCCGTGTCCAGCGCTGTAGCCTGGTATCAACAGAAACCAGGAAAAGCTCCGAAGCTTCTGATTTACT | | SQSVSSAVAWYQQKPGKAPKWYSASSLY | |
| | CGGCATCCAGCCTCTACTCTGGAGTCCCTTCTCGCTTCTCTGGTAGCCGTTCCGGGACGGATTTCACTCTGACCATCAGCAGTCTGC | | SGVPSRFSGSRSGTDFTLTISSLQPEDFATY | |
| | AGCCGGAAGACTTCGCAACTTATTACTGTCAGCAACATTGGTCTTACCCGATCACGTTCGGACAGGGTACCAAGGTGGAGATCAA | | YCQQHWSYPITFGQGTKVEIKLEDKTHTKV | |
| | ACTCGAGgacaaaactcacacaAAAGTTGAGCCCAAATCTTCTgataagacccataatTGCCCACCGTGCCCAGCACCTGAACTCCTGGGGG | | EPKSSDKTHNCPPCPAPELLGGPSVFLFPPK | |
| | GACCGTCAGTCTTCCTCTTCCCCCCAAAACCCAAGGACACCCTCATGATCTCCCGGACCCCTGAGGTCACATGCGTGGTGGTGGAC | | PKDTLMISRTPEVICVVVDVSHEDPEVKFN | |
| | GTGAGCCACGAAGACCCTGAGGTCAAGTrCAACTGGTACGTGGACGGCGTGGAGGTGCATAATGCCAAGACAAAGCCGCGGGAG | | WYVDGVEVFINAKTKPREEQYNSTYRVVS | |
| | GAGCAGTACAACAGCACGTACCGTGTGGTCAGCGTCCTCACCGTCCTGCACCAGGACTGGCTGAATGGCAAGGAGTACAAGTGCA | | VLTVLHQDWLNGKEYKCKVSNKALPAPIE | |
| | AGGTCTCCAACAAAGCCCTCCCAGCCCCCATCGAGAAAACCATCTCCAAAGCCAAAGGGCAGCCCCGAGAACCACAGGTGTACAC | | KTISKAKGQPREPQVYTLPPIRELMTSNQV | |
| | CCTGCCCCCAATCCGGGAGCTGATGACCAGCAACCAGGTCAGCCTGAGCTGCGCCGTCAAAGGCTTCTATCCCAGCGACATCGCC | | SLSCAVKGFYPSDIAVEWESNGQPENNYK | |
| | GTGGAGTGGGAGAGCAATGGGCAGCCGGAGAACAACTACAAGACCACGCCTCCCGTGCTGGACTCCGACGGCTCCTTCTTCCTCG | | TTPPVLDSDGSFFLVSKLTVDKSRWQQGN | |
| | TGAGCAAGCTCACCGTGGACAAGAGCAGGTGGCAGCAGGGGAACGTCTTCTCATGCTCCGTGATGCATGAGGCTCTGCACAACCA | | VFSCSVMHEALHNHYTQKSLSLSPGKSGSE | |
| | CTACACGCAGAAGAGCCTCTCCCTGTCTCCGGGTAAAAGCGGCAGCGAGACTCCCGGGACCTCAGAGTCCGCCACACCCGAAAGT | | TPGTSESATPESGGGEVQLVESGGGLVQP | |
| | GGTGGCGGAGAGGTTCAGCTGGTGGAGTCTGGCGGTGGCCTGGTGCAGCCAGGGGGCTCACTCCGTTTGTCCTGTGCAGCTTCT | | GGSLRLSCAASGFNISSYYIHWVROAPGKG | |
| | GGCTTCAACATCTCTTCTTATTATATCCACTGGGTGCGTCAGGCCCCGGGTAAGGGCCTGGAATGGGTTGCATCTATTTATTCTTCT | | LEWVASIYSSYGYTSYADSVKGRFTISADTS | |
| | TATGGCTATACTTCTTATGCCGATAGCGTCAAGGGCCGTTTCACTATAAGCGCAGACACATCCAAAAACACAGCCTACCTACAAAT | | KNTAYLQMNSLRAEDTAVYYCARTVRGSK | |
| | GAACAGCTTAAGAGCTGAGGACACTGCCGTCTATTATTGTGCTCGCACTGTTCGTGGATCCAAAAAACCGTACTTCTCTGGTTGGG | | KPYFSGWAMDYWGQGTLVTVSSGGGGS | |
| | CTATGGACTACTGGGGTCAAGGAACCCTGGTCACCGTCTCCTCGGGTGGAGGTGGCAGTGATATCCAGATGACCCAGTCCCCGAG | | DIQMTQSPSSLSASVGDRVTITCRASQSVS | |
| | CTCCCTGTCCGCCTCTGTGGGCGATAGGGTCACCATCACCTGCCGTGCCAGTCAGTCCGTGTCCAGCGCTGTAGCCTGGTATCAAC | | SAVAWYQQKPGKAPKLLIYSASSLYSGVPS | |
| | AGAAACCAGGAAAAGCTCCGAAGCTTCTGATTTACTCGGCATCCAGCCTCTACTCTGGAGTCCCTTCTCGCTTCTCTGGTAGCCGTT | | RFSGSRSGTDFTLTISSLQPEDFATYYCQQY | |
| | CCGGGACGGATTTCACTCTGACCATCAGCAGTCTGCAGCCGGAAGACTTCGCAACTTATTACTGTCAGCAATACTCTTGGGGTCCG | | SWGPFTFGQGTKVEIK | |
| | TTCACGTTCGGACAGGGTACCAAGGTGGAGATCAAA | | | |
| 5081- | GAGGTTCAGCTGGTGGAGTCTGGCGGTGGCCTGGTGCAGCCAGGGGGCTCACTCCGTTTGTCCTGTGCAGCTTCTGGCTTCAACC | 33 | EVQLVESGGGLVQPGGSLEILSCAASGFNLS | 34 |
| knob- | TCTCTTATTATTATATGCACTGGGTGCGTCAGGCCCCGGGTAAGGGCCTGGAATGGGTTGCATCTATTTATTCTTATTCTGGCTATA | | YYYMHWVRQAPGKGLEWVASIYSYSGYT | |
| 2539- | CTTATTATGCCGATAGCGTCAAGGGCCGTTTCACTATAAGCGCAGACACATCCAAAAACACAGCCTACCTACAAATGAACAGCTTA | | YYADSVKGRFTISADTSKNTAYLQMNSLRA | |
| 2542 | AGAGCTGAGGACACTGCCGTCTATTATTGTGCTCGCTTCTTTCGCTTGGGCTTTTGACTACTGGGGTCAAGGAACCCTGGTCACC | | EDTAVYYCARSSFAWAFDYWGQGTLVTV | |
| | GTCTCCTCGGGTGGAGGTGGCAGTGATATCCAGATGACCCAGTCCCCGAGCTCCCTGTCCGCCTCTGTGGGCGATAGGGTCACCA | | SSGGGGSDIQMTQSPSSLSASVGDRVTITC | |
| | TCACCTGCCGTGCCAGTCAGTCCGTGTCCAGCGCTGTAGCCTGGTATCAACAGAAACCAGGAAAAGCTCCGAAGCTTCTGATTTAC | | RASQSVSSAVAWYQQKPGKAPKLLIYSASS | |
| | TCGGCATCCAGCCTCTACTCTGGAGTCCCTTCTCGCTTCTCTGGTAGCCGTTCCGGGACGGATTTCACTCTGACCATCAGCAGTCTG | | LYSGVPSRFSGSRSGTDFTLTISSLQPEDFAT | |
| | CAGCCGGAAGACTTCGCAACTTATTACTGTCAGCAAGGTGGTTGGGGTCCGTTCACGTTCGGACAGGGTACCAAGGTGGAGATCA | | YYCQQGGWGPFTFGQGTKVEIKLEDKTHT | |
| | AACTCGAGgacaaaactcacacaAAAGTGGAGCCCAAAACTTCTgataagacccatactTGCCCACCGTGCCCAGCACCTGAACTCCTGGGG | | KVEPKTSDKTHTCPPCPAPELLGGPSVFLFP | |
| | GGACCGTCAGTCTTCCTCTTCCCCCCAAAACCCAAGGACACCCTCATGATCTCCCGGACCCCTGAGGTCACATGCGTGGTGGTGGA | | PKPKDILMISRTPEVTCVVVDVSHEDPEVK | |
| | CGTGAGCCACGAAGACCCTGAGGTCAAGTTCAACTGGTACGTGGACGGCGTGGAGGTGCATAATGCCAAGACAAAGCCGCGCGA | | FNWYVDGVEVHNAKTKPREEQYNSTYRV | |
| | GGAGCAGTACAACAGCACGTACCGTGTGGTCAGCGTCCTCACCGTCCTGCACCAGGACTGGCTGAATGGCAAGGAGTACAAGTGC | | VSVLTVLHQDWLNGKEYKCKVSNKALPAP | |
| | AAGGTCTCCAACAAAGCCCTCCCAGCCCCCATCGAGAAAACCATCTCCAAAGCCAAAGGGCAGCCCCGAGAACCAATGGTGTTTG | | IEKTISKAKGQPREPMVFDLPPSREEMTKN | |
| | ACCTGCCCCCATCCCGGGAGGAGATGACCAAGAACCAGGTCAGCCTGTGGTGCATGGTCAAGGGCTTCTATCCCAGCGACATCGC | | QVSLWCMVKGFYPSDIAVEWESNGOPEN | |
| | CGTGGAGTGGGAGAGCAATGGGCAGCCGGAGAACAACTACAAGACCACGCCTCCCGTGCTGGACTCCGACGGCTCCTTCTTCCTG | | NYKTTPPVLDSDGSFFLYSKLTVDKSRWQQ | |
| | TACAGCAAGCTCACCGTGGACAAGAGCCGCTGGCAGCAGGGGAACGTCTTCTCATGCTCCGTGATGCATGAGGCTCTGCACAACC | | GNVFSCSVMHEALHNHYTQKSLSLSPGKS | |
| | ACTACACGCAGAAGAGCCTCTCCCTGTCTCCGGGTAAAAGCGGCAGCGAGACTCCCGGGACCTCAGAGTCCGCCACACCCGAAAG | | GSETPGTSESATPESGGGEVQLVESGGGLV | |
| | TGGTGGCGGAGAGGTTCAGCTGGTGGAGTCTGGCGGTGGCCTGGTGCAGCCAGGGGGCTCACTCCGTTTGTCCTGTGCAGCTTCT | | IDPGGSLRLSCAASGFNISYSSIHWVROAPG | |
| | GGCTTCAACATCTCTTATTCTTCTATCCACTGGGTGCGTCAGGCCCCGGGTAAGGGCCTGGAATGGGTTGCATATATTTCTTCTTAT | | KGLEWVAYISSYYGYIYYADSVKGRFTISAD | |
| | TATGGCTATACTTATTATGCCGATAGCGTCAAGGGCCGTTTCACTATAAGCGCAGACACATCCAAAAACACAGCCTACCTACAAAT | | TSKNTAYLQMNSLRAEDTAVYYCARAHYF | |
| | GAACAGCTTAAGAGCTGAGGACACTGCCGTCTATTATTGTGCTCGCGCTCATTACTTCCCGTGGGCTGGTGCTATGGACTACTGGG | | PWAGAMDYWGQGTLVIVSSGGGGSDIQ | |
| | GTCAAGGAACCCTGGTCACCGTCTCCTCGGGTGGAGGTGGCAGTGATATCCAGATGACCCAGTCCCCGAGCTCCCTGTCCGCCTCT | | MTQSPSSLSASVGDRVTITCRASQSVSSAV | |
| | GTGGGCGATAGGGTCACCATCACCTGCCGTGCCAGTCAGTCCGTGTCCAGCGCTGTAGCCTGGTATCAACAGAAACCAGGAAAAG | | AWYQQKPGKAPKWYSASSLYSGVPSRFS | |
| | CTCCGAAGCTTCTGATTTACTCGGCATCCAGCCTCTACTCTGGAGTCCCTTCTCGCTTCTCTGGTAGCCGTTCCGGGACGGATTTCA | | GSRSGTDFILTISSLQPEDFATYYCQQYYW | |
| | CTCTGACCATCAGCAGTCTGCAGCCGGAAGACTTCGCAACTTATTACTGTCAGCAATACTACTGGCCGATCACGTTCGGACAGGGT | | PITFGQGTKVEIK | |
| | ACCAAGGTGGAGATCAAA | | | |
| 5081- | GAGGTTCAGCTGGTGGAGTCTGGCGGTGGCCTGGTGCAGCCAGGGGGCTCACTCCGTTTGTCOGTGCAGCTTCTGGCTTCAACC | 35 | EVCILVESGGGLVQPGGSLRLSCAASGENLS | 36 |
| hole- | TCTCTTATTATTATATGCACTGGGTGCGTCAGGCCCCGGGTAAGGGCCTGGAATGGGTTGCATCTATTTATTCTTATTCTGGCTATA | | YYYMHWVRQAPGKGLEWVASIYSYSGYT | |
| 2539- | CTTATTATGCCGATAGCGTCAAGGGCCGTTTCACTATAAGCGCAGACACATCCAAAAACACAGCCTACCTACAAATGAACAGCTTA | | YYADSVKGRFTISADISKNTAYLQMNSLRA | |
| 2542 | AGAGCTGAGGACACTGCCGTCTATTATTGTGCTCGCTCTTCTTTCGCTTGGGCTTTTGACTACTGGGGTCAAGGAACCCTGGTCACC | | EDTAVYYCARSSFAWAFDYWGQGTLVTV | |
| | GTCTCCTCGGGTGGAGGTGGCAGTGATATCCAGATGACCCAGTCCCCGAGCTCCCTGTCCGCCTCTGTGGGCGATAGGGTCACCA | | SSGGGGSDIQMTQSPSSLSASVGDRVTITC | |
| | TCACCTGCCGTGCCAGTCAGTCCGTGTCCAGCGCTGTAGCCTGGTATCAACAGAAACCAGGAAAAGCTCCGAAGCTTCTGATTTAC | | RASQSVSSAVAWYQQKPGKAPKLLIYSASS | |
| | TCGGCATCCAGCCTCTACTCTGGAGTCCCTTCTCGCTTCTCTGGTAGCCGTTCCGGGACGGATTTCACTCTGACCATCAGCAGTCTG | | LYSGVPSRFSGSRSGTDFTLTISSLQPEDFAT | |
| | CAGCCGGAAGACTTCGCAACTTATTACTGTCAGCAAGGTGGTTGGGGTCCGTTCACGTTCGGACAGGGTACCAAGGTGGAGATCA | | YYCQQGGWGPFTFGQGTKVEIKLEDKTHT | |
| | AACTCGAGgacaaaactcacacaAAAGTTGAGCCCAAATCTTCTgataagacccataatTGCCCACCGTGCCCAGCACCTGAACTCCTGGGG | | KVEPKSSDKTHNCPPCPAPELLGGPSVFLF | |
| | GGACCGTCAGTCTTCCTCTTCCCCCCAAAACCCAAGGACACCCTCATGATCTCCCGGACCCCTGAGGTCACATGCGTGGTGGTGGA | | PPKPKDTLMISRTFEVICVVVDVSHEDPEV | |
| | CGTGAGCCACGAAGACCCTGAGGTCAAGTTCAACTGGTACGTGGACGGCGTGGAGGTGCATAATGCCAAGACAAAGCCGCGGGA | | KFNWYVDGVEVHNAKTKPREEQYNSTYR | |
| | GGAGCAGTACAACAGCACGTACCGTGTGGTCAGCGTCCTCACCGTCCTGCACCAGGACTGGCTGAATGGCAAGGAGTACAAGTGC | | VVSVLTVLHQDWLNGKEYKCKVSNKALPA | |
| | AAGGTCTCCAACAAAGCCCTCCCAGCCCCCATCGAGAAAACCATCTCCAAAGCCAAAGGGCAGCCCCGAGAACCACAGGTGTACA | | PIEKTISKAKGQPREPQVYTLPPIRELMTSN | |
| | CCCTGCCCCCAATCCGGGAGCTGATGACCAGCAACCAGGTCAGCCTGAGCTGCGCCGTCAAAGGCUCTATCCCAGCGACATCGCC | | QVSLSCAVKGFYPSDIAVEWESNGQPENN | |
| | GTGGAGTGGGAGAGCAATGGGCAGCCGGAGAACAACTACAAGACCACGCCTCCCGTGCTGGACTCCGACGGCTCCTTCTTCCTCG | | YKTTPPVLDSDGSFFLVSKLTVDKSRWQQ | |
| | TGAGCAAGCTCACCGTGGACAAGAGCAGGTGGCAGCAGGGGAACGTCTTCTCATGCTCCGTGATGCATGAGGCTCTGCACAACCA | | GNVFSCSVMHEALHNHYTQKSLSLSPGKS | |
| | CTACACGCAGAAGAGCCTCTCCCTGTCTCCGGGTAAAAGCGGCAGCGAGACTCCCGGGACCTCAGAGTCCGCCACACCCGAAAGT | | GSETPGTSESATPESGGGEVQLVESGGGLV | |
| | GGTGGCGGAGAGGTTCAGCTGGTGGAGTCTGGCGGTGGCCTGGTGCAGCCAGGGGGCTCACTCCGTTTGTCCTGTGCAGCTTCT | | QPGGSLRLSCAASGFNISSYYIHWVRQAPG | |
| | GGCTTCAACATCTCTTCTTATTATATCCACTGGGTGCGTCAGGCCCCGGGTAAGGGCCTGGAATGGGTTGCATCTATTTATTCTTCT | | KGLEWVASIYSSYGYTSYADSVKGRFTISAD | |
| | TATGGCTATACTTCTTATGCCGATAGCGTCAAGGGCCGTTTCACTATAAGCGCAGACACATCCAAAAACACAGCCTACCTACAAAT | | TSKNTAYLQMNSLRAEDTAVYYCARTVRG | |
| | GAACAGCTTAAGAGCTGAGGACACTGCCGTCTATTATTGTGCTCGCACTGTTCGTGGATCCAAAAAACCGTACTTCTCTGGrrGGG | | SKKPYFSGWAMDYWGQGTLVTVSSGGG | |
| | CTATGGACTACTGGGGTCAAGGAACCCTGGTCACCGTCTCCTCGGGTGGAGGTGGCAGTGATATCCAGATGACCCAGTCCCCGAG | | GSDIQMTQSPSSLSASVGDRVTITCRASQS | |
| | CTCCCTGTCCGCCTCTGTGGGCGATAGGGTCACCATCACCTGCCGTGCCAGTCAGTCCGTGTCCAGCGCTGTAGCCTGGTATCAAC | | VSSAVAWYQQKPGKAPKLLIYSASSLYSGV | |
| | AGAAACCAGGAAAAGCTCCGAAGCTTCTGATTTACTCGGCATCCAGCCTCTACTCTGGAGTCCCTTCTCGCTTCTCTGGTAGCCGTT | | PSRFSGSRSGTDFTLTISSLQPEDFATYYCQ | |
| | CCGGGACGGATTTCACTCTGACCATCAGCAGTCTGCAGCCGGAAGACTTCGCAACTTATTACTGTCAGCAATACTCTTGGGGTCCG | | QYSWGPFTFGQGTKVEIK | |
| | TTCACGTTCGGACAGGGTACCAAGGTGGAGATCAAA | | | |
| 2928- | GAGGTTCAGCTGGTGGAGTCTGGCGGTGGCCTGGTGCAGCCAGGGGGCTCACTCCGTTTGTCCTGTGCAGCTTCTGGCTTCAACA | 37 | EVCILVESGGGLVQPGGSLRLSCAASGFNIS | 38 |
| knob- | TCTCTTATTCTTCTATCCACTGGGTGCGTCAGGCCCCGGGTAAGGGCCTGGAATGGGTTGCATCTATTTATCCTTCTTATAGCTCTA | | YSSIHWVRQAPGKGLEWVASIYPSYSSTYY | |
| 2539- | CTTATTATGCCGATAGCGTCAAGGGCCGTTTCACTATAAGCGCAGACACATCCAAAAACACAGCCTACCTACAAATGAACAGCTTA | | ADSVKGRFTISADTSKNTAYLQMNSLRAED | |
| 2542 | AGAGCTGAGGACACTGCCGTCTATTATTGTGCTCGCTACTACGCTATGGACTACTGGGGTCAAGGAACCCTGGTCACCGTCTCCTC | | TAVYYCARYYAMDYWGQGTLVTVSSGGG | |
| | GGGTGGAGGTGGCAGTGATATCCAGATGACCCAGTCCCCGAGCTCCCTGTCCGCCTCTGTGGGCGATAGGGTCACCATCACCTGC | | GSDIQMTQSPSSLSASVGDRVTITCRASQS | |
| | CGTGCCAGTCAGTCCGTGTCCAGCGCTGTAGCCTGGTATCAACAGAAACCAGGAAAAGCTCCGAAGCTTCTGATTTACTCGGCATC | | VSSAVAWYQQKPGKAPKLLIYSASSLYSGV | |
| | CAGCCTCTACTCTGGAGTCCCTTCTCGCTTCTCTGGTAGCCGTTCCGGGACGGATTTCACTCTGACCATCAGCAGTCTGCAGCCGGA | | PSRFSGSRSGTDFTLTISSLQPEDFATYYCQ | |
| | AGACTTCGCAACTTATTACTGTCAGCAAGCTTTCTACTACCCGATCACGTTCGGACAGGGTACCAAGGTGGAGATCAAACTCGAGg | | QAFYYPITFGQGTKVEIKLEDKTHTKVEPKT | |
| | acaaaactcacacaAAAGTGGAGCCCAAAACTTCTgataagacccatactTGCCCACCGTGCCCAGCACCTGAACTCCTGGGGGGACCGTC | | SDKTHTCPPCPAPELLGGPSVFLFPPKPKDT | |
| | AGTCTTCCTCTTCCCCCCAAAACCCAAGGACACCCTCATGATCTCCCGGACCCCTGAGGTCACATGCGTGGTGGTGGACGTGAGCC | | LMISRTPEVTCWVDVSHEDPEVKENWYV | |
| | ACGAAGACCCTGAGGTCAAGTTCAACTGGTACGTGGACGGCGTGGAGGTGCATAATGCCAAGACAAAGCCGCGCGAGGAGCAGT | | DGVEVHNAKTKPREEQYNSTYRVVSVLIV | |
| | ACAACAGCACGTACCGTGTGGTCAGCGTCCTCACCGTCCTGCACCAGGACTGGCTGAATGGCAAGGAGTACAAGTGCAAGGTCTC | | LHQDWLNGKEYKCKVSNKALPAPIEKTISK | |
| | CAACAAAGCCCTCCCAGCCCCCATCGAGAAAACCATCTCCAAAGCCAAAGGGCAGCCCCGAGAACCAATGGTGTTTGACCTGCCCC | | AKGQPREPMVFDLPPSREEMTKNQVSLW | |
| | CATCCCGGGAGGAGATGACCAAGAACCAGGTCAGCCTGTGGTGCATGGTCAAGGGCTTCTATCCCAGCGACATCGCCGTGGAGT | | CMVKGFYPSDIAVEWESNGQPENNYKTTP | |
| | GGGAGAGCAATGGGCAGCCGGAGAACAACTACAAGACCACGCCTCCCGTGCTGGACTCCGACGGCTCCTTCTTCCTGTACAGCAA | | PVLDSOGSFFLYSKLTVDKSRWQQGNVFS | |
| | GCTCACCGTGGACAAGAGCCGCTGGCAGCAGGGGAACGTCTTCTCATGCTCCGTGATGCATGAGGCTCTGCACAACCACTACACG | | CSVMHEALHNHYTQKSLSLSPGKSGSETP | |
| | CAGAAGAGCCTCTCCCTGTCTCCGGGTAAAAGCGGCAGCGAGACTCCCGGGACCTCAGAGTCCGCCACACCCGAAAGTGGTGGC | | GTSESATPESGGGEVQLVESGGGLVQPGG | |
| | GGAGAGGTTCAGCTGGTGGAGTCTGGCGGTGGCCTGGTGCAGCCAGGGGGCTCACTCCGTTTGTCCTGTGCAGCTTCTGGCTTCA | | SLRLSCAASGFNISYSSIHWVRQAPGKGLE | |
| | ACATCTCTTATTCTTCTATCCACTGGGTGCGTCAGGCCCCGGGTAAGGGCCTGGAATGGGTTGCATATATTTCTTCTTATTATGGCT | | WVAYISSYYGYTYYADSVKGRFTISADTSK | |
| | ATACTTATTATGCCGATAGCGTCAAGGGCCGTTTCACTATAAGCGCAGACACATCCAAAAACACAGCCTACCTACAAATGAACAGC | | NTAYLQMNSLRAEDTAVYYCARAHYFPW | |
| | TTAAGAGCTGAGGACACTGCCGTCTATTATTGTGCTCGCGCTCATTACTTCCCGTGGGCTGGTGCTATGGACTACTGGGGTCAAGG | | AGAMDYWGQGTLVTVSSGGGGSDIQMT | |
| | AACCCTGGTCACCGTCTCCTCGGGTGGAGGTGGCAGTGATATCCAGATGACCCAGTCCCCGAGCTCCCTGTCCGCCTCTGTGGGC | | QSPSSLSASVGDRVTITCRASQSVSSAVAW | |
| | GATAGGGTCACCATCACCTGCCGTGCCAGTCAGTCCGTGTCCAGCGCTGTAGCCTGGTATCAACAGAAACCAGGAAAAGCTCCGA | | YQQKPGKAPKLLIYSASSLYSGVPSRFSGSR | |
| | AGCTTCTGATTTACTCGGCATCCAGCCTCTACTCTGGAGTCCCTTCTCGCTTCTCTGGTAGCCGTTCCGGGACGGATTTCACTCTGA | | SGTDFTLTISSLQPEDFATYYCQQYYWPITF | |
| | CCATCAGCAGTCTGCAGCCGGAAGACTTCGCAACTTATTACTGTCAGCAATACTACTGGCCGATCACGTTCGGACAGGGTACCAAG | | GQGTKVEIK | |
| | GTGGAGATCAAA | | | |
| 2928- | GAGGTTCAGCTGGTGGAGTCTGGCGGTGGCCTGGTGCAGCCAGGGGGCTCACTCCGTTTGTCCTGTGCAGCTTCTGGCTTCAACA | 39 | EVQLVESGGGLVQPGGSLRLSCAASGFNIS | 40 |
| hole- | TCTCTTATTCTTCTATCCACTGGGTGCGTCAGGCCCCGGGTAAGGGCCTGGAATGGGTTGCATCTATTTATCCTTCTTATAGCTCTA | | YSSIHWVRQAPGKGLEWVASIYPSYSSTYY | |
| 2539- | CTTATTATGCCGATAGCGTCAAGGGCCGTTTCACTATAAGCGCAGACACATCCAAAAACACAGCCTACCTACAAATGAACAGCTTA | | ADSVKGRFTISADTSKNTAYLQMNSLRAED | |
| 2542 | AGAGCTGAGGACACTGCCGTCTATTATTGTGCTCGCTACTACGCTATGGACTACTGGGGTCAAGGAACCCTGGTCACCGTCTCCTC | | TAVYYCARYYAMDYWGQGTLVTVSSGGG | |
| | GGGTGGAGGTGGCAGTGATATCCAGATGACCCAGTCCCCGAGCTCCCTGTCCGCCTCTGTGGGCGATAGGGTCACCATCACCTGC | | GSDIQMTQSPSSLSASVGDRVTITCRASQS | |
| | CGTGCCAGTCAGTCCGTGTCCAGCGCTGTAGCCTGGTATCAACAGAAACCAGGAAAAGCTCCGAAGCTTCTGATTTACTCGGCATC | | VSSAVAWYQQKPGKAPKLLIYSASSLYSGV | |
| | CAGCCTCTACTCTGGAGTCCCTTCTCGCTTCTCTGGTAGCCGTTCCGGGACGGATTTCACTCTGACCATCAGCAGTCTGCAGCCGGA | | PSRFSGSRSGTDFTLTISSLQPEDFATYYCQ | |
| | AGACTTCGCAACTTATTACTGTCAGCAAGCTTTCTACTACCCGATCACGTTCGGACAGGGTACCAAGGTGGAGATCAAACTCGAGg | | QAFYYPITFGQGTKVEIKLEDKTHTKVEPKS | |
| | acaaaactcacacaAAAGTTGAGCCCAAATCTTCTgataagacccataatTGCCCACCGTGCCCAGCACCTGAACTCCTGGGGGGACCGTCA | | SDKTHNCPPCPAPELLGGPSVFLFPPKPKD | |
| | GTCTTCCTCTTCCCCCCAAAACCCAAGGACACCCTCATGATCTCCCGGACCCCTGAGGTCACATGCGTGGTGGTGGACGTGAGCCA | | TLMISRTPEVTCVVVDVSHEDPEVKFNWY | |
| | CGAAGACCCTGAGGTCAAGTTCAACTGGTACGTGGACGGCGTGGAGGTGCATAATGCCAAGACAAAGCCGCGGGAGGAGCAGT | | VDGVEVHNAKTKPREEQYNSTYRVVSVLT | |
| | ACAACAGCACGTACCGTGTGGTCAGCGTCCTCACCGTCCTGCACCAGGACTGGCTGAATGGCAAGGAGTACAAGTGCAAGGTCTC | | VLHQDWLNGKEYKCKVSNKALPAPIEKTIS | |
| | CAACAAAGCCCTCCCAGCCCCCATCGAGAAAACCATCTCCAAAGCCAAAGGGCAGCCCCGAGAACCACAGGTGTACACCCTGCCC | | KAKGQPREPQVYTLPPIRELMTSNQVSLSC | |
| | CCAATCCGGGAGCTGATGACCAGCAACCAGGTCAGCCTGAGCTGCGCCGTCAAAGGCTTCTATCCCAGCGACATCGCCGTGGAGT | | AVKGFYPSDIAVEWESNGQPENNYKTTPP | |
| | GGGAGAGCAATGGGCAGCCGGAGAACAACTACAAGACCACGCCTCCCGTGCTGGACTCCGACGGCTCCTTCTTCCTCGTGAGCAA | | VLDSDGSFFLVSKUTVDKSRWQQGNVFSC | |
| | GCTCACCGTGGACAAGAGCAGGTGGCAGCAGGGGAACGTCTTCTCATGCTCCGTGATGCATGAGGCTCTGCACAACCACTACACG | | SVMHEALHNHYTQKSLSLSPGKSGSETPGT | |
| | CAGAAGAGCCTCTCCCTGTCTCCGGGTAAAAGCGGCAGCGAGACTCCCGGGACCTCAGAGTCCGCCACACCCGAAAGTGGTGGC | | SESATPESGGGEVQLVESGGGLVQPGGSL | |
| | GGAGAGGTTCAGCTGGTGGAGTCTGGCGGTGGCCTGGTGCAGCCAGGGGGCTCACTCCGTTTGTCCTGTGCAGCTTCTGGCTTCA | | RLSCAASGFNISSYYIHWVROAPGKGLEW | |
| | ACATCTCTTCTTATTATATCCACTGGGTGCGTCAGGCCCCGGGTAAGGGCCTGGAATGGGTTGCATCTATTTATTCTTCTTATGGCT | | VASIYSSYGYTSYADSVKGRFTISADTSKNT | |
| | ATACTTCTTATGCCGATAGCGTCAAGGGCCGTTTCACTATAAGCGCAGACACATCCAAAAACACAGCCTACCTACAAATGAACAGC | | AYLQMNSLRAEDTAVYYCARTVRGSKKPY | |
| | TTAAGAGCTGAGGACACTGCCGTCTATTATTGTGCTCGCACTGTTCGTGGATCCAAAAAACCGTACTTCTCTGGTTGGGCTATGGA | | FSGWAMDYWGQGTLVTVSSGGGGSDIQ | |
| | CTACTGGGGTCAAGGAACCUGGTCACCGTCTCCTCGGGTGGAGGTGGCAGTGATATCCAGATGACCCAGTCCCCGAGCTCCCTG | | MTQSPSSLSASVGDRVTITCRASQSVSSAV | |
| | TCCGCCTCTGTGGGCGATAGGGTCACCATCACCTGCCGTGCCAGTCAGTCCGTGTCCAGCGCTGTAGCCTGGTATCAACAGAAACC | | AWYQQKPGKAPKWYSASSLYSGVPSRFS | |
| | AGGAAAAGCTCCGAAGCTTCTGATTTACTCGGCATCCAGCCTCTACTCTGGAGTCCCTTCTCGCTTCTCTGGTAGCCGTTCCGGGAC | | GSRSGTDFTLTISSLQPEDFATYYCQQYSW | |
| | GGATTTCACTCTGACCATCAGCAGTCTGCAGCCGGAAGACTTCGCAACTTATTACTGTCAGCAATACTCTTGGGGTCCGTTCACGTT | | GPFTFGQGTKVEIK | |
| | CGGACAGGGTACCAAGGTGGAGATCAAA | | | |
| 5019- | GAGGTTCAGCTGGTGGAGTCTGGCGGTGGCCTGGTGCAGCCAGGGGGCTCACTCCGTTTGTCCTGTGCAGCTTCTGGCTTCAACA | 41 | EVQLVESGGGLVQPGGSLRLSCAASGFNIG | 42 |
| knob- | TCGGTTCTTCTTCTATCCACTGGGTGCGTCAGGCCCCGGGTAAGGGCCTGGAATGGGTTGCATCTATTTATTCTGCTTTTGCCTCTA | | SSSIHWVRQAPGKGLEWVASIYSAFASTSY | |
| 2459- | CTTCTTATGCCGATAGCGTCAAGGGCCGTTTCACTATAAGCGCAGACACATCCAAAAACACAGCCTACCTACAAATGAACAGCTTA | | ADSVKGRFTISADTSKNTAYLQMNSLRAED | |
| 2460 | AGAGCTGAGGACACTGCCGTCTATTATTGTGCTCGCTACCATTTCCCGTTCGGTTTTGCTTTGGACTACTGGGGTCAAGGAACCCT | | TAVYYCARYHFPFGFALDYWGQGTLVTVS | |
| | GGTCACCGTCTCCTCGGGTGGAGGTGGCAGTGATATCCAGATGACCCAGTCCCCGAGCTCCCTGTCCGCCTCTGTGGGCGATAGG | | SGGGGSDIQMTQSPSSLSASVGDRVTITCR | |
| | GTCACCATCACCTGCCGTGCCAGTCAGTCCGTGTCCAGCGCTGTAGCCTGGTATCAACAGAAACCAGGAAAAGCTCCGAAGCTTCT | | ASQSVSSAVAWYQQKPGKAPKLLIYSASSL | |
| | GATTTACTCGGCATCCAGCCTCTACTCTGGAGTCCCTTCTCGCTTCTCTGGTAGCCGTTCCGGGACGGATTTCACTCTGACCATCAG | | YSGVPSRFSGSRSGTDFTLTISSLQPEDFAT | |
| | CAGTCTGCAGCCGGAAGACTTCGCAACTTATTACTGTCAGCAAGGTGTTTACCTGTTCACGTTCGGACAGGGTACCAAGGTGGAG | | YYCQQGVYLFTFGQGTKVEIKLEDKTHTKV | |
| | ATCAAACTCGAGgataaaactccacaAAAGTGGAGCCCAAAACTTCTgataagacccatactTGCCCACCGTGCCCAGCACCTGAACTCCT | | EPKTSDKTHTCPPCPAPELLGGPSVFLFPPK | |
| | GGGGGGACCGTCAGTCTTCCTCTTCCCCCCAAAACCCAAGGACACCCTCATGATCTCCCGGACCCCTGAGGTCACATGCGTGGTGG | | PKDTLMISRTPEVTCVVVDVSHEDPEVKFN | |
| | TGGACGTGAGCCACGAAGACCCTGAGGTCAAGTTCAACTGGTACGTGGACGGCGTGGAGGTGCATAATGCCAAGACAAAGCCGC | | WYVDGVEVHNAKTKPREEQYNSTYRVVS | |
| | GCGAGGAGCAGTACAACAGCACGTACCGTGTGGTCAGCGTCCTCACCGTCCTGCACCAGGACTGGCTGAATGGCAAGGAGTACA | | VLTVLHQDWLNGKEYKCKVSNKALPAPIE | |
| | AGTGCAAGGTCTCCAACAAAGCCCTCCCAGCCCCCATCGAGAAAACCATCTCCAAAGCCAAAGGGCAGCCCCGAGAACCAATGGT | | KTISKAKGQPREPMVFDLPPSREEMTKNQ | |
| | GTTTGACCTGCCCCCATCCCGGGAGGAGATGACCAAGAACCAGGTCAGCCTGTGGTGCATGGTCAAGGGCTTCTATCCCAGCGAC | | VSLWCMVKGFYPSDIAVEWESNGQPENN | |
| | ATCGCCGTGGAGTGGGAGAGCAATGGGCAGCCGGAGAACAACTACAAGACCACGCCTCCCGTGCTGGACTCCGACGGCTCCTTCT | | YKTTPPVLDSDGSFFLYSKLTVOKSRWQQG | |
| | TCCTGTACAGCAAGCTCACCGTGGACAAGAGCCGCTGGCAGCAGGGGAACGTCTTCTCATGCTCCGTGATGCATGAGGCTCTGCA | | NVFSCSVMHEALHNHYTQKSLSLSPGKSG | |
| | CAACCACTACACGCAGAAGAGCCTCTCCCTGTCTCCGGGTAAAAGCGGCAGCGAGACTCCCGGGACCTCAGAGTCCGCCACACCC | | SETPGTSESATPESGGGEVQLVESGGGLVQ | |
| | GAAAGTGGTGGCGGAGAGGTTCAGCTGGTGGAGTCTGGCGGTGGCCTGGTGCAGCCAGGGGGCTCACTCCGTTTGTCCTGTGCA | | PGGSLRLSCAASGFNFSSSSIHWVRQAPGK | |
| | GCTTCTGGCTTCAACTTTTCTTCTTCTTCTATACACTGGGTGCGTCAGGCCCCGGGTAAGGGCCTGGAATGGGTTGCATCTATTTCT | | GLEWVASISSSYGYTYYADSVKGRFT1SADT | |
| | TCTTCTTATGGCTATACTTATTATGCCGATAGCGTCAAGGGCCGTTTCACTATAAGCGCAGACACATCCAAAAACACAGCCTACCTA | | SKNTAYLQMNSLRAEDTAVYYCARGGSGV | |
| | CAAATGAACAGCTTAAGAGCTGAGGACACTGCCGTCTATTATTGTGCTCGCGGTGGTTCTGGTGTTTCTCATTACGGTTCTGTTTAC | | SHYGSVYYSWWALDYWGQGTLVTVSSGG | |
| | TACTCTTGGTGGGCTTTGGACTACTGGGGTCAAGGAACCCTGGTCACCGTCTCCTCGGGTGGAGGTGGCAGTGATATCCAGATGA | | GGSDIQMTQSPSSLSASVGDRVTITCRASQ | |
| | CCCAGTCCCCGAGCTCCCTGTCCGCCTCTGTGGGCGATAGGGTCACCATCACCTGCCGTGCCAGTCAGTCCGTGTCCAGCGCTGTA | | SVSSAVAWYQQKPGKAPKLLIYSASSLYSG | |
| | GCCTGGTATCAACAGAAACCAGGAAAAGCTCCGAAGCTTCTGATTTACTCGGCATCCAGCCTCTACTCTGGAGTCCCTTCTCGCTTC | | VPSRFSGSRSGTDFTLTISSLQPEDFATYYC | |
| | TCTGGTAGCCGTTCCGGGACGGATTTCACTCTGACCATCAGCAGTCTGCAGCCGGAAGACTTCGCAACTTATTACTGTCAGCAAGC | | QQASYAPITFGQGTKVEIK* | |
| | TTCTTACGCTCCGATCACGTTCGGACAGGGTACCAAGGTGGAGATCAAA | | | |
| 5019- | GAGGTTCAGCTGGTGGAGTCTGGCGGTGGCCTGGTGCAGCCAGGGGGCTCACTCCGTTTGTCCTGTGCAGCTTCTGGCTTCAACA | 43 | EVOLVESGGGLVQPGGSLRLSCAASGFNIG | 44 |
| hole- | TCGGTTCTTCTTCTATCCACTGGGTGCGTCAGGCCCCGGGTAAGGGCCTGGAATGGGTTGCATCTATTTATTCTGCTTTTGCCTCTA | | SSSIHWVRQAPGKGLEWVASIYSAFASTSY | |
| 2459- | CTTCTTATGCCGATAGCGTCAAGGGCCGTTTCACTATAAGCGCAGACACATCCAAAAACACAGCCTACCTACAAATGAACAGCTTA | | ADSVKGRFTISADTSKNTAYLQMNSLRAED | |
| 2460 | AGAGCTGAGGACACTGCCGTCTATTATTGTGCTCGCTACCATTTCCCGTTCGGTTTTGCTTTGGACTACTGGGGTCAAGGAACCCT | | TAVYYCARYHFPFGFALDYWGQGTLVTVS | |
| | GGTCACCGTCTCCTCGGGTGGAGGTGGCAGTGATATCCAGATGACCCAGTCCCCGAGCTCCCTGTCCGCCTCTGTGGGCGATAGG | | SGGGGSDIQMTQSPSSLSASVGDRVTITCR | |
| | GTCACCATCACCTGCCGTGCCAGTCAGTCCGTGTCCAGCGCTGTAGCCTGGTATCAACAGAAACCAGGAAAAGCTCCGAAGCTTCT | | ASQSVSSAVAWYQQKPGKAPKWYSASSL | |
| | GATTTACTCGGCATCCAGCCTCTACTCTGGAGTCCCTTCTCGCTTCTCTGGTAGCCGTTCCGGGACGGATTTCACTCTGACCATCAG | | YSGVPSRFSGSRSGTDFTLTISSLQPEDFAT | |
| | CAGTCTGCAGCCGGAAGACTTCGCAACTTATTACTGTCAGCAAGGTGTTTACCTGTTCACGTTCGGACAGGGTACCAAGGTGGAG | | YYCQQGVYLFTFGQGTKVEIKLEDKTHTKV | |
| | ATCAAACTCGAGgacaaaactcacacaAAAGTTGAGCCCAAATCTTCTgataagacccataatTGCCCACCGTGCCCAGCACCTGAACTCCT | | EPKSSDKTHNCPPCPAPELLGGPSVFLFPPK | |
| | GGGGGGACCGTCAGTCTTCCTTTCCCCCCAAAACCCAAGGACACCCTCATGATCTCCCGGACCCCTGAGGTCACATGCGTGGTGG | | PKDTLMISRTPEVTCVVVDVSHEDPEVKFN | |
| | TGGACGTGAGCCACGAAGACCCTGAGGTCAAGTTCAACTGGTACGTGGACGGCGTGGAGGTGCATAATGCCAAGACAAAGCCGC | | WYVDGVEVHNAKTKPREEQYNSTYRVVS | |
| | GGGAGGAGCAGTACAACAGCACGTACCGTGTGGTCAGCGTCCTCACCGTCCTGCACCAGGACTGGCTGAATGGCAAGGAGTACA | | VLTVLHQDWLNGKEYKCKVSNKALPAPIE | |
| | AGTGCAAGGTCTCCAACAAAGCCCTCCCAGCCCCCATCGAGAAAACCATCTCCAAAGCCAAAGGGCAGCCCCGAGAACCACAGGT | | KTISKAKGQPREPQVYTLPPIRELMTSNQV | |
| | GTACACCCTGCCCCCAATCCGGGAGCTGATGACCAGCAACCAGGTCAGCCTGAGCTGCGCCGTCAAAGGCTTCTATCCCAGCGAC | | SLSCAVKGFYPSDIAVEWESNGQPENNYK | |
| | ATCGCCGTGGAGTGGGAGAGCAATGGGCAGCCGGAGAACAACTACAAGACCACGCCTCCCGTGCTGGACTCCGACGGCTCCTTCT | | TTPPVLDSDGSFFLVSKLTVDKSRWQQGN | |
| | TCCTCGTGAGCAAGCTCACCGTGGACAAGAGCAGGTGGCAGCAGGGGAACGTCTTCTCATGCTCCGTGATGCATGAGGCTCTGCA | | VFSCSVMHEALHNHYTQKSLSLSPGKSGSE | |
| | CAACCACTACACGCAGAAGAGCCTCTCCCTGTCTCCGGGTAAAAGCGGCAGCGAGACTCCCGGGACCTCAGAGTCCGCCACACCC | | TPGTSESATPESGGGEVQLVESGGGLVQP | |
| | GAAAGTGGTGGCGGAGAGGTTCAGCTGGTGGAGTCTGGCGGTGGCCTGGTGCAGCCAGGGGGCTCACTCCGTTTGTCCTGTGCA | | GGSLRLSCAASGFNLSYYYMHWVRQAPG | |
| | GCTTCTGGCTTCAACCTCTCTTATTATTATATGCACTGGGTGCGTCAGGCCCCGGGTAAGGGCCTGGAATGGGTTGCATCTATTTAT | | KGLEWVASIYSSYGYTYYADSVKGRFTISAD | |
| | TCTTCTTATGGCTATACTTATTATGCCGATAGCGTCAAGGGCCGTTTCACTATAAGCGCAGACACATCCAAAAACACAGCCTACCTA | | TSKNTAYLQMNSLRAEDTAVYYCARWSH | |
| | CAAATGAACAGCTTAAGAGCTGAGGACACTGCCGTCTATTATTGTGCTCGCTGGTCTCATGTTTCTGGTCATTACTCTGGTATGGAC | | VSGHYSGMDYWGQGTLVTVSSGGGGSDI | |
| | TACTGGGGTCAAGGAACCCTGGTCACCGTCTCCTCGGGTGGAGGTGGCAGTGATATCCAGATGACCCAGTCCCCGAGCTCCCTGT | | QMTQSPSSLSASVGDRVTITCRASQSVSSA | |
| | CCGCCTCTGTGGGCGATAGGGTCACCATCACCTGCCGTGCCAGTCAGTCCGTGTCCAGCGCTGTAGCCTGGTATCAACAGAAACCA | | VAWYQQKPGKAPKWYSASSLYSGVPSRF | |
| | GGAAAAGCTCCGAAGCTTCTGATTTACTCGGCATCCAGCCTCTACTCTGGAGTCCCTTCTCGCTTCTCTGGTAGCCGTTCCGGGACG | | SGSRSGTDFTLTISSLQPEDFATYYCQQSSY | |
| | GATTTCACTCTGACCATCAGCAGTCTGCAGCCGGAAGACTTCGCAACTTATTACTGTCAGCAATCTTCTTATTCTCTGATCACGTTCG | | SLITFGQGTKVEIK* | |
| | GACAGGGTACCAAGGTGGAGATCAAA | | | |
| 2890- | GAGGTTCAGCTGGTGGAGTCTGGCGGTGGCCTGGTGCAGCCAGGGGGCTCACTCCGTTTGTCCTGTGCAGCTTCTGGCTTCAACA | 76 | EVQLVESGGGLVQPGGSLRLSCAASGFNIY | 77 |
| knob- | TCTATTATTCTTCTATCCACTGGGTGCGTCAGGCCCCGGGTAAGGGCCTGGAATGGGTTGCATCTATTTATCCTTATTATGGCTATA | | YSSIHWVRQAPGKGLEWVASIYPYYGYTYY | |
| 2539- | CTTATTATGCCGATAGCGTCAAGGGCCGTTTCACTATAAGCGCAGACACATCCAAAAACACAGCCTACCTACAAATGAACAGCTTA | | ADSVKGRFTISADTSKNTAYLQMNSLRAED | |
| 2542 | AGAGCTGAGGACACTGCCGTCTATTATTGTGCTCGCTACTACCATTACGGTTTGGACTACTGGGGTCAAGGAACCCTGGTCACCGT | | TAVYYCARYYHYGLDYWGQGTLVTVSSGG | |
| | CTCCTCGGGTGGAGGTGGCAGTGATATCCAGATGACCCAGTCCCCGAGCTCCCTGTCCGCCTCTGTGGGCGATAGGGTCACCATC | | GGSDIQMTQSPSSLSASVGDRVTITCRASQ | |
| | ACCTGCCGTGCCAGTCAGTCCGTGTCCAGCGCTGTAGCCTGGTATCAACAGAAACCAGGAAAAGCTCCGAAGCTTCTGATTTACTC | | SVSSAVAWYQQKPGKAPKWYSASSLYSG | |
| | GGCATCCAGCCTCTACTCTGGAGTCCCTTCTCGCTTCTCTGGTAGCCGTTCCGGGACGGATTTCACTCTGACCATCAGCAGTCTGCA | | VPSRFSGSRSGTDFTLTISSLQPEDFATYYC | |
| | GCCGGAAGACTTCGCAACTTATTACTGTCAGCAATCTTACTGGCATTCTTACCTGATCACGTTCGGACAGGGTACCAAGGTGGAGA | | QQSYWHSYLITFGQGTKVEIKLEDKTHTKV | |
| | TCAAACTCGAGgacaaaactcacacaAAAGTGGAGCCCAAAACTTCTgataagacccatactTGCCCACCGTGCCCAGCACCTGAACTCCTG | | EPKTSDKTHTCPPCPAPELLGGPSVFLFPPK | |
| | GGGGGACCGTCAGTCTTCCTCTTCCCCCCAAAACCCAAGGACACCCTCATGATCTCCCGGACCCCTGAGGTCACATGCGTGGTGGT | | PKDTLMISRTPEVICVVVDVSHEDPEVKFN | |
| | GGACGTGAGCCACGAAGACCCTGAGGTCAAGTTCAACTGGTACGTGGACGGCGTGGAGGTGCATAATGCCAAGACAAAGCCGCG | | WYVDGVEVHNAKTKPREEQYNSTYRVVS | |
| | CGAGGAGCAGTACAACAGCACGTACCGTGTGGTCAGCGTCCTCACCGTCCTGCACCAGGACTGGCTGAATGGCAAGGAGTACAA | | VLTVLHQDWLNGKEYKCKVSNKALPAPIE | |
| | GTGCAAGGTCTCCAACAAAGCCCTCCCAGCCCCCATCGAGAAAACCATCTCCAAAGCCAAAGGGCAGCCCCGAGAACCAATGGTG | | KTISKAKGQPREPMVFDLPPSREEMTKNQ | |
| | TTTGACCTGCCCCCATCCCGGGAGGAGATGACCAAGAACCAGGTCAGCCTGTGGTGCATGGTCAAGGGCTTCTATCCCAGCGACA | | VSLWCMVKGFYPSDIAVEWESNGQPENN | |
| | TCGCCGTGGAGTGGGAGAGCAATGGGCAGCCGGAGAACAACTACAAGACCACGCCTCCCGTGCTGGACTCCGACGGCTCCTTCTT | | YKTTPPVLDSDGSFFLYSKLTVDKSRWQQG | |
| | CCTGTACAGCAAGCTCACCGTGGACAAGAGCCGCTGGCAGCAGGGGAACGTCTTCTCATGCTCCGTGATGCATGAGGCTCTGCAC | | NVFSCSVMHEALHNHYTQKSLSLSPGKSG | |
| | AACCACTACACGCAGAAGAGCCTCTCCCTGTCTCCGGGTAAAAGCGGCAGCGAGACTCCCGGGACCTCAGAGTCCGCCACACCCG | | SETPGTSESATPESGGGEVQLVESGGGLVQ | |
| | AAAGTGGTGGCGGAGAGGTTCAGCTGGTGGAGTCTGGCGGTGGCCTGGTGCAGCCAGGGGGCTCACTCCGTTTGTCCTGTGCAG | | PGGSLRLSCAASGFNISYSSIHWVRQAPGK | |
| | CTTCTGGCTTCAACATCTCTTATTCTTCTATCCACTGGGTGCGTCAGGCCCCGGGTAAGGGCCTGGAATGGGTTGCATATATTTCTT | | GLEWVAYISSYYGYTYYADSVKGRFTISADT | |
| | CTTATTATGGCTATACTTATTATGCCGATAGCGTCAAGGGCCGTTTCACTATAAGCGCAGACACATCCAAAAACACAGCCTACCTAC | | SKNTAYLQMNARAEDTAVYYCARAHYFP | |
| | AAATGAACAGCTrAAGAGCTGAGGACACTGCCGTCTATTATTGTGCTCGCGCTCATTACTTCCCGTGGGCTGGTGCTATGGACTAC | | WAGAMDYWGQGTLVTVSSGGGGSDIQ | |
| | TGGGGTCAAGGAACCCTGGTCACCGTCTCCTCGGGTGGAGGTGGCAGTGATATCCAGATGACCCAGTCCCCGAGCTCCCTGTCCG | | MTQSPSSLSASVGDRVTITCRASQSVSSAV | |
| | CCTCTGTGGGCGATAGGGTCACCATCACCTGCCGTGCCAGTCAGTCCGTGTCCAGCGCTGTAGCCTGGTATCAACAGAAACCAGG | | AWYQQKPGKAPKLLIMSSLYSGVPSRFS | |
| | AAAAGCTCCGAAGCTTCTGATTTACTCGGCATCCAGCCTCTACTCTGGAGTCCCTTCTCGCTTCTCTGGTAGCCGTTCCGGGACGGA | | GSRSGTDFILTISSLQPEDFATYYCQQYYW | |
| | TTTCACTCTGACCATCAGCAGTCTGCAGCCGGAAGACTTCGCAACTTATTACTGTCAGCAATACTACTGGCCGATCACGTTCGGACA | | PITFGQGTKVEIK* | |
| | GGGTACCAAGGTGGAGATCAAA | | | |
| 2890- | GAGGTTCAGCTGGTGGAGTCTGGCGGTGGCCTGGTGCAGCCAGGGGGCTCACTCCGTTTGTCCTGTGCAGCTTCTGGCTTCAACA | 78 | EVQLVESGGGLVQPGGSLRLSCAASGFNIY | 79 |
| hole- | TCTATTATTCTTCTATCCACTGGGTGCGTCAGGCCCCGGGTAAGGGCCTGGAATGGGTTGCATCTATTTATCCTTATTATGGCTATA | | YSSIHWVRQAPGKGLEWVASIYPYYGYTYY | |
| 2539- | CTTATTATGCCGATAGCGTCAAGGGCCGTTTCACTATAAGCGCAGACACATCCAAAAACACAGCCTACCTACAAATGAACAGCTTA | | ADSVKGRFTISADTSKNTAYLQMNSLRAED | |
| 2542 | AGAGCTGAGGACACTGCCGTCTATTATTGTGCTCGCTACTACCATTACGGTTTGGACTACTGGGGTCAAGGAACCCTGGTCACCGT | | TAVYYCARYYHYGLDYWGQGTLVTVSSGG | |
| | CTCCTCGGGTGGAGGTGGCAGTGATATCCAGATGACCCAGTCCCCGAGCTCCCTGTCCGCCTCTGTGGGCGATAGGGTCACCATC | | GGSDIQMTQSPSSLSASVGDRVTITCRASQ | |
| | ACCTGCCGTGCCAGTCAGTCCGTGTCCAGCGCTGTAGCCTGGTATCAACAGAAACCAGGAAAAGCTCCGAAGCTTCTGATTTACTC | | SVSSAVAWYQQKPGKAPKLUYSASSLYSG | |
| | GGCATCCAGCCTCTACTCTGGAGTCCCTTCTCGCTTCTCTGGTAGCCGTTCCGGGACGGATTTCACTCTGACCATCAGCAGTCTGCA | | VPSRFSGSRSGTDFTLTISSLQPEDFATYYC | |
| | GCCGGAAGACTTCGCAACTTATTACTGTCAGCAATCTTACTGGCATTCTTACCTGATCACGTTCGGACAGGGTACCAAGGTGGAGA | | QQSYWHSYLITFGQGTKVEIKLEDKTHTKV | |
| | TCAAACTCGAGgacaaaactcacacaAAAGTTGAGCCCAAATCTTCTgataagacccataatTGCCCACCGTGCCCAGCACCTGAACTCCTG | | EPKSSDKTHNCPPCPAPELLGGPSVFLFPPK | |
| | GGGGGACCGTCAGTCTTCCTCTTCCCCCCAAAACCCAAGGACACCCTCATGATCTCCCGGACCCCTGAGGTCACATGCGTGGTGGT | | PKDTLMISRTPEVTCWVDVSHEDPEVKFN | |
| | GGACGTGAGCCACGAAGACCCTGAGGTCAAGTTCAACTGGTACGTGGACGGCGTGGAGGTGCATAATGCCAAGACAAAGCCGCG | | WYVDGVEVHNAKTKPREEQYNSTYRWS | |
| | GGAGGAGCAGTACAACAGCACGTACCGTGTGGTCAGCGTCCTCACCGTCCTGCACCAGGACTGGCTGAATGGCAAGGAGTACAA | | VLTVLHQDWLNGKEYKCKVSNKALPAPIE | |
| | GTGCAAGGTCTCCAACAAAGCCCTCCCAGCCCCCATCGAGAAAACCATCTCCAAAGCCAAAGGGCAGCCCCGAGAACCACAGGTG | | KTISKAKGQPREPQVYTLPPIRELMTSNQV | |
| | TACACCCTGCCCCCAATCCGGGAGCTGATGACCAGCAACCAGGTCAGCCTGAGCTGCGCCGTCAAAGGCTTCTATCCCAGCGACAT | | SLSCAVKGFYPSDIAVEWESNGQPENNYK | |
| | CGCCGTGGAGTGGGAGAGCAATGGGCAGCCGGAGAACAACTACAAGACCACGCCTCCCGTGCTGGACTCCGACGGCTCCTTCT1C | | TTPPVLDSDGSFFLVSKLTVDKSRWQQGN | |
| | CTCGTGAGCAAGCTCACCGTGGACAAGAGCAGGTGGCAGCAGGGGAACGTCTTCTCATGCTCCGTGATGCATGAGGCTCTGCACA | | VFSCSVMHEALHNHYTQKSLSLSPGKSGSE | |
| | ACCACTACACGCAGAAGAGCCTCTCCCTGTCTCCGGGTAAAAGCGGCAGCGAGACTCCCGGGACCTCAGAGTCCGCCACACCCGA | | TPGTSESATPESGGGEVQLVESGGGLVQP | |
| | AAGTGGTGGCGGAGAGGTTCAGCTGGTGGAGTCTGGCGGTGGCCTGGTGCAGCCAGGGGGCTCACTCCGTTTGTCCTGTGCAGC | | GGSLRLSCAASGFNISSYYIHWVRQAPGKG | |
| | TTCTGGCTTCAACATCTCTTCTTATTATATCCACTGGGTGCGTCAGGCCCCGGGTAAGGGCCTGGAATGGGTTGCATCTATTTATTC | | LEWVASIYSSYGYTSYADSVKGRFTISADTS | |
| | TTCTTATGGCTATACTTCTTATGCCGATAGCGTCAAGGGCCGTTTCACTATAAGCGCAGACACATCCAAAAACACAGCCTACCTACA | | KNTAYLQMNSLRAEDTAVYYCARTVRGSK | |
| | AATGAACAGCTTAAGAGCTGAGGACACTGCCGTCTATTATTGTGCTCGCACTGTTCGTGGATCCAAAAAACCGTACTTCTCTGGTT | | KPYFSGWAMDYWGQGTLVTVSSGGGGS | |
| | GGGCTATGGACTACTGGGGTCAAGGAACCCTGGTCACCGTCTCCTCGGGTGGAGGTGGCAGTGATATCCAGATGACCCAGTCCCC | | DIQMTQSPSSLSASVGDRVTITCRASQSVS | |
| | GAGCTCCCTGTCCGCCTCTGTGGGCGATAGGGTCACCATCACCTGCCGTGCCAGTCAGTCCGTGTCCAGCGCTGTAGCCTGGTATC | | SAVAWYQQKPGKAPKLLIYSASSLYSGVPS | |
| | AACAGAAACCAGGAAAAGCTCCGAAGCTTCTGATTTACTCGGCATCCAGCCTCTACTCTGGAGTCCCTTCTCGCTTCTCTGGTAGCC | | RFSGSRSGTDFILTISSLOPEDFATYYCQQY | |
| | GTTCCGGGACGGATTTCACTCTGACCATCAGCAGTCTGCAGCCGGAAGACTTCGCAACTTATTACTGTCAGCAATACTCTTGGGGT | | SWGPFTFGQGTKVEIK* | |
| | CCGTTCACGTTCGGACAGGGTACCAAGGTGGAGATCAAA | | | |
| 12735- | GAGGTTCAGCTGGTGGAGTCTGGCGGTGGCCTGGTGCAGCCAGGGGGCTCACTCCGTTTGTCCTGTGCAGCTTCTGGCTTCAACA | 80 | EVQLVESGGGLVQPGGSLRLSCAASGFNIS | 81 |
| knob- | TCTCTTCTTCTTCTATGCACTGGGTGCGTCAGGCCCCGGGTAAGGGCCTGGAATGGGTTGCATCTATTTATTCTTATTATGGCTCTA | | SSSMHWVRQAPGKGLEWVASIYSYYGSTY | |
| 2539- | CTTATTATGCCGATAGCGTCAAGGGCCGTTTCACTATAAGCGCAGACACATCCAAAAACACAGCCTACCTACAAATGAACAGCTTA | | YADSVKGRFTISADTSKNTAYLQMNSLRAE | |
| 2542 | AGAGCTGAGGACACTGCCGTCTATTATTGTGCTCGCTGGTACGGTATGGACTACTGGGGTCAAGGAACCCTGGTCACCGTCTCCTC | | DTAVYYCARWYGMDYWGQGTLVTVSSG | |
| | GGGTGGAGGTGGCAGTGATATCCAGATGACCCAGTCCCCGAGCTCCCTGTCCGCCTCTGTGGGCGATAGGGTCACCATCACCTGC | | GGGSDIQMTQSPSSLSASVGDRVTITCRAS | |
| | CGTGCCAGTCAGTCCGTGTCCAGCGCTGTAGCCTGGTATCAACAGAAACCAGGAAAAGCTCCGAAGCTTCTGATTTACTCGGCATC | | QSVSSAVAWYQQKPGKAPKLUYSASSLYS | |
| | CAGCCTCTACTCTGGAGTCCCTTCTCGCTTCTCTGGTAGCCGTTCCGGGACGGATTTCACTCTGACCATCAGCAGTCTGCAGCCGGA | | GVPSRFSGSRSGTDFTLTISSLQPEDFATYY | |
| | AGACTTCGCAACTTATTACTGTCAGCAACCGGGTTCTTGGTACTTCCCGCCGATCACGTTCGGACAGGGTACCAAGGTGGAGATCA | | CQQPGSWYFPPITFGQGTKVEIKLEDKTHT | |
| | AACTCGAGgacaaaactcacacaAAAGTGGAGCCCAAAACTTCTgataagacccatactTGCCCACCGTGCCCAGCACCTGAACTCCTGGGG | | KVEPKTSDKTHTCPPCPAPELLGGPSVFLFP | |
| | GGACCGTCAGTCTTCCTCTTCCCCCCAAAACCCAAGGACACCCTCATGATCTCCCGGACCCCTGAGGTCACATGCGTGGTGGTGGA | | PKPKDTLMISRTPEVTCVVVDVSHEDPEVK | |
| | CGTGAGCCACGAAGACCCTGAGGTCAAGTTCAACTGGTACGTGGACGGCGTGGAGGTGCATAATGCCAAGACAAAGCCGCGCGA | | FNWYVDGVEVHNAKTKPREEQYNSTYRV | |
| | GGAGCAGTACAACAGCACGTACCGTGTGGTCAGCGTCCTCACCGTCCTGCACCAGGACTGGCTGAATGGCAAGGAGTACAAGTGC | | VSVLTVLHQDWLNGKEYKCKVSNKALPAP | |
| | AAGGTCTCCAACAAAGCCCTCCCAGCCCCCATCGAGAAAACCATCTCCAAAGCCAAAGGGCAGCCCCGAGAACCAATGGTGTTTG | | IEKTISKAKGQPREPMVFDLPPSREEMTKN | |
| | ACCTGCCCCCATCCCGGGAGGAGATGACCAAGAACCAGGTCAGCCTGTGGTGCATGGTCAAGGGCTTCTATCCCAGCGACATCGC | | QVSLWCMVKGFYPSDIAVEWESNGQPEN | |
| | CGTGGAGTGGGAGAGCAATGGGCAGCCGGAGAACAACTACAAGACCACGCCTCCCGTGCTGGACTCCGACGGCTCCTTCTTCCTG | | NYKTTPPVLDSDGSFFLYSKLTVDKSRWQQ | |
| | TACAGCAAGCTCACCGTGGACAAGAGCCGCTGGCAGCAGGGGAACGTCTTCTCATGCTCCGTGATGCATGAGGCTCTGCACAACC | | GNVFSCSVMHEALHNHYTQKSLSLSPGKS | |
| | ACTACACGCAGAAGAGCCTCTCCCTGTCTCCGGGTAAAAGCGGCAGCGAGACTCCCGGGACCTCAGAGTCCGCCACACCCGAAAG | | GSETPGTSESATPESGGGEVQLVESGGGLV | |
| | TGGTGGCGGAGAGGTTCAGCTGGTGGAGTCTGGCGGTGGCCTGGTGCAGCCAGGGGGCTCACTCCGTTTGTCCTGTGCAGCTTCT | | QPGGSLRLSCAASGFNISYSSIHWVROAPG | |
| | GGCTTCAACATCTCTTATTCTTCTATCCACTGGGTGCGTCAGGCCCCGGGTAAGGGCCTGGAATGGGTTGCATATATTTCTTCTTAT | | KGLEWVAYISSYYGYTYYADSVKGRFTISAD | |
| | TATGGCTATACTTATrATGCCGATAGCGTCAAGGGCCGTTTCACTATAAGCGCAGACACATCCAAAAACACAGCCTACCTACAAAT | | TSKNTAYLQMNSLRAEDTAVYYCARAHYF | |
| | GAACAGCTTAAGAGCTGAGGACACTGCCGTCTATTATTGTGCTCGCGCTCATTACTTCCCGTGGGCTGGTGCTATGGACTACTGGG | | PWAGAMDYWGQGTLVTVSSGGGGSDIQ | |
| | GTCAAGGAACCCTGGTCACCGTCTCCTCGGGTGGAGGTGGCAGTGATATCCAGATGACCCAGTCCCCGAGCTCCCTGTCCGCCTCT | | MTQSPSSLSASVGDRVTITCRASQSVSSAV | |
| | GTGGGCGATAGGGTCACCATCACCTGCCGTGCCAGTCAGTCCGTGTCCAGCGCTGTAGCCTGGTATCAACAGAAACCAGGAAAAG | | AWYQQKPGKAPKLLIYSASSLYSGVPSRFS | |
| | CTCCGAAGCTTCTGATTTACTCGGCATCCAGCCTCTACTCTGGAGTCCCTTCTCGCTTCTCTGGTAGCCGTTCCGGGACGGATTTCA | | GSRSGTDFTLTISSLCIPEDFATYYCQQYYW | |
| | CTCTGACCATCAGCAGTCTGCAGCCGGAAGACTTCGCAACTTATTACTGTCAGCAATACTACTGGCCGATCACGTTCGGACAGGGT | | PITFGQGTKVEIK* | |
| | ACCAAGGTGGAGATCAAA | | | |
| 12735- | GAGGTTCAGCTGGTGGAGTCTGGCGGTGGCCTGGTGCAGCCAGGGGGCTCACTCCGTTTGTCCTGTGCAGCTTCTGGCTTCAACA | 82 | EVQLVESGGGLVQPGGSLRLSCAASGFNIS | 83 |
| hole- | TCTCTTCTTCTTCTATGCACTGGGTGCGTCAGGCCCCGGGTAAGGGCCTGGAATGGGTTGCATCTATTTATTCTTATTATGGCTCTA | | SSWHWVROAPGKGLEWVASIYSYYGSTY | |
| 2539- | CTTATTATGCCGATAGCGTCAAGGGCCGTTTCACTATAAGCGCAGACACATCCAAAAACACAGCCTACCTACAAATGAACAGCTTA | | YADSVKGRFTISADTSKNTAYLQMNSLRAE | |
| 2542 | AGAGCTGAGGACACTGCCGTCTATTATTGTGCTCGCTGGTACGGTATGGACTACTGGGGTCAAGGAACCCTGGTCACCGTCTCCTC | | DTAVYYCARWYGMDYWGQGTLVTVSSG | |
| | GGGTGGAGGTGGCAGTGATATCCAGATGACCCAGTCCCCGAGCTCCCTGTCCGCCTCTGTGGGCGATAGGGTCACCATCACCTGC | | GGGSDIQMTQSPSSLSASVGDRVTITCRAS | |
| | CGTGCCAGTCAGTCCGTGTCCAGCGCTGTAGCCTGGTATCAACAGAAACCAGGAAAAGCTCCGAAGCTTCTGATTTACTCGGCATC | | QSVSSAVAWYQQKPGKAPKLLIYSASSLYS | |
| | CAGCCTCTACTCTGGAGTCCCTTCTCGCTTCTCTGGTAGCCGTTCCGGGACGGATTTCACTCTGACCATCAGCAGTCTGCAGCCGGA | | GVPSRFSGSRSGTDFTLTISSLQPEDFATYY | |
| | AGACTTCGCAACTTATTACTGTCAGCAACCGGGTTCTTGGTACTTCCCGCCGATCACGTTCGGACAGGGTACCAAGGTGGAGATCA | | CQQPGSWYFPPITFGQGTKVEIKLEDKTHT | |
| | AACTCGAGgacaaaactcacacaAAAGTTGAGCCCAAATCTTCTgataagacccataatTGCCCACCGTGCCCAGCACCTGAACTCCTGGGG | | KVEPKSSDKTHNCPPCPAPELLGGPSVFLF | |
| | GGACCGTCAGTCTTCCTCTTCCCCCCAAAACCCAAGGACACCCTCATGATCTCCCGGACCCCTGAGGTCACATGCGTGGTGGTGGA | | PPKPKDTLMISRTPDITCVVVDVSHEDPEV | |
| | CGTGAGCCACGAAGACCCTGAGGTCAAGTTCAACTGGTACGTGGACGGCGTGGAGGTGCATAATGCCAAGACAAAGCCGCGGGA | | KFNWYVDGVEVHNAKTKPREEQYNSTYR | |
| | GGAGCAGTACAACAGCACGTACCGTGTGGTCAGCGTCCTCACCGTCCTGCACCAGGACTGGCTGAATGGCAAGGAGTACAAGTGC | | VVSVLTVLHQDWLNGKEYKCKVSNKALPA | |
| | AAGGTCTCCAACAAAGCCCTCCCAGCCCCCATCGAGAAAACCATCTCCAAAGCCAAAGGGCAGCCCCGAGAACCACAGGTGTACA | | PIEKTISKAKGQPREPQVYTLPPIRELMTSN | |
| | CCCTGCCCCCAATCCGGGAGCTGATGACCAGCAACCAGGTCAGCCTGAGCTGCGCCGTCAAAGGCTTCTATCCCAGCGACATCGCC | | QVSLSCAVKGFYPSDIAVEWESNGQPENN | |
| | GTGGAGTGGGAGAGCAATGGGCAGCCGGAGAACAACTACAAGACCACGCCTCCCGTGCTGGACTCCGACGGCTCCTTCTTCCTCG | | YKTTPPVLDSDGSFFLVSKLTVDKSRWQQ | |
| | TGAGCAAGCTCACCGTGGACAAGAGCAGGTGGCAGCAGGGGAACGTCTTCTCATGCTCCGTGATGCATGAGGCTCTGCACAACCA | | GNVFSCSVMHEALHNHYTQKSLSLSPGKS | |
| | CTACACGCAGAAGAGCCTCTCCCTGTCTCCGGGTAAAAGCGGCAGCGAGACTCCCGGGACCTCAGAGTCCGCCACACCCGAAAGT | | GSETPGTSESATPESGGGEVQLVESGGGLV | |
| | GGTGGCGGAGAGGTTCAGCTGGTGGAGTCTGGCGGTGGCCTGGTGCAGCCAGGGGGCTCACTCCGTTTGTCCTGTGCAGCTTCT | | QPGGSLRLSCAASGFNISSYYIHWVROAPG | |
| | GGCTTCAACATCTCTTCTTATTATATCCACTGGGTGCGTCAGGCCCCGGGTAAGGGCCTGGAATGGGTTGCATCTATTTATTCTTCT | | KGLEWVASIYSSYGYTSYADSVKGRFTISAD | |
| | TATGGCTATACTTCTTATGCCGATAGCGTCAAGGGCCGTTTCACTATAAGCGCAGACACATCCAAAAACACAGCCTACCTACAAAT | | TSKNTAYLQMNSLRAEDTAVYYCARTVRG | |
| | GAACAGCTTAAGAGCTGAGGACACTGCCGTCTATTATTGTGCTCGCACTGTTCGTGGATCCAAAAAACCGTACTTCTCTGGTTGGG | | SKKPYFSGWAMDYWGQGTLVTVSSGGG | |
| | CTATGGACTACTGGGGTCAAGGAACCCTGGTCACCGTCTCCTCGGGTGGAGGTGGCAGTGATATCCAGATGACCCAGTCCCCGAG | | GSDIQMTQSPSSLSASVGDRVTITCRASQS | |
| | CTCCCTGTCCGCCTCTGTGGGCGATAGGGTCACCATCACCTGCCGTGCCAGTCAGTCCGTGTCCAGCGCTGTAGCCTGGTATCAAC | | VSSAVAWYQQKPGKAPKLLIYSASSLYSGV | |
| | AGAAACCAGGAAAAGCTCCGAAGCTTCTGATTTACTCGGCATCCAGCCTCTACTCTGGAGTCCCTTCTCGCTTCTCTGGTAGCCGTT | | PSRFSGSRSGTDFTLTISSLQPEDFATYYCQ | |
| | CCGGGACGGATTTCACTCTGACCATCAGCAGTCTGCAGCCGGAAGACTTCGCAACTTATTACTGTCAGCAATACTCTTGGGGTCCG | | QYSWGPFTFGQGTKVEIK* | |
| | TTCACGTTCGGACAGGGTACCAAGGTGGAGATCAAA | | | |
| 5027- | GAGGTTCAGCTGGTGGAGTCTGGCGGTGGCCTGGTGCAGCCAGGGGGCTCACTCCETTTGTCCTGTGCAGCTTCTGGCTTCAACT | 89 | EVQLVESGGGLVQPGGSLRLSCMSGFNSS | 94 |
| knob- | CCTCTTTTTATTTTATGCACTGGGTGCGTCAGGCCCCGGGTAAGGGCCTGGAATGGGTTGCAACTGTTTATCCTTATCTTGACTATA | | FVFMHWVROAPGKGLEWVATVYPYLDYT | |
| 2539- | CTTATTATGCCGATAGCGTCAAGGGCCGTTTCACTATAAGCGCAGACACATCCAAAAACACAGCCTACCTACAAATGAACAGCTTA | | YVADSVKGRFTISADTSKNTAYLQMNSLRA | |
| 2542 | AGAGCTGAGGACACTGCCGTCTATTATTGTGCTCGCGCGTTTCCGGGTTCTTACCATCCTATGGACTACTGGGGTCAAGGAACCCT | | EDTAVYYCARAFPGSYHPMDYWGQGTLV | |
| | GGTCACCGTCTCCTCGGGTGGAGGTGGCAGTGATATCCAGATGACCCAGTCCCCGAGCTCCCTGTCCGCCTCTGTGGGCGATAGG | | TVSSGGGGSDIQMTQSPSSLSASVGDRVTI | |
| | GTCACCATCACCTGCCGTGCCAGTCAGTCCGTGTCCAGCGCTGTAGCCTGGTATCAACAGAAACCAGGAAAAGCTCCGAAGCTTCT | | TCRASQSVSSAVAWYQQKPGKAPKLLIYSA | |
| | GATTTACTCGGCATCCAGCCTCTACTCTGGAGTCCCTTCTCGCTTCTCTGGTAGCCGTTCCGGGACGGATTTCACTCTGACCATCAG | | SSLYSGVPSRFSGSRSGTDFTLTISSLQPEDF | |
| | CAGTCTGCAGCCGGAAGACTTCGCAACTTATTACTGTCAGCAATCTTCTTATTCTCTGATCACGTTCGGACAGGGTACCAAGGTGG | | ATYYCQQSSYSLITFGQGTKVEIKLEDKTFIT | |
| | AGATCAAACTCGAGgacaaaactcacacaAAAGTGGAGCCCAAAACTTCTgataagacccatactTGCCCACCGTGCCCAGCACCTGAACTC | | KVEPKTSDKTHTCPPCPAPELLGGPSVFLFP | |
| | CTGGGGGGACCGTCAGTCTTCCTCTTCCCCCCAAAACCCAAGGACACCCTCATGATCTCCCGGACCCCTGAGGTCACATGCGTGGT | | PKPKDTLMISRTPEVICVVVDVSHEDPEVK | |
| | GGTGGACGTGAGCCACGAAGACCCTGAGGTCAAGTTCAACTGGTACGTGGACGGCGTGGAGGTGCATAATGCCAAGACAAAGCC | | FNWYVDGVEVHNAKTKPREEQYNSTYRV | |
| | GCGCGAGGAGCAGTACAACAGCACGTACCGTGTGGTCAGCGTCCTCACCGTCCTGCACCAGGACTGGCTGAATGGCAAGGAGTA | | VSVITVLHQDWLNGKEYKCKVSNKALPAP | |
| | CAAGTGCAAGGTCTCCAACAAAGCCCTCCCAGCCCCCATCGAGAAAACCATCTCCAAAGCCAAAGGGCAGCCCCGAGAACCAATG | | IEKTISKAKGQPREPMVFDLPPSREEMTKN | |
| | GTGTTTGACCTGCCCCCATCCCGGGAGGAGATGACCAAGAACCAGGTCAGCCTGTGGTGCATGGTCAAGGGCTTCTATCCCAGCG | | QVSLWCMVKGFYPSDIAVEWESNGQPEN | |
| | ACATCGCCGTGGAGTGGGAGAGCAATGGGCAGCCGGAGAACAACTACAAGACCACGCCTCCCGTGCTGGACTCCGACGGCTCCT | | NYKTTPPVLDSDGSFFLYSKLTVDKSRWQQ | |
| | TCTTCCTGTACAGCAAGCTCACCGTGGACAAGAGCCGCTGGCAGCAGGGGAACGTCTTCTCATGCTCCGTGATGCATGAGGCTCT | | GNVFSCSVMHEALHNHYTQKSLSLSIDGKS | |
| | GCACAACCACTACACGCAGAAGAGCCTCTCCCTGTCTCCGGGTAAAAGCGGCAGCGAGACTCCCGGGACCTCAGAGTCCGCCACA | | GSETPGTSESATPESGGGEVQLVESGGGLV | |
| | CCCGAAAGTGGTGGCGGAGAGGTTCAGCTGGTGGAGTCTGGCGGTGGCCTGGTGCAGCCAGGGGGCTCACTCCGTTTGTCCTGT | | QPGGSLRLSCAASGFNISYSSIHWVROAPG | |
| | GCAGCTTCTGGCTTCAACATCTCTTATTCTTCTATCCACTGGGTGCGTCAGGCCCCGGGTAAGGGCCTGGAATGGGTTGCATATAT | | KGLEWVAYISSYYGYTYYADSVKGRFTISAD | |
| | TTCTTCTTATTATGGCTATACTTATTATGCCGATAGCGTCAAGGGCCGTTTCACTATAAGCGCAGACACATCCAAAAACACAGCCTA | | TSKNTAYLQMNSLRAEDTAVYYCARAHYF | |
| | CCTACAAATGAACAGCTTAAGAGCTGAGGACACTGCCGTCTATTATTGTGCTCGCGCTCATTACTTCCCGTGGGCTGGTGCTATGG | | PWAGAMDYWGQGTLVTVSSGGGGSDIQ | |
| | ACTACTGGGGTCAAGGAACCCTGGTCACCGTCTCCTCGGGTGGAGGTGGCAGTGATATCCAGATGACCCAGTCCCCGAGCTCCCT | | MTQSPSSLSASVGDRVTITCRASQSVSSAV | |
| | GTCCGCCTCTGTGGGCGATAGGGTCACCATCACCTGCCGTGCCAGTCAGTCCGTOCCAGCGCTGTAGCCTGGTATCAACAGAAAC | | AWYQQKPGKAPKLLIYSASSLYSGVPSRFS | |
| | CAGGAAAAGCTCCGAAGCTTCTGATTTACTCGGCATCCAGCCTCTACTCTGGAGTCCCTTCTCGCTTCTCTGGTAGCCGTTCCGGGA | | GSMGTDFILTISSLQPEDFATYYCQQYYW | |
| | CGGATTTCACTCTGACCATCAGCAGTCTGCAGCCGGAAGACTTCGCAACTTATTACTGTCAGCAATACTACTGGCCGATCACGTTCG | | PITFGQGTKVEIK* | |
| | GACAGGGTACCAAGGTGGAGATCAAATGA | | | |
| 5027- | GAGGTTCAGCTGGTGGAGTCTGGCGGTGGCCTGGTGCAGCCAGGGGGCTCACTCCGTTTGTCCTGTGCAGCTTCTGGCTTCAACT | 92 | EVQLVESGGGLVQPGGSLRLSCAASGFNSS | 93 |
| hole- | CCTCTTTTTATTTTATGCACTGGGTGCGTCAGGCCCCGGGTAAGGGCCTGGAATGGGTTGCAACTGTTTATCCTTATCTTGACTATA | | FYFMHWVROAPGKGLEWVATVYPYLDYT | |
| 2539- | CTTATTATGCCGATAGCGTCAAGGGCCGTTTCACTATAAGCGCAGACACATCCAAAAACACAGCCTACCTACAAATGAACAGCTTA | | YYADSVKGRFTISADTSKNTAYLQMNSLRA | |
| 2542 | AGAGCTGAGGACACTGCCGTCTATTATTGTGCTCGCGCGTTTCCGGGTTCTTACCATCCTATGGACTACTGGGGTCAAGGAACCCT | | EDTAVYYCARAFPGSYHPMDYWGQGTLV | |
| | GGTCACCGTCTCCTCGGGTGGAGGTGGCAGTGATATCCAGATGACCCAGTCCCCGAGCTCCCTGTCCGCCTCTGTGGGCGATAGG | | TVSSGGGGSDIQMTQSPSSLSASVGDRVTI | |
| | GTCACCATCACCTGCCGTGCCAGTCAGTCCGTGTCCAGCGCTGTAGCCTGGTATCAACAGAAACCAGGAAAAGCTCCGAAGCTTCT | | TCRASQSVSSAVAWYQQKPGKAPKLLIYSA | |
| | GATTTACTCGGCATCCAGCCTCTACTCTGGAGTCCCTTCTCGCTTCTCTGGTAGCCGTTCCGGGACGGATTTCACTCTGACCATCAG | | SSLYSGVPSRFSGSRSGTDFTLTISSLOPEDF | |
| | CAGTCTGCAGCCGGAAGACTTCGCAACTTATTACTGTCAGCAATCTTCTTATTCTCTGATCACGTTCGGACAGGGTACCAAGGTGG | | ATYYCQQSSYSLITFGQGTKVEIKLEDKTHT | |
| | AGATCAAACTCGAGgacaaaactcacacaAAAGTTGAGCCCAAATCTTCTgataagacccataatTGCCCACCGTGCCCAGCACCTGAACTC | | KVEPKSSDKTHNCPPCPAPELLGGPSVFLF | |
| | CTGGGGGGACCGTCAGTCTTCCTCTTCCCCCCAAAACCCAAGGACACCCTCATGATCTCCCGGACCCCTGAGGTCACATGCGTGGT | | PPKPKDTLMISRTPEVICVVVDVSHEDPEV | |
| | GGTGGACGTGAGCCACGAAGACCCTGAGGTCAAGTTCAACTGGTACGTGGACGGCGTGGAGGTGCATAATGCCAAGACAAAGCC | | KFNWYVDGVEVHNAKTKPREEQYNSTYR | |
| | GCGGGAGGAGCAGTACAACAGCACGTACCGTGTGGTCAGCGTCCTCACCGTCCTGCACCAGGACTGGCTGAATGGCAAGGAGTA | | VVSVLTVLHQDWLNGKEYKCKVSNKALPA | |
| | CAAGTGCAAGGTCTCCAACAAAGCCCTCCCAGCCCCCATCGAGAAAACCATCTCCAAAGCCAAAGGGCAGCCCCGAGAACCACAG | | PIEKTISKAKGQPREPQVYTLPPIRELMTSN | |
| | GTGTACACCCTGCCCCCAATCCGGGAGCTGATGACCAGCAACCAGGTCAGCCTGAGCTGCGCCGTCAAAGGCTTCTATCCCAGCG | | QVSLSCAVKGFYPSDIAVEWESNGQPENN | |
| | ACATCGCCGTGGAGTGGGAGAGCAATGGGCAGCCGGAGAACAACTACAAGACCACGCCTCCCGTGCTGGACTCCGACGGCTCCT | | YKTTPPVLDSDGSFFIVSKLTVDKSRWQQ | |
| | TCTTCCTCGTGAGCAAGCTCACCGTGGACAAGAGCAGGTGGCAGCAGGGGAACGTCTTCTCATGCTCCGTGATGCATGAGGCTCT | | GNVFSCSVMHEALHNHYTQKSLSLSPGKS | |
| | GCACAACCACTACACGCAGAAGAGCCTCTCCCTGTCTCCGGGTAAAAGCGGCAGCGAGACTCCCGGGACCTCAGAGTCCGCCACA | | GSETPGTSESATPESGGGEVQLVESGGGLV | |
| | CCCGAAAGTGGTGGCGGAGAGGTTCAGCTGGTGGAGTCTGGCGGTGGCCTGGTGCAGCCAGGGGGCTCACTCCGTTTGTCCTGT | | QPGGSLRLSCAASGFNISSYYIHWVRQAPG | |
| | GCAGCTTCTGGCTTCAACATCTCTTCTTATTATATCCACTGGGTGCGTCAGGCCCCGGGTAAGGGCCTGGAATGGGTTGCATCTAT | | KGLEWVASIYSSYGYTSYADSVKGRFTISAD | |
| | TTATTCTTCTTATGGCTATACTTCTTATGCCGATAGCGTCAAGGGCCGTTTCACTATAAGCGCAGACACATCCAAAAACACAGCCTA | | TSKNTAYLQMNSLRAEDTAVYYCARTVRG | |
| | CCTACAAATGAACAGCTTAAGAGCTGAGGACACTGCCGTCTATTATTGTGCTCGCACTGTTCGTGGATCCAAAAAACCGTACTTCTC | | SKKPYFSGWAMDYWGQGTLVTVSSGGG | |
| | TGGTTGGGCTATGGACTACTGGGGTCAAGGAACCCTGGTCACCGTCTCCTCGGGTGGAGGTGGCAGTGATATCCAGATGACCCAG | | GSDIQMTQSPSSLSASVGDRVTITCRASQS | |
| | TCCCCGAGCTCCCTGTCCGCCTCTGTGGGCGATAGGGTCACCATCACCTGCCGTGCCAGTCAGTCCGTGTCCAGCGCTGTAGCCTG | | VSSAVAWYQQKPGKAPKLLIYSASSLYSGV | |
| | GTATCAACAGAAACCAGGAAAAGCTCCGAAGCTTCTGATTTACTCGGCATCCAGCCTCTACTCTGGAGTCCCTTCTCGCTTCTCTGG | | PSRFSGSRSGTDFILTISSLQPEDFATYYCQ | |
| | TAGCCGTTCCGGGACGGATTTCACTCTGACCATCAGCAGTCTGCAGCCGGAAGACTTCGCAACTTATTACTGTCAGCAATACTCTT | | QYSWGPFTFGQGTKVEIK* | |
| | GGGGTCCGTTCACGTTCGGACAGGGTACCAAGGTGGAGATCAAATGA | | | |

TABLE 1B

| | N- | | | | | N- | | | | |
|---|---|---|---|---|---|---|---|---|---|---|
| | fusion | SEQ | | SEQ | VH- | fusion | SEQ | | SEQ | |
| | VH | ID | | ID | VL | VL | ID | | ID | |
| ID | (VH1) | NO: | | NO: | linker | (VL2) | NO: | | NO: | Fc fusion |
| 5019- | 5019 | 45 | GAGGTTCAGCTGG | 46 | GGTGGA | 5019 | 47 | GATATCCAGATG | 48 | CTCGAGgacaaaactcacacaAAAGTGGAGCC |
| knob- | | | TGGAGTCTGGCGG | | GGTGGC | | | ACCCAGTCCCCGA | | CAAAACTTCTgataagacccatactTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTOTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACATCGGTTCT | | | | | GTCAGTCCGTGTC | | CACGAAGACCCTGAGGTCAAGTTCAACTG |
| | | | TCTTCTATCCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGCGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATCTATTTATTCTG | | | | | TGATTTACTCGGC | | GGCAAGGAGTACAAGTGCAAGGTCTCCAA |
| | | | CTTTTGCCTCTACT | | | | | ATCCAGCCTCTAC | | CAAAGCCCTCCCAGCCCCCATCGAGAAAA |
| | | | TCTTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCAATGGTGTTTGACCTGCCCCCATCC |
| | | | TTTCACTATAAGCG | | | | | TAGCCGTTCCGG | | CGGGAGGAGATGACCAAGAACCAGGTCA |
| | | | CAGACACATCCAA | | | | | GACGGAACACT | | GCCTGTGGTGCATGGTCAAGGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTGTACAGCAAGCTCACCG |
| | | | TATTATTGTGCTCG | | | | | CAAGGTGTTTACC | | TGGACAAGAGCCGCTGGCAGCAGGGGAA |
| | | | CTACCATTTCCCGT | | | | | TGTTCACGTTCGG | | CGTCTTCTCATGCTCCGTGATGCATGAGGC |
| | | | TCGGTTTTGCTTTG | | | | | ACAGGGTACCAA | | TCTGCACAACCACTACACGCAGAAGAGCC |
| | | | GACTACTGGGGTC | | | | | GGTGGAGATCAA | | TCTCCCTGTCTCCGGGTAAAAGCGGCAGC |
| | | | AAGGAACCCTGGT | | | | | A | | GAGACTCCCGGGACCTCAGAGTCCGCCAC |
| | | | CACCGTCTCCTCG | | | | | | | ACCCGAAAGTGGTGGCGGA |
| 5019- | 5019 | 45 | GAGGTTCAGCTGG | 46 | GGTGGA | 5019 | 47 | GATATCCAGATG | 56 | CTCGAGgacaaaactcacacaAAAGTTGAGCC |
| hole- | | | TGGAGTCTGGCGG | | GGTGGC | | | ACCCAGTCCCCGA | | CAAATCTTCTgataagacccataatTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACATCGGTTCT | | | | | GTCAGTCCGTGTC | | CACGAAGACCCTGAGGTCAAGTTCAACTG |
| | | | TCTTCTATCCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGGGAGGAGCAGTA |
| | | | CCGGGTAAGGGCC | | | | | AAACCAGGAAAA | | CAACAGCACGTACCGTOGGTCAGCGTCC |
| | | | TGGAATGGGTTGC | | | | | GCTCCGAAGCTTC | | TCACCGTCCTGCACCAGGACTGGCTGAAT |
| | | | ATCTATTTATTCTG | | | | | TGATTTACTCGGC | | GGCAAGGAGTACAAGTGCAAGGTCTCCAA |
| | | | CTTTTGCCTCTACT | | | | | ATCCAGCCTCTAC | | CAAAGCCCTCCCAGCCCCCATCGAGAAAA |
| | | | TCTTATGCCGATA | | | | | TCTGGAGTCCCTT | | CCATCTCCAA4GCCAAAGGGCAGCCCCGA |
| | | | GCGTCAAGGGCCG | | | | | CTCGCTTCTCTGG | | GAACCACAGGTGTACACCCTGCCCCCAAT |
| | | | TTTCACTATAAGCG | | | | | TAGCCGTTCCGG | | CCGGGAGCTGATGACCAGCAACCAGGTCA |
| | | | CAGACACATCCAA | | | | | GACGGATTTCACT | | GCCTGAGCTGCGCCGTCAAAGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTCGTGAGCAAGCTCACCG |
| | | | TATTATTGTGCTCG | | | | | CAAGGTGTTTACC | | TGGACAAGAGCAGGTGGCAGCAGGGGAA |
| | | | CTACCATTTCCCGT | | | | | TGTTCACGTTCGG | | CGTCTTCTCATGCTCCGTGATGCATGAGGC |
| | | | TCGGTTTTGCTTTG | | | | | ACAGGGTACCAA | | TCTGCACAACCACTACACGCAGAAGAGCC |
| | | | GACTACTGGGGTC | | | | | GGTGGAGATCAA | | TCTCCCTGTCTCCGGGTAAAAGCGGCAGC |
| | | | AAGGAACCCTGGT | | | | | A | | GAGACTCCCGGGACCTCAGAGTCCGCCAC |
| | | | CACCGTCTCCTCG | | | | | | | ACCCGAAAGTGGTGGCGGA |
| 5019- | 5019 | 45 | GAGGTTCAGCTGG | 46 | GGTGGA | 5019 | 47 | GATATCCAGATG | 51 | CTCGAGgacaaaactcacacaAAAGTTGAGCC |
| Fc- | | | TGGAGTCTGGCGG | | GGTGGC | | | ACCCAGTCCCCGA | | CAAATCTTCTgataagacccatacttgcccaccgtg |
| 2539 | | | TGGCCTGGTGCAG | | AGT | | | GCTCCCTGTCCGC | | cccagcacctgaactcctgggsggaccgtcagtcttcct |
| | | | CCAGGGGGCTCAC | | | | | CTCTGTGGGCGA | | cttccccccaaaacccaaggacaccctcatgatctccc |
| | | | TCCGTTTGTCCTGT | | | | | TAGGGTCACCATC | | ggacccctgaggtcacatgcgtggtggtggacgtgagc |
| | | | GCAGCTTCTGGCT | | | | | ACCTGCCGTGCCA | | cacgaagaccctgaggtcaagttcaactggtacgtgga |
| | | | TCAACATCGGTTCT | | | | | GTCAGTCCGTGTC | | cggcgtggaggtgcataatgccaagacaaagccgcgg |
| | | | TCTTCTATCCACTG | | | | | CAGCGCTGTAGC | | gaggagcagtacaacagcacgtaccgtgtggtcagcg |
| | | | GGTGCGTCAGGCC | | | | | CTGGTATCAACAG | | tatcaccgtcctgcaccaggactggctgaatggcaag |
| | | | CCGGGTAAGGGCC | | | | | AAACCAGGAAAA | | gagtacaagtgcaaggtctccaacaaagccctcccag |
| | | | TGGAATGGGTTGC | | | | | GCTCCGAAGCTTC | | cccccatcgagaaaaccatctccaaagccaaagggca |
| | | | ATCTATTTATTCTG | | | | | TGATTTACTCGGC | | gccccgagaaccacaggtgtacaccctgcccccattct |
| | | | CTTTTGCCTCTACT | | | | | ATCCAGCCTCTAC | | gggaggagatgaccaagaaccaggtcagcctgacctg |
| | | | TCTTATGCCGATA | | | | | TCTGGAGTCCCTT | | cctggtcaaaggatctatcccagcgacatcgccgtgg |
| | | | GCGTCAAGGGCCG | | | | | CTCGCTTCTCTGG | | agtgggagagcaatgggcagccggagaacaactaca |
| | | | TTTCACTATAAGCG | | | | | TAGCCGTTCCGG | | agaccacgcctcccgtgctggactccgacggctccttct |
| | | | CAGACACATCCAA | | | | | GACGGATTTCACT | | tcctctacagcaagctcaccgtggacaagagcaggtgg |
| | | | AAACACAGCCTAC | | | | | CTGACCATCAGCA | | cagcaggggaacgtcttctcatgctccgtgatgcacga |
| | | | CTACAAATGAACA | | | | | GTCTGCAGCCGG | | ggctctgcacaaccactacacgcagaagagcctctccc |
| | | | GCTTAAGAGCTGA | | | | | AAGACTTCGCAAC | | tgtctccgggtaaaAGCGGCAGCGAGACTCCC |
| | | | GGACACTGCCGTC | | | | | TTATTACTGTCAG | | GGGACCTCAGAGTCCGCCACACCCGAAAG |
| | | | TATTATTGTGCTCG | | | | | CAAGGTGTTTACC | | TGGTGGCGGA |
| | | | CTACCATTTCCCGT | | | | | TGTTCACGTTCGG | | |
| | | | TCGGTTTTGCTTTG | | | | | ACAGGGTACCAA | | |
| | | | GACTACTGGGGTC | | | | | GGTGGAGATCAA | | |
| | | | AAGGAACCCTGGT | | | | | A | | |
| | | | CACCGTCTCCTCG | | | | | | | |
| 5019- | 5019 | 45 | GAGGTTCAGCTGG | 46 | GGTGGA | 5019 | 47 | GATATCCAGATG | 51 | CTCGAGgacaaaactcacacaAAAGTTGAGCC |
| Fc- | | | TGGAGTCTGGCGG | | GGTGGC | | | ACCCAGTCCCCGA | | CAAATCTTCTgataagacccatacttgcccaccgtg |
| 2542 | | | TGGCCTGGTGCAG | | AGT | | | GCTCCCTGTCCGC | | cccagcacctgaactcctggggggaccgtcagtcttcct |
| | | | CCAGGGGGCTCAC | | | | | CTCTGTGGGCGA | | cttccccccaaaacccaaggacaccctcatgatctccc |
| | | | TCCGTTTGTCCTGT | | | | | TAGGGTCACCATC | | ggacccctgaggtcacatgcgtggtggtggacgtgagc |
| | | | GCAGCTTCTGGCT | | | | | ACCTGCCGTGCCA | | cacgaagaccctgaggtcaagttcaactggtacgtgga |
| | | | TCAACATCGGTTCT | | | | | GTCAGTCCGTGTC | | cggcgtggaggtgcataatgccaagacaaagccgcgg |
| | | | TCTTCTATCCACTG | | | | | CAGCGCTGTAGC | | gaggagcagtacaacagcacgtaccgtgtggtcagcg |
| | | | GGTGCGTCAGGCC | | | | | CTGGTATCAACAG | | tcctcaccgtcctgcaccaggactggctgaatggcaag |
| | | | CCGGGTAAGGGCC | | | | | AAACCAGGAAAA | | gagtacaagtgcaaggtctccaacaaagccctcccag |
| | | | TGGAATGGGTTGC | | | | | GCTCCGAAGCTTC | | cccccatcgagaaaatcatctccaaagccaaagggca |
| | | | ATCTATTTATTCTG | | | | | TGATTTACTCGGC | | gccccgagaaccacaggtgtacaccctgcccccatccc |
| | | | CTTTTGCCTCTACT | | | | | ATCCAGCCTCTAC | | gggaggagatgaccaagaaccaggtcagcctgacctg |
| | | | TCTTATGCCGATA | | | | | TCTGGAGTCCCTT | | cctggtcaaaggcttctatcccagcgacatcgccgtgg |
| | | | GCGTCAAGGGCCG | | | | | CTCGCTTCTCTGG | | agtgggagagcaatgggcagccggagaacaactaca |
| | | | TTTCACTATAAGCG | | | | | TAGCCGTTCCGG | | agaccacgcctcccgtgctggactctgacggctccttct |
| | | | CAGACACATCCAA | | | | | GACGGATTTCACT | | tcctctacagcaagctcaccgtggacaagagcaggtgg |
| | | | AAACACAGCCTAC | | | | | CTGACCATCAGCA | | cagcaggggaacgtcttctcatgctccgtgatgcacga |
| | | | CTACAAATGAACA | | | | | GTCTGCAGCCGG | | ggctctgcacaaccactacacgcagaagagcctctccc |
| | | | GCTTAAGAGCTGA | | | | | AAGACTTCGCAAC | | tgtctccgggtaaaAGCGGCAGCGAGACTCCC |
| | | | GGACACTGCCGTC | | | | | TTATTACTGTCAG | | GGGACCTCAGAGTCCGCCACACCCGAAAG |
| | | | TATTATTGTGCTCG | | | | | CAAGGTGTTTACC | | TGGTGGCGGA |
| | | | CTACCATTTCCCGT | | | | | TGTTCACGTTCGG | | |
| | | | TCGGTTTTGCTTTG | | | | | ACAGGGTACCAA | | |
| | | | GACTACTGGGGTC | | | | | GGTGGAGATCAA | | |
| | | | AAGGAACCCTGGT | | | | | A | | |
| | | | CACCGTCTCCTCG | | | | | | | |
| 5038- | 5038 | 54 | GAGGTTCAGCTGG | 46 | GGTGGA | 5038 | 55 | GATATCCAGATG | 48 | CTCGAGgacaaaactcacacaAAAGTGGAGCC |
| knob- | | | TGGAGTCTGGCGG | | GGTGGC | | | ACCCAGTCCCCGA | | CAAAACTTCTgataagacccatacTTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACATCTCTTAT | | | | | GTCAGTCCGTGTC | | CACGAAGACCCTGAGGTCAAGTTCAACTG |
| | | | TATTATATGCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGCGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATCTATTTATTCTT | | | | | TGATTTACTCGGC | | GGCAAGGAGTACAAGTGCAAGGTCTCCAA |
| | | | ATTATGGCTATACT | | | | | ATCCAGCCTCTAC | | CAAAGCCCTCCCAGCCCCCATCGAGAAAA |
| | | | TATTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCAATGGTGTACACCTGCCCCCATCC |
| | | | TTTCACTATAAGCG | | | | | TAGCCGTTCCGG | | CGGGAGGAGATGACCAAGAACCAGGTCA |
| | | | CAGACACATCCAA | | | | | GACGGATTTCACT | | GCCTGTGGTGCATGGTCAAGGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTGTACAGCAAGCTCACCG |
| | | | TATTATTGTGCTCG | | | | | CAACATCCGTGGT | | TGGACAAGAGCCGCTGGCAGCAGGGGAA |
| | | | CTCTTCTTTCTCTT | | | | | CTGGTGGTTACCT | | CGTCTTCTCATGCTCCGTGATGCATGAGGC |
| | | | GGGCTATGGACTA | | | | | GATCACGTTCGG | | TCTGCACAACCACTACACGCAGAAGAGCC |
| | | | CTGGGGTCAAGGA | | | | | ACAGGGTACCAA | | TCTCCCTGTCTCCGGGTAAAAGCGGCAGC |
| | | | ACCCTGGTCACCG | | | | | GGTGGAGATCAA | | GAGACTCCCGGGACCTCAGAGTCCGCCAC |
| | | | TCTCCTCG | | | | | A | | ACCCGAAAGTGGTGGCGGA |
| 5038- | 5038 | 54 | GAGGTTCAGCTGG | 46 | GGTGGA | 5038 | 55 | GATATCCAGATG | 56 | CTCGAGgacaaaactcacacaAAAGTTGAGCC |
| hole- | | | TGGAGTCTGGCGG | | GGTGGC | | | ACCCAGTCCCCGA | | CAAATCTTCTgataagacccataatTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACATCTCTTAT | | | | | GTCAGTCCGTGTC | | CACGAAGACCCTGAGGTCAAGTTCAACTG |
| | | | TATTATATGCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGGGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATCTATTTATTCTT | | | | | TGATTTACTCGGC | | GGCAAGGAGTACAAGTGCAAGGTCTCCAA |
| | | | ATTATGGCTATACT | | | | | ATCCAGCCTCTAC | | CAAAGCCCTCCCAGCCCCCATCGAGAAAA |
| | | | TATTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCACAGGTGTACACCCTGCCCCCAAT |
| | | | TTTCACTATAAGCG | | | | | TAGCCGTTCCGG | | CLGGGAGCTGATGACCAGCAACCAGGTCA |
| | | | CAGACACATCCAA | | | | | GACGGATTTCACT | | GCCTGAGCTGCGCCGTCAAAGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCLGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTCGTGAGCAAGCTCACCG |
| | | | TATTATTGTGCTCG | | | | | CAACATCCGTGGT | | TGGACAAGAGCAGGTGGCAGCAGGGGAA |
| | | | CTCTTCTTTCTCTT | | | | | CTGGTGGTTACCT | | CGTCTTCTCATGCTCCGTGATGCATGAGGC |
| | | | GGGCTATGGACTA | | | | | GATCACGTTCGG | | TCTGCACAACCACTACACGCAGAAGAGCC |
| | | | CTGGGGTCAAGGA | | | | | ACAGGGTACCAA | | TCTCCCTGTCTCCGGGTAAAAGCGGCAGC |
| | | | ACCCTGGTCACCG | | | | | GGTGGAGATCAA | | GAGACTCCCGGGACCTCAGAGTCCGCCAC |
| | | | TCTCCTCG | | | | | A | | ACCCGAAAGTGGTGGCGGA |
| 5044- | 5044 | 57 | GAGGTTCAGCTGG | 46 | GGTGGA | 5044 | 58 | GATATCCAGATG | 48 | CTCGAGgacaaaactcacacaAAAGTGGAGCC |
| knob- | | | TGGAGTCTGGCGG | | GGTGGC | | | ACCCAGTCCCCGA | | CAAAACTTCTgataagacccatactTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACCTCTCTTCT | | | | | GTCAGTCCGTGTC | | CACGAAGACCCTGAGGTCAAGTTCAACTG |
| | | | TATTCTATGCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGCGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATATATTTCTTCTT | | | | | TGATTTACTCGGC | | GGCAAGGAGTACAAGTGCAAGGTCTCCAA |
| | | | ATTATGGCTATACT | | | | | ATCCAGCCTCTAC | | CAAAGCCCTCCCAGCCCCCATCGAGAAAA |
| | | | TATTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCAATGGTGTTTGACCTGCCCCCATCC |
| | | | TTTCACTATAAGCG | | | | | TAGCCGTTCCGG | | CGGGAGGAGATGACCAAGAACCAGGTCA |
| | | | CAGACACATCCAA | | | | | GACGGATTTCACT | | GCCTGTGGTGCATGGTCAAGGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTGTACAGCAAGCTCACCG |
| | | | TATTATTGTGCTCG | | | | | CAATGGTACTACG | | TGGACAAGAGCCGCTGGCAGCAGGGGAA |
| | | | CCCGGCTCCGGGT | | | | | CTCCGATCACGTT | | CGTCTTCTCATGCTCCGTGATGCATGAGGC |
| | | | CATTGGGGTTTTG | | | | | CGGACAGGGTAC | | TCTGCACAACCACTACACGCAGAAGAGCC |
| | | | ACTACTGGGGTCA | | | | | CAAGGTGGAGAT | | TCTCCCTGTCTCCGGGTAAAAGCGGCAGC |
| | | | AGGAACCCTGGTC | | | | | CAAA | | GAGACTCCCGGGACCTCAGAGTCCGCCAC |
| | | | ACCGTCTCCTCG | | | | | | | ACCCGAAAGTGGTGGCGGA |
| 5044- | 5044 | 57 | GAGGTTCAGCTGG | 46 | GGTGGA | 5044 | 58 | GATATCCAGATG | 56 | CTCGAGgacaaaactcacacaAAAGTTGAGCC |
| hole- | | | TGGAGTCTGGCGG | | GGTGGC | | | ACCCAGTCCCCGA | | CAAATCTTCTgataagacccataatTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACATCTCTTAT | | | | | GTCAGTCCGTGTC | | CACGAAGACCCTGAGGTCAAGTTCAACTG |
| | | | TATTCTATGCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGGGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATATATTTCTTCTT | | | | | TGATTTACTCGGC | | GGCAAGGAGTACAAGTGCAAGGTCTCCAA |
| | | | ATTATGGCTATACT | | | | | ATCCAGCCTCTAC | | CAAAGCCCTCCCAGCCCCCATCGAGAAAA |
| | | | TATTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCACAGGTGTACACCCTGCCCCCAAT |
| | | | TTTCACTATAAGCG | | | | | TAGCCGTTCCGG | | CCGGGAGCTGATGACCAGCAACCAGGTCA |
| | | | CAGACACATCCAA | | | | | GACGGATTTCACT | | GCCTGAGCTGCGCCGTCAAAGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTCGTGAGCAAGCTCACCG |
| | | | TATTATTGTGCTCG | | | | | CAATGGTACTACG | | TGGACAAGAGCAGGTGGCAGCAGGGGAA |
| | | | CCCGGCTCCGGGT | | | | | CTCCGATCACGTT | | CGTCTTCTCATGCTCCGTGATGCATGAGGC |
| | | | CATTGGGGTTTTG | | | | | CGGACAGGGTAC | | TCTGCACAACCACTACACGCAGAAGAGCC |
| | | | ACTACTGGGGTCA | | | | | CAAGGTGGAGAT | | TCTCCCTGTCTCCGGGTAAAAGCGGCAGC |
| | | | AGGAACCCTGGTC | | | | | CAAA | | GAGACTCCCGGGACCTCAGAGTCCGCCAC |
| | | | ACCGTCTCCTCG | | | | | | | ACCCGAAAGTGGTGGCGGA |
| 5048- | 5048 | 59 | GAGGTGCAGCTGG | 46 | GGTGGA | 5048 | 60 | GATATCCAGATG | 48 | CTCGAGgacaaaactcacacaAAAGTGGAGCC |
| knob- | | | TGGAGTCTGGCGG | | GGTGGC | | | ACCCAGTCCCCGA | | CAAAACTTCTgataagacccatactTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACATCTCTTAT | | | | | GTCAGTCCGTGTC | | CACGAAGACCC7GAGGTCAAGTTCAACTG |
| | | | GGTGCGTCAGGCC | | | | | CTGGTATCAACAG | | CCAAGACAAAGCCGCGCGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATCTATTTCTTCTT | | | | | TGATTTACTCGGC | | GGCAAGGAGTACAAGTGCAAGGTCTCCAA |
| | | | ATTATGGCTCTACT | | | | | ATCCAGCCTCTAC | | CAAAGCCCTCCCAGCCCCCATCGAGAAAA |
| | | | TATTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCAATGGTGTTTGACCTGCCCCCATCC |
| | | | TTTCACTATAAGCG | | | | | TAGCCGTTCCGG | | CGGGAGGAGATGACCAAGAACCAGGTCA |
| | | | CAGACACATCCAA | | | | | GACGGATTTCACT | | GCCTGTGGTGCATGGTCAAGGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGCAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTGTACAGCAAGCTCACCG |
| | | | TATTATTGTGCTCG | | | | | CAACATTACTCTG | | TGGACAAGAGCCGCTGGCAGCAGGGGAA |
| | | | CTCTTGGTGGGCT | | | | | TTTACGCTTCTCT | | CGTCTTCTCATGCTCCGTGATGCATGAGGC |
| | | | TGGGCTTTTGACT | | | | | GATCACGTTCGG | | TCTGCACAACCACTACACGCAGAAGAGCC |
| | | | ACTGGGGTCAAGG | | | | | ACAGGGTACCAA | | TCTCCCTGTCTCCGGGTAAAAGCGGCAGC |
| | | | AACCCTGGTCACC | | | | | GGTGGAGATCAA | | GAGACTCCCGGGACCTCAGAGTCCGCCAC |
| | | | GTCTCCTCG | | | | | A | | ACCCGAAAGTGGTGGCGGA |
| 5048- | 5048 | 59 | GAGGTTCAGCTGG | 46 | GGTGGA | 5048 | 60 | GATATCCAGATG | 56 | CTCGAGgacaaaactcacacaAAAGTTGAGCC |
| hole- | | | TGGAGTCTGGCGG | | GGTGGC | | | ACCCAGTCCCCGA | | CAAATCTTCTgataagacccataatTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACATCTCTTAT | | | | | GTCAGTCCGTGTC | | CACGAAGACCCTGAGGTCAAGTTCAACTG |
| | | | TATTATATGCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGGGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATCTATTTCTTCTT | | | | | TGATTTACTCGGC | | GGCAAGGAGTACAAGTGCAAGGTCTCCAA |
| | | | ATTATGGCTCTACT | | | | | ATCCAGCCTCTAC | | CAAAGCCCTCCCAGCCCCCATCGAGAAAA |
| | | | TATTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCACAGGTGTACACCCTGCCCCCAAT |
| | | | TTTCACTATAAGCG | | | | | TAGCCGTTCCGG | | CCGGGAGCTGATGACCAGCAACCAGGTCA |
| | | | CAGACACATCCAA | | | | | GACGGATTTCACT | | GCCTGAGCTGCGCCGTCAAAGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTCGTGAGCAAGCTCACCG |
| | | | TATTATTGTGCTCG | | | | | CAACATTACTCTG | | TGGACAAGAGCAGGTGGCAGCAGGGGAA |
| | | | CTCTTGGTGGGCT | | | | | TTTACGCTTCTCT | | CGTCTTCTCATGCTCCGTGATGCATGAGGC |
| | | | TGGGCTTTTGACT | | | | | GATCACGTTCGG | | TCTGCACAACCACTACACGCAGAAGAGCC |
| | | | ACTGGGGTCAAGG | | | | | ACAGGGTACCAA | | TCTCCCTGTCTCCGGGTAAAAGCGGCAGC |
| | | | AACCCTGGTCACC | | | | | GGTGGAGATCAA | | GAGACTCCCGGGACCTCAGAGTCCGCCAC |
| | | | GTCTCCTCG | | | | | A | | ACCCGAAAGTGGTGGCGGA |
| 5062- | 5062 | 61 | GAGGTTCAGCTGG | 46 | GGTGGA | 5062 | 62 | GATATCCAGATG | 48 | CTCGAGgacaaaactcacacaAAAGTGGAGCC |
| knob- | | | TGGAGTCTGGCGG | | GGTGGC | | | ACCCAGTCCCCGA | | CAAAACTTCTgataagacccatactTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCTTC | | | | | ACCTGCCGTGCCA | | AGGTCACATGCGTGGTGGTGGACGTGAGC |
| | | | AACatctcttattat | | | | | GTCAGTCCGTGTC | | CACGAAGACCCTGAGGTCAAGTTCAACTG |
| | | | tatatcCACTGGGTG | | | | | CAGCGCTGTAGC | | GTACGTGGACGGCGTGGAGGTGCATAATG |
| | | | CGTCAGGCCCCGG | | | | | CTGGTATCAACAG | | CCAAGACAAAGCCGCGCGAGGAGCAGTA |
| | | | GTAAGGGCCTGGA | | | | | AAACCAGGAAAA | | CAACAGCACGTACCGTGTGGTCAGCGTCC |
| | | | ATGGGTTGCATCT | | | | | GCTCCGAAGCTTC | | TCACCGTCCTGCACCAGGACTGGCTGAAT |
| | | | ATTTATTCTTCTTC | | | | | TGATTTACTCGGC | | GGCAAGGAGTACAAGTGCAAGGTCTCCAA |
| | | | TAGCTATACTTATT | | | | | ATCCAGCCTCTAC | | CAAAGCCCTCCCAGCCCCCATCGAGAAAA |
| | | | ATGCCGATAGCGT | | | | | TCTGGAGTCCCTT | | CCATCTCCAAAGCCAAAGGGCAGCCCCGA |
| | | | CAAGGGCCGTTTC | | | | | CTCGCTTCTCTGG | | GAACCAATGGTGTTTGACCTGCCCCCATCC |
| | | | ACTATAAGCGCAG | | | | | TAGCCGTTCCGG | | CGGGAGGAGATGACCAAGAACCAGGTCA |
| | | | ACACATCCAAAAA | | | | | GACGGATTTCACT | | GCCTGTGGTGCATGGTCAAGGGCTTCTAT |
| | | | CACAGCCTACCTA | | | | | CTGACCATCAGCA | | CCCAGCGACATCGCCGTGGAGTGGGAGA |
| | | | CAAATGAACAGCT | | | | | GTCTGCAGCCGG | | GCAATGGGCAGCCGGAGAACAACTACAA |
| | | | TAAGAGCTGAGGA | | | | | AAGACTTCGCAAC | | GACCACGCCTCCCGTGCTGGACTCCGACG |
| | | | CACTGCCGTCTATT | | | | | TTATTACTGTCAG | | GCTCCTTCTTCCTGTACAGCAAGCTCACCG |
| | | | ATTGTGCTCGCTCT | | | | | CAATCTGGTTGGT | | TGGACAAGAGCCGCTGGCAGCAGGGGAA |
| | | | TCTTACGCTTGGG | | | | | GGGGTGTTTCTCT | | CGTCTTCTCATGCTCCGTGATGCATGAGGC |
| | | | CTATTGACTACTG | | | | | GATCACGTTCGG | | TCTGCACAACCACTACACGCAGAAGAGCC |
| | | | GGGTCAAGGAACC | | | | | ACAGGGTACCAA | | TCTCCCTGTCTCCGGGTAAAAGCGGCAGC |
| | | | CTGGTCACCGTCT | | | | | GGTGGAGATCAA | | GAGACTCCCGGGACCTCAGAGTCCGCCAC |
| | | | CCTCG | | | | | A | | ACCCGAAAGTGGTGGCGGA |
| 5062- | 5062 | 61 | GAGGTTCAGCTGG | 46 | GGTGGA | 5052 | 62 | GATATCCAGATG | 56 | CTCGAGgacaaaactcacacaAAAGTTGAGCC |
| hole- | | | TGGAGTCTGGCGG | | GGTGGC | | | ACCCAGTCCCCGA | | CAAATCTTCTgataagacccataatTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCTTC | | | | | ACCTGCCGTGCCA | | AGGTCACATGCGTGGTGGTGGACGTGAGC |
| | | | AACatctcttattat | | | | | GTCAGTCCGTGTC | | CACGAAGACCCTGAGGTCAAGTTCAACTG |
| | | | tatatcCACTGGGTG | | | | | CAGCGCTGTAGC | | GTACGTGGACGGCGTGGAGGTGCATAATG |
| | | | CGTCAGGCCCCGG | | | | | CTGGTATCAACAG | | CCAAGACAAAGCCGCGGGAGGAGCAGTA |
| | | | GTAAGGGCCTGGA | | | | | AAACCAGGAAAA | | CAACAGCACGTACCGTGTGGTCAGCGTCC |
| | | | ATGGGTTGCATCT | | | | | GCTCCGAAGCTTC | | TCACCGTCCTGCACCAGGACTGGCTGAAT |
| | | | ATTTATTCTTCTTC | | | | | TGATTTACTCGGC | | GGCAAGGAGTACAAGTGCAAGGTCTCCAA |
| | | | TAGCTATACTTATT | | | | | ATCCAGCCTCTAC | | CAAAGCCCTCCCAGCCCCCATCGAGAAAA |
| | | | ATGCCGATAGCGT | | | | | TCTGGAGTCCCTT | | CCATCTCCAAAGCCAAAGGGCAGCCCCGA |
| | | | CAAGGGCCGTTTC | | | | | CTCGCTTCTCTGG | | GAACCACAGGTGTACACCCTGCCCCCAAT |
| | | | ACTATAAGCGCAG | | | | | TAGCCGTTCCGG | | CCGGGAGCTGATGACCAGCAACCAGGTCA |
| | | | ACACATCCAAAAA | | | | | GACGGATTTCACT | | GCCTGAGCTGCGCCGTCAAAGGCTTCTAT |
| | | | CACAGCCTACCTA | | | | | CTGACCATCAGCA | | CCCAGCGACATCGCCGTGGAGTGGGAGA |
| | | | CAAATGAACAGCT | | | | | GTCTGCAGCCGG | | GCAATGGGCAGCCGGAGAACAACTACAA |
| | | | TAAGAGCTGAGGA | | | | | AAGACTTCGCAAC | | GACCACGCCTCCCGTGCTGGACTCCGACG |
| | | | CACTGCCGTCTATT | | | | | TTATTACTGTCAG | | GCTCCTTCTTCCTCGTGAGCAAGCTCACCG |
| | | | ATTGTCCTCGCTCT | | | | | CAATCTGGTTGGT | | TGGACAAGAGCAGGTGGCAGCAGGGGAA |
| | | | TCTTACGCTTGGG | | | | | GGGGTGTTTCTCT | | CGTCTTCTCATGCTCCGTGATGCATGAGGC |
| | | | CTATTGACTACTG | | | | | GATCACGTTCGG | | TCTGCACAACCACTACACGCAGAAGAGCC |
| | | | GGGTCAAGGAACC | | | | | ACAGGGTACCAA | | TCTCCCTGTCTCCGGGTAAAAGCGGCAGC |
| | | | CTGGTCACCGTCT | | | | | GGTGGAGATCAA | | GAGACTCCCGGGACCTCAGAGTCCGCCAC |
| | | | CCTCG | | | | | A | | ACCCGAAAGTGGTGGCGGA |
| 5063- | 5063 | 63 | GAGGTTCAGCTGG | 46 | GGTGGA | 5063 | 64 | GATATCCAGATG | 48 | CTCGAGgacanactcacacaAAAGTGGAGCC |
| knob- | | | TGGAGTCTGGCGG | | GGTGGC | | | ACCCAGTCCCCGA | | CAAAACTTCTgataagacccatactTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACATCTCTTAT | | | | | GTCAGTCCGTGTC | | CACGAAGACCCTGAGGTCAAGTTCAACTG |
| | | | TATTATATCCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGCGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATCTATTTATCCTT | | | | | TGATTTACTCGGC | | GGCAAGGAGTACAAGTGCAAGGTCTCCAA |
| | | | CTTCTGGCTATACT | | | | | ATCCAGCCTCTAC | | CAAAGCCCTCCCAGCCCCCATCGAGAAAA |
| | | | TATTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCAATGGTGTTTGACCTGCCCCCATCC |
| | | | TTTCACTATAAGCG | | | | | TAGCCGTTCCGG | | CGGGAGGAGATGACCAAGAACCAGGTCA |
| | | | CAGACACATCCAA | | | | | GACGGATTTCACT | | GCCTGTGGTGCATGGTCAAGGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTGTACAGCAAGCTCACCG |
| | | | TATTATTGTGCTCG | | | | | CAATCTTACGCTG | | TGGACAAGAGCCGCTGGCAGCAGGGGAA |
| | | | CTCTTCTTTCTACT | | | | | CTTACCTGTTCAC | | CGTCTTCTCATGCTCCGTGATGCATGAGGC |
| | | | GGGCTATGGACTA | | | | | GTTCGGACAGGG | | TCTGCACAACCACTACACGCAGAAGAGCC |
| | | | CTGGGGTCAAGGA | | | | | TACCAAGGTGGA | | TCTCCCTGTCTCCGGGTAAAAGCGGCAGC |
| | | | ACCCTGGTCACCG | | | | | GATCAAA | | GAGACTCCCGGGACCTCAGAGTCCGCCAC |
| | | | TCTCCTCG | | | | | | | ACCCGAAAGTGGTGGCGGA |
| 5063- | 5063 | 63 | GAGGTTCAGCTGG | 46 | GGTGGA | 5063 | 64 | GATATCCAGATG | 56 | CTCGAGgacaaaactcacacaAAAGTTGAGCC |
| hole- | | | TGGAGTCTGGCGG | | GGTGGC | | | ACCCAGTCCCCGA | | CAAATCTTCTgataagacccataatTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACATCTCTTAT | | | | | GTCAGTCCGTGTC | | CACGAAGACCCTGAGGTCAAGTTCAACTG |
| | | | TATTATATCCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGGGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATCTATTTATCCTT | | | | | TGATTTACTCGGC | | GGCAAGGAGTACAAGTGCAAGGTCTCCAA |
| | | | CTTCTGGCTATACT | | | | | ATCCAGCCTCTAC | | CAAAGCCCTCCCAGCCCCCATCGAGAAAA |
| | | | TATTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCACAGGTGTACACCCTGCCCCCAAT |
| | | | TTTCACTATAAGCG | | | | | TAGCCGTTCCGG | | CCGGGAGCTGATGACCAGCAACCAGGTCA |
| | | | CAGACACATCCAA | | | | | GACGGATTTCACT | | GCCTGAGCTGCGCCGTCAAAGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTCGTGAGCAAGCTCACCG |
| | | | TATTATTGTGCTCG | | | | | CAATCTTACGCTG | | TGGACAAGAGCAGGTGGCAGCAGGGGAA |
| | | | CTCTTCTTTCTACT | | | | | CTTACCTGTTCAC | | CGTCTTCTCATGCTCCGTGATGCATGAGGC |
| | | | GGGCTATGGACTA | | | | | GTTCGGACAGGG | | TCTGCACAACCACTACACGCAGAAGAGCC |
| | | | CTGGGGTCAAGGA | | | | | TACCAAGGTGGA | | TCTCCCTGTCTCCGGGTAAAAGCGGCAGC |
| | | | ACCCTGGTCACCG | | | | | GATCAAA | | GAGACTCCCGGGACCTCAGAGTCCGCCAC |
| | | | TCTCCTCG | | | | | | | ACCCGAAAGTGGTGGCGGA |
| 5080- | 5080 | 65 | GAGGTTCAGCTGG | 46 | GGTGGA | 5080 | 66 | GATATCCAGATG | 48 | CTCGAGganaaacttacacaAAAGTGGAGCC |
| knob- | | | TGGAGTCTGGCGG | | GGTGGC | | | ACCCAGTCCCCGA | | CAAAACTTCTgataagaoccatactTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACATCTCTTAT | | | | | GTCAGTCCGTGTC | | CACGAAGACCCTGAGGTCAAGTTCAACTG |
| | | | TATTCTATGCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGCGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATCTATTTCTTCTT | | | | | TGATTTACTCGGC | | GGCAAGGAGTACAAGTGCAAGGTCTCCAA |
| | | | ATTATAGCTCTACT | | | | | ATCCAGCCTCTAC | | CAAAGCCCTCCCAGCCCCCATCGAGAAAA |
| | | | TCTTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCAATGGTGTTTGACCTGCCCCCATCC |
| | | | TTTCACTATAAGCG | | | | | TAGCCGTTCCGG | | CGGGAGGAGATGACCAAGAACCAGGTCA |
| | | | CAGACACATCCAA | | | | | GACGGATTTCACT | | GCCTGTGGTGCATGGTCAAGGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTGTACAGCAAGCTCACCG |
| | | | TATTATTGTGCTCG | | | | | CAACATTGGTCTT | | TGGACAAGAGCCGCTGGCAGCAGGGGAA |
| | | | CTTCTGGTACCCG | | | | | ACCCGATCACGTT | | CGTCTTCTCATGCTCCGTGATGCATGAGGC |
| | | | GGTATGGACTACT | | | | | CGGACAGGGTAC | | TCTGCACAACCACTACACGCAGAAGAGCC |
| | | | GGGGTCAAGGAAC | | | | | CAAGGTGGAGAT | | TCTCCCTGTCTCCGGGTAAAAGCGGCAGC |
| | | | CCTGGTCACCGTC | | | | | CAAA | | GAGACTCCCGGGACCTCAGAGTCCGCCAC |
| | | | TCCTCG | | | | | | | ACCCGAAAGTGGTGGCGGA |
| 5080- | 5080 | 65 | GAGGTTCAGCTGG | 46 | GGTGGA | 5080 | 66 | GATATCCAGATG | 56 | CTCGAGgacaaaactcacacaAAAGTTGAGCC |
| hole- | | | TGGAGTCTGGCGG | | GGTGGC | | | ACCCAGTCCCCGA | | CAAATCTTCTgataagacccataatTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACATCTCTTAT | | | | | GTCAGTCCGTGTC | | CACGAAGACCCTGAGGTCAAGTTCAACTG |
| | | | TATTCTATGCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGGGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATCTATTTCTTCTT | | | | | TGATTTACTCGGC | | GGCAAGGAGTACAAGTGCAAGGTCTCCAA |
| | | | ATTATAGCTCTACT | | | | | ATCCAGCCTCTAC | | CAAAGCCCTCCCAGCCCCCATCGAGAAAA |
| | | | TCTTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCACAGGTGTACACCCTGCCCCCAAT |
| | | | TTTCACTATAAGCG | | | | | TAGCCGTTCCGG | | CCGGGAGCTGATGACCAGCAACCAGGTCA |
| | | | CAGACACATCCAA | | | | | GACGGATTTCACT | | GCCTGAGCTGCGCCGTCAAAGGCTTCTAT |
| | | | TTTACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTCGTGAGCAAGCTCACCG |
| | | | TATTATTGTGCTCG | | | | | CAACATTGGTCTT | | TGGACAAGAGCAGGTGGCAGCAGGGGAA |
| | | | CTTCTGGTACCCG | | | | | ACCCGATCACGTT | | CGTCTTCTCATGCTCCGTGATGCATGAGGC |
| | | | GGTATGGACTACT | | | | | CGGACAGGGTAC | | TCTGCACAACCACTACACGCAGAAGAGCC |
| | | | GGGGTCAAGGAAC | | | | | CAAGGTGGAGAT | | TCTCCCTGTCTCCGGGTAAAAGCGGCAGC |
| | | | CCTGGTCACCGTC | | | | | CAAA | | GAGACTCCCGGGACCTCAGAGTCCGCCAC |
| | | | TCCTCG | | | | | | | ACCCGAAAGTGGTGGCGGA |
| 5081- | 5081 | 67 | GAGGTTCAGCTGG | 46 | GGTGGA | 5081 | 68 | GATATCCAGATG | 48 | CTCGAGgacaaaactcacacaAAAGTGGAGCC |
| knob- | | | TGGAGTCTGGCGG | | GGTGGC | | | ACCCAGTCCCCGA | | CAAAACTTCTgataagacccatactTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACCTCTCTTAT | | | | | GTCAGTCCGTGTC | | CACGAAGACCCTGAGGTCAAGTTCAACTG |
| | | | TATTATATGCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGCGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATCTATTTATTCTT | | | | | TGATTTACTCGGC | | GGCAAGGAGTACAAGTGCAAGGTCTCCAA |
| | | | ATTCTGGCTATACT | | | | | ATCCAGCCTCTAC | | CAAAGCCCTCCCAGCCCCCATCGAGAAAA |
| | | | TATTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCAATGGTGTTTGACCTGCCCCCATCC |
| | | | TTTCACTATAAGCG | | | | | TAGCCGTTCCGG | | CGGGAGGAGATGACCAAGAACCAGGTCA |
| | | | CAGACACATCCAA | | | | | GACGGATTTCACT | | GCCTGTGGTGCATGGTCAAGGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTGTACAGCAAGCTCACCG |
| | | | TATTATTGTGCTCG | | | | | CAAGGTGGTTGG | | TGGACAAGAGCCGCTGGCAGCAGGGGAA |
| | | | CTCTTCTTTCGCTT | | | | | GGTCCGTTCACGT | | CGTCTTCTCATGCTCCGTGATGCATGAGGC |
| | | | GGGCTTTTGACTA | | | | | TCGGACAGGGTA | | TCTGCACAACCACTACACGCAGAAGAGCC |
| | | | CTGGGGTCAAGGA | | | | | CCAAGGTGGAGA | | TCTCCCTGTCTCCGGGTAAAAGCGGCAGC |
| | | | ACCCTGGTCACCG | | | | | TCAAA | | GAGACTCCCGGGACCTCAGAGTCCGCCAC |
| | | | TCTCCTCG | | | | | | | ACCCGAAAGTGGTGGCGGA |
| 5081- | 5081 | 67 | GAGGTTCAGCTGG | 46 | GGTGGA | 5081 | 68 | GATATCCAGATG | 56 | CTCGAGgacaaaactcacacaAAAGTTGAGCC |
| hole- | | | TGGAGTCTGGCGG | | GGTGGC | | | ACCCAGTCCCCGA | | CAAATCTTCTgataagacccataatTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACCTCTCTTAT | | | | | GTCAGTCCGTGTC | | CACGAAGACCCTGAGGTCAAGTTCAACTG |
| | | | TATTATATGCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGGGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATCTATTTATTCTT | | | | | TGATTTACTCGGC | | GGCAAGGAGTACAAGTGCAAGGTCTCCAA |
| | | | ATTCTGGCTATACT | | | | | ATCCAGCCTCTAC | | CAAAGCCCTCCCAGCCCCCATCGAGAAAA |
| | | | TATTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCACAGGTGTACACCCTGCCCCCAAT |
| | | | TTTCACTATAAGCG | | | | | TAGCCGTTCCGG | | CCGGGAGCTGATGACCAGCAACCAGGTCA |
| | | | CAGACACATCCAA | | | | | GACGGATTTCACT | | GCCTGAGCTGCGCCGTCAAAGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTCGTGAGCAAGCTCACCG |
| | | | TATTATTGTGCTCG | | | | | CAAGGTGGTTGG | | TGGACAAGAGCAGGTGGCAGCAGGGGAA |
| | | | CTCTTCTTTCGCTT | | | | | GGTCCGTTCACGT | | CGTCTTCTCATGCTCCGTGATGCATGAGGC |
| | | | GGGCTTTTGACTA | | | | | TCGGACAGGGTA | | TCTGCACAACCACTACACGCAGAAGAGCC |
| | | | CTGGGGTCAAGGA | | | | | CCAAGGTGGAGA | | TCTCCCTGTCTCCGGGTAAAAGCGGCAGC |
| | | | ACCCTGGTCACCG | | | | | TCAAA | | GAGACTCCCGGGACCTCAGAGTCCGCCAC |
| | | | TCTCCTCG | | | | | | | ACCCGAAAGTGGTGGCGGA |
| 2928- | 2928 | 69 | GAGGTTCAGCTGG | 46 | GGTGGA | 2928 | 70 | GATATCCAGATG | 48 | CTCGAGgacaaaactcacacaAAAGTGGAGCC |
| knob- | | | TGGAGTCTGGCGG | | GGTGGC | | | ACCCAGTCCCCGA | | CAAAACTTCTgataagacccatactTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACATCTCTTAT | | | | | GTCAGTCCGTGTC | | CACGAAGACCCTGAGGTCAAGTTCAACTG |
| | | | TCTTCTATCCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGCGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATCTATTTATCCTT | | | | | TGATTTACTCGGC | | GGCAAGGAGTACAAGTGCAAGGTCTCCAA |
| | | | CTTATAGCTCTACT | | | | | ATCCAGCCTCTAC | | CAAAGCCCTCCCAGCCCCCATCGAGAAAA |
| | | | TATTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCAATGGTGTTTGACCTGCCCCCATCC |
| | | | TTTCACTATAAGCG | | | | | TAGCCGTTCCGG | | CGGGAGGAGATGACCAAGAACCAGGTCA |
| | | | CAGACACATCCAA | | | | | GACGGATTTCACT | | GCCTGTGGTGCATGGTCAAGGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTGTACAGCAAGCTCACCG |
| | | | TATTATTGTGCTCG | | | | | CAAGCTTTCTACT | | TGGACAAGAGCCGCTGGCAGCAGGGGAA |
| | | | CTACTACGCTATG | | | | | ACCCGATCACGTT | | CGTCTTCTCATGCTCCGTGATGCATGAGGC |
| | | | GACTACTGGGGTC | | | | | CGGACAGGGTAC | | TCTGCACAACCACTACACGCAGAAGAGCC |
| | | | AAGGAACCCTGGT | | | | | CAAGGTGGAGAT | | TCTCCCTGTCTCCGGGTAAAAGCGGCAGC |
| | | | CACCGTCTCCTCG | | | | | CAAA | | GAGACTCCCGGGACCTCAGAGTCCGCCAC |
| | | | | | | | | | | ACCCGAAAGTGGTGGCGGA |
| 2928- | 2928 | 69 | GAGGTTCAGCTGG | 46 | GGTGGA | 2928 | 70 | GATATCCAGATG | 56 | CTCGAGgacaaaactcacacaAAAGTTGAGCC |
| hole- | | | TGGAGTCTGGCGG | | GGTGGC | | | ACCCAGTCCCCGA | | CAAATCTTCTgataagacccataatTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACATCTCTTAT | | | | | GTCAGTCCGTGTC | | CACGAAGACCCTGAGGTCAAGTTCAACTG |
| | | | TCTTCTATCCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGGGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATCTATTTATCCTT | | | | | TGATTTACTCGGC | | GGCAAGGAGTACAAGTGCAAGGTCTCCAA |
| | | | CTTATAGCTCTACT | | | | | ATCCAGCCTCTAC | | CAAAGCCCTCCCAGCCCCCATCGAGAAAA |
| | | | TATTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCACAGGTGTACACCCTGCCCCCAAT |
| | | | TTTCACTATAAGCG | | | | | TAGCCGTTCCGG | | CCGGGAGCTGATGACCAGCAACCAGGTCA |
| | | | CAGACACATCCAA | | | | | GACGGATTTCACT | | GCCTGAGCTGCGCCGTCAAAGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTCGTGAGCAAGCTCACCG |
| | | | TATTATTGTGCTCG | | | | | CAAGCTTTCTACT | | TGGACAAGAGCAGGTGGCAGCAGGGGAA |
| | | | CTACTACGCTATG | | | | | ACCCGATCACGTT | | CGTCTTCTCATGCTCCGTGATGCATGAGGC |
| | | | GACTACTGGGGTC | | | | | CGGACAGGGTAC | | TCTGCACAACCACTACACGCAGAAGAGCC |
| | | | AAGGAACCCTGGT | | | | | CAAGGTGGAGAT | | TCTCCCTGTCTCCGGGTAAAAGCGGCAGC |
| | | | CACCGTCTCCTCG | | | | | CAAA | | GAGACTCCCGGGACCTCAGAGTCCGCCAC |
| | | | | | | | | | | ACCCGAAAGTGGTGGCGGA |
| 5019- | 5019 | 45 | GAGGTTCAGCTGG | 46 | GGTGGA | 5019 | 47 | GATATCCAGATG | 48 | CTCGAGgacaaaactcacacaAAAGTGGAGCC |
| knob- | | | TGGAGTCTGGCGG | | GGTGGC | | | ACCCAGTCCCCGA | | CAAAACTTCTgataagacccatactTGCCCACC |
| 2459- | | | TGGCCTGGTGCAG | | AGT | | | GCTCCCTGTCCGC | | GTGCCCAGCACCTGAACTCCTGGGGGGAC |
| 2460 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACATCGGTTCT | | | | | GTCAGTCCGTGTC | | CACGAAGACCCTGAGGTCAAGTTCAACTG |
| | | | TCTTCTATCCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGCGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATCTATTTATTCTG | | | | | TGATTTACTCGGC | | GGCAAGGAGTACAAGTGCAAGGTCTCCAA |
| | | | CTTTTGCCTCTACT | | | | | ATCCAGCCTCTAC | | CAAAGCCCTCCCAGCCCCCATCGAGAAAA |
| | | | TCTTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCAATGGTGTTTGACCTGCCCCCATCC |
| | | | TTTCACTATAAGCG | | | | | TAGCCGTTCCGG | | CGGGAGGAGATGACCAAGAACCAGGTCA |
| | | | CAGACACATCCAA | | | | | GACGGATTTCACT | | GCCTGTGGTGCATGGTCAAGGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTGTACAGCAAGCTCACCG |
| | | | TATTATTGTGCTCG | | | | | CAAGGTGTTTACC | | TGGACAAGAGCCGCTGGCAGCAGGGGAA |
| | | | CTACCATTTCCCGT | | | | | TGTTCACGTTCGG | | CGTCTTCTCATGCTCCGTGATGCATGAGGC |
| | | | TCGGTTTTGCTTTG | | | | | ACAGGGTACCAA | | TCTGCACAACCACTACACGCAGAAGAGCC |
| | | | GACTACTGGGGTC | | | | | GGTGGAGATCAA | | TCTCCCTGTCTCCGGGTAAAAGCGGCAGC |
| | | | AAGGAACCCTGGT | | | | | A | | GAGACTCCCGGGACCTCAGAGTCCGCCAC |
| | | | CACCGTCTCCTCG | | | | | | | ACCCGAAAGTGGTGGCGGA |
| 5019- | 5019 | 45 | GAGGTTCAGCTGG | 46 | GGTGGA | 5019 | 47 | GATATCCAGATG | 56 | CTCGAGgacaaaactcacacaAAAGTTGAGCC |
| hole- | | | TGGAGTCTGGCGG | | GGTGGC | | | ACCCAGTCCCCGA | | CAAATCTTCTgataagacccataatTGCCCACC |
| 2459- | | | TGGCCTGGTGCAG | | AGT | | | GCTCCCTGTCCGC | | GTGCCCAGCACCTGAACTCCTGGGGGGAC |
| 2460 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACATCGGTTCT | | | | | GTCAGTCCGTGTC | | CACGAAGACCCTGAGGTCAAGTTCAACTG |
| | | | TCTTCTATCCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGGGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATCTATTTATTCTG | | | | | TGATTTACTCGGC | | GGCAAGGAGTACAAGTGCAAGGTCTCCAA |
| | | | CTTTTGCCTCTACT | | | | | ATCCAGCCTCTAC | | CAAAGCCCTCCCAGCCCCCATCGAGAAAA |
| | | | TCTTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCACAGGTGTACACCCTGCCCCCAAT |
| | | | TTTCACTATAAGCG | | | | | TAGCCGTTCCGG | | CCGGGAGCTGATGACCAGCAACCAGGTCA |
| | | | CAGACACATCCAA | | | | | GACGGATTTCACT | | GCCTGAGCTGCGCCGTCAAAGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTCGTGAGCAAGCTCACCG |
| | | | TATTATTGTGCTCG | | | | | CAAGGTGTTTACC | | TGGACAAGAGCAGGTGGCAGCAGGGGAA |
| | | | CTACCATTTCCCGT | | | | | TGTTCACGTTCGG | | CGTCTTCTCATGCTCCGTGATGCATGAGGC |
| | | | TCGGTTTTGCTTTG | | | | | ACAGGGTACCAA | | TCTGCACAACCACTACACGCAGAAGAGCC |
| | | | GACTACTGGGGTC | | | | | GGTGGAGATCAA | | TCTCCCTGTCTCCGGGTAAAAGCGGCAGC |
| | | | AAGGAACCCTGGT | | | | | A | | GAGACTCCCGGGACCTCAGAGTCCGCCAC |
| | | | CACCGTCTCCTCG | | | | | | | ACCCGAAAGTGGTGGCGGA |
| 2890- | 2890 | 84 | GAGGTTCAGCTGG | 46 | GGTGGA | 2890 | 85 | GATATCCAGATG | 48 | CTCGAGgaceaaactcacacaAAAGTGGAGCC |
| knob- | | | TGGAGTCTGGCGG | | GGTGGC | | | ACCCAGTCCCCGA | | CAAAACTTCTgataagacccatactTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACATCTATTAT | | | | | GTCAGTCCGTGTC | | CACGAAGACCCTGAGGTCAAGTTCAACTG |
| | | | TCTTCTATCCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGCGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATCTATTTATCCTT | | | | | TGATTTACTCGGC | | GGCAAGGAGTACAAGTGCAAGGTCTCCAA |
| | | | ATTATGGCTATACT | | | | | ATCCAGCCTCTAC | | CAAAGCCCTCCCAGCCCCCATCGAGAAAA |
| | | | TATTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCAATGGTGTTTGACCTGCCCCCATCC |
| | | | TTTCACTATAAGCG | | | | | TAGCCGTTCCGG | | CGGGAGGAGATGACCAAGAACCAGGTCA |
| | | | CAGACACATCCAA | | | | | GACGGATTTCACT | | GCCTGTGGTGCATGGTCAAGGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTGTACAGCAAGCTCACCG |
| | | | TATTATTGTGCTCG | | | | | CAATCTTACTGGC | | TGGACAAGAGCCGCTGGCAGCAGGGGAA |
| | | | CTACTACCATTACG | | | | | ATTCTTACCTGAT | | CGTCTTCTCATGCTCCGTGATGCATGAGGC |
| | | | GTTTGGACTACTG | | | | | CACGTTCGGACA | | TCTGCACAACCACTACACGCAGAAGAGCC |
| | | | GGGTCAAGGAACC | | | | | GGGTACCAAGGT | | TCTCCCTGTCTCCGGGTAAAAGCGGCAGC |
| | | | CTGGTCACCGTCT | | | | | GGAGATCAAA | | GAGACTCCCGGGACCTCAGAGTCCGCCAC |
| | | | CCTCG | | | | | | | ACCCGAAAGTGGTGGCGGA |
| 2890- | 2890 | 84 | GAGGTTCAGCTGG | 46 | GGTGGA | 2890 | 85 | GATATCCAGATG | 56 | CTCGAGgacaaaactcacacaAAAGTTGAGCC |
| hole- | | | TGGAGTCTGGCGG | | GGTGGC | | | ACCCAGTCCCCGA | | CAAATCTTCTgataagacccataatTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACATCTATTAT | | | | | GTCAGTCCGTGTC | | CACGAAGACCCTGAGGTCAAGTTCAACTG |
| | | | TCTTCTATCCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGGGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATCTATTTATCCTT | | | | | TGATTTACTCGGC | | GGCAAGGAGTACAAGTGCAAGGTCTCCAA |
| | | | ATTATGGCTATACT | | | | | ATCCAGCCTCTAC | | CAAAGCCCTCCCAGCCCCCATCGAGAAAA |
| | | | TATTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCACAGGTGTACACCCTGCCCCCAAT |
| | | | TTTCACTATAAGCG | | | | | TAGCCGTTCCGG | | CCGGGAGCTGATGACCAGCAACCAGGTCA |
| | | | CAGACACATCCAA | | | | | GACGGATTTCACT | | GCCTGAGCTGCGCCGTCAAAGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTCGTGAGCAAGCTCACCG |
| | | | TATTATTGTGCTCG | | | | | CAATCTTACTGGC | | TGGACAAGAGCAGGTGGCAGCAGGGGAA |
| | | | CTACTACCATTACG | | | | | ATTCTTACCTGAT | | CGTCTTCTCATGCTCCGTGATGCATGAGGC |
| | | | GTTTGGACTACTG | | | | | CACGTTCGGACA | | TCTGCACAACCACTACACGCAGAAGAGCC |
| | | | GGGTCAAGGAACC | | | | | GGGTACCAAGGT | | TCTCCCTGTCTCCGGGTAAAAGCGGCAGC |
| | | | CTGGTCACCGTCT | | | | | GGAGATCAAA | | GAGACTCCCGGGACCTCAGAGTCCGCCAC |
| | | | CCTCG | | | | | | | ACCCGAAAGTGGTGGCGGA |
| 12735- | 12735 | 86 | GAGGTTCAGCTGG | 46 | GGTGGA | 12735 | 87 | GATATCCAGATG | 48 | CTCGAGgacaaaactcacacaAAAGTGGAGCC |
| knob- | | | TGGAGTCTGGCGG | | GGTGGC | | | ACCCAGTCCCCGA | | CAAAACTTCTgataagacccatactTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACATCTCTTCT | | | | | GTCAGTCCGTGTC | | CACGAAGACCCTGAGGTCAAGTTCAACTG |
| | | | TCTTCTATGCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGCGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATCTATTTATTCTT | | | | | TGATTTACTCGGC | | GGCAAGGAGTACAAGTGCAAGGTCTCCAA |
| | | | ATTATGGCTCTACT | | | | | ATCCAGCCTCTAC | | CAAAGCCCTCCCAGCCCCCATCGAGAAAA |
| | | | TATTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCAATGGTGTTTGACCTGCCCCCATCC |
| | | | TTTCACTATAAGCG | | | | | TAGCCGTTCCGG | | CGGGAGGAGATGACCAAGAACCAGGTCA |
| | | | CAGACACATCCAA | | | | | GACGGATTTCACT | | GCCTGTGGTGCATGGTCAAGGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTGTACAGCAAGCTCACCG |
| | | | TATTATTGTGCTCG | | | | | CAACCGGGTTCTT | | TGGACAAGAGCCGCTGGCAGCAGGGGAA |
| | | | CTGGTACGGTATG | | | | | GGTACTTCCCGCC | | CGTCTTCTCATGCTCCGTGATGCATGAGGC |
| | | | GACTACTGGGGTC | | | | | GATCACGTTCGG | | TCTGCACAACCACTACACGCAGAAGAGCC |
| | | | AAGGAACCCTGGT | | | | | ACAGGGTACCAA | | TCTCCCTGTCTCCGGGTAAAAGCGGCAGC |
| | | | CACCGTCTCCTCG | | | | | GGTGGAGATCAA | | GAGACTCCCGGGACCTCAGAGTCCGCCAC |
| | | | | | | | | A | | ACCCGAAAGTGGTGGCGGA |
| 12735- | 12735 | 86 | GAGGTTCAGCTGG | 46 | GGTGGA | 12735 | 87 | GATATCCAGATG | 56 | CTCGAGgacaaaactcacacaAAAGTTGAGCC |
| hole- | | | TGGAGTCTGGCGG | | GGTGGC | | | ACCCAGTCCCCGA | | CAAATCTTCTgataagacccataatTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACATCTCTTCT | | | | | GTCAGTCCGTGTC | | CACGAAGACCCTGAGGTCAAGTTCAACTG |
| | | | TCTTCTATGCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGGGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | ATCTATTTATTCTT | | | | | TGATTTACTCGGC | | GGCAAGGAGTACAAGTGCAAGGTCTCCAA |
| | | | ATTATGGCTCTACT | | | | | ATCCAGCCTCTAC | | CAAAGCCCTCCCAGCCCCCATCGAGAAAA |
| | | | TATTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCACAGGTGTACACCCTGCCCCCAAT |
| | | | TTTCACTATAAGCG | | | | | TAGCCGTTCCGG | | CCGGGAGCTGATGACCAGCAACCAGGTCA |
| | | | CAGACACATCCAA | | | | | GACGGATTTCACT | | GCCTGAGCTGCGCCGTCAAAGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTCGTGAGCAAGCTCACCG |
| | | | TATTATTGTGCTCG | | | | | CAACCGGGTTCTT | | TGGACAAGAGCAGGTGGCAGCAGGGGAA |
| | | | CTGGTACGGTATG | | | | | GGTACTTCCCGCC | | CGTCTTCTCATGCTCCGTGATGCATGAGGC |
| | | | GACTACTGGGGTC | | | | | GATCACGTTCGG | | TCTGCACAACCACTACACGCAGAAGAGCC |
| | | | AAGGAACCCTGGT | | | | | ACAGGGTACCAA | | TCTCCCTGTCTCCGGGTAAAAGCGGCAGC |
| | | | CACCGTCTCCTCG | | | | | GGTGGAGATCAA | | GAGACTCCCGGGACCTCAGAGTCCGCCAC |
| | | | | | | | | A | | ACCCGAAAGTGGTGGCGGA |
| 5027- | 5027 | 88 | GAGGTTCAGCTGG | 46 | GGTGGA | 5027 | 89 | GATATCCAGATG | 56 | CTCGAGgacaaaactcacataAAAGTTGAGCC |
| hole- | | | TGGAGTCTGGCGG | | GGTGGC | | | ACCCAGTCCCCGA | | CAAATCTTCTgataagacccataatTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACTCCTCTTTT | | | | | GTCAGTCCGTGTC | | CACGAAGACCCTGAGGTCAAGTTCAACTG |
| | | | TATTTTATGCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGGGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | AACTGTTTATCCTT | | | | | TGATTTACTCGGC | | GGCAAGGAGTACAAGTGCAAGGTCTCCAA |
| | | | ATCTTGACTATACT | | | | | ATCCAGCCTCTAC | | CAAAGCCCTCCCAGCCCCCATCGAGAAAA |
| | | | TATTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCACAGGTGTACACCCTGCCCCCAAT |
| | | | TTTCACTATAAGCG | | | | | TAGCCGTTCCGG | | CCGGGAGCTGATGACCAGCAACCAGGTCA |
| | | | CAGACACATCCAA | | | | | GACGGATTTCACT | | GCCTGAGCTGCGCCGTCAAAGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTCGTGAGCAAGCTCACCG |
| | | | TATTATTGTGCTCG | | | | | CAATCTTCTTATT | | TGGACAAGAGCAGGTGGCAGCAGGGGAA |
| | | | CGCGTTTCCGGGT | | | | | CTCTGATCACGTT | | CGTCTTCTCATGCTCCGTGATGCATGAGGC |
| | | | TCTTACCATCCTAT | | | | | CGGACAGGGTAC | | TCTGCACAACCACTACACGCAGAAGAGCC |
| | | | GGACTACTGGGGT | | | | | CAAGGTGGAGAT | | TCTCCCTGTCTCCGGGTAAAAGCGGCAGC |
| | | | CAAGGAACCCTGG | | | | | CAAA | | GAGACTCCCGGGACCTCAGAGTCCGCCAC |
| | | | TCACCGTCTCCTCG | | | | | | | ACCCGAAAGTGGTGGCGGA |
| 5027- | 5027 | 90 | GAGGTTCAGCTGG | 46 | GGTGGA | 5027 | 91 | GATATCCAGATG | 48 | CTCGAGgacaaaactcacacaAAAGTGGAGCC |
| knob- | | | TGGAGTCTGGCGG | | GGTGGC | | | ACCCAGTCCCCGA | | CAAAACTTCTgataagacccatactTGCCCACC |
| 2539- | | | TGGCCTGGTGCAG | | AGT | | | GCTCCCTGTCCGC | | GTGCCCAGCACCTGAACTCCTGGGGGGAC |
| 2542 | | | CCAGGGGGCTCAC | | | | | CTCTGTGGGCGA | | CGTCAGTCTTCCTCTTCCCCCCAAAACCCA |
| | | | TCCGTTTGTCCTGT | | | | | TAGGGTCACCATC | | AGGACACCCTCATGATCTCCCGGACCCCTG |
| | | | GCAGCTTCTGGCT | | | | | ACCTGCCGTGCCA | | AGGTCACATGCGTGGTGGTGGACGTGAGC |
| | | | TCAACTCCTCTTTT | | | | | GTCAGTCCGTGTC | | CACGAAGACCCTGAGGTCAAGTTCAACTG |
| | | | TATTTTATGCACTG | | | | | CAGCGCTGTAGC | | GTACGTGGACGGCGTGGAGGTGCATAATG |
| | | | GGTGCGTCAGGCC | | | | | CTGGTATCAACAG | | CCAAGACAAAGCCGCGCGAGGAGCAGTA |
| | | | CCGGGTAAGGGCC | | | | | AAACCAGGAAAA | | CAACAGCACGTACCGTGTGGTCAGCGTCC |
| | | | TGGAATGGGTTGC | | | | | GCTCCGAAGCTTC | | TCACCGTCCTGCACCAGGACTGGCTGAAT |
| | | | AACTGTTTATCCTT | | | | | TGATTTACTCGGC | | GGCAAGGAGTACAAGTGCAAGGTCTCCAA |
| | | | ATCTTGACTATACT | | | | | ATCCAGCCTCTAC | | CAAAGCCCTCCCAGCCCCCATCGAGAAAA |
| | | | TATTATGCCGATA | | | | | TCTGGAGTCCCTT | | CCATCTCCAAAGCCAAAGGGCAGCCCCGA |
| | | | GCGTCAAGGGCCG | | | | | CTCGCTTCTCTGG | | GAACCAATGGTGTTTGACCTGCCCCCATCC |
| | | | TTTCACTATAAGCG | | | | | TAGCCGTTCCGG | | CGGGAGGAGATGACCAAGAACCAGGTCA |
| | | | CAGACACATCCAA | | | | | GACGGATTTCACT | | GCCTOGGTGCATGGTCAAGGGCTTCTAT |
| | | | AAACACAGCCTAC | | | | | CTGACCATCAGCA | | CCCAGCGACATCGCCGTGGAGTGGGAGA |
| | | | CTACAAATGAACA | | | | | GTCTGCAGCCGG | | GCAATGGGCAGCCGGAGAACAACTACAA |
| | | | GCTTAAGAGCTGA | | | | | AAGACTTCGCAAC | | GACCACGCCTCCCGTGCTGGACTCCGACG |
| | | | GGACACTGCCGTC | | | | | TTATTACTGTCAG | | GCTCCTTCTTCCTGTACAGCAAGCTCACCG |
| | | | TATTATTGTGCTCG | | | | | CAATCTTCTTATT | | TGGACAAGAGCCGCTGGCAGCAGGGGAA |
| | | | CGCGTTTCCGGGT | | | | | CTCTGATCACGTT | | CGTCTTCTCATGCTCCGTGATGCATGAGGC |
| | | | TCTTACCATCCTAT | | | | | CGGACAGGGTAC | | TCTGCACAACCACTACACGCAGAAGAGCC |
| | | | GGACTACTGGGGT | | | | | CAAGGTGGAGAT | | TCTCCCTGTCTCCGGGTAAAAGCGGCAGC |
| | | | CAAGGAACCCTGG | | | | | CAAA | | GAGACTCCCGGGACCTCAGAGTCCGCCAC |
| | | | TCACCGTCTCCTCG | | | | | | | ACCCGAAAGTGGTGGCGGA |

| | C- | | | | | C- | | |
|---|---|---|---|---|---|---|---|---|
| | fusion | SEQ | | SEQ | VH- | fusion | SEQ | |
| | VH | ID | | ID | VL | VL | ID | |
| ID | (VH3) | NO: | | NO: | linker | (VH4) | NO: | |
| 5019- | 2539 | 49 | GAGGTTCAGCTGGTG | 46 | GGTGGA | 2542 | 50 | GATATCCAGATG |
| knob- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTGG | | | | | GATAGGGTCACC |
| | | | CTTCAACATCTCTTATT | | | | | ATCACCTGCCGT |
| | | | CTTCTATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATATATTTCT | | | | | CAACAGAAACCA |
| | | | TCTTATTATGGCTATA | | | | | GGAAAAGCTCCG |
| | | | CTTATTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCGCTC | | | | | CTGACCATCAGC |
| | | | ATTACTTCCCGTGGGC | | | | | AGTCTGCAGCCG |
| | | | TGGTGCTATGGACTAC | | | | | GAAGACTTCGCA |
| | | | TGGGGTCAAGGAACC | | | | | ACTTATTACTGTC |
| | | | CTGGTCACCGTCTCCT | | | | | AGCAATACTACT |
| | | | CG | | | | | GGCCGATCACGT |
| | | | | | | | | TCGGACAGGGTA |
| | | | | | | | | CCAAGGTGGAG |
| | | | | | | | | ATCAAATGA |
| 5019- | 2542 | 53 | GAGGTTCAGCTGGTG | 46 | GGTGGA | 2539 | 52 | GATATCCAGATG |
| hole- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTCTT | | | | | ATCACCTGCCGT |
| | | | ATTATATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATCTATTTAT | | | | | CAACAGAAACCA |
| | | | TCTTCTTATGGCTATA | | | | | GGAAAAGCTCCG |
| | | | CTTCTTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCACTG | | | | | CTGACCATCAGC |
| | | | TTCGTGGATCCAAAAA | | | | | AGTCTGCAGCCG |
| | | | ACCGTACTTCTCTGGT | | | | | GAAGACTTCGCA |
| | | | TGGGCTATGGACTACT | | | | | ACTTATTACTGTC |
| | | | GGGGTCAAGGAACCC | | | | | AGCAATACTCTT |
| | | | TGGTCACCGTCTCCTC | | | | | GGGGTCCGTTCA |
| | | | G | | | | | CGTTCGGACAGG |
| | | | | | | | | GTACCAAGGTGG |
| | | | | | | | | AGATCAAATGA |
| 5019- | 2539 | 49 | GAGGTTCAGCTGGTG | 46 | GGTGGA | 2539 | 52 | GATATCCAGATG |
| Fc- | | | GAGTCTGGCGGTGGC | | GGTGGC | | | ACCCAGTCCCCG |
| 2539 | | | CTGGTGCAGCCAGGG | | AGT | | | AGCTCCCTGTCC |
| | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTATT | | | | | ATCACCTGCCGT |
| | | | CTTCTATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATATATTTCT | | | | | CAACAGAAACCA |
| | | | TCTTATTATGGCTATA | | | | | GGAAAAGCTCCG |
| | | | CTTATTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCGCTC | | | | | CTGACCATCAGC |
| | | | ATTACTTCCCGTGGGC | | | | | AGTCTGCAGCCG |
| | | | TGGTGCTATGGACTAC | | | | | GAAGACTTCGCA |
| | | | TGGGGTCAAGGAACC | | | | | ACTTATTACTGTC |
| | | | CTGGTCACCGTCTCCT | | | | | AGCAATACTCTT |
| | | | CG | | | | | GGGGTCCGTTCA |
| | | | | | | | | CGTTCGGACAGG |
| | | | | | | | | GTACCAAGGTGG |
| | | | | | | | | AGATCAAATGA |
| 5019- | 2542 | 53 | GAGGTTCAGCTGGTG | 46 | GGTGGA | 2542 | 50 | GATATCCAGATG |
| Fc- | | | GAGTCTGGCGGTGGC | | GGTGGC | | | ACCCAGTCCCCG |
| 2542 | | | CTGGTGCAGCCAGGG | | AGT | | | AGCTCCCTGTCC |
| | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTCTT | | | | | ATCACCTGCCGT |
| | | | ATTATATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATCTATGTAT | | | | | CAACAGAAACCA |
| | | | TCTTCUATGGCTATA | | | | | GGAAAAGCTCCG |
| | | | CTTCTTATGCCGATAG | | | | | AAGCTTCTGATT |
| | | | CGTCAAGGGCCGUTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATGTCACT |
| | | | TATTGTGCTCGCACTG | | | | | CTGACCATCAGC |
| | | | TTCGTGGATCCAAAAA | | | | | AGTCTGCAGCCG |
| | | | ACCGTACTTCTCTGGT | | | | | GAAGACTTCGCA |
| | | | TGGGCTATGGACTACT | | | | | ACTTATTACTGTC |
| | | | GGGGTCAAGGAACCC | | | | | AGCAATACTACT |
| | | | TGGTCACCGTCTCCTC | | | | | GGCCGATCACGT |
| | | | G | | | | | TCGGACAGGGTA |
| | | | | | | | | CCAAGGTGGAG |
| | | | | | | | | ATCAAATGA |
| 5038- | 2539 | 49 | GAGGTTCAGCTGGTG | 46 | GGTGGA | 2542 | 50 | GATATCCAGATG |
| knob- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTATT | | | | | ATCACCTGCCGT |
| | | | ATTATATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATATATTTCT | | | | | CAACAGAAACCA |
| | | | TCTTATTATGGCTATA | | | | | GGAAAAGCTCCG |
| | | | CTTATTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCGCTC | | | | | CTGACCATCAGC |
| | | | ATTACTTCCCGTGGGC | | | | | AGTCTGCAGCCG |
| | | | TGGTGCTATGGACTAC | | | | | GAAGACTTCGCA |
| | | | TGGGGTCAAGGAACC | | | | | ACTTATTACTGTC |
| | | | CTGGTCACCGTCTCCT | | | | | AGCAATACTACT |
| | | | CG | | | | | GGCCGATCACGT |
| | | | | | | | | TCGGACAGGGTA |
| | | | | | | | | CCAAGGTGGAG |
| | | | | | | | | ATCAAATGA |
| 5038- | 2542 | 53 | GAGGTTCAGCTGGTG | 46 | GGTGGA | 2539 | 52 | GATATCCAGATG |
| hole- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTCTT | | | | | ATCACCTGCCGT |
| | | | ATTATATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATCTATTTAT | | | | | CAACAGAAACCA |
| | | | TCTTCTTATGGCTATA | | | | | GGAAAAGCTCCG |
| | | | CTTCTTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCACTG | | | | | CTGACCATCAGC |
| | | | TTCGTGGATCCAAAAA | | | | | AGTCTGCAGCCG |
| | | | ACCGTACTTCTCTGGT | | | | | GAAGACTTCGCA |
| | | | TGGGCTATGGACTACT | | | | | ACTTATTACTGTC |
| | | | GGGGTCAAGGAACCC | | | | | AGCAATACTCTT |
| | | | TGGTCACCGTCTCCTC | | | | | GGGGTCCGTTCA |
| | | | G | | | | | CGTTCGGACAGG |
| | | | | | | | | GTACCAAGGTGG |
| | | | | | | | | AGATCAAATGA |
| 5044- | 2539 | 49 | GAGGTTCAGCTGGTG | 46 | GGTGGA | 2542 | 50 | GATATCCAGATG |
| knob- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTATT | | | | | ATCACCTGCCGT |
| | | | CTTCTATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATATATTTCT | | | | | CAACAGAAACCA |
| | | | TCTTATTATGGCTATA | | | | | GGAAAAGCTCCG |
| | | | CTTATTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCGCTC | | | | | CTGACCATCAGC |
| | | | ATTACTTCCCGTGGGC | | | | | AGTCTGCAGCCG |
| | | | TGGTGCTATGGACTAC | | | | | GAAGACTTCGCA |
| | | | TGGGGTCAAGGAACC | | | | | ACTTATTACTGTC |
| | | | CTGGTCACCGTCTCCT | | | | | AGCAATACTACT |
| | | | CG | | | | | GGCCGATCACGT |
| | | | | | | | | TCGGACAGGGTA |
| | | | | | | | | CCAAGGTGGAG |
| | | | | | | | | ATCAAATGA |
| 5044- | 2542 | 53 | GAGGTTCAGCTGGTG | 46 | GGTGGA | 2539 | 52 | GATATCCAGATG |
| hole- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTCTT | | | | | ATCACCTGCCGT |
| | | | ATTATATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATCTATTTAT | | | | | CAACAGAAACCA |
| | | | TCTTCTTATGGCTATA | | | | | GGAAAAGCTCCG |
| | | | CTTCTTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCACTG | | | | | CTGACCATCAGC |
| | | | TTCGTGGATCCAAAAA | | | | | AGTCTGCAGCCG |
| | | | ACCGTACTTCTCTGGT | | | | | GAAGACTTCGCA |
| | | | TGGGCTATGGACTACT | | | | | ACTTATTACTGTC |
| | | | GGGGTCAAGGAACCC | | | | | AGCAATACTCTT |
| | | | TGGTCACCGTCTCCTC | | | | | GGGGTCCGTTCA |
| | | | G | | | | | CGTTCGGACAGG |
| | | | | | | | | GTACCAAGGTGG |
| | | | | | | | | AGATCAAATGA |
| 5048- | 2539 | 49 | GAGGTTCAGCTGGTG | 46 | GGTGGA | 2542 | 50 | GATATCCAGATG |
| knob- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTATT | | | | | ATCACCTGCCGT |
| | | | GCGTCAGGCCCCGGG | | | | | GTGTCCAGCGCT |
| | | | TAAGGGCCTGGAATG | | | | | GTAGCCTGGTAT |
| | | | GGTTGCATATATTTCT | | | | | CAACAGAAACCA |
| | | | TCTTATTATGGCTATA | | | | | GGAAAAGCTCCG |
| | | | CTTATTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCGCTC | | | | | CTGACCATCAGC |
| | | | ATTACTTCCCGTGGGC | | | | | AGTCTGCAGCCG |
| | | | TGGTGCTATGGACTAC | | | | | GAAGACTTCGCA |
| | | | TGGGGTCAAGGAACC | | | | | ACTTATTACTGTC |
| | | | CTGGTCACCGTCTCCT | | | | | AGCAATACTACT |
| | | | CG | | | | | GGCCGATCACGT |
| | | | | | | | | TCGGACAGGGTA |
| | | | | | | | | CCAAGGTGGAG |
| | | | | | | | | ATCAAATGA |
| 5048- | 2542 | 53 | GAGGTTCAGCTGGTG | 46 | GGTGGA | 2539 | 52 | GATATCCAGATG |
| hole- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTCTT | | | | | ATCACCTGCCGT |
| | | | ATTATATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATCTATTTAT | | | | | CAACAGAAACCA |
| | | | TCTTCTTATGGCTATA | | | | | GGAAAAGCTCCG |
| | | | CTTCTTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCACTG | | | | | CTGACCATCAGC |
| | | | TTCGTGGATCCAAAAA | | | | | AGTCTGCAGCCG |
| | | | ACCGTACTTCTCTGGT | | | | | GAAGACTTCGCA |
| | | | TGGGCTATGGACTACT | | | | | ACTTATTACTGTC |
| | | | GGGGTCAAGGAACCC | | | | | AGCAATACTCTT |
| | | | TGGTCACCGTCTCCTC | | | | | GGGGTCCGTTCA |
| | | | G | | | | | CGTTCGGACAGG |
| | | | | | | | | GTACCAAGGTGG |
| | | | | | | | | AGATCAAATGA |
| 5062- | 2539 | 49 | GAGGTTCAGCTGGTG | 46 | GGTGGA | 2542 | 50 | GATATCCAGATG |
| knob- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTATT | | | | | ATCACC7GCCGT |
| | | | CTTCTATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATATATTTCT | | | | | CAACAGAAACCA |
| | | | TCTTATTATGGCTATA | | | | | GGAAAAGCTCCG |
| | | | CTTATTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCGCTC | | | | | CTGACCATCAGC |
| | | | ATTACTTCCCGTGGGC | | | | | AGTCTGCAGCCG |
| | | | TGGTGCTATGGACTAC | | | | | GAAGACTTCGCA |
| | | | TGGGGTCAAGGAACC | | | | | ACTTATTACTGTC |
| | | | CTGGTCACCGTCTCCT | | | | | AGCAATACTACT |
| | | | CG | | | | | GGCCGATCACGT |
| | | | | | | | | TCGGACAGGGTA |
| | | | | | | | | CCAAGGTGGAG |
| | | | | | | | | ATCAAATGA |
| 5062- | 2542 | 53 | GAGGTTCAGCTGGTG | 46 | GGTGGA | 2539 | 52 | GATATCCAGATG |
| hole- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTCTT | | | | | ATCACCTGCCGT |
| | | | ATTATATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATCTATTTAT | | | | | CAACAGAAACCA |
| | | | TCTTCTTATGGCTATA | | | | | GGAAAAGCTCCG |
| | | | CTTCTTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCACTG | | | | | CTGACCATCAGC |
| | | | TTCGTGGATCCAAAAA | | | | | AGTCTGCAGCCG |
| | | | ACCGTACTTCTCTGGT | | | | | GAAGACTTCGCA |
| | | | TGGGCTATGGACTACT | | | | | ACTTATTACTGTC |
| | | | GGGGTCAAGGAACCC | | | | | AGCAATACTCTT |
| | | | TGGTCACCGTCTCCTC | | | | | GGGGTCCGTTCA |
| | | | G | | | | | CGTTCGGACAGG |
| | | | | | | | | GTACCAAGGTGG |
| | | | | | | | | AGATCAAATGA |
| 5063- | 2539 | 49 | GAGGTTCAGCTGGTG | 46 | GGTGGA | 2542 | 50 | GATATCCAGATG |
| knob- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTATT | | | | | ATCACCTGCCGT |
| | | | CTTCTATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATATATTTCT | | | | | CAACAGAAACCA |
| | | | TCTTATTATGGCTATA | | | | | GGAAAAGCTCCG |
| | | | CTTATTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCGCTC | | | | | CTGACCATCAGC |
| | | | ATTACTTCCCGTGGGC | | | | | AGTCTGCAGCCG |
| | | | TGGTGCTATGGACTAC | | | | | GAAGACTTCGCA |
| | | | TGGGGTCAAGGAACC | | | | | ACTTATTACTGTC |
| | | | CTGGTCACCGTCTCCT | | | | | AGCAATACTACT |
| | | | CG | | | | | GGCCGATCACGT |
| | | | | | | | | TCGGACAGGGTA |
| | | | | | | | | CCAAGGTGGAG |
| | | | | | | | | ATCAAATGA |
| 5063- | 53 | 2542 | GAGGTTCAGCTGGTG | 46 | GGTGGA | 2539 | 52 | GATATCCAGATG |
| hole- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTCTT | | | | | ATCACCTGCCGT |
| | | | ATTATATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATCTATTTAT | | | | | CAACAGAAACCA |
| | | | TCTTCTTATGGCTATA | | | | | GGAAAAGCTCCG |
| | | | CTTCTTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCACTG | | | | | CTGACCATCAGC |
| | | | TTCGTGGATCCAAAAA | | | | | AGTCTGCAGCCG |
| | | | ACCGTACTTCTCTGGT | | | | | GAAGACTTCGCA |
| | | | TGGGCTATGGACTACT | | | | | ACTTATTACTGTC |
| | | | GGGGTCAAGGAACCC | | | | | AGCAATACTCTT |
| | | | TGGTCACCGTCTCCTC | | | | | GGGGTCCGTTCA |
| | | | G | | | | | CGTTCGGACAGG |
| | | | | | | | | GTACCAAGGTGG |
| | | | | | | | | AGATCAAATGA |
| 5080- | 2539 | 49 | GAGGTTCAGCTGGTG | 46 | GGTGGA | 2542 | 50 | GATATCCAGATG |
| knob- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTATT | | | | | ATCACCTGCCGT |
| | | | CTTCTATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATATATTTCT | | | | | CAACAGAAACCA |
| | | | TCTTATTATGGCTATA | | | | | GGAAAAGCTCCG |
| | | | CTTATTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCUCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCGCTC | | | | | CTGACCATCAGC |
| | | | ATTACTTCCCGTGGGC | | | | | AGTCTGCAGCCG |
| | | | TGGTGCTATGGACTAC | | | | | GAAGACTTCGCA |
| | | | TGGGGTCAAGGAACC | | | | | ACTTATTACTGTC |
| | | | CTGGTCACCGTCTCCT | | | | | AGCAATACTACT |
| | | | CG | | | | | GGCCGATCACGT |
| | | | | | | | | TCGGACAGGGTA |
| | | | | | | | | CCAAGGTGGAG |
| | | | | | | | | ATCAAATGA |
| 5080- | 2542 | 53 | GAGGTTCAGCTGGTG | 46 | GGTGGA | 2539 | 52 | GATATCCAGATG |
| hole- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTCTT | | | | | ATCACCTGCCGT |
| | | | ATTATATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATCTATTTAT | | | | | CAACAGAAACCA |
| | | | TCTTCTTATGGCTATA | | | | | GGAAAAGCTCCG |
| | | | CTTCTTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCACTG | | | | | CTGACCATCAGC |
| | | | TTCGTGGATCCAAAAA | | | | | AGTCTGCAGCCG |
| | | | ACCGTACTTCTCTGGT | | | | | GAAGACTTCGCA |
| | | | TGGGCTATGGACTACT | | | | | ACTTATTACTGTC |
| | | | GGGGTCAAGGAACCC | | | | | AGCAATACTCTT |
| | | | TGGTCACCGTCTCCTC | | | | | GGGGTCCGTTCA |
| | | | G | | | | | CGTTCGGACAGG |
| | | | | | | | | GTACCAAGGTGG |
| | | | | | | | | AGATCAAATGA |
| 5081- | 2539 | 49 | GAGGTTCAGCTGGTG | 46 | GGTGGA | 2542 | 50 | GATATCCAGATG |
| knob- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTATT | | | | | ATCACCTGCCGT |
| | | | CTTCTATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATATATTTCT | | | | | CAACAGAAACCA |
| | | | TCTTATTATGGCTATA | | | | | GGAAAAGCTCCG |
| | | | CTTATTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCGCTC | | | | | CTGACCATCAGC |
| | | | ATTACTTCCCGTGGGC | | | | | AGTCTGCAGCCG |
| | | | TGGTGCTATGGACTAC | | | | | GAAGACTTCGCA |
| | | | TGGGGTCAAGGAACC | | | | | ACTTATTACTGTC |
| | | | CTGGTCACCGTCTCCT | | | | | AGCAATACTACT |
| | | | CG | | | | | GGCCGATCACGT |
| | | | | | | | | TCGGACAGGGTA |
| | | | | | | | | CCAAGGTGGAG |
| | | | | | | | | ATCAAATGA |
| 5081- | 2542 | 53 | GAGGTTCAGCTGGTG | 46 | GGTGGA | 2539 | 52 | GATATCCAGATG |
| hole- | | | GAGTCTGGCGGTGGC | | GGTGGC | | | ACCCAGTCCCCG |
| 2539- | | | TGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTCTT | | | | | ATCACCTGCCGT |
| | | | ATTATATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATCTATTTAT | | | | | CAACAGAAACCA |
| | | | TCTTCTTATGGCTATA | | | | | GGAAAAGCTCCG |
| | | | CTTCTTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCACTG | | | | | CTGACCATCAGC |
| | | | TTCGTGGATCCAAAAA | | | | | AGTCTGCAGCCG |
| | | | ACCGTACTTCTCTGGT | | | | | GAAGACTTCGCA |
| | | | TGGGCTATGGACTACT | | | | | ACTTATTACTGTC |
| | | | GGGGTCAAGGAACCC | | | | | AGCAATACTCTT |
| | | | TGGTCACCGTCTCCTC | | | | | GGGGTCCGTTCA |
| | | | G | | | | | CGTTCGGACAGG |
| | | | | | | | | GTACCAAGGTGG |
| | | | | | | | | AGATCAAATGA |
| 2928- | 2539 | 49 | GAGGTTCAGCTGGTG | 46 | GGTGGA | 2542 | 50 | GATATCCAGATG |
| knob- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTATT | | | | | ATCACCTGCCGT |
| | | | CTTCTATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATATATTTCT | | | | | CAACAGAAACCA |
| | | | TCTTATTATGGCTATA | | | | | GGAAAAGCTCCG |
| | | | CTTATTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCGCTC | | | | | CTGACCATCAGC |
| | | | ATTACTTCCCGTGGGC | | | | | AGTCTGCAGCCG |
| | | | TGGTGCTATGGACTAC | | | | | GAAGACTTCGCA |
| | | | TGGGGTCAAGGAACC | | | | | ACTTATTACTGTC |
| | | | CTGGTCACCGTCTCCT | | | | | AGCAATACTACT |
| | | | CG | | | | | GGCCGATCACGT |
| | | | | | | | | TCGGACAGGGTA |
| | | | | | | | | CCAAGGTGGAG |
| | | | | | | | | ATCAAATGA |
| 2928- | 2542 | 53 | GAGGTTCAGCTGGTG | 46 | GGTGGA | 2539 | 52 | GATATCCAGATG |
| hole- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTCTT | | | | | ATCACCTGCCGT |
| | | | ATTATATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATCTATTTAT | | | | | CAACAGAAACCA |
| | | | TCTTCTTATGGCTATA | | | | | GGAAAAGCTCCG |
| | | | CTTCTTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCACTG | | | | | CTGACCATCAGC |
| | | | TTCGTGGATCCAAAAA | | | | | AGTCTGCAGCCG |
| | | | ACCGTACTTCTCTGGT | | | | | GAAGACTTCGCA |
| | | | TGGGCTATGGACTACT | | | | | ACTTATTACTGTC |
| | | | GGGGTCAAGGAACCC | | | | | AGCAATACTCTT |
| | | | TGGTCACCGTCTCCTC | | | | | GGGGTCCGTTCA |
| | | | G | | | | | CGTTCGGACAGG |
| | | | | | | | | GTACCAAGGTGG |
| | | | | | | | | AGATCAAATGA |
| 5019- | 2460 | 71 | GAGGTTCAGCTGGTG | 46 | GGTGGA | 2459 | 72 | GATATCCAGATG |
| knob- | | | GAGTCTGGCGGTGGC | | GGTGGC | | | ACCCAGTCCCCG |
| 2459- | | | CTGGTGCAGCCAGGG | | AGT | | | AGCTCCCTGTCC |
| 2460 | | | GGCTCACTCCGTTTGT | | | | | GCCTCTGTGGGC |
| | | | CCTGTGCAGCTTCTGG | | | | | GATAGGGTCACC |
| | | | CTTCAACTTTTCTTCTT | | | | | ATCACCTGCCGT |
| | | | CTTCTATACACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATCTATTTCT | | | | | CAACAGAAACCA |
| | | | TCTTCTTATGGCTATA | | | | | GGAAAAGCTCCG |
| | | | CTTATTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCGGTG | | | | | CTGACCATCAGC |
| | | | GTTCTGGTGTTTCTCA | | | | | AGTCTGCAGCCG |
| | | | TTACGGTTCTGTTTAC | | | | | GAAGACTTCGCA |
| | | | TACTCTTGGTGGGCTT | | | | | ACTTATTACTGTC |
| | | | TGGACTACTGGGGTC | | | | | AGCAAGCTTCTT |
| | | | AAGGAACCCTGGTCA | | | | | ACGCTCCGATCA |
| | | | CCGTCTCCTCG | | | | | CGTTCGGACAGG |
| | | | | | | | | GTACCAAGGTGG |
| | | | | | | | | AGATCAAA |
| 5019- | 2459 | 73 | GAGGTTCAGCTGGTG | 46 | GGTGGA | 2460 | 74 | GATATCCAGATG |
| hole- | | | GAGTCTGGCGGTGGC | | GGTGGC | | | ACCCAGTCCCCG |
| 2459- | | | CTGGTGCAGCCAGGG | | AGT | | | AGCTCCCTGTCC |
| 2460 | | | GGCTCACTCCGTTTGT | | | | | GCCTCTGTGGGC |
| | | | CCTGTGCAGCTTCTGG | | | | | GATAGGGTCACC |
| | | | CTTCAACCTCTCTTATT | | | | | ATCACCTGCCGT |
| | | | ATTATATGCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATCTATTTAT | | | | | CAACAGAAACCA |
| | | | TCTTCTTATGGCTATA | | | | | GGAAAAGCTCCG |
| | | | CTTATTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCTGGT | | | | | CTGACCATCAGC |
| | | | CTCATGTTTCTGGTCA | | | | | AGTCTGCAGCCG |
| | | | TTACTCTGGTATGGAC | | | | | GAAGACTTCGCA |
| | | | TACTGGGGTCAAGGA | | | | | ACTTATTACTGTC |
| | | | ACCCTGGTCACCGTCT | | | | | AGCAATCTTCTT |
| | | | CCTCG | | | | | ATTCTCTGATCA |
| | | | | | | | | CGTTCGGACAGG |
| | | | | | | | | GTACCAAGGTGG |
| | | | | | | | | AGATCAAA |
| 2890- | 2539 | 49 | GAGGTTCAGCTGGTG | 46 | GGTGGA | 2542 | 50 | GATATCCAGATG |
| knob- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTATT | | | | | ATCACCTGCCGT |
| | | | CTTCTATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATATATTTCT | | | | | CAACAGAAACCA |
| | | | TCTTATTATGGCTATA | | | | | GGAAAAGCTCCG |
| | | | CTTATTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCUCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCGCTC | | | | | CTGACCATCAGC |
| | | | ATTACTTCCCGTGGGC | | | | | AGTCTGCAGCCG |
| | | | TGGTGCTATGGACTAC | | | | | GAAGACTTCGCA |
| | | | TGGGGTCAAGGAACC | | | | | ACTTATTACTGTC |
| | | | CTGGTCACCGTCTCCT | | | | | AGCAATACTACT |
| | | | CG | | | | | GGCCGATCACGT |
| | | | | | | | | TCGGACAGGGTA |
| | | | | | | | | CCAAGGTGGAG |
| | | | | | | | | ATCAAATGA |
| 2890- | 2542 | 53 | GAGGTTCAGCTGGTG | 46 | GGTGGA | 2539 | 52 | GATATCCAGATG |
| hole- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTCTT | | | | | ATCACCTGCCGT |
| | | | ATTATATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATCTATTTAT | | | | | CAACAGAAACCA |
| | | | TCTTCTTATGGCTATA | | | | | GGAAAAGCTCCG |
| | | | CTTCTTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCACTG | | | | | CTGACCATCAGC |
| | | | TTCGTGGATCCAAAAA | | | | | AGTCTGCAGCCG |
| | | | ACCGTACTTCTCTGGT | | | | | GAAGACTTCGCA |
| | | | TGGGCTATGGACTACT | | | | | ACTTATTACTGTC |
| | | | GGGGTCAAGGAACCC | | | | | AGCAATACTCTT |
| | | | TGGTCACCGTCTCCTC | | | | | GGGGTCCGTTCA |
| | | | G | | | | | CGTTCGGACAGG |
| | | | | | | | | GTACCAAGGTGG |
| | | | | | | | | AGATCAAATGA |
| 12735- | 2539 | 49 | GAGGTTCAGCTGGTG | 46 | GGTGGA | 2542 | 50 | GATATCCAGATG |
| knob- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTATT | | | | | ATCACCTGCCGT |
| | | | CTTCTATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATATATTTCT | | | | | CAACAGAAACCA |
| | | | TCTTATTATGGCTATA | | | | | GGAAAAGCTCCG |
| | | | CTTATTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCGCTC | | | | | CTGACCATCAGC |
| | | | ATTACTCCCGTGGGC | | | | | AGTCTGCAGCCG |
| | | | TGGTGCTATGGACTAC | | | | | GAAGACTTCGCA |
| | | | TGGGGTCAAGGAACC | | | | | ACTTATTACTGTC |
| | | | CTGGTCACCGTCTCCT | | | | | AGCAATACTACT |
| | | | CG | | | | | GGCCGATCACGT |
| | | | | | | | | TCGGACAGGGTA |
| | | | | | | | | CCAAGGTGGAG |
| | | | | | | | | ATCAAATGA |
| 12735- | 2542 | 53 | GAGGTTCAGCTGGTG | 46 | GGTGGA | 2539 | 52 | GATATCCAGATG |
| hole- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTCTT | | | | | ATCACCTGCCGT |
| | | | ATTATATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATCTATTTAT | | | | | CAACAGAAACCA |
| | | | TCTTCTTATGGCTATA | | | | | GGAAAAGCTCCG |
| | | | CTTCTTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCACTG | | | | | CTGACCATCAGC |
| | | | TTCGTGGATCCAAAAA | | | | | AGTCTGCAGCCG |
| | | | ACCGTACTTCTCTGGT | | | | | GAAGACTTCGCA |
| | | | TGGGCTATGGACTACT | | | | | ACTTATTACTGTC |
| | | | GGGGTCAAGGAACCC | | | | | AGCAATACTCTT |
| | | | TGGTCACCGTCTCCTC | | | | | GGGGTCCGTTCA |
| | | | G | | | | | CGTTCGGACAGG |
| | | | | | | | | GTACCAAGGTGG |
| | | | | | | | | AGATCAAATGA |
| 5027- | 2542 | 53 | GAGGTTCAGCTGGTG | 46 | GGTGGA | 2539 | 52 | GATATCCAGATG |
| hole- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTCTT | | | | | ATCACCTGCCGT |
| | | | ATTATATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATCTATTTAT | | | | | CAACAGAAACCA |
| | | | TCTTCTTATGGCTATA | | | | | GGAAAAGCTCCG |
| | | | CTTCTTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCACTG | | | | | CTGACCATCAGC |
| | | | TTCGTGGATCCAAAAA | | | | | AGTCTGCAGCCG |
| | | | ACCGTACTTCTCTGGT | | | | | GAAGACTTCGCA |
| | | | TGGGCTATGGACTACT | | | | | ACTTATTACTGTC |
| | | | GGGGTCAAGGAACCC | | | | | AGCAATACTCTT |
| | | | TGGTCACCGTCTCCTC | | | | | GGGGTCCGTTCA |
| | | | G | | | | | CGTTCGGACAGG |
| | | | | | | | | GTACCAAGGTGG |
| | | | | | | | | AGATCAAATGA |
| 5027- | 2539 | 49 | GAGGTTCAGCTGGTG | 46 | GGTGGA | 2542 | 50 | GATATCCAGATG |
| knob- | | | GAGTCTGGCGGTGGC | | GGTGGC | | | ACCCAGTCCCCG |
| 2539- | | | CTGGTGCAGCCAGGG | | AGT | | | AGCTCCCTGTCC |
| 2542 | | | GGCTCACTCCGTTTGT | | | | | GCCTCTGTGGGC |
| | | | CCTGTGCAGCTTCTGG | | | | | GATAGGGTCACC |
| | | | CTTCAACATCTCTTATT | | | | | ATCACCTGCCGT |
| | | | CTTCTATCCACTGGGT | | | | | GCCAGTCAGTCC |
| | | | GCGTCAGGCCCCGGG | | | | | GTGTCCAGCGCT |
| | | | TAAGGGCCTGGAATG | | | | | GTAGCCTGGTAT |
| | | | GGTTGCATATATTTCT | | | | | CAACAGAAACCA |
| | | | TCTTATTATGGCTATA | | | | | GGAAAAGCTCCG |
| | | | CTTATTATGCCGATAG | | | | | AAGCTTCTGATT |
| | | | CGTCAAGGGCCGTTTC | | | | | TACTCGGCATCC |
| | | | ACTATAAGCGCAGAC | | | | | AGCCTCTACTCT |
| | | | ACATCCAAAAACACAG | | | | | GGAGTCCCTTCT |
| | | | CCTACCTACAAATGAA | | | | | CGCTTCTCTGGT |
| | | | CAGCTTAAGAGCTGA | | | | | AGCCGTTCCGGG |
| | | | GGACACTGCCGTCTAT | | | | | ACGGATTTCACT |
| | | | TATTGTGCTCGCGCTC | | | | | CTGACCATCAGC |
| | | | ATTACTTCCCGTGGGC | | | | | AGTCTGCAGCCG |
| | | | TGGTGCTATGGACTAC | | | | | GAAGACTTCGCA |
| | | | TGGGGTCAAGGAACC | | | | | ACTTATTACTGTC |
| | | | CTGGTCACCGTCTCCT | | | | | AGCAATACTACT |
| | | | CG | | | | | GGCCGATCACGT |
| | | | | | | | | TCGGACAGGGTA |
| | | | | | | | | CCAAGGTGGAG |
| | | | | | | | | ATCAAATGA |

TABLE 2

| Antibody | Alternate | | Co-Receptor |
|---|---|---|---|
| No. | name | FZD recognized | recognized |
| 2746 | F⁶ | FZD6 | |
| 2747 | F^{6.1} | FZD6 | |
| 2864 | F^{P1} | FZD1, FZD2, FZD4, FZD5, FZD7, | |
| | | FZD8 | |
| 2870 | F^{P2} | FZD1, FZD2, FZD4, FZD5, FZD7, | |
| | | FZD8 | |
| 2876 | F^{2.1} | FZD2 | |
| 2886 | F^{2/7} | FZD2, FZD7(?) | |
| 2890 | F² | FZD2 | |
| 2928 | F⁵ | FZD5 | |
| 2939 | F^{P3} | FZD1, FZD2, FZD4, FZD5, FZD7, | |
| | | FZD8 | |
| 2969 | F^{9/10} | FZD9, FZD10 | |
| 2974 | F^{9/10.1} | FZD9, FZD10 | |
| 5019 | F^{P} | FZD1, FZD2, FZD4, FZD5, FZD7, | |
| | | FZD8 | |
| 5027 | F⁴ | FZD4 | |
| 5038 | F^{4.1} | FZD4 | |
| 5044 | F^{4.4} | FZD4 | |
| 5048 | F^{4.7} | FZD4 | |
| 5056 | F^{P4} | FZD1, FZD2, FZD4, FZD6, FZD7, | |
| | | FZD8 | |
| 5062 | F^{4.2} | FZD4 | |
| 5063 | F^{4.5} | FZD4 | |
| 5067 | F^{P4} | FZD1, FZD2, FZD4, FZD5, FZD7, | |
| | | FZD8 | |
| 5075 | F^{P5} | FZD1, FZD2, FZD4, FZD5, FZD7, | |
| | | FZD8 | |
| 5076 | F^{P7} | FZD1, FZD2, FZD4, FZD5, FZD7, | |
| | | FZD8 | |
| 5080 | F^{4.3} | FZD4 | |
| 5081 | F^{4.6} | FZD4 | |
| 12735 | F⁷ | FZD7 | |
| 2459 | L5¹ | | LRP5-W1 |
| 2460 | L5³ | | LRP5-W3 |
| 2539 | L6³ | | LRP6-W3 |
| 2540 | | | LRP6-W3 |
| 2542 | L6¹ | | LRP6-W1 |

TABLE 3

| Nomenclature | Description |
|---|---|
| 5019-2539 Bi- | A bispecific immunoglobulin with a first binding domain for FZD derived from |
| IgG | antibody 5019 and a second biding domain for the Wnt3a binding site on LRP6, |
| | derived from antibody 2539, wherein both binding domains are on the same side of |
| | the Fc domain |
| 5019-2542 Bi- | A bispecific immunoglobulin with a first binding domain for FZD derived from |
| IgG | antibody 5019 and a second binding domain for the Wnt1 binding site on LRP6, |
| | derived from antibody 2542, wherein both binding domains are on the same side of |
| | the Fc domain |
| 5019-2539-K/H | A bispecific diabody comprising an Fc domain in a knob in hole configuration and |
| (FZD/LRP6-W3) | one binding domain comprising a binding site for FZD derived from antibody 5019 |
| | and a binding site for the Wnt3a binding site on LRP6 that is derived from antibody |
| | 2539 |
| 5019-2542-K/H | A bispecific diabody comprising an Fc domain in a knob in hole configuration and |
| (FZD/LRP6-W1) | one binding domain having a binding site for FZD derived from antibody 5019 and a |
| | binding site for the Wnt1 binding site on LRP6 that is derived from antibody 2542 |
| 5019-Fc-2539 | A tetravalent binding molecule comprising an Fc domain and a binding domain for |
| | FZD and a binding domain for LRP6, wherein the binding domain for FZD is in a |
| | diabody configuration that is bivalent, monospecific and derived from antibody |
| | 5019, and the binding domain for LRP6 is in a diabody configuration that is bivalent |
| | and monspecific for binding the Wnt 3a binding site and derived from antibody 2539 |
| 5019-Fc-2542 | A tetravalent binding molecule comprising an Fc domain and a binding domain for |
| | FZD and a binding domain for LRP6, wherein the binding domain for FZD is in a |
| | diabody configuration that is bivalent, monospecific and derived from antibody |
| | 5019, and the binding domain for LRP6 is in a diabody configuration that is bivalent |
| | and monspecific for binding the Wnt 1 binding site and derived from antibody 2542 |
| 5019Ag | A tetravalent binding molecule comprising an Fc domain and a binding domain for |
| 5019-K/H-2539- | FZD and a binding domain for LRP6, wherein the FC domain is in a knob in hole |
| 2542 | configuration, the binding domain for FZD is in a diabody configuration that is |
| | bivalent, monospecific and derived from antibody 5019, and the binding domain for |
| | LRP6 is in a diabody configuration that is bivalent and bispecific binding the Wnt1 |
| | and Wnt 3a binding sites on LRP6 and is derived from antibodies 2542 and 2539 |
| 5019-K/H-2539- | A tetravalent binding molecule comprising an Fc domain and a binding domain for |
| MBP | FZD and a binding domain comprising a binding site for LRP6 and a Maltose |
| | Binding Protein (MBP), wherein the FC domain is in a knob in hole configuration, |
| | the binding domain for FZD is in a diabody configuration that is bivalent, |
| | monospecific and derived from antibody 5019, and the binding domain having a |
| | binding site for LRP6 is in a diabody configuration having a binding site for LRP6 |
| | derived from antibody 2539 and a binding site for the Maltose Binding Protein. |
| 5038Ag | A tetravalent binding molecule comprising an Fc domain and a binding domain for a |
| 5038-K/H-2539- | FZD and a binding domain for LRP6, wherein the FC domain is in a knob in hole |
| 2542 | configuration, the binding domain for the FZD is in a diabody configuration that is |
| | bivalent, monospecific, and derived from antibody 5039, and the binding domain for |
| | LRP6 is in a diabody configuration that is bivalent and bispecific binding the Wnt1 |
| | and Wnt 3a binding sites on LRP6 and is derived from antibodies 2542 and 2539 |
| 5044Ag | A tetravalent binding molecule comprising an Fc domain and a binding domain for a |
| 5044-K/H-2539- | FZD and a binding domain for LRP6, wherein the FC domain is in a knob in hole |
| 2542 | configuration, the binding domain for the FZD is in a diabody configuration that is |
| | bivalent, monospecific and derived from antibody 5044, and the binding domain for |
| | LRP6 is in a diabody configuration that is bivalent and bispecific binding the Wnt1 |
| | and Wnt 3a binding sites on LRP6 and is derived from antibodies 2542 and 2539 |
| 5048Ag | A tetravalent binding molecule comprising an Fc domain and a binding domain for a |
| 5048-K/H-2539- | FZD and a binding domain for LRP6, wherein the FC domain is in a knob in hole |
| 2542 | configuration, the binding domain for the FZD is in a diabody configuration that is |
| | bivalent, monospecific and derived from antibody 50448, and the binding domain |
| | for LRP6 is in a diabody configuration that is bivalent and bispecific binding the |
| | Wnt1 and Wnt 3a binding sites on LRP6 and is derived from antibodies 2542 and |
| | 2539 |
| 5063Ag | A tetravalent binding molecule comprising an Fc domain and a binding domain for a |
| 5063-K/H-2539- | FZD and a binding domain for LRP6, wherein the FC domain is in a knob in hole |
| 2542 | configuration, the binding domain for the FZD is in a diabody configuration that is |
| | bivalent, monospecific and derived from antibody 5063, and the binding domain for |
| | LRP6 is in a diabody configuration that is bivalent and bispecific binding the Wnt1 |
| | and Wnt 3a binding sites on LRP6 and is derived from antibodies 2542 and 2539 |
| 2890Ag | A tetravalent binding molecule comprising an Fc domain and a binding domain for a |
| 2890-K/H-2539- | FZD2 and a binding domain for LRP6, wherein the FC domain is in a knob in hole |
| 2542 | configuration, the binding domain for the FZD2 is in a diabody configuration that is |
| | bivalent, monospecific and derived from antibody 2890, and the binding domain for |
| | LRP6 is in a diabody configuration that is bivalent and bispecific binding the Wnt1 |
| | and Wnt 3a binding sites on LRP6 and is derived from antibodies 2542 and 2539. |
| 12735Ag | A tetravalent binding molecule comprising an Fc domain and a binding domain for a |
| 12735-K/H-2539- | FZD7 and a binding domain for LRP6, wherein the FC domain is in a knob in hole |
| 2542 | configuration, the binding domain for the FZD is in a diabody configuration that is |
| | bivalent, monospecific and derived from antibody 12735, and the binding domain |
| | for LRP6 is in a diabody configuration that is bivalent and bispecific binding the |
| | Wnt1 and Wnt 3a binding sites on LRP6 and is derived from antibodies 2542 and |
| | 2539 |
| 5080Ag | A tetravalent binding molecule comprising an Fc domain and a binding domain for a |
| 5080-K/H-2539- | FZD and a binding domain for LRP6, wherein the FC domain is in a knob in hole |
| 2542 | configuration, the binding domain for the FZD is in a diabody configuration that is |
| | bivalent, monospecific and derived from antibody 5080, and the binding domain for |
| | LRP6 is in a diabody configuration that is bivalent and bispecific binding the Wnt1 |
| | and Wnt 3a binding sites on LRP6 and is derived from antibodies 2542 and 2539 |
| 5081Ag | A tetravalent binding molecule comprising an Fc domain and a binding domain for |
| 5081-K/H-2539- | FZD and a binding domain for LRP6, wherein the FC domain is in a knob in hole |
| 2542 | configuration, the binding domain for FZD is in a diabody configuration that is |
| | bivalent, monospecific and derived from antibody 5081, and the binding domain for |
| | LRP6 is in a diabody configuration that is bivalent and bispecific binding the Wnt1 |
| | and Wnt 3a binding sites on LRP6 and is derived from antibodies 2542 and 2539 |
| 2876Ag | A tetravalent binding molecule comprising an Fc domain and a binding domain for a |
| | FZD and a binding domain for LRP6, wherein the Fc domain is in a knob in hole |
| | configuration, the binding domain for the FZD is in a diabody configuration that is |
| | bivalent, monospecific and derived from antibody 2876, and the binding domain for |
| | LRP6 is in a diabody configuration that is bivalent and bispecific binding the Wnt1 |
| | and Wnt 3a binding sites on LRP6 and is derived from antibodies 2542 and 2539 |
| 2890Ag | A tetravalent binding molecule comprising an Fc domain and a binding domain for a |
| | FZD and a binding domain for LRP6, wherein the FC domain is in a knob in hole |
| | configuration, the binding domain for the FZD is in a diabody configuration that is |
| | bivalent, monospecific, and derived from antibody 2890, and the binding domain for |
| | LRP6 is in a diabody configuration that is bivalent and bispecific binding the Wnt1 |
| | and Wnt 3a binding sites on LRP6 and is derived from antibodies 2542 and 2539 |
| 2886Ag | A tetravalent binding molecule comprising an Fc domain and a binding domain for a |
| | FZD and a binding domain for LRP6, wherein the FC domain is in a knob in hole |
| | configuration, the binding domain for the FZD is in a diabody configuration that is |
| | bivalent and derived from antibody 2886, and the binding domain for LRP6 is in a |
| | diabody configuration that is bivalent and bispecific binding the Wnt1 and Wnt 3a |
| | binding sites on LRP6 and is derived from antibodies 2542 and 2539 |
| 2747Ag | A tetravalent binding molecule comprising an Fc domain and a binding domain for a |
| | FZD and a binding domain for LRP6, wherein the FC domain is in a knob in hole |
| | configuration, the binding domain for the FZD is in a diabody configuration that is |
| | bivalent, derived from antibody 2747, and the binding domain for LRP6 is in a |
| | diabody configuration that is bivalent and bispecific binding the Wnt1 and Wnt 3a |
| | binding sites on LRP6 and is derived from antibodies 2542 and 2539 |
| 2969Ag | A tetravalent binding molecule comprising an Fc domain and a binding domain for a |
| | FZD and a binding domain for LRP6, wherein the FC domain is in a knob in hole |
| | configuration, the binding domain for the FZD is in a diabody configuration that is |
| | bivalent, derived from antibody 2969, and the binding domain for LRP6 is in a |
| | diabody configuration that is bivalent and bispecific binding the Wnt1 and Wnt 3a |
| | binding sites on LRP6 and is derived from antibodies 2542 and 2539 |
| 2974Ag | A tetravalent binding molecule comprising an Fc domain and a binding domain for a |
| | FZD and a binding domain for LRP6, wherein the FC domain is in a knob in hole |
| | configuration, the binding domain for the FZD is in a diabody configuration that is |
| | bivalent, derived from antibody 2074, and the binding domain for LRP6 is in a |
| | diabody configuration that is bivalent and bispecific binding the Wnt1 and Wnt 3a |
| | binding sites on LRP6 and is derived from antibodies 2542 and 2539 |
| Homodiabody | A diabody comprising an Fc domain and two binding sites for the Wnt3a binding |
| 2539-Fc | site on LRP6 that is derived from antibody 2539 |
| Homodiabody | A diabody comprising an Fc domain and two binding sites for the Wnt1 binding site |
| 2542-Fc | on LRP6 that is derived from antibody 2542 |
| FP + P − L61 + 3 | A tetravalent binding molecule comprising an Fc domain and a binding domain for |
| | FZD and a binding domain for LRP6, wherein the FC domain is in a knob in hole |
| | configuration, wherein the binding domain for FZD is in a diabody configuration |
| | that is bivalent, monospecific and derived from antibody 5019, and the binding |
| | domain for LRP6 is in a diabody configuration that is bivalent and bispecific binding |
| | the Wnt1 and Wnt 3a binding sites on LRP6 and is derived from antibodies 2542 |
| | and 2539 |
| FP* + P* − L61 + 3 | A tetravalent binding molecule comprising an Fc domain and a binding domain for |
| | FZD and a binding domain for LRP6, wherein the FC domain is in a knob in hole |
| | configuration, wherein the binding domain for FZD is in a scFV configuration such |
| | that the binding domain is bivalent, monospecific, and the scFv is derived from |
| | antibody 5019, and the binding domain for LRP6 is in a diabody configuration |
| | bivalent and bispecific binding the Wnt1 and Wnt 3a binding sites on LRP6 and is |
| | derived from antibodies 2542 and 2539 |
| FP + P − L61* + 3* | A tetravalent binding molecule comprising an Fc domain and a binding domain for |
| | FZD and a binding domain for LRP6, wherein the FC domain is in a knob in hole |
| | configuration, wherein the binding domain for FZD is a diabody configuration that |
| | is bivalent and monospecific and derived from antibody 5019, and the binding |
| | domain for LRP6 is in a scFv configuration that is bispecific for binding the Wnt1 |
| | and Wnt 3a binding sites on LRP6 and the scFv is derived from antibodies 2542 and |
| | 2539 |
| FP* + P* − L61* + 3* | A tetravalent binding molecule comprising an Fc domain and a binding domain for |
| | FZD and a binding domain for LRP6, wherein the FC domain is in a knob in hole |
| | configuration, wherein the binding domain for FZD is in a scFv configuration that is |
| | bivalent, monospecific, and derived from antibody 5019, and the binding domain for |
| | LRP6 is in a scFv configuration that is bivalent and bispecific for binding to the |
| | Wnt1 and Wnt 3a binding sites on LRP6 and is derived from antibodies 2542 and |
| | 2539 |

1. A method for activating a Wnt signaling pathway in a cell, said method comprising contacting a cell having a Frizzled2 (FZD2) receptor or Frizzled7 (FZD7) and a Wnt co-receptor with a multivalent binding molecule, wherein the multivalent binding molecule comprises
(a) an Fc domain, or fragment thereof comprising a CH3 domain, having a C-terminus and an N-terminus,
(b) (i) a FZD2 binding domain having at least two binding sites wherein at least one binding site binds to the FZD2 receptor and comprises a light-chain variable domain (VL) that is 50%, 55%, 60%, 75%, 80%, 85%, 90%, 95%, 96%, 97%, 98% or 99% identical to a VL of 2890-hole-2539-2542 (having the amino acid sequence encoded by SEQ ID NO: 85) or CDRs of the VL of 2890-hole-2539-2542, and comprises a heavy-chain variable domain (VH) comprising VH of 2890-hole-2542 (having the amino acid sequence encoded by SEQ ID NO: 84), or CDRs of the VHs of 2890-hole-2539-2542 or 12735-hole-2539-2542, or (ii) a FZD7 binding domain having at least two binding sites wherein at least one binding site binds to the FZD7 receptor and comprises a light-chain variable domain (VL) that is 50%, 55%, 60%, 75%, 80%, 85%, 90%, 95%, 96%, 97%, 98% or 99% identical to a VL of 12735-hole-2539-2542 (having the amino acid sequence encoded by SEQ ID NO: 87) or CDRs of the VHs of 12735-hole-2539-2542 and comprises a heavy-chain variable domain (VH) comprising VH of 12735-hole-2539-2542 (having the amino acid sequence encoded by SEQ ID NO: 86), or CDRs of the VHs of 12735-hole-2539-2542, and
(c) a Wnt co-receptor domain having at least two binding sites wherein at least one binding site binds to the Wnt co-receptor,
wherein the FZD2 or FZD7 binding domain is attached to one terminus of the Fc domain or one terminus of the fragment thereof, and the Wnt co-receptor binding domain is attached to the other terminus of the Fc domain or the other terminus of the fragment thereof.

2. The method of claim 1, wherein the FZD2 or FZD7 binding domain comprises,
(a)(i) a diabody that binds the FZD2 receptor, said diabody comprising two peptides each peptide comprising a heavy-chain variable domain (VH) linked to a light-chain variable domain (VL) wherein the VH and the VL from one peptide pair to the VL and VH of the other peptide thereby forming the diabody, and wherein the VL comprises the VL of 2890-hole-2539-2542 (having the amino acid sequence encoded by SEQ ID NO: 85) or CDRs of the VL of 2890-hole-2539-2542, and the VH comprises the VH of 2890-hole-2542 (having the amino acid sequence encoded by SEQ ID NO: 84), or CDRs of the VH of 2890-hole-2539-2542, or (ii) a diabody that binds the FZD7 receptor, said diabody comprising two peptides each peptide comprising a heavy-chain variable domain (VH) linked to a light-chain variable domain (VL) wherein the VH and the VL from one peptide pair to the VL and VH of the other peptide thereby forming the diabody, and wherein the VL comprises the VL of 12735-hole-2539-2542 (having the amino acid sequence encoded by SEQ ID NO: 87) or CDRs of the VL of 12735-hole-2539-2542, and the VH comprises the VH of 12735-hole-2539-2542 (having the amino acid sequence encoded by SEQ ID NO: 86), or CDRs of the VH of 12735-hole-2539-2542, or
(b) (i) an scFv comprising a VL comprising a VL of 2890-hole-2539-2542 or (having the amino acid sequence encoded by SEQ ID NO: 85) or CDRs of the VL of 2890-hole-2539-2542 and a VH region comprising the VH of 2890-hole-2542 (having the amino acid sequence encoded by SEQ ID NO: 84), or CDRs of the VHs of 2890-hole-2539-2542 o, and binds the FZD2 receptor, or (ii) an scFv comprising a VL comprising a VL of 12735-hole-2539-2542 (having an amino acid sequence encoded by SEQ ID NO: 87) or CDRs of the VL of 2890-hole-2539-2542 or 12735-hole-2539-2542 and a VH region comprising the VH of 12735-hole-2539-2542 (having an amino acid sequence encoded by SEQ ID NO: 86), or CDRs of the VHs of 12735-hole-2539-2542, and binds the FZD7 receptor and
the Wnt co-receptor binding domain comprises
(c) a diabody that binds the Wnt co receptor, said diabody comprising two peptides each peptide comprising a heavy-chain variable domain (VH) linked to a light-chain variable domain (VL) wherein the VH and the VL from one peptide pair to the VL and VH of the other peptide thereby forming the diabody, or,
(de) an scFv comprising VL and VH regions that binds the co-receptor, or
(e) an endogenous ligand of the co-receptor or a fragment of such ligand that binds the co-receptor.

3. The method of claim 1, wherein the Wnt co-receptor binding domain binds to a Wnt ligand binding site on the Wnt co-receptor.

4. The method of claim 3, wherein the Wnt co-receptor binding domain binds to Wnt3 and/or Wnt1 binding sites.

5. The method of claim 1, wherein the Fc domain is an IgG Fc domain.

6. A multivalent binding molecule, wherein the multivalent binding molecule comprises
(a) an Fc domain, or fragment thereof comprising a CH3 domain, having a C-terminus and an N-terminus,
(b) (i) a FZD2 binding domain having at least two binding sites wherein at least one binding site comprises a light-chain variable domain (VL) comprising VL of 2890-hole-2539-2542 (having the amino acid sequence encoded by SEQ ID NO: 85) or CDRs of the VL of 2890-hole-2539-2542, and comprises a heavy-chain variable domain (VH) comprising VH of 2890-hole-2542 (having the amino acid sequence encoded by SEQ ID NO: 84), or CDRs of the VHs of 2890-hole-2539-2542, and binds to the FZD2 receptor, or (ii) a FZD7 binding domain having at least two binding sites wherein at least one binding site comprises a light-chain variable domain (VL) comprising VL 12735-hole-2539-2542 (having the amino acid sequence encoded by SEQ ID NO:87) or CDRs of the VL of 12735-hole-2539-2542, and comprises a heavy-chain variable domain (VH) comprising VH of 12735-hole-2539-2542 (having the amino acid sequence encoded by SEQ ID NO: 86), or CDRs of the VHs of 2890-hole-2539-2542 or 12735-hole-2539-2542, and binds to the FZD7 receptor and
(c) a Wnt co-receptor binding domain having at least two binding sites wherein at least one binding site binds to the Wnt co-receptor,
wherein the FZD2 or FZD7 binding domain is attached to one terminus of the Fc domain and the Wnt co-receptor binding domain is attached to the other terminus of the Fc domain.

7. The multivalent binding molecule of claim 6, wherein the FZD2 or FZD7 binding domain comprises,
(a) (i) a diabody that binds the FZD2 receptor, said diabody comprising two peptides each peptide comprising a heavy-chain variable domain (VH) linked to a light-chain variable domain (VL) wherein the VH and the VL from one peptide pair to the VL and VH of the other peptide thereby forming the diabody, and wherein the VL comprises the VL of 2890-hole-2539-2542 (having an amino acid sequence encoded by SEQ ID NO: 85) or CDRs of the VL of 2890-hole-2539-2542, and the VH comprises the VH of 2890-hole-2542 (encoded by SEQ ID NO: 84), or CDRs of the VH of 2890-hole-2539-2542, or (ii) a diabody that binds the FZD7 receptor, said diabody comprising two peptides each peptide comprising a heavy-chain variable domain (VH) linked to a light-chain variable domain (VL) wherein the VH and the VL from one peptide pair to the VL and VH of the other peptide thereby forming the diabody, and wherein the VL comprises the VL of 12735-hole-2539-2542 (having the amino acid sequence encoded by SEQ ID NO: 87) or CDRs of the VL of 12735-hole-2539-2542, and the VH comprises the VH of 12735-hole-2539-2542 (having the amino acid sequence encoded by SEQ ID NO: 86), or CDRs of the VH of 12735-hole-2539-2542, or
(b) (i) an scFv comprising VL and VH regions that bind the FZD2 receptor, wherein the VL comprises the VL of 2890-hole-2539-2542 (having the amino acid sequence encoded by SEQ ID NO: 85) or CDRs of the VL of 2890-hole-2539-2542 or 12735-hole-2539-2542, and the VH comprises the VH of 2890-hole-2542 (having the amino acid sequence encoded by SEQ ID NO: 86), or CDRs of the VH of 2890-hole-2539-2542, or (ii) an scFv comprising VL and VH regions that bind the FZD7 receptor, wherein the VL comprises the VL of 12735-hole-2539-2542 (having the amino acid sequence encoded by SEQ ID NO: 87) or CDRs of the VL of 12735-hole-2539-2542, and the VH comprises the VH of 12735-hole-2539-2542 (having the amino acid sequence encoded by SEQ ID NO: 86), or CDRs of the VH of 12735-hole-2539-2542, and
the Wnt co-receptor binding domain comprises
(d) a diabody that binds the coreceptor, said diabody comprising two peptides each peptide comprising a heavy-chain variable domain (VH) linked to a light-chain variable domain (VL) wherein the VH and the VL from one peptide pair to the VL and VH of the other peptide thereby forming the diabody, wherein the VL comprises the VL of 2890-hole-2539-2542, 2890-knob-2539-2542, 12735-hole-2539-2542 or 12735-knob-2539-2542 (having the amino acid sequence encoded by SEQ ID NO: 50 or 52) or CDRs of the VL of 2890-hole-2539-2542 or 12735-hole-2539-2542 or 12735-knob-2539-2542, and the VH comprises the VH of 2890-hole-2542, 2890-knob-2542, 12735-hole-2539-2542 or 12735-knob-2539-2542 (having the amino acid sequence encoded by SEQ ID NO: 49 or 53), or CDRs of the VH of 2890-hole-2539-2542 or 12735-hole-2539-2542, or,
(e) an scFv comprising VL and VH regions that bind the co-receptor, wherein the VL comprises the VL of 2890-hole-2539-2542, 2890-knob-2539-2542 or 12735-hole-2539-2542, or 12735-knob-2539-2542 (having the amino acid sequence encoded by SEQ ID NO: 50 or 52) or CDRs of the VL of 2890-hole-2539-2542 or 12735-hole-2539-2542, and the VH comprises the VH of 2890-hole-2542, 2890-hole-2539-2542 or 12735-hole-2539-2542 or 12735-knob-2539-2542 (having the amino acid sequence encoded by SEQ ID NO: 49 or 53), or CDRs of the VH of 2890-hole-2539-2542 or 12735-hole-2539-2542.

8. The multivalent binding molecule of claim 6, wherein at least one of the binding domains is bispecific.

9. The multivalent binding molecule of claim 6, comprising a first peptide comprising SEQ ID NO: 77 and a second peptide comprises SEQ ID NO: 79 and binds FZD2.

10. The multivalent binding molecule of 6, comprising a first peptide comprising SEQ ID NO: 81 and a second peptide comprising SEQ ID NO: 83 and binds FZD7.

11. The multivalent binding molecule of claim 6, comprising a first peptide consisting essentially of SEQ ID NO: 77 and a second peptide consisting essentially of SEQ ID NO: 79 and binds FZD2.

12. The multivalent binding molecule of 6, comprising a first peptide consisting essentially of SEQ ID NO: 81 and a second peptide consisting essentially of SEQ ID NO: 83.

13. A pharmaceutical composition comprising a multivalent binding molecule of claim 6 and a pharmaceutically acceptable carrier.

14. A method for enhancing tissue regeneration in a subject in need thereof, or treating a subject having a condition associated with reduced Wnt signaling comprising administering a multivalent binding molecule of claim 6 to the subject in an amount sufficient to enhance tissue regeneration or alleviate symptoms associated with the condition.

15. The method of claim 14, wherein the tissue is bone tissue or intestinal tissue.

16. A method for facilitating the interaction of a FZD2 or FZD7 receptor and a Wnt co-receptor on a cell thereby activating a Wnt signaling pathway in the cell comprising,
a) selecting an Fc domain, or fragment thereof comprising a CH3 domain, having a C-terminus and an N-terminus
b) linking a bivalent FZD2 or FZD7 receptor binding domain comprising the VL that binds the FZD2 receptor of 2890-hole-2539-2542 or the FZD7 receptor of 12735-hole-2539-2542 (having the amino acid sequence encoded by SEQ ID NO 85 or 87 respectively) or CDRs of the VL of 2890-hole-2539-2542 or 12735-hole-2539-2542, and the VH comprises the VH of 2890-hole-2542 or 12735-hole-2539-2542 (having the amino acid sequence encoded by SEQ ID NO: 84 or 86 respectively), or CDRs of the VH of 2890-hole-2539-2542 or 12735-hole-2539-2542, on one terminus of the Fc domain and linking a bivalent Wnt co-receptor binding domain on the other terminus of the Fc domain thereby forming a tetravalent binding molecule;
c) contacting said tetravalent binding molecule with the cell expressing said FZD2 or FZD7 receptor and Wnt co-receptor under conditions wherein the tetravalent binding molecule binds to the FZD2 or FZD7 receptor and the Wnt co-receptor thereby activating the Wnt signaling pathway.

17. The method of claim 16, wherein the bivalent FZD2 or FZD7 receptor binding domain comprises a diabody comprising the VL of 2890-hole-2539-2542 or of 12735-hole-2539-2542 (having the amino acid sequence encoded by SEQ ID NO 85 or 87) or CDRs of the VL of 2890-hole-2539-2542 or of 12735-hole-2539-2542, and the VH comprises the VH of 2890-hole-2542 or of 12735-hole-2539-2542 (having the amino acid sequence encoded by SEQ ID NO 84 or 86), or CDRs of the VH of 2890-hole-2539-2542 or of 12735-hole-2539-2542 and the bivalent Wnt receptor binding domain comprises a diabody that binds a Wnt co-receptor.

18. The method of claim 17, wherein the diabody that binds a Wnt co-receptor binds to one or both of Wnt1 or Wnt3a binding sites on the Wnt co-receptor.
